(19) United States
(12) Patent Application Publication
(10) Pub. No.: US 2024/0360105 A1
(43) Pub. Date: Oct. 31, 2024

(54) PROCESS FOR POLYAMIDE SYNTHESIS

(71) Applicant: Design Therapeutics, Inc., Carlsbad, CA (US)

(72) Inventor: Chengzhi ZHANG, Carlsbad, CA (US)

(21) Appl. No.: 18/294,494

(22) PCT Filed: Aug. 3, 2022
(86) PCT No.: PCT/US2022/039278

§ 371 Date: Feb. 1, 2024

Related U.S. Application Data
(60) Provisional application No. 63/229,235, filed on Aug. 4, 2021.

(51) Int. Cl. C07D 403/14; C08G 69/28

(52) U.S. Cl. CPC C07D 403/14; C08G 69/28

(57) ABSTRACT

The disclosure relates to synthetic methods for producing polyamides, for example pyrrole and imidazole containing polyamides of Formula (I) or (II), or a pharmaceutically acceptable salts thereof. These polyamides bind genes having expanded oligonucleotide repeat sequences, which thereby modulates transcription. The disclosure further provides methods of modulation of the transcription of such DNA, and the use of polyamides as therapeutic agents to treat diseases associated with such DNA.

### CROSS REFERENCE TO RELATED APPLICATIONS

This application claims the benefit of U.S. Provisional Application No. 63/229,235, filed Aug. 4, 2021, which is hereby incorporated by reference in its entirety.

### FIELD OF THE DISCLOSURE

The disclosure relates to the field of polyamides containing pyrroles and imidazoles which bind DNA having a oligonucleotide repeat sequences.

### SUMMARY OF THE DISCLOSURE

The disclosure relates to synthetic methods for producing polyamides, for example pyrrole and imidazole containing polyamides. These polyamides bind genes having expanded oligonucleotide repeat sequences, which thereby modulates transcription. The disclosure further provides methods of modulation of the transcription of such DNA, and the use of polyamides as therapeutic agents to treat diseases associated with such DNA.

Oligonucleotide (e.g., dinucleotide, trinucleotide, tetranucleotide, pentanucleotide, and hexanucleotide) repeat disorders (e.g., trinucleotide repeat disorders) are due to genomic stretches of DNA (i.e., deoxyribonucleic acid) that contain an oligonucleotide repeat sequence (i.e., “repeat”) which is contiguously repeated (e.g., as many as 1700 times, or even more). As appreciated by one of skill in the art, recitation of an oligonucleotide sequence herein also contemplates the (Watson-Crick) complementary sequence, which of necessity is present in the opposite sense strand of duplex DNA within a genome. The term “trinucleotide repeat” refers to a trinucleotide (e.g., GAA, and the like) that is multiply repeated in a contiguous region in a gene. Expansion and hyper-expansion can occur within both introns and exons of a gene as well as regions of the genome not associated with a gene.

Many diseases are characterized by expanded oligonucleotide repeat sequences at different locations and degrees of expansion within the genome. It is well known in the art that such repeats can occur throughout all genomic sequences. However, if a repeat is present in a gene, expansion of the repeat can result in a defective gene product and an associated disease. Additionally, the presence of a repeat expansion in a gene can reduce transcription of the gene, leading to disease due to lack of the associated protein gene product (i.e., loss-of-function). As an example of a loss-of-function disease associated with hyper-expansion, 98% of humans suffering from Friedreich's ataxia have a hyper-expansion of a GAA triplet (i.e., trinucleotide) in the first intron of the frataxin gene. Without wishing to be bound by theory, it is generally understood that hyper-expansion of the GAA triplet in the human FXN gene results in decreased transcription and resulting lower levels of frataxin, which decrease results in disease. Diseases associated with expanded oligonucleotide repeat sequences are for example, Friedrich's ataxia (FA or FRDA), Myotonic dystrophy (DM) types 1 and 2, Fuch's endothelial dystrophy, Amyotrophic lateral sclerosis (ALS), Spinocerebellar ataxia type 31 (SCA31), and Fragile X (FXS).

Friedreich's ataxia is an autosomal recessive neurodegenerative disorder caused by mutations in the fxn gene, which encodes the protein frataxin (FXN), an iron-binding mitochondrial protein involved in electron transport and metabolism. In most subjects with FA, a GAA trinucleotide repeat (from about 66 to over 1000 trinucleotides) is included in the first intron of fxn, and this hyper expansion is responsible for the observed pathology. Hyper expansion of the GAA repeats results in reduced expression of FXN.

Myotonic dystrophy type 2 (DM2) originates with a defect in the cnbp gene, also known as znf9. The gene codes for a protein known as CCHC-type zinc finger nucleic acid binding protein. This protein comprises zinc finger domains that are believed to bind to nucleic acids. Normally, the cnbp gene contains nucleotide quartet CCTG, repeated fewer than 26 times. Subjects with DM2 have a mutation of this gene in which the CCTG quartet is repeated 75 to over 10,000 times. The excessive repeats lead to overproduction of the cnbp mRNA, which aggregates within the cell and disrupts production of other proteins. This disruption mechanism accounts for the muscular weakness and other symptoms of DM2.

Spinocerebellar ataxia type 31 (SCA31) is an adult-onset neurodegenerative disease showing progressive cerebellar ataxia mainly affecting Purkinje cells. SCA31 is a subtype of the spinocerebellar ataxia family of diseases, which is associated with variable extracerebellar neurological features, including pyramidal tract signs, extrapyramidal signs, ophthalmoparesis, and sensory disturbances. In particular, SCA31 is characterized by nystagmus (involuntary movement of eyes), dysarthria (slurred or slowed speech), reduced pallesthesia (ability to sense vibration), and auditory difficulties. SCA31 has been linked to the presence of insertion repeats on chromosome 16q22.1, more specifically at the “brain expressed, associated with Nedd4” (“bean”) and thymidine kinase 2 (“tk2”) genes, which are on opposite strands and are transcribed in opposite directions. Insertions of between 2.5 and 3.8 kb have been observed. In one patient, the TGGAA sequence was repeated, with over 100 copies identified. The length of the insertion inversely correlates with age of onset. RNA foci containing UGGAA repeats have been observed in cell nuclei of SCA31 subjects; therefore, the presence of TGGAA repeats is implicated as the causative factor for SCA31 pathogenesis, very possibly through a gain-of-toxic-function mechanism.

Fragile X syndrome and fragile XE syndrome are X-linked genetic diseases that are characterized by developmental impairment. Fragile X syndrome is caused by a mutation in the fmr1 gene. The FMRP protein that is coded by the fmr1 gene plays a role in neuronal development, particularly in the formation of synapses. FMRP is thought to assist transport of mRNA from the nucleus, and thus facilitate translation. The fmr1 gene comprises a number of CGG repeats. Normally, the fmr1 promoter contains up to about 50 copies of the CGG repeat; subjects with the disease can have several hundred copies of this repeat. This repeat is associated with the presence of a so-called “CpG island”, which undergoes cytosine methylation, resulting in diminished gene transcription, and subsequent reduction in FMRP production.

Fragile XE syndrome is caused by a mutation in the fmr2 gene, also known as the aff2 gene. The gene codes for the AFF2 protein, which is thought to behave as a transcriptional activator. The gene is expressed primarily in the placenta, and in the adult and fetal brain. The fmr2 gene comprises a number of CGG repeats. Normally, the fmr2 promoter contains up to about 40 copies of the CGG repeat; subjects with the disease can have more than 200 copies of this repeat. As a result of this expanded repeat sequence, expression of the AFF2 protein is silenced.

Fragile X-associated tremor/ataxia syndrome (FXTAS) is caused by excess fmr1 mRNA in the cells of afflicted subjects, particularly brain and nerve cells. The excess mRNA is caused by a high count of CGG repeats in the 5′ UTR region of the fmr1 gene. Normally, the UTR contains up to about 50 copies of the CGG repeat; subjects with the disease can have up to 200 copies of this repeat. The high repeat count leads to improper regulation of transcription of the gene, causing the excess mRNA production. This excess mRNA is believed responsible for many of the clinical symptoms of FTAXS, due perhaps to aggregation of the mRNA that is observed in subjects. Paradoxically, despite the increased quantity of fmr1 mRNA in afflicted individuals, production of the translation product, fragile X mental retardation protein (FMRP) is unchanged or decreased, with some behavioral symptoms of FXTAS thought to be due to these decreased FMRP levels.

Amyotrophic lateral sclerosis (ALS) is a degenerative disease characterized by muscle atrophy, loss of muscle mass, and decreased ability to control motion (ataxia). Although the majority of ALS cases are sporadic, with no identifiable risk factor or genetic basis, a fraction of ALS cases are inherited. The presence of a GGGGCC repeat sequence in the non-coding region of the c9orf72 gene is associated with 25% to 40% of inherited ALS cases, as well as a smaller fraction of sporadic cases. This genetic defect is also observed in a disease termed frontal-temporal lobe dementia (FTD, also referred to as frontotemporal lobar degeneration with TDP-43 pathology, or FTLD-TDP), which is characterized by atrophy of the frontal-temporal lobe. Certain individuals present with symptoms of both diseases, a condition which is termed ALS-FTD. Healthy individuals typically display 2-23 of these hexanucleotide repeats; in FTD/ALS patients, this number is estimated at 700-1600 repeat units. RNA foci comprising the hexanucleotide repeat have been identified in individuals afflicted with FTD, suggesting that the presence of the defective RNA may be responsible for the observed phenotype.

Fuchs' endothelial dystrophy of Fuchs' endothelial corneal dystrophy (FECD) is a non-inflammatory, sporadic or autosomal dominant, dystrophy involving the endothelial layer of the cornea. With Fuchs' dystrophy the cornea begins to swell causing glare, halos, and reduced visual acuity. The damage to the cornea in Fuchs' endothelial dystrophy can be so severe as to cause corneal blindness. Fuchs' dystrophy has been classified into early-onset (first decade) and late-onset (fourth to the fifth decade) with a predominance of females in the latter. Early-onset Fuchs' has Collagen type 8 α2 chain involvement. Late-onset is characterized by Transcription factor 4, Transcription factor 8 (TCF8), ATP/GTP binding protein-like 1 (AGBL1), lipoxygenase homology domain 1 (LOXHD1), solute carrier family 4 member 11 (SLC4A11) gene and Transforming growth factor-β-induced and clusterin involvement.

There remains an unmet need to provide an effective treatment for a disease or disorder which is characterized by the presence of an excessive count of a nucleotide repeat (e.g. the trinucleotide repeat sequence CGG) in a target gene. Therefore, facile synthetic routes to polyamides compounds are needed.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE DISCLOSURE

In an aspect, provided herein is a process for the synthesis of polyamides comprising pyrroles and imidazoles. In some embodiments, the process comprising the steps of synthesizing a compound of Formula (I) or (II), or a pharmaceutically acceptable salt thereof. In some embodiments, the compound of Formula (I) or (II) is a polyamide comprising pyrroles and/or imidazoles.

### Process for Synthesis

In an aspect, provided herein is a process for synthesizing a polyamide of Formula (I), or a pharmaceutically acceptable salt thereof, comprising reacting intermediate 24 with intermediate 34 to obtain a polyamide of Formula (I), the process comprising the steps:

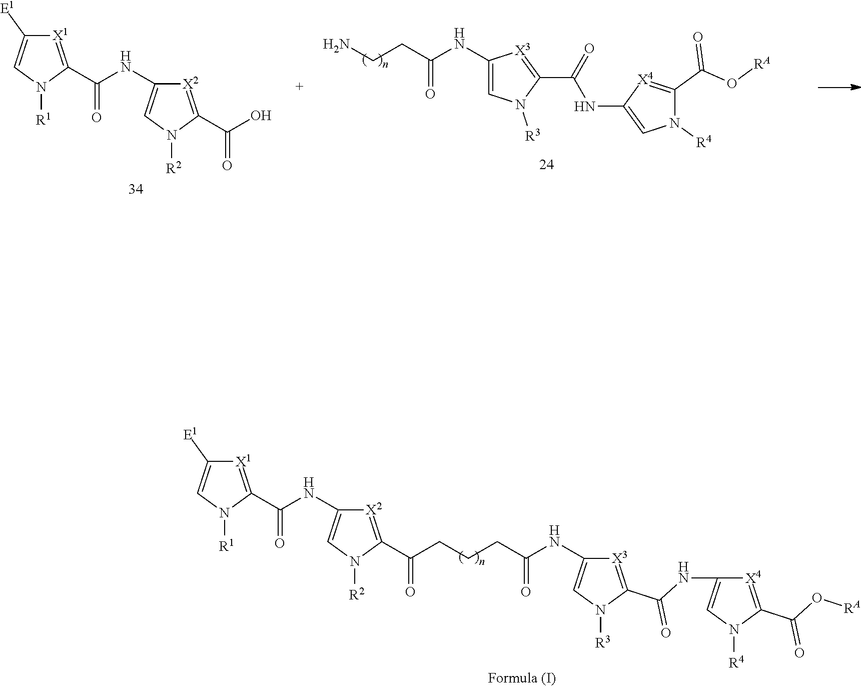

wherein,
n is 1, 2, or 3;
X¹, X², X³, and X⁴ are each independently CH or N;
E is L-R^{B}, wherein
   L is a bond, —O—, —NH—, —C(O)—, —NHC(O)—, or —NHC(O)NH—;
   R^{B} is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, or one or more AA; wherein each AA is independently a naturally occurring amino acid;
R^{A} is H or C₁-C₆ alkyl; and
R¹, R², R³, and R⁴ are each independently H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₁-C₂₀ hydroxyalkyl, optionally substituted C₁-C₂₀ haloalkyl, optionally substituted C₃-C₈ cycloalkyl, or optionally substituted 3 to 8-membered heterocycloalkyl.

In some embodiments, synthesizing intermediate 26 proceeds by the following the process comprising steps:

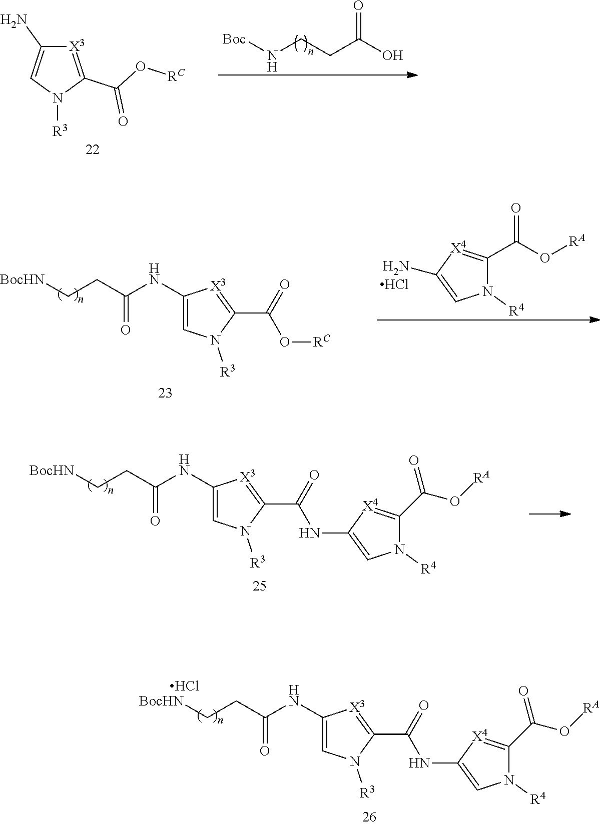

wherein, R^{C} is H or C₁-C₆ alkyl.

In some embodiments, synthesizing intermediate 34 proceeds by the following the process comprising steps:

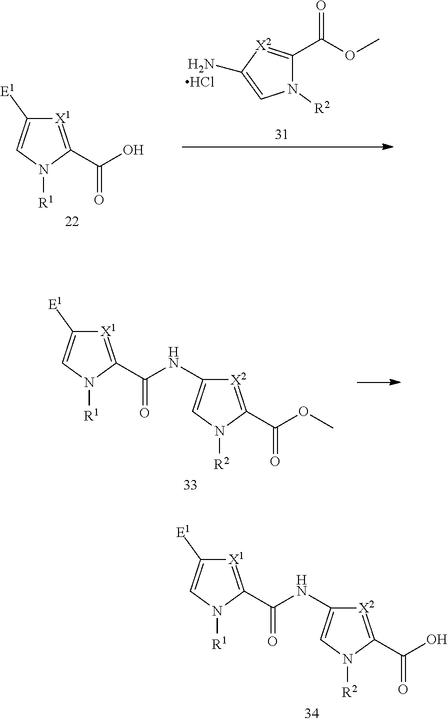

In some embodiments, the process optionally further comprising the synthesis of a polyamide of Formula (II), or a pharmaceutically acceptable salt thereof, the process comprising the steps:

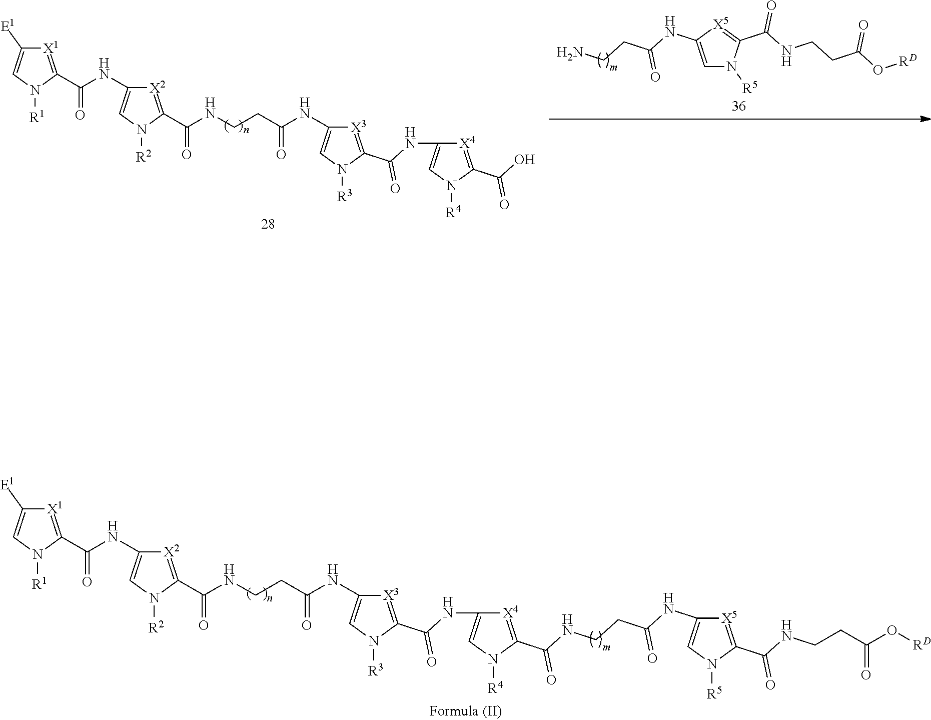

wherein,
X⁵ is CH or N;
R⁵ is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₁-C₂₀ hydroxyalkyl, optionally substituted C₁-C₂₀ haloalkyl, optionally substituted C₃-C₈ cycloalkyl, or optionally substituted 3 to 8-membered heterocycloalkyl;
R^{D} is H or C₁-C₆ alkyl; and
m is 1, 2, or 3.

In another aspect, provided herein is a process for synthesizing a polyamide of Formula (II), or a pharmaceutically acceptable salt thereof, comprising reacting intermediate 28 with intermediate 36 to obtain a polyamide of Formula (II), the process comprising:

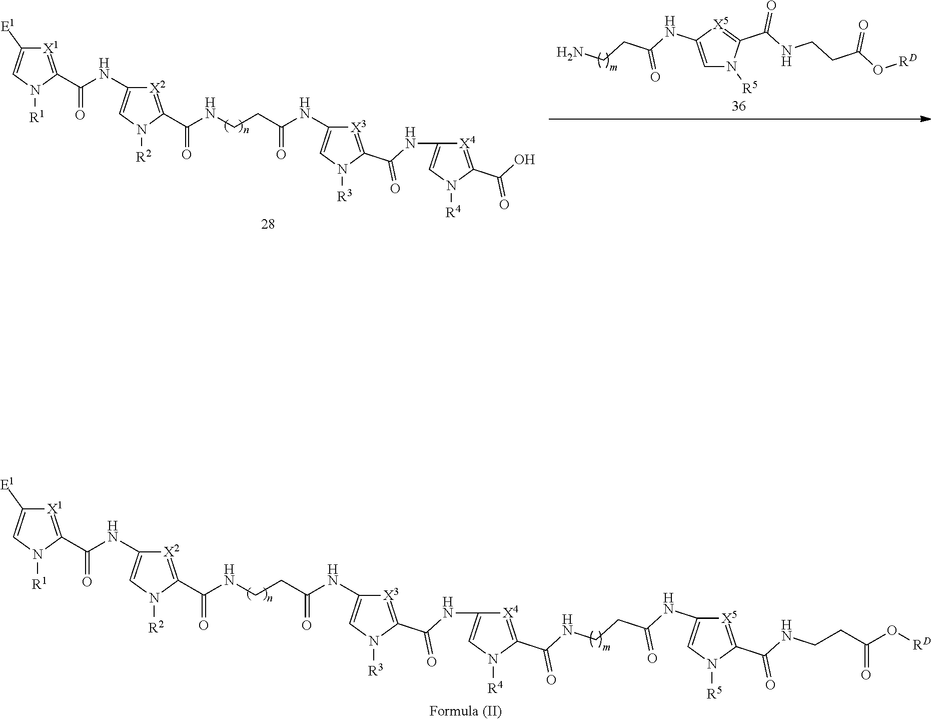

wherein,
n is 1, 2, or 3;
m is 1, 2, or 3;
X¹, X², X³, X⁴, and X⁵ are each independently CH or N;
E¹ is L-R^{B}, wherein
   L is a bond, —O—, —NH—, —C(O)—, —NHC(O)—, or —NHC(O)NH—;
   R^{B} is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, or one or more AA; wherein each AA is independently a naturally occurring amino acid;
R^{D} is H or C₁-C₆ alkyl; and
R¹, R², R³, R⁴, and R⁵ are each independently H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₁-C₂₀ hydroxyalkyl, optionally substituted C₁-C₂₀ haloalkyl, optionally substituted C₃-C₈ cycloalkyl, or optionally substituted 3 to 8-membered heterocycloalkyl.

In some embodiments, X¹ is CH. In some embodiments, X¹ is N.

In some embodiments, X² is CH. In some embodiments, X² is N.

In some embodiments, X³ is CH. In some embodiments, X³ is N.

In some embodiments, X⁴ is CH. In some embodiments, X⁴ is N.

In some embodiments, X¹ and X³ are each N; and X² and X⁴ are each CH. In some embodiments, X¹ and X³ are each CH; and X² and X⁴ are each N.

In some embodiments, X¹ is N; and X², X³, and X⁴ are each CH. In some embodiments, X² is N; and X¹, X³, and X⁴ are each CH. In some embodiments, X³ is N; and X¹, X², and X⁴ are each CH. In some embodiments, X⁴ is N; and X¹, X², and X³ are each CH.

In some embodiments, X¹, X², X³ and X⁴ are each N. In some embodiments, X¹, X², X³ and X⁴ are each CH.

In some embodiments, X⁵ is N. In some embodiments, X⁵ is CH.

In some embodiments, R¹, R², R³, and R⁴ are each independently H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₁-C₂₀ hydroxyalkyl, optionally substituted C₁-C₂₀ haloalkyl, optionally substituted C₃-C₈ cycloalkyl, or optionally substituted 3 to 8-membered heterocycloalkyl, wherein each is optionally substituted with one or more amido, C₁-C₆ alkyl, C₂-C₆ alkynyl, azido, amino, halogen, C₁-C₆ haloalkyl, C₁-C₆ hydroxyalkyl, hydroxy, guanidyl, nitro, oxo (=O), phosphorous hydroxide, polyethylene glycol (PEG), or AA, wherein AA is a naturally occurring amino acid. In some embodiments, R¹, R², R³, and R⁴ are each independently H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₁-C₂₀ hydroxyalkyl, optionally substituted C₁-C₂₀ haloalkyl, optionally substituted C₂-C₂₀ heteroalkenyl, optionally substituted C₃-C₈ cycloalkyl, or optionally substituted 3 to 8-membered heterocycloalkyl, wherein each is optionally substituted with one or more —F, —CN, —NH₂, —N₃, —OH, CF₃, —OP(O)(OH)₂, —OP(O)(OCH₃)₂, —OP(O)(OCH₃)(OH), —OP(O)₂OH, or —NHC(NH₂)₂. In some embodiments, R¹, R², R³, and R⁴ are each independently substituted with —F, —CN, —NH₂, —N₃, —OH, or CF₃.

In some embodiments, R¹, R², R³, R⁴, and R⁵ are each independently H or an optionally substituted C₁-C₂₀ alkyl, optionally substituted C₂-C₂₀ alkenyl, optionally substituted C₂-C₂₀ alkynyl, or optionally substituted C₁-C₂₀ heteroalkyl. In some embodiments, R¹, R², R³, R⁴, and R⁵ are each independently H or an optionally substituted C₁-C₂₀ alkyl, or optionally substituted C₁-C₂₀ heteroalkyl.

In some embodiments, R¹, R², R³, R⁴, and R⁵ are each independently H or an optionally substituted C₁-C₂₀ alkyl. In some embodiments, R¹, R², R³, R⁴, and R⁵ are each independently an optionally substituted C₁-C₂₀ alkyl. In some embodiments, R¹, R², R³, R⁴, and R⁵ are each independently optionally substituted C₁-C₁₂ alkyl. In some embodiments, R¹, R², R³, R⁴, and R⁵ are each independently optionally substituted C₁-C₆ alkyl. In some embodiments, the alkyl is a branched or straight chained alkyl. In some embodiments, R¹, R², R³, R⁴, and R⁵ are each independently methyl, ethyl, n-propyl, iso-propyl, n-butyl, sec-butyl, iso-butyl, or tert-butyl. In some embodiments, R¹, R², R³, R⁴, and R⁵ are each independently methyl, ethyl, iso-propyl, sec-butyl, iso-butyl, or tert-butyl. In some embodiments, R¹, R², R³, R⁴, and R⁵ are each independently methyl or iso-propyl. In some embodiments, R¹, R², R³, R⁴, and R⁵ are each methyl. In some embodiments, R¹, R², R³, R⁴, and R⁵ are each iso-propyl.

In some embodiments, R¹, R², R³, R⁴, and R⁵ are each H.

In some embodiments, R¹, R², R³, R⁴, and R⁵ are each independently an optionally substituted C₁-C₂₀ heteroalkyl. In some embodiments, the heteroalkyl is polyethylene glycol (PEG). In some embodiments, R^{B} is PEG₁₋₂₀. In some embodiments, R^{B} is

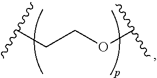

wherein p is 1-20. In some embodiments, p is 1-18, 1-16, 1-14, 1-12, 1-10, 1-8, 1-6, or 1-4. In some embodiments, p is 15, 14, 13, 12, 11, 10, 9, 8, 7, 6, 5, 4, 3, 2, or 1. In some embodiments, p is 10. In some embodiments, p is 9. In some embodiments, p is 8. In some embodiments, p is 7. In some embodiments, p is 6. In some embodiments, p is 5. In some embodiments, p is 4. In some embodiments, p is 3. In some embodiments, p is 2.

In some embodiments, R¹, R², R³, R⁴, and R⁵ are each independently an optionally substituted C₃-C₅ cycloalkyl or optionally substituted 3 to 8-membered heterocycloalkyl. In some embodiments, R¹, R², R³, R⁴, and R⁵ are each independently an optionally substituted C₃-C₈ cycloalkyl. In some embodiments, the cycloalkyl is a polycyclic cycloalkyl. In some embodiments, the cycloalkyl is polycyclic cycloalkyl that is spiro, fused, or bridged. In some embodiments, the cycloalkyl is a cyclopropyl, cyclobutyl, cyclopentyl, cyclohexyl, or cycloheptyl. In some embodiments, the cycloalkyl is a cyclopentyl or cyclohexyl. In some embodiments, the cycloalkyl is a cyclopropyl. In some embodiments, the cycloalkyl is a cyclobutyl. In some embodiments, the cycloalkyl is a cyclopentyl. In some embodiments, the cycloalkyl is a cyclohexyl.

In some embodiments, R¹, R², R³, R⁴, and R⁵ are each independently an optionally substituted 3 to 8-membered heterocycloalkyl. In some embodiments, the heterocycoalkyl is monocyclic heterocycloalkyl. In some embodiments, the heterocycloalkyl is polycyclic heterocycloalkyl. In some embodiments, the heterocycloalkyl is a 4-membered, 5-membered, or 6-membered heterocycloalkyl where the heteroatom is selected from N, O, or S. In some embodiments, the heterocycloalkyl is a 4-membered heterocycloalkyl wherein the heteroatom is N or O. In some embodiments, the heterocycloalkyl is a 5-membered heterocycloalkyl comprising a one or two heteroatom selected from N and O. In some embodiments, the heterocycloalkyl is a 6-membered heterocycloalkyl comprising one or two heteroatom selected from N and O. In some embodiments, the heterocycloalkyl is pyrrolidine, piperidine, or morpholine.

In some embodiments, E¹ is L-R^{B}, wherein L is a bond; and R^{B} is H.

In some embodiments, E¹ is L-R^{B}, wherein L is —NHC(O)—; and R^{B} is an optionally substituted C₁-C₂₀ alkyl or optionally substituted C₁-C₂₀ heteroalkyl.

In some embodiments, L is a bond, —O—, —NH—, —C(O)—, —NHC(O)—, or —NHC(O)NH. In some embodiments, L is a bond. In some embodiments, L is —O—. In some embodiments, L is —NH—. In some embodiments, L is —C(═O)—. In some embodiments, L is —NHC(O)—. In some embodiments, L is —NHC(O)NH—.

In some embodiments, R^{A} is H or C₁-C₆ alkyl. In some embodiments, R^{A} is C₁-C₆ alkyl. In some embodiments, the alkyl is a branched or straight chain alkyl. In some embodiments, R^{A} is methyl, ethyl, n-propyl, iso-propyl, n-butyl, iso-butyl, sec-butyl, or tert-butyl. In some embodiments, R^{A} is methyl or ethyl. In some embodiments, R^{A} is methyl. In some embodiments, R^{A} is ethyl. In some embodiments, R^{A} is H.

In some embodiments, R^{B} is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, or one or more AA, wherein each is optionally substituted with one or more amido, C₁-C₆ alkyl, C₂-C₆ alkynyl, azido, amino, halogen, C₁-C₆ haloalkyl, C₁-C₆ hydroxyalkyl, hydroxy, guanidyl, nitro, oxo (=O), phosphorous hydroxide, polyethylene glycol (PEG), or AA, wherein AA is a naturally occurring amino acid. In some embodiments, R^{B} is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, or one or more AA, wherein each is optionally substituted with one or more —F, —CN, —NH₂, —N₃, —OH, CF₃, —CHF₂, —OP(O)(OH)₂, —OP(O)(OCH₃)₂, —OP(O)(OCH₃)(OH), —OP(O)₂OH, —NHC(NH₂)₂, or —NHC(NCH₃)₂. In some embodiments, R^{B} is optionally substituted with one or more F, —CN, —NH₂, —N₃, —OH, CF₃, or —CHF₂. In some embodiments, R^{B} is optionally substituted with —NHC(NH₂)₂ or —NHC(NCH₃)₂.

In some embodiments, R^{B} is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, or one or more AA; wherein each AA is independently a naturally occurring amino acid. In some embodiments, R^{B} is H or optionally substituted C₁-C₂₀ alkyl or optionally substituted C₁-C₂₀ heteroalkyl. In some embodiments, R^{B} is optionally substituted C₁-C₂₀ alkyl. In some embodiments, R^{B} is optionally substituted C₁-C₁₂ alkyl. In some embodiments, R^{B} is optionally substituted C₁-C₆ alkyl. In some embodiments, R^{B} is a branched or straight chained alkyl. In some embodiments, R^{B} is an unsubstituted alkyl.

In some embodiments, R^{B} is optionally substituted C₁-C₂₀ heteroalkyl. In some embodiments, the heteroalkyl is polyethylene glycol (PEG). In some embodiments, R^{B} is PEG₁₋₂₀. In some embodiments, R^{B} is

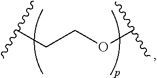

wherein p is 1-20. In some embodiments, p is 1-18, 1-16, 1-14, 1-12, 1-10, 1-8, 1-6, or 1-4. In some embodiments, p is 15, 14, 13, 12, 11, 10, 9, 8, 7, 6, 5, 4, 3, 2, or 1. In some embodiments, p is 10. In some embodiments, p is 9. In some embodiments, p is 8. In some embodiments, p is 7. In some embodiments, p is 6. In some embodiments, p is 5. In some embodiments, p is 4. In some embodiments, p is 3. In some embodiments, p is 2.

In some embodiments, R^{B} is H.

In some embodiments, R^{B} is one or more AA, wherein each AA is independently a naturally occurring amino acid. In some embodiments, R^{B} is 1-5 AA. In some embodiments, R^{B} is one, two, or three AA.

In some embodiments, R^{C} is H or C₁-C₆ alkyl. In some embodiments, R^{C} is C₁-C₆ alkyl. In some embodiments, the alkyl is a branched or straight chain alkyl. In some embodiments, R^{C} is methyl, ethyl, n-propyl, iso-propyl, n-butyl, iso-butyl, sec-butyl, or tert-butyl. In some embodiments, R^{C} is methyl or ethyl. In some embodiments, R^{C} is methyl. In some embodiments, R^{C} is ethyl. In some embodiments, R^{C} is H.

In some embodiments, R^{D} is H or C₁-C₆ alkyl. In some embodiments, R^{D} is C₁-C₆ alkyl. In some embodiments, the alkyl is a branched or straight chain alkyl. In some embodiments, R^{D} is methyl, ethyl, n-propyl, iso-propyl, n-butyl, iso-butyl, sec-butyl, or tert-butyl. In some embodiments, R^{D} is methyl or ethyl. In some embodiments, R^{D} is methyl. In some embodiments, R^{D} is ethyl. In some embodiments, R^{D} is H.

In some embodiments, each AA is independently a naturally occurring amino acid. In some embodiments, each AA is independently selected from arginine, lysine, tyrosine, serine, or threonine. In some embodiments, each AA is independently selected from arginine and lysine. In some embodiments, each AA is independently selected from arginine. In some embodiments, each AA is independently selected from lysine.

In some embodiments, n is 1. In some embodiments, n is 2. In some embodiments, n is 3.

In some embodiments, m is 1. In some embodiments, m is 2. In some embodiments, m is 3.

In some embodiments, n is 1 and m is 1. In some embodiments, n is 2 and m is 1. In some embodiments, n is 1 and m is 2. In some embodiments, n is 2 and m is 2.

Any combination of the groups described above for the various variables is contemplated herein. Throughout the specification, groups and substituents thereof are chosen by one skilled in the field to provide stable moieties and polyamides.

The polyamides of the present disclosure may be prepared by the methods disclosed herein. In some embodiments, the terms “process”, “synthesis”, and “synthetic methods” are interchangeable and relate to a method of making the polyamides disclosed herein. In some embodiments, the process is a chemical synthesis. In some embodiments, the process is a solution phase or solution base synthesis. In some embodiments, the reaction steps are performed in a solution and/or free from a solid support.

In some embodiments, the process is not a solid phase synthesis. In some embodiments, the synthesis does not involve one or more steps where a solid phase or a solid support is used.

In some embodiments, the process provides for a convergent synthesis. For example, the polyamides or Formula (I) are synthesized from intermediates 24 and 34 which are each synthetized in parallel and then combined to produce the final product. A convergent synthesis can provide advantages in that the overall route may be shorter since each linear sequence is shorter. Additionally some chemistries may not be amenable to solid phase synthesis.

In another aspect, provided herein is a polyamide produced by any of the methods disclosed herein, or a pharmaceutically acceptable salt thereof. In some embodiments, is a polyamide of Formula (I) or a pharmaceutically acceptable salt thereof, produced by the methods described herein. In some embodiments, is a polyamide of Formula (II), or a pharmaceutically acceptable salt thereof, produced by the methods described herein.

“Yield” as used herein, means the ratio of the actual product weight and the theoretical product weight calculated from the amount of starting materials of a reaction. In some embodiments, the yield is of a polyamide of Formula (I) or (II), or a pharmaceutically acceptable salt thereof, is about 50%, about 60%, about 70%, about 80%, about 90%, about 95%, about 96%, about 97%, about 98%, or about 99%. In some embodiments, the yield is about 60%. In some embodiments, the yield is about 70%. In some embodiments, the yield is about 80%. In some embodiments, the yield is about 90%. In some embodiments, the yield is about 95%.

In some embodiments, the yield of a polyamide of Formula (I) or (II), or a pharmaceutically acceptable salt thereof, is at least about 50%, about 60%, about 70%, about 80%, about 90%, about 95%, about 96%, about 97%, about 98%, or about 99%. In some embodiments, the yield is at least about 60%. In some embodiments, the yield is at least about 70%. In some embodiments, the yield is at least about 80%. In some embodiments, the yield is at least about 90%.

The purity of a polyamides disclosed herein (e.g. a polyamides of Formula (I) or (II), or a pharmaceutically acceptable salt thereof) is above about 90.0%, about 95.0%, about 96.0%, about 97.0%, about 98.0%, about 99.0%, about 99.5%, about 99.8%, or about 99.9%. In some embodiments, the purity is about 90.0%. In some embodiments, the purity is about 95.0%. In some embodiments, the purity is about 96.0%. In some embodiments, the purity is about 97.0%. In some embodiments, the purity is about 98.0%. In some embodiments, the purity is about 99.0%. In some embodiments, the purity is about 99.5%. In some embodiments, the purity is about 99.8%. In some embodiments, the purity is about 99.9%. Purity is calculated as the amount of the product (e.g. a polyamide of Formula (I) or (II)) divided by the total amount of product including all impuries. Purity can be assessed for each reaction step of any of the synthetic schemes disclosed herein. The purity of any of the polyamides or intermediates may be determined by any suitable method such as HPLC, GC (gas chromatography), mass specification, or NMR.

Polyamides prepared from the synthesis methods disclosed herein (e.g. the polyamides of Formula (I) or (II), or pharmaceutically acceptable salts thereof), are substantially free of impurities. Chemical impurities may include starting materials, intermediates, solvents, reagents and/or any byproduct. The level of impurities may be less than about 5%, about 2%, about 1%, about 0.5%, about 0.2% or about 0.1%. In some embodiments, the level of impuries is less than about 5%. In some embodiments, the level of impuries is less than about 2%. In some embodiments, the level of impuries is less than about 1%. In some embodiments, the level of impuries is less than about 0.5%. In some embodiments, the level of impurities is less than about 0.2%. In some embodiments, the level of impurities is less than about 0.1%.

### Polyamide Compounds

In an aspect, provided herein are polyamides comprising pyrroles and/or imidazoles.

In some embodiments, the polyamide can selectively bind to DNA which will affect a target gene. The form of the polyamide selected can vary based on the target gene. The polyamide can be a linear polyamide, a hairpin polyamide, a H-pin polyamide, an overlapped polyamide, a slipped polyamide, a cyclic polyamide, a tandem polyamide, or an extended polyamide. In some embodiments, the polyamide is a linear polyamide. In some embodiments, the polyamide is a hairpin polyamide.

The polyamide described herein are comprised of monomer subunits linked together through an amide bond. The polyamides described herein can include one or more subunits selected from the group consisting of:

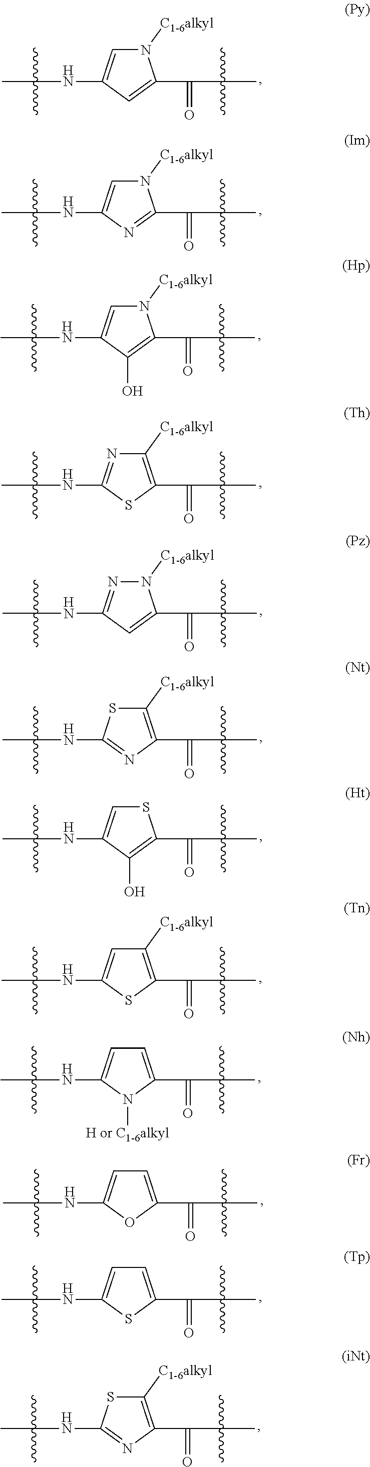

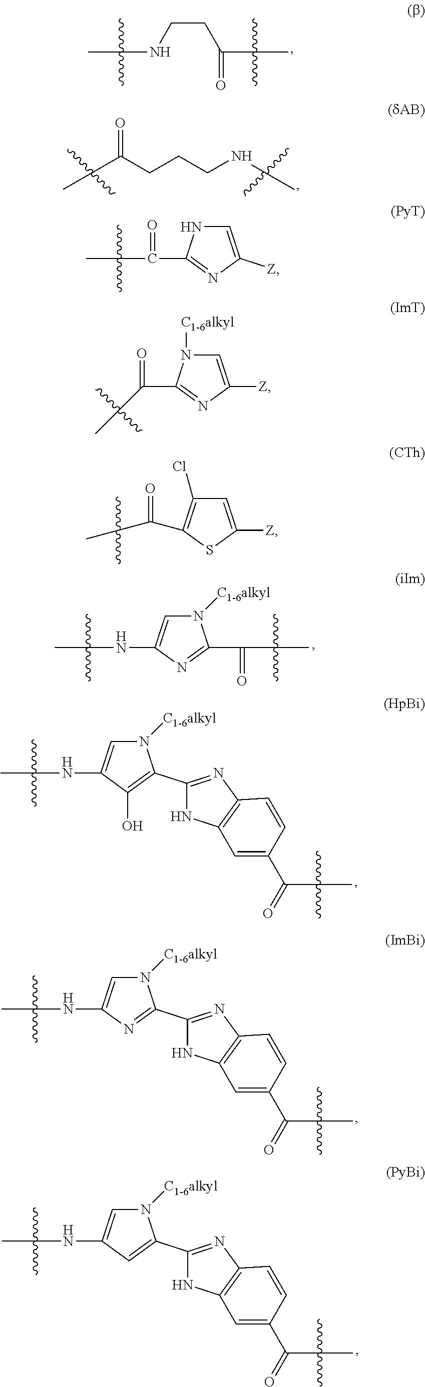

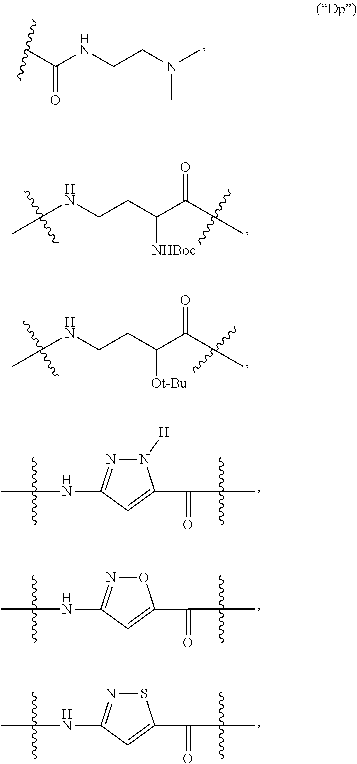

—NH-benzopyrazinylene-CO—, —NH-phenylene-CO—, —NH-pyridinylene-CO—, —NH-piperidinylene-CO—, —NH-pyrimidinylene-CO—, —NH-anthracenylene-CO—, —NH-quinolinylene-CO—, and

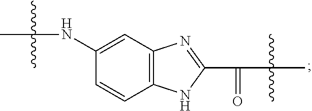

wherein Z is H, NH₂, C₁₋₆ alkyl, C_{1_6} haloalkyl or C₁₋₆ alkyl-NH₂.

In some embodiments, Py is

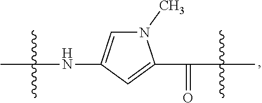 [SMILES: *Nc1cc(C(*)=O)n(C)c1]

### Im is 0

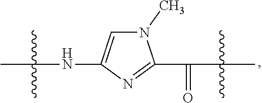 [SMILES: *Nc1cn(C)c(C(*)=O)n1]

### Hp is

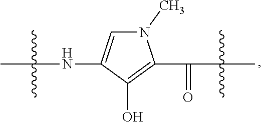 [SMILES: *Nc1cn(C)c(C(*)=O)c1O]

### Th is

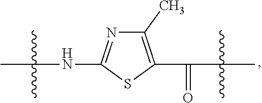 [SMILES: *Nc1nc(C)c(C(*)=O)s1]

### Pz is

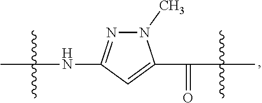 [SMILES: *Nc1cc(C(*)=O)n(C)n1]

### Nt is

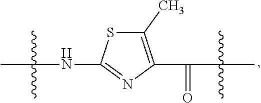 [SMILES: *Nc1nc(C(*)=O)c(C)s1]

### Tn is

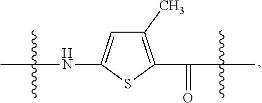 [SMILES: *Nc1cc(C)c(C(*)=O)s1]

### Nh is

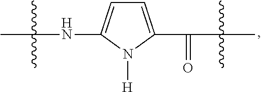 [SMILES: *Nc1ccc(C(*)=O)[nH]1]

iNt is

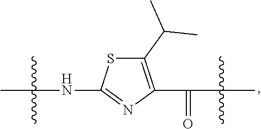 [SMILES: *Nc1nc(C(*)=O)c(C(C)C)s1]

iIm is

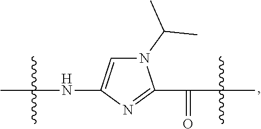 [SMILES: *Nc1cn(C(C)C)c(C(*)=O)n1]

### HpBi is

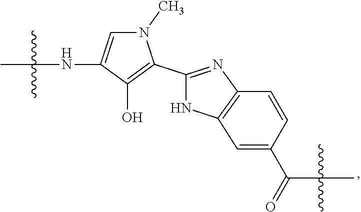 [SMILES: *Nc1cn(C)c(-c2nc3ccc(C(*)=O)cc3[nH]2)c1O]

### ImBi is

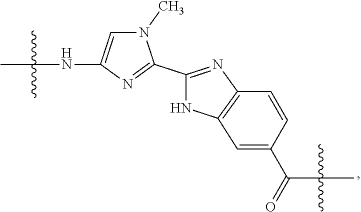 [SMILES: *Nc1cn(C)c(-c2nc3ccc(C(*)=O)cc3[nH]2)n1]

### PyBi is

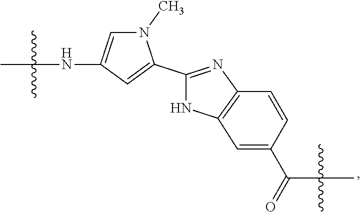 [SMILES: *Nc1cc(-c2nc3ccc(C(*)=O)cc3[nH]2)n(C)c1]

### Dp is

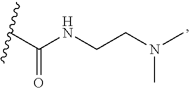 [SMILES: *C(=O)NCCN(C)C]

### —NH-benzopyrazinylene-CO— is

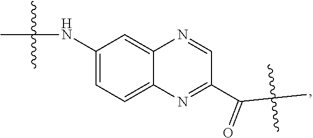 [SMILES: *Nc1ccc2nc(C(*)=O)cnc2c1]

### —NH-phenylene-CO— is

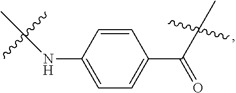 [SMILES: *Nc1ccc(C(*)=O)cc1]

### —NH-pyridinylene-CO— is

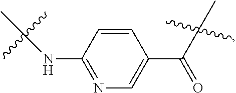 [SMILES: *Nc1ccc(C(*)=O)cn1]

### —NH-piperidinylene-CO— is

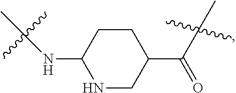 [SMILES: *NC1CCC(C(*)=O)CN1]

### —NH-pyrazinylene-CO— is

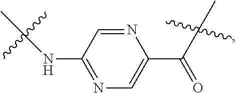 [SMILES: *Nc1cnc(C(*)=O)cn1]

### —NH-anthracenylene-CO— is

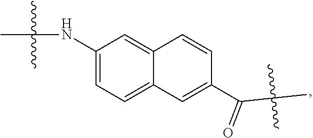 [SMILES: *Nc1ccc2cc(C(*)=O)ccc2c1]

### and —NH-quinolinylene-CO— is

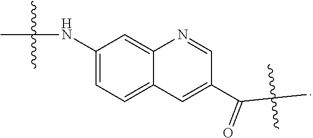 [SMILES: *Nc1ccc2cc(C(*)=O)cnc2c1]

In some embodiments, the polyamide described herein comprises one or more subunits selected from the group consisting of optionally substituted N-methylpyrrole, optionally substituted N-methylimidazole, β-alanine, and β-alanine. In some embodiments, the polyamide described herein comprises one or more subunits selected from the group consisting of optionally substituted N-methylpyrrole, optionally substituted N-methylimidazole, and β-alanine.

In some embodiments, the monomer subunit, when positioned as a terminal unit, does not have an amine, carbonyl, or a carboxylic acid group at the terminal. The amine or carboxylic acid group in the terminal is replaced by a hydrogen. For example, Py, when used as a terminal unit, is understood to have the structure of

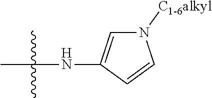

(e.g,

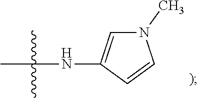 [SMILES: *Nc1ccn(C)c1]

and Im, when positioned as a terminal unit, is understood to have the structure of

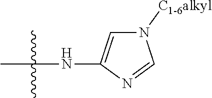

(e.g,

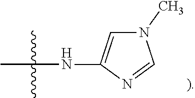 [SMILES: *Nc1cn(C)cn1]

In addition, when Py or Im is used as a terminal unit, Py and Im can be respectively replaced by PyT

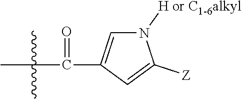

(e.g.,

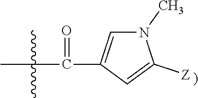

### and ImT

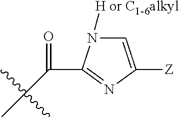

(e.g.,

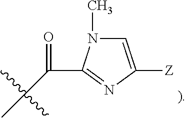

In some embodiments, the polyamide is a compound of Formula (I) or (II), or a pharmaceutically acceptable salt thereof. In some embodiments, the polyamide is a compounds selected from Table 1, or a pharmaceutically acceptable salt thereof.

TABLE 1
Representative polyamides of the disclosure.

| Name | Structure |
|---|---|
| 1 | 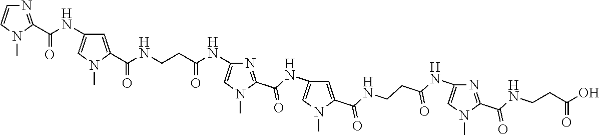 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1] |
| 2 | 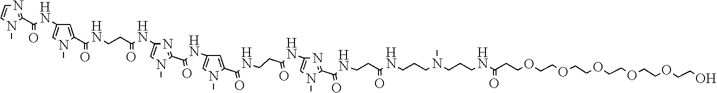 [SMILES: CN(CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C)CCCNC(=O)CCOCCOCCOCCOCCOCCO] |
| 3 | 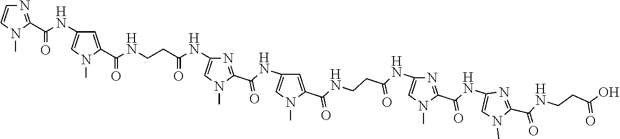 [SMILES: Cn1cc(NC(=O)c2nccn2C)cc1C(=O)NCCC(=O)Nc1cn(C)c(C(=O)Nc2cc(C(=O)NCCC(=O)Nc3cn(C)c(C(=O)Nc4cn(C)c(C(=O)NCCC(=O)O)n4)n3)n(C)c2)n1] |
| 4 | 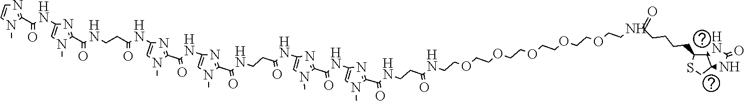 [SMILES: Cn1cc(NC(=O)c2nccn2C)nc1C(=O)NCCC(=O)Nc1cn(C)c(C(=O)Nc2cn(C)c(C(=O)NCCC(=O)Nc3cn(C)c(C(=O)Nc4cn(C)c(C(=O)NCCC(=O)NCCOCCOCCOCCOCCOCCNC(=O)CCCC[C@@H]5SC[C@@H]6NC(=O)N[C@@H]65)n4)n3)n2)n1] |
| 5 | 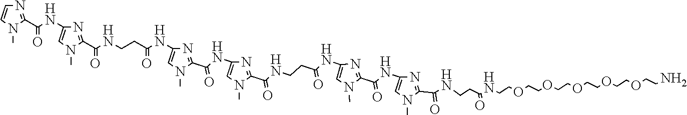 [SMILES: Cn1cc(NC(=O)c2nccn2C)nc1C(=O)NCCC(=O)Nc1cn(C)c(C(=O)Nc2cn(C)c(C(=O)NCCC(=O)Nc3cn(C)c(C(=O)Nc4cn(C)c(C(=O)NCCC(=O)NCCOCCOCCOCCOCCOCCN)n4)n3)n2)n1] |
| 6 | 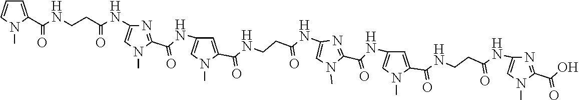 [SMILES: Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)O)n1] |
| 7 | 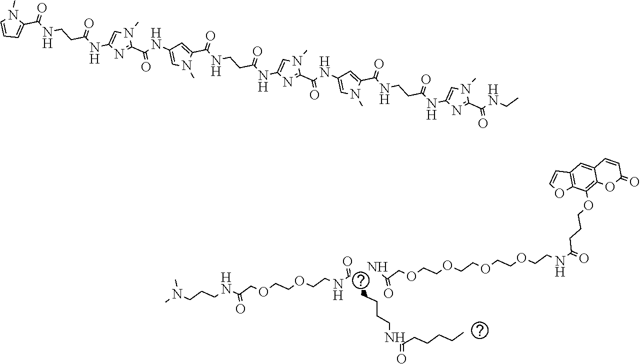 |
| 8 | 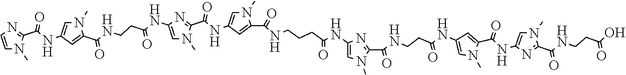 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1] |
| 9 | 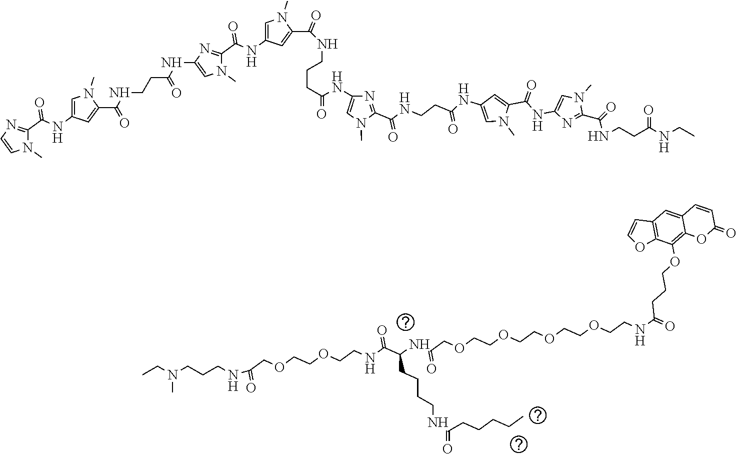 |
| 10 | 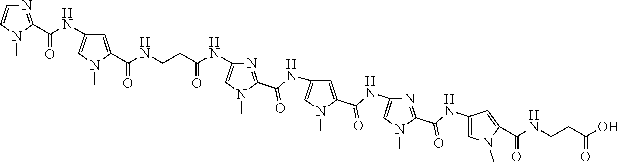 [SMILES: Cn1cc(NC(=O)c2nc(NC(=O)c3cc(NC(=O)c4nc(NC(=O)CCNC(=O)c5cc(NC(=O)c6nccn6C)cn5C)cn4C)cn3C)cn2C)cc1C(=O)NCCC(=O)O] |
| 11 | 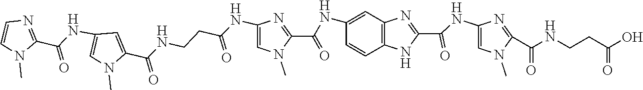 [SMILES: Cn1cc(NC(=O)c2nccn2C)cc1C(=O)NCCC(=O)Nc1cn(C)c(C(=O)Nc2ccc3[nH]c(C(=O)Nc4cn(C)c(C(=O)NCCC(=O)O)n4)nc3c2)n1] |
| 12 | 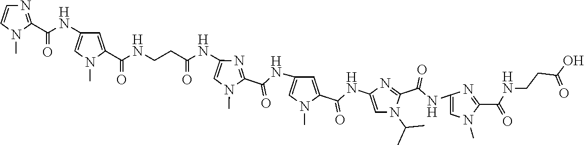 [SMILES: CC(C)n1cc(NC(=O)c2cc(NC(=O)c3nc(NC(=O)CCNC(=O)c4cc(NC(=O)c5nccn5C)cn4C)cn3C)cn2C)nc1C(=O)Nc1cn(C)c(C(=O)NCCC(=O)O)n1] |
| 13 | 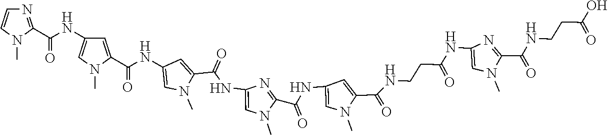 [SMILES: Cn1cc(NC(=O)c2nc(NC(=O)c3cc(NC(=O)c4cc(NC(=O)c5nccn5C)cn4C)cn3C)cn2C)cc1C(=O)NCCC(=O)Nc1cn(C)c(C(=O)NCCC(=O)O)n1] |
| 14 | 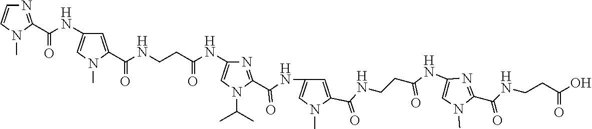 [SMILES: CC(C)n1cc(NC(=O)CCNC(=O)c2cc(NC(=O)c3nccn3C)cn2C)nc1C(=O)Nc1cc(C(=O)NCCC(=O)Nc2cn(C)c(C(=O)NCCC(=O)O)n2)n(C)c1] |
| 15 | 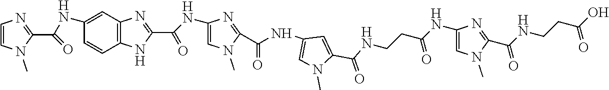 [SMILES: Cn1cc(NC(=O)c2nc(NC(=O)c3nc4cc(NC(=O)c5nccn5C)ccc4[nH]3)cn2C)cc1C(=O)NCCC(=O)Nc1cn(C)c(C(=O)NCCC(=O)O)n1] |
| 16 | 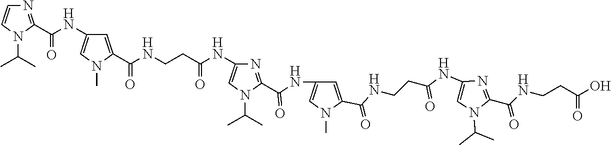 [SMILES: CC(C)n1cc(NC(=O)CCNC(=O)c2cc(NC(=O)c3nc(NC(=O)CCNC(=O)c4cc(NC(=O)c5nccn5C(C)C)cn4C)cn3C(C)C)cn2C)nc1C(=O)NCCC(=O)O] |
| 17 | 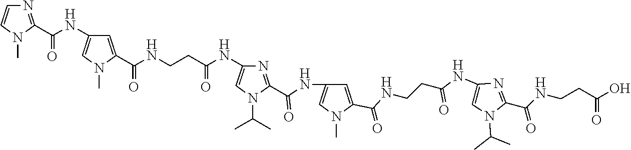 [SMILES: CC(C)n1cc(NC(=O)CCNC(=O)c2cc(NC(=O)c3nc(NC(=O)CCNC(=O)c4cc(NC(=O)c5nccn5C)cn4C)cn3C(C)C)cn2C)nc1C(=O)NCCC(=O)O] |
| 18 | 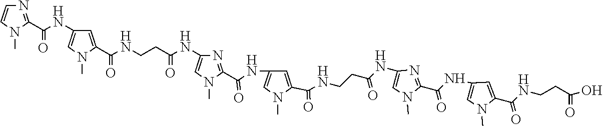 [SMILES: Cn1cc(NC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)cc1C(=O)NCCC(=O)O] |
| 19 | 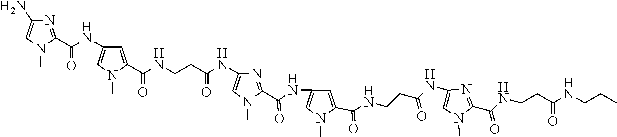 [SMILES: CCCNC(=O)CCNC(=O)c1nc(NC(=O)CCNC(=O)c2cc(NC(=O)c3nc(NC(=O)CCNC(=O)c4cc(NC(=O)c5nc(N)cn5C)cn4C)cn3C)cn2C)cn1C] |
| 20 | 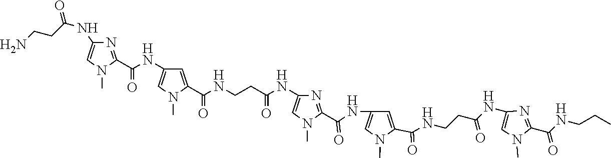 [SMILES: CCCNC(=O)c1nc(NC(=O)CCNC(=O)c2cc(NC(=O)c3nc(NC(=O)CCNC(=O)c4cc(NC(=O)c5nc(NC(=O)CCN)cn5C)cn4C)cn3C)cn2C)cn1C] |
| 21 | 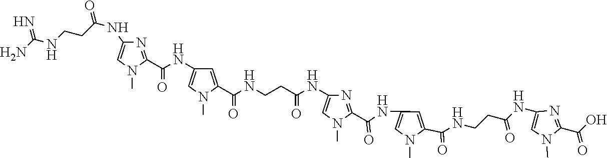 [SMILES: Cn1cc(NC(=O)c2nc(NC(=O)CCNC(=O)c3cc(NC(=O)c4nc(NC(=O)CCNC(=N)N)cn4C)cn3C)cn2C)cc1C(=O)NCCC(=O)Nc1cn(C)c(C(=O)O)n1] |
| 22 | 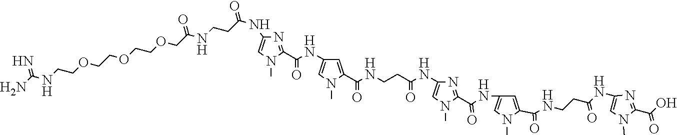 [SMILES: Cn1cc(NC(=O)c2nc(NC(=O)CCNC(=O)c3cc(NC(=O)c4nc(NC(=O)CCNC(=O)COCCOCCOCCNC(=N)N)cn4C)cn3C)cn2C)cc1C(=O)NCCC(=O)Nc1cn(C)c(C(=O)O)n1] |
| 23 | 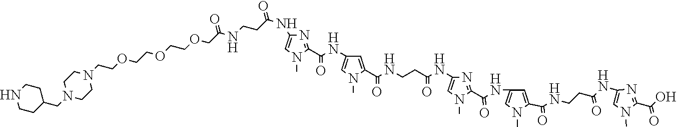 [SMILES: Cn1cc(NC(=O)c2nc(NC(=O)CCNC(=O)c3cc(NC(=O)c4nc(NC(=O)CCNC(=O)COCCOCCOCCN5CCN(CC6CCNCC6)CC5)cn4C)cn3C)cn2C)cc1C(=O)NCCC(=O)Nc1cn(C)c(C(=O)O)n1] |
| 24 | 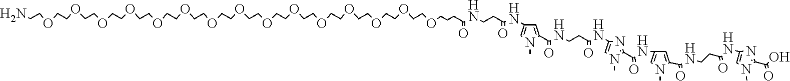 [SMILES: Cn1cc(NC(=O)c2nc(NC(=O)CCNC(=O)c3cc(NC(=O)CCNC(=O)CCCOCCOCCOCCOCCOCCOCCOCCOCCOCCOCCOCCOCCOCCOCCOCCN)cn3C)cn2C)cc1C(=O)NCCC(=O)Nc1cn(C)c(C(=O)O)n1] |
| 25 | 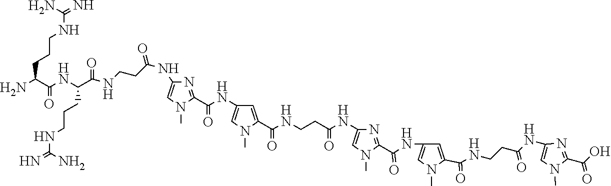 [SMILES: Cn1cc(NC(=O)c2nc(NC(=O)CCNC(=O)c3cc(NC(=O)c4nc(NC(=O)CCNC(=O)[C@H](CCCNC(=N)N)NC(=O)[C@@H](N)CCCNC(=N)N)cn4C)cn3C)cn2C)cc1C(=O)NCCC(=O)Nc1cn(C)c(C(=O)O)n1] |
| 26 | 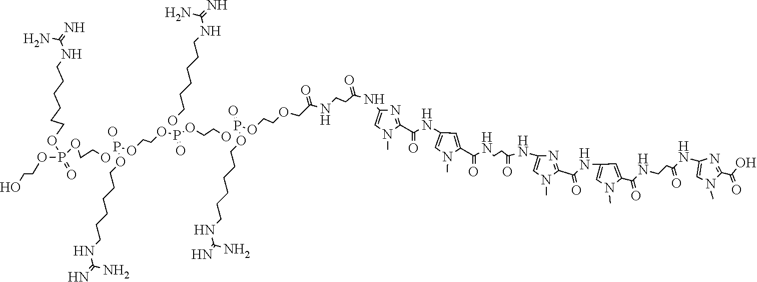 [SMILES: Cn1cc(NC(=O)c2nc(NC(=O)CCNC(=O)c3cc(NC(=O)c4nc(NC(=O)CCNC(=O)COCCOP(=O)(OCCCCCCNC(=N)N)OCCOP(=O)(OCCCCCCNC(=N)N)OCCOP(=O)(OCCCCCCNC(=N)N)OCCOP(=O)(OCCO)OCCCCCCNC(=N)N)cn4C)cn3C)cn2C)cc1C(=O)NCCC(=O)Nc1cn(C)c(C(=O)O)n1] |
| 27 | 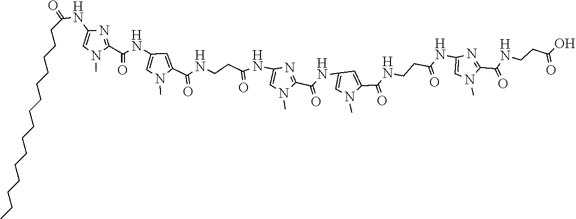 [SMILES: CCCCCCCCCCCCCCCC(=O)Nc1cn(C)c(C(=O)Nc2cc(C(=O)NCCC(=O)Nc3cn(C)c(C(=O)Nc4cc(C(=O)NCCC(=O)Nc5cn(C)c(C(=O)NCCC(=O)O)n5)n(C)c4)n3)n(C)c2)n1] |
| 28 | 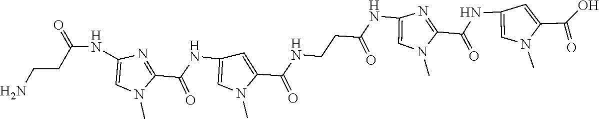 [SMILES: Cn1cc(NC(=O)c2nc(NC(=O)CCNC(=O)c3cc(NC(=O)c4nc(NC(=O)CCN)cn4C)cn3C)cn2C)cc1C(=O)O] |
| 29 | 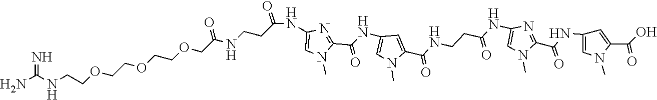 [SMILES: Cn1cc(NC(=O)c2nc(NC(=O)CCNC(=O)c3cc(NC(=O)c4nc(NC(=O)CCNC(=O)COCCOCCOCCNC(=N)N)cn4C)cn3C)cn2C)cc1C(=O)O] |
| 30 | 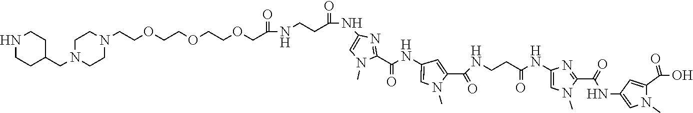 [SMILES: Cn1cc(NC(=O)c2nc(NC(=O)CCNC(=O)c3cc(NC(=O)c4nc(NC(=O)CCNC(=O)COCCOCCOCCN5CCN(CC6CCNCC6)CC5)cn4C)cn3C)cn2C)cc1C(=O)O] |
| 31 | 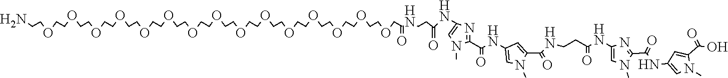 [SMILES: Cn1cc(NC(=O)c2nc(NC(=O)CCNC(=O)c3cc(NC(=O)c4nc(NC(=O)CCNC(=O)COCCOCCOCCOCCOCCOCCOCCOCCOCCOCCOCCOCCOCCOCCOCCN)cn4C)cn3C)cn2C)cc1C(=O)O] |
| 32 | 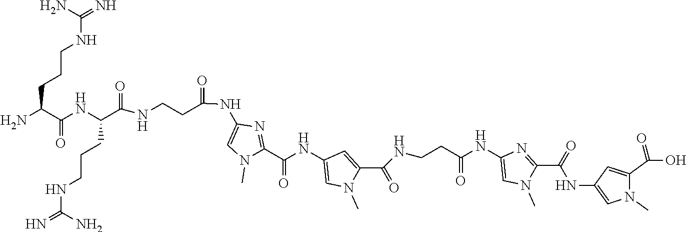 [SMILES: Cn1cc(NC(=O)c2nc(NC(=O)CCNC(=O)c3cc(NC(=O)c4nc(NC(=O)CCNC(=O)[C@H](CCCNC(=N)N)NC(=O)[C@@H](N)CCCNC(=N)N)cn4C)cn3C)cn2C)cc1C(=O)O] |
| 33 | 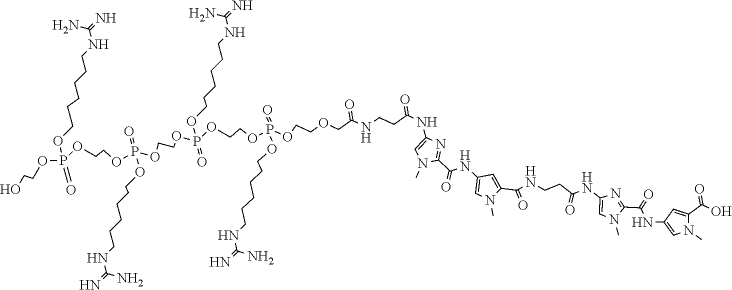 [SMILES: Cn1cc(NC(=O)c2nc(NC(=O)CCNC(=O)c3cc(NC(=O)c4nc(NC(=O)CCNC(=O)COCCOP(=O)(OCCCCCCNC(=N)N)OCCOP(=O)(OCCCCCCNC(=N)N)OCCOP(=O)(OCCCCCCNC(=N)N)OCCOP(=O)(OCCO)OCCCCCCNC(=N)N)cn4C)cn3C)cn2C)cc1C(=O)O] |
| 34 | 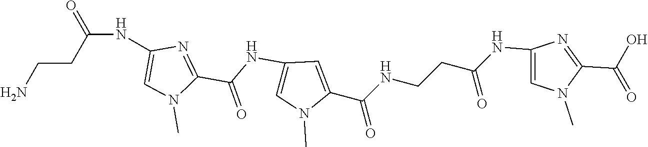 [SMILES: Cn1cc(NC(=O)c2nc(NC(=O)CCN)cn2C)cc1C(=O)NCCC(=O)Nc1cn(C)c(C(=O)O)n1] |
| 35 | 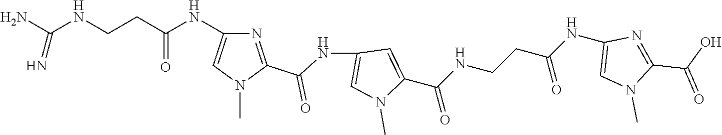 [SMILES: Cn1cc(NC(=O)c2nc(NC(=O)CCNC(=N)N)cn2C)cc1C(=O)NCCC(=O)Nc1cn(C)c(C(=O)O)n1] |
| 36 | 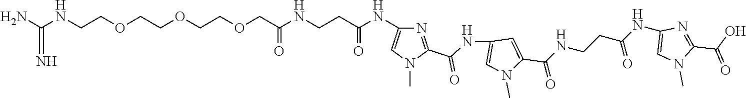 [SMILES: Cn1cc(NC(=O)c2nc(NC(=O)CCNC(=O)COCCOCCOCCNC(=N)N)cn2C)cc1C(=O)NCCC(=O)Nc1cn(C)c(C(=O)O)n1] |
| 37 | 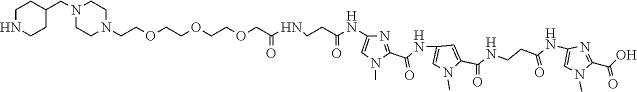 [SMILES: Cn1cc(NC(=O)c2nc(NC(=O)CCNC(=O)COCCOCCOCCN3CCN(CC4CCNCC4)CC3)cn2C)cc1C(=O)NCCC(=O)Nc1cn(C)c(C(=O)O)n1] |
| 38 | 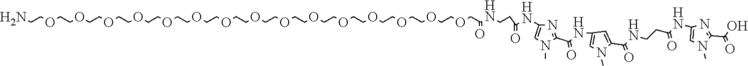 [SMILES: Cn1cc(NC(=O)c2nc(NC(=O)CCNC(=O)COCCOCCOCCOCCOCCOCCOCCOCCOCCOCCOCCOCCOCCOCCOCCN)cn2C)cc1C(=O)NCCC(=O)Nc1cn(C)c(C(=O)O)n1] |
| 39 | 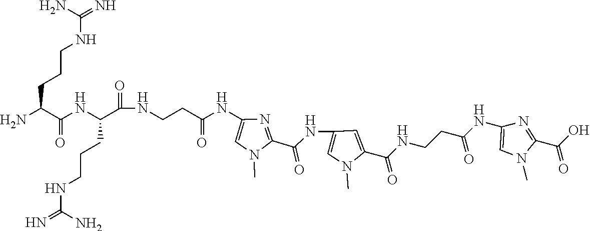 [SMILES: Cn1cc(NC(=O)c2nc(NC(=O)CCNC(=O)[C@H](CCCNC(=N)N)NC(=O)[C@@H](N)CCCNC(=N)N)cn2C)cc1C(=O)NCCC(=O)Nc1cn(C)c(C(=O)O)n1] |
| 40 | 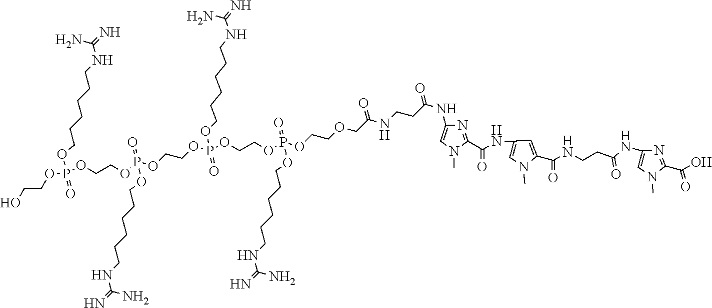 [SMILES: Cn1cc(NC(=O)c2nc(NC(=O)CCNC(=O)COCCOP(=O)(OCCCCCCNC(=N)N)OCCOP(=O)(OCCCCCCNC(=N)N)OCCOP(=O)(OCCCCCCNC(=N)N)OCCOP(=O)(OCCO)OCCCCCCNC(=N)N)cn2C)cc1C(=O)NCCC(=O)Nc1cn(C)c(C(=O)O)n1] |
| 41 | 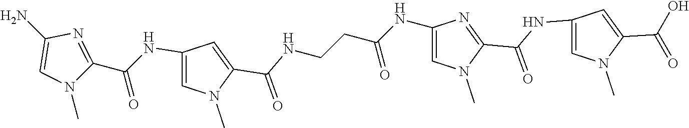 [SMILES: Cn1cc(NC(=O)c2nc(NC(=O)CCNC(=O)c3cc(NC(=O)c4nc(N)cn4C)cn3C)cn2C)cc1C(=O)O] |
| 42 | 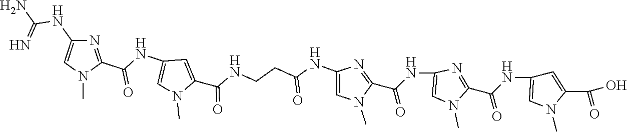 [SMILES: Cn1cc(NC(=O)c2nc(NC(=O)c3nc(NC(=O)CCNC(=O)c4cc(NC(=O)c5nc(NC(=N)N)cn5C)cn4C)cn3C)cn2C)cc1C(=O)O] |
| 43 | 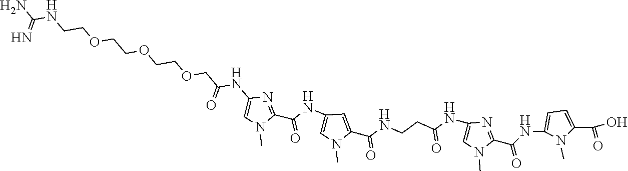 [SMILES: Cn1cc(NC(=O)c2nc(NC(=O)COCCOCCOCCNC(=N)N)cn2C)cc1C(=O)NCCC(=O)Nc1cn(C)c(C(=O)Nc2ccc(C(=O)O)n2C)n1] |
| 44 | 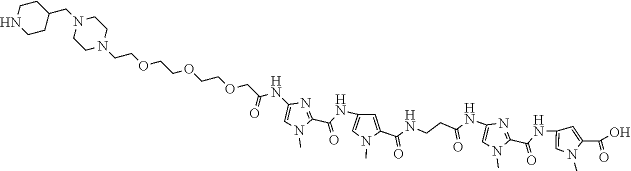 [SMILES: Cn1cc(NC(=O)c2nc(NC(=O)CCNC(=O)c3cc(NC(=O)c4nc(NC(=O)COCCOCCOCCN5CCN(CC6CCNCC6)CC5)cn4C)cn3C)cn2C)cc1C(=O)O] |
| 45 | 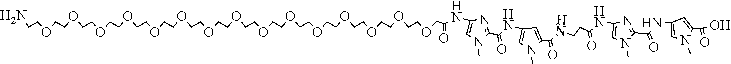 [SMILES: Cn1cc(NC(=O)c2nc(NC(=O)CCNC(=O)c3cc(NC(=O)c4nc(NC(=O)COCCOCCOCCOCCOCCOCCOCCOCCOCCOCCOCCOCCOCCOCCOCCN)cn4C)cn3C)cn2C)cc1C(=O)O] |
| 46 | 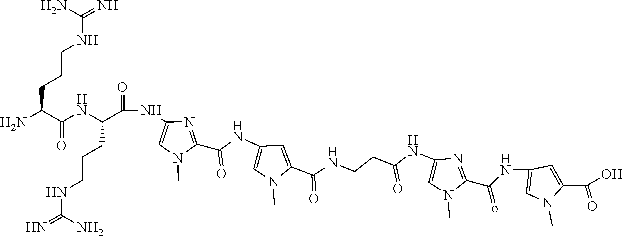 |
| 47 | 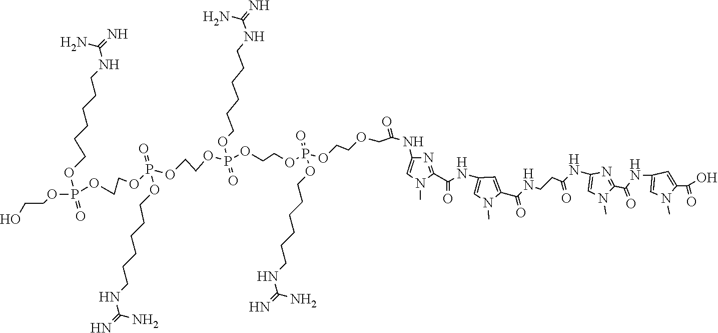 [SMILES: Cn1cc(NC(=O)c2nc(NC(=O)CCNC(=O)c3cc(NC(=O)c4nc(NC(=O)COCCOP(=O)(OCCCCCCNC(=N)N)OCCOP(=O)(OCCCCCCNC(=N)N)OCCOP(=O)(OCCCCCCNC(=N)N)OCCOP(=O)(OCCO)OCCCCCCNC(=N)N)cn4C)cn3C)cn2C)cc1C(=O)O] |
| 48 | 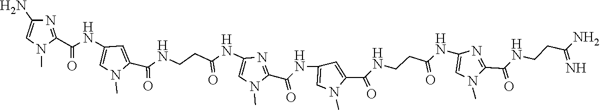 [SMILES: Cn1cc(NC(=O)c2nc(NC(=O)CCNC(=O)c3cc(NC(=O)c4nc(N)cn4C)cn3C)cn2C)cc1C(=O)NCCC(=O)Nc1cn(C)c(C(=O)NCCC(=N)N)n1] |
| 49 | 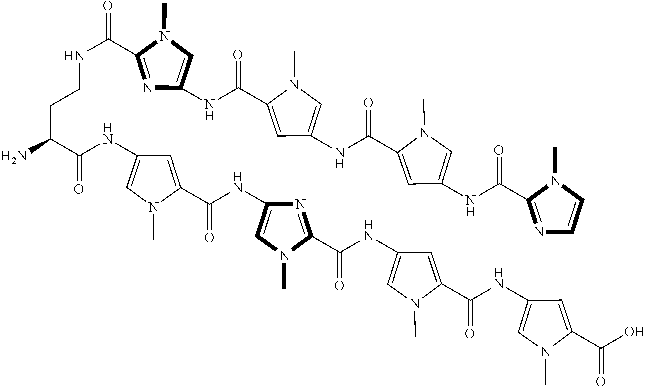 |
| 50 | 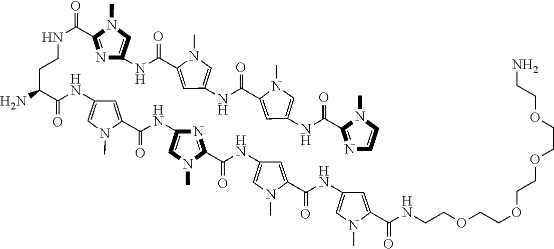 |
| 51 | 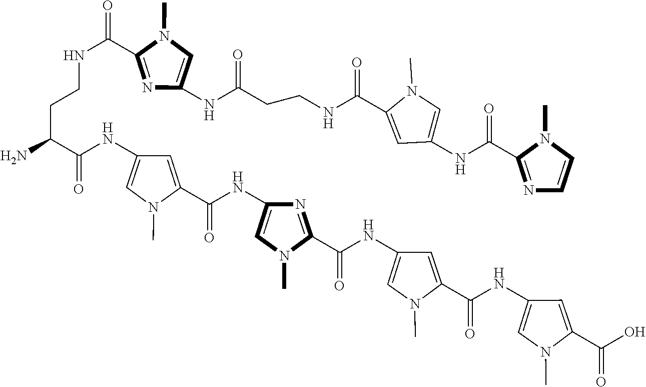 |
| 52 | 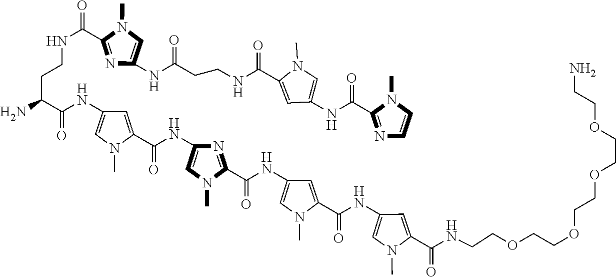 |
| 53 | 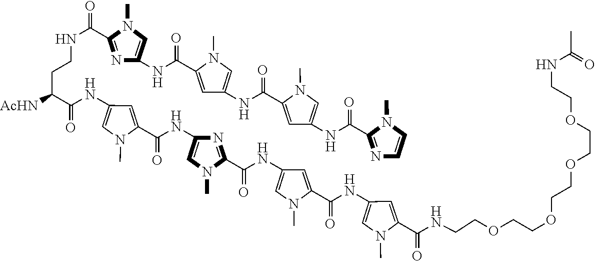 |
| 54 | 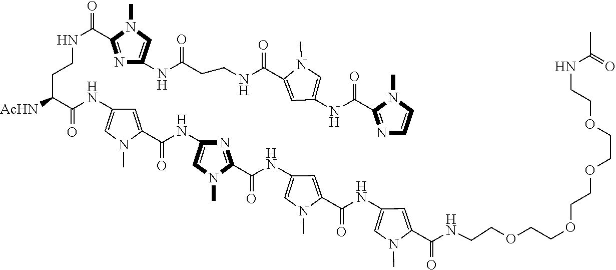 |
| 55 | 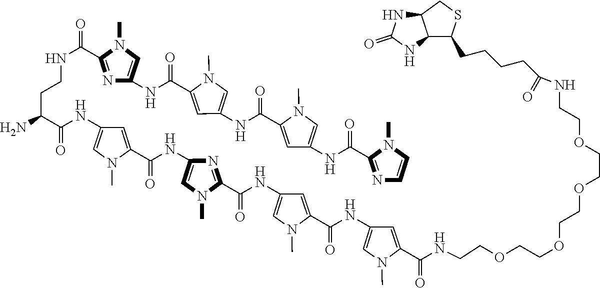 |
| 56 | 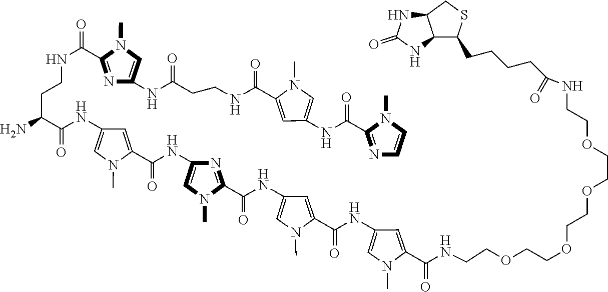 |
| 57 | 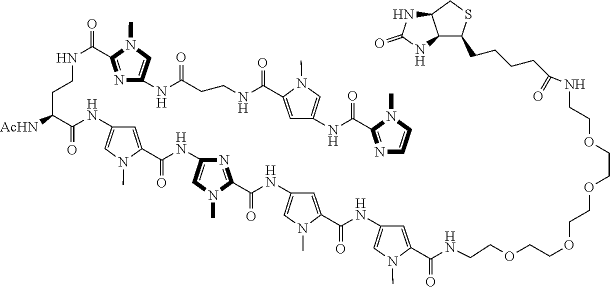 |
| 58 | 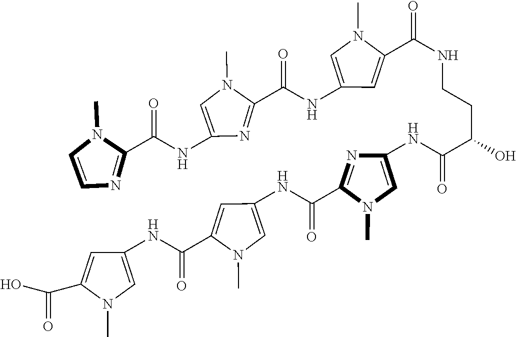 |
| 59 | 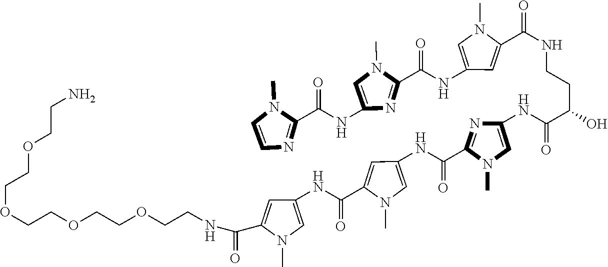 |
| 60 | 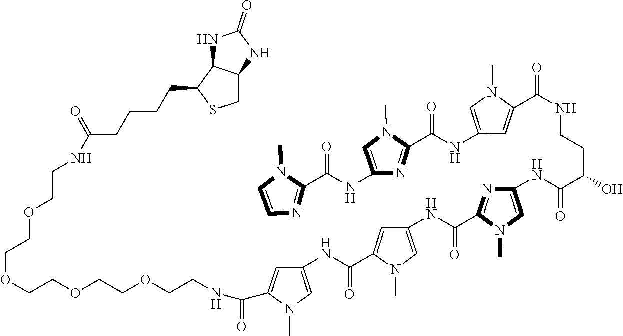 |
| 61 | 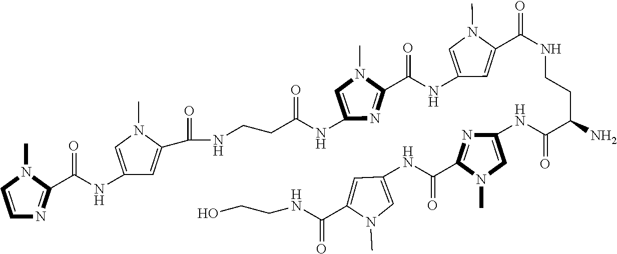 |
| 62 | 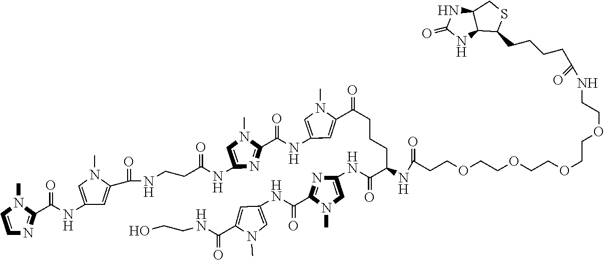 |
| 63 | 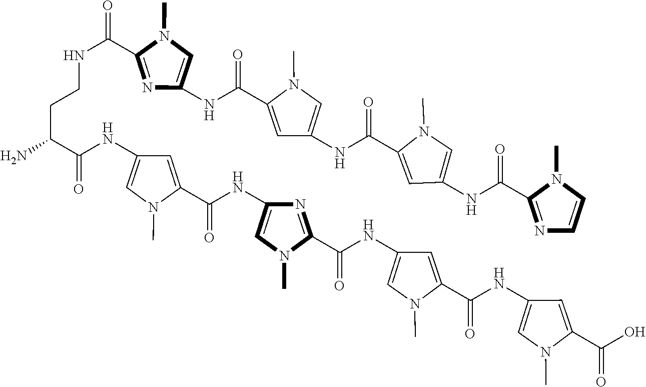 |
| 64 | 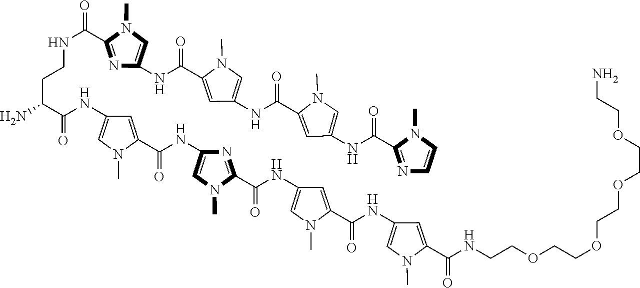 |
| 65 | 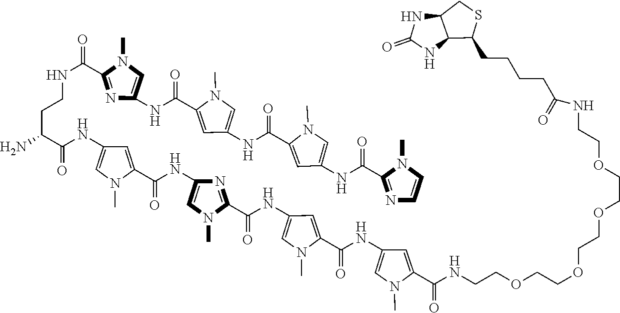 |
| 66 | 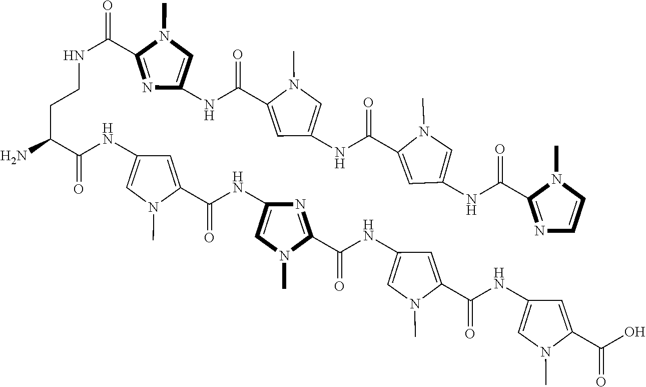 |
| 67 | 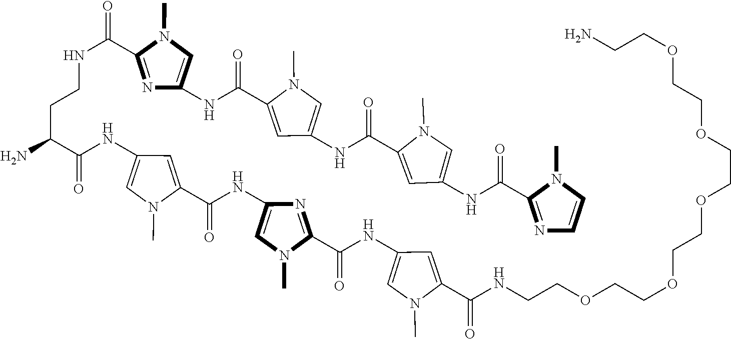 |
| 68 | 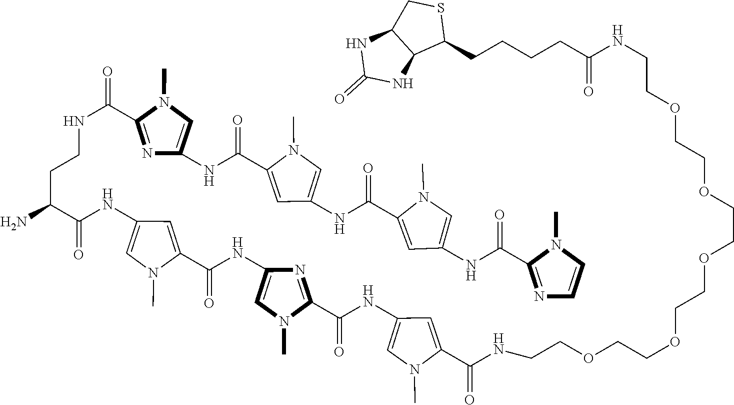 |
| 69 | 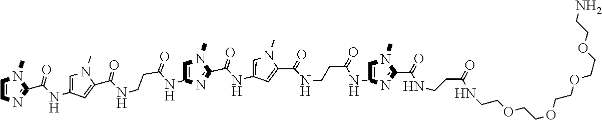 |
| 70 | 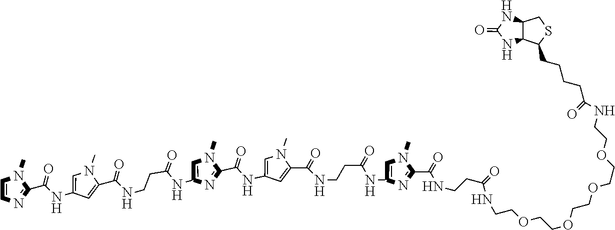 |
| 71 | 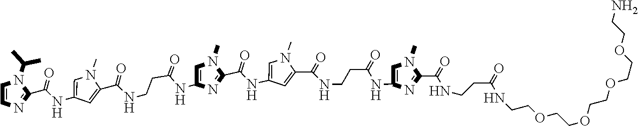 |
| 72 | 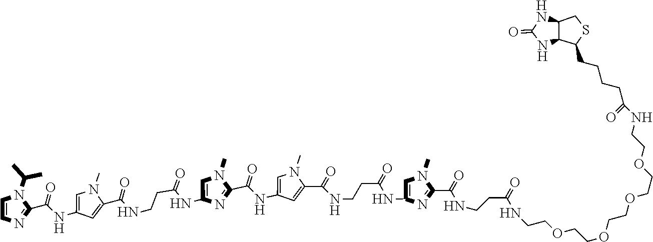 |
| 73 | 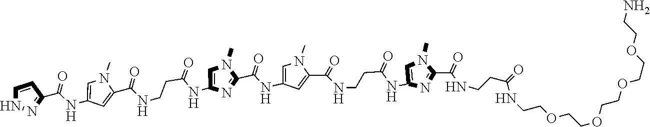 |
| 74 | 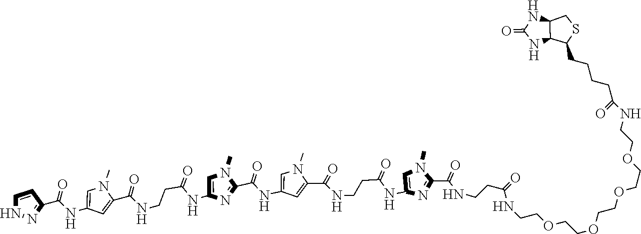 |
| 75 | 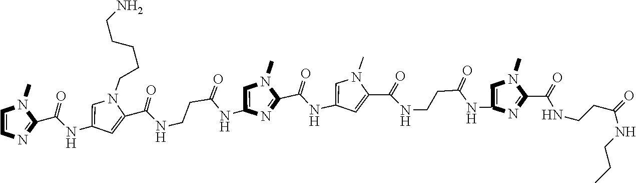 |
| 76 | 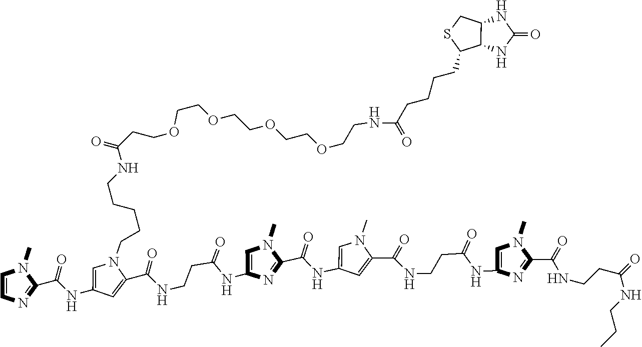 |
| 77 | 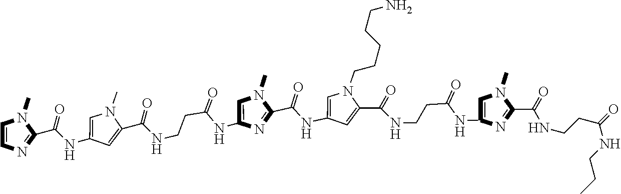 |
| 78 | 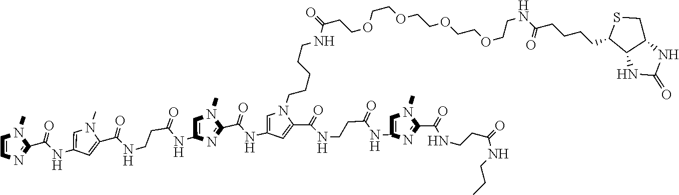 |
| 79 | 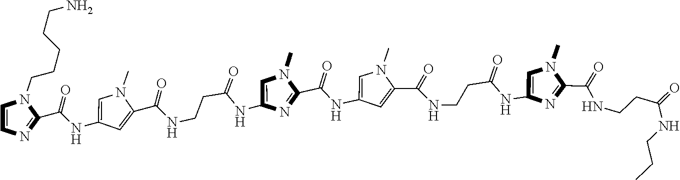 |
| 80 | 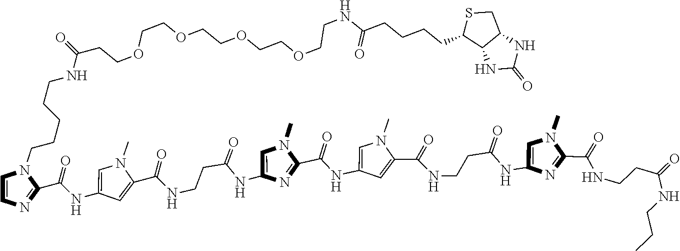 |
| 81 | 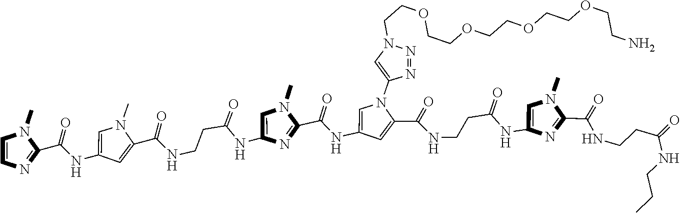 |
| 82 | 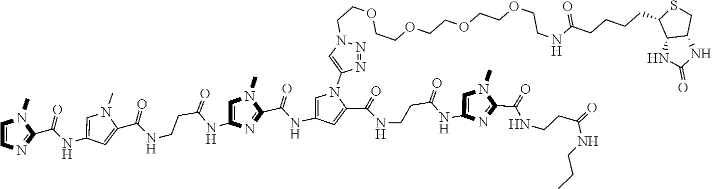 |
| 83 | 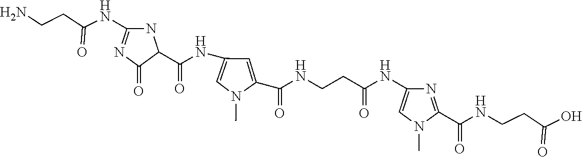 [SMILES: Cn1cc(NC(=O)C2NC(NC(=O)CCN)=NC2=O)cc1C(=O)NCCC(=O)Nc1cn(C)c(C(=O)NCCC(=O)O)n1] |
| 84 | 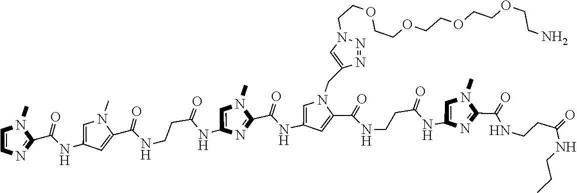 |
| 85 | 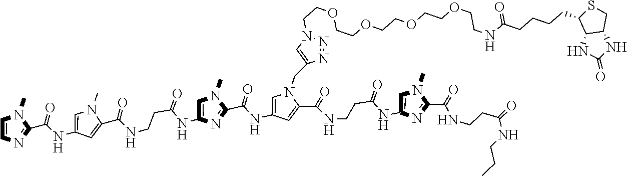 |
| 86 | 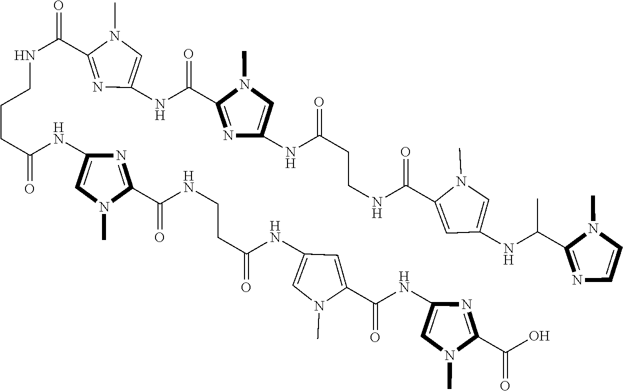 |
| 87 | 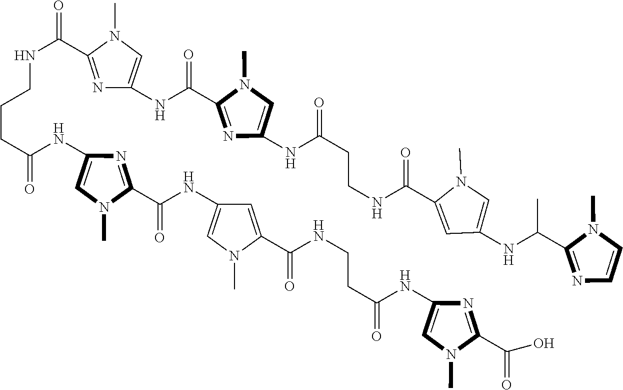 |
| 88 | 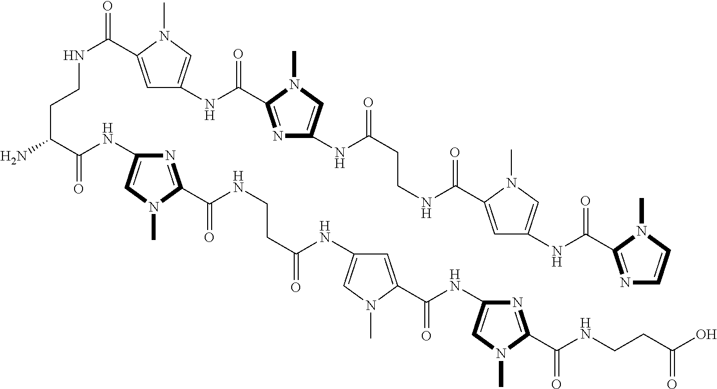 |
| 89 | 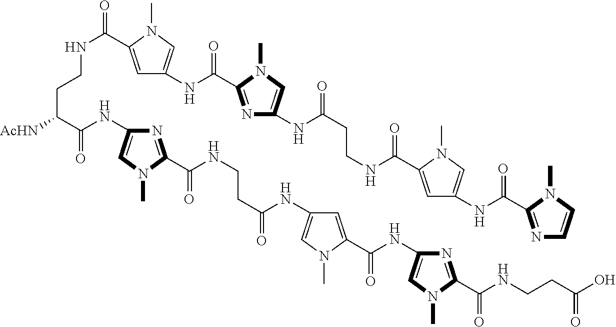 |
| 90 | 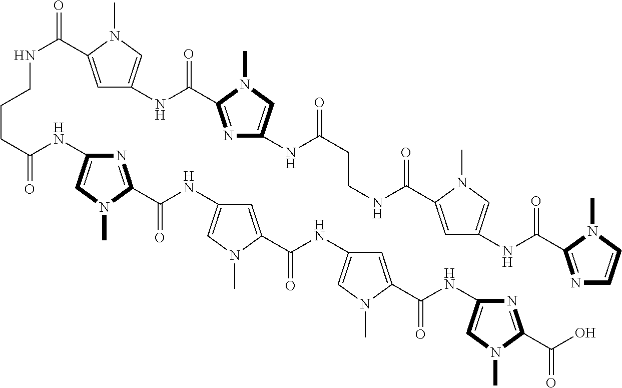 |
| 91 | 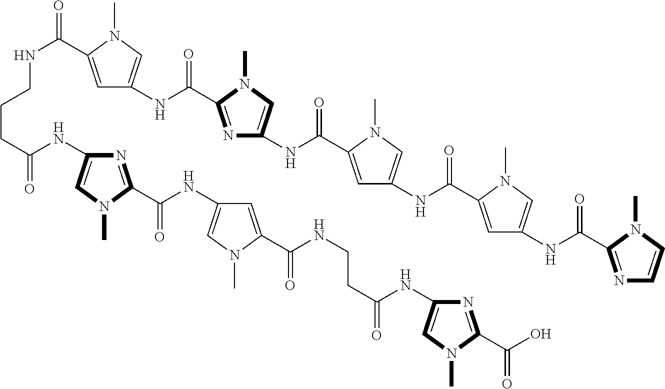 |
| 92 | 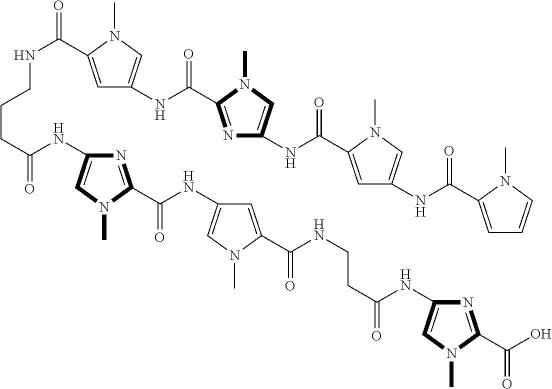 |
| 93 | 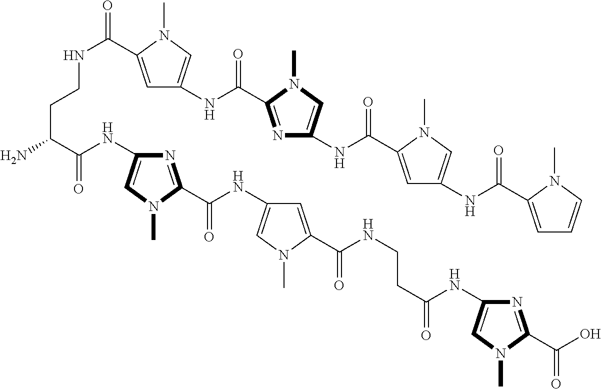 |
| 94 | 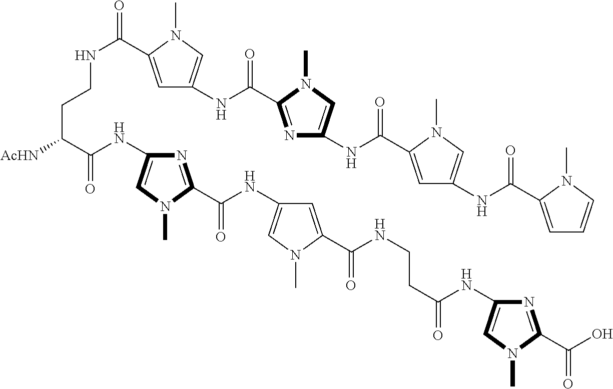 |
| 95 | 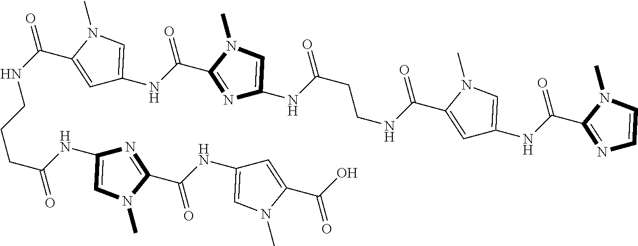 |
| 96 | 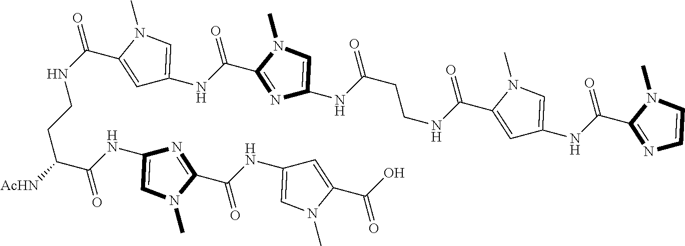 |
| 97 | 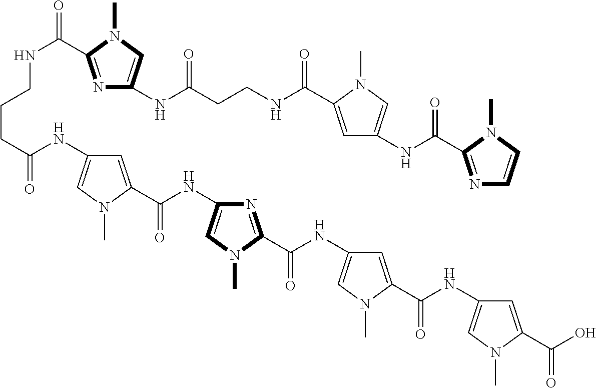 |
| 98 | 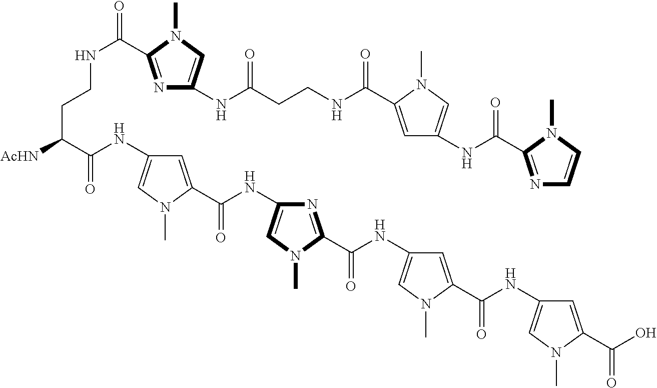 |
| 99 | 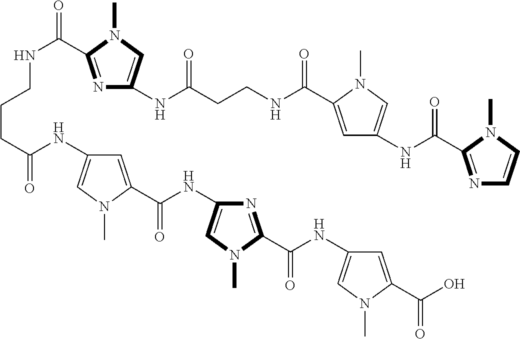 |
| 100 | 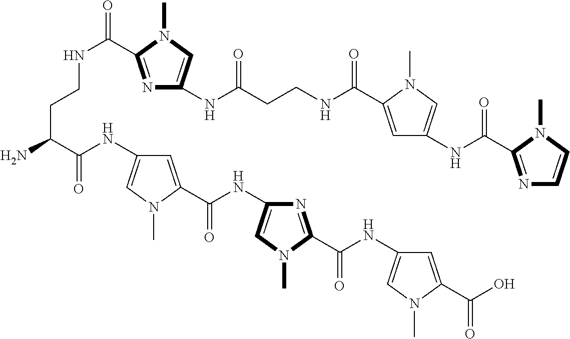 |
| 101 | 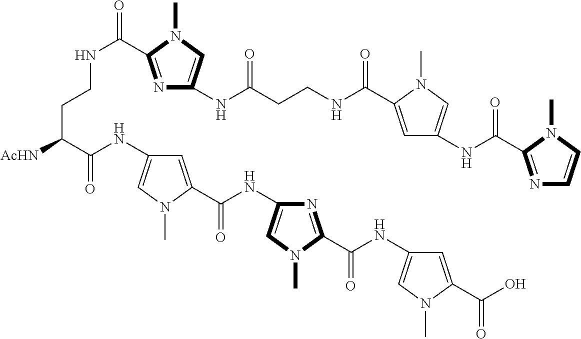 |
| 102 | 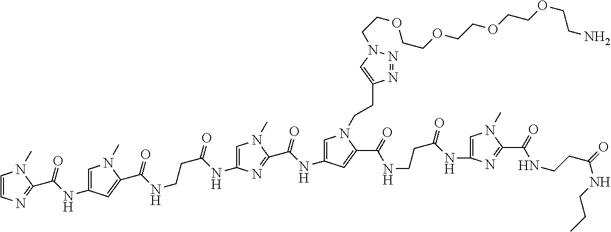 [SMILES: CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2CCc2cn(CCOCCOCCOCCOCCN)nn2)cn1C] |
| 103 | 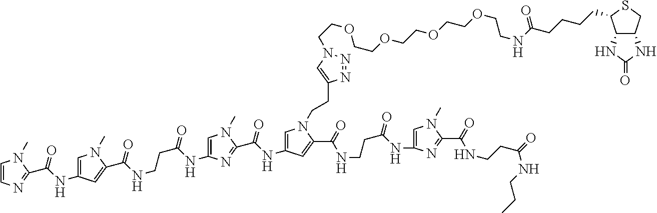 [SMILES: CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2CCc2cn(CCOCCOCCOCCOCCNC(=O)CCCC[C@@H]3SC[C@@H]4NC(=O)N[C@@H]43)nn2)cn1C] |
| 104 | 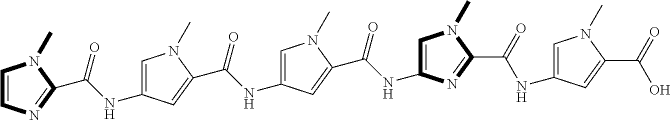 |
| 105 | 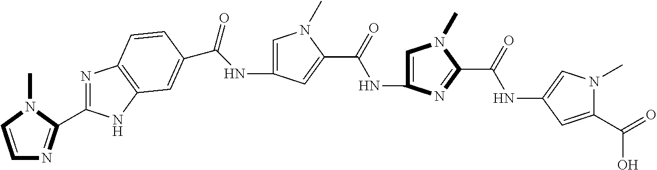 |
| 106 | 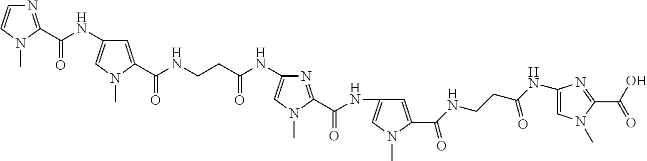 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)O)n1] |
| 107 | 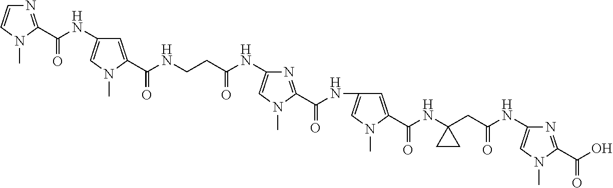 [SMILES: Cn1cc(NC(=O)c2nccn2C)cc1C(=O)NCCC(=O)Nc1cn(C)c(C(=O)Nc2cc(C(=O)NC3(CC(=O)Nc4cn(C)c(C(=O)O)n4)CC3)n(C)c2)n1] |
| 108 | 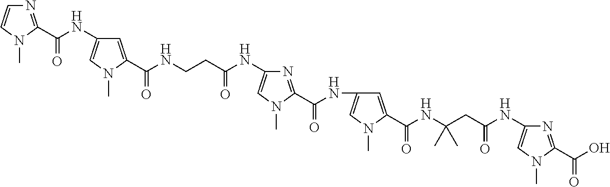 [SMILES: Cn1cc(NC(=O)c2nccn2C)cc1C(=O)NCCC(=O)Nc1cn(C)c(C(=O)Nc2cc(C(=O)NC(C)(C)CC(=O)Nc3cn(C)c(C(=O)O)n3)n(C)c2)n1] |
| 109 | 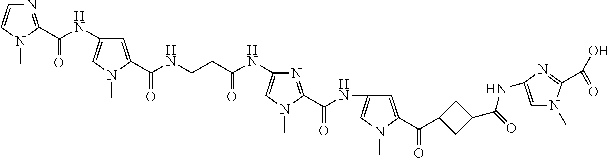 [SMILES: Cn1cc(NC(=O)c2nccn2C)cc1C(=O)NCCC(=O)Nc1cn(C)c(C(=O)Nc2cc(C(=O)C3CC(C(=O)Nc4cn(C)c(C(=O)O)n4)C3)n(C)c2)n1] |
| 110 | 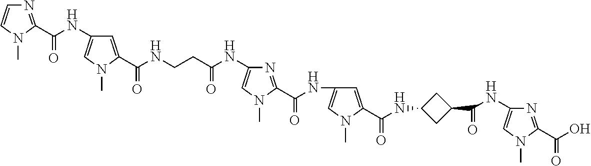 [SMILES: Cn1cc(NC(=O)c2nccn2C)cc1C(=O)NCCC(=O)Nc1cn(C)c(C(=O)Nc2cc(C(=O)N[C@H]3C[C@H](C(=O)Nc4cn(C)c(C(=O)O)n4)C3)n(C)c2)n1] |
| 111 | 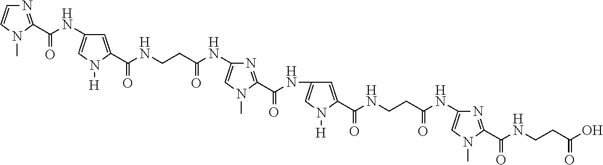 [SMILES: Cn1cc(NC(=O)CCNC(=O)c2cc(NC(=O)c3nc(NC(=O)CCNC(=O)c4cc(NC(=O)c5nccn5C)c[nH]4)cn3C)c[nH]2)nc1C(=O)NCCC(=O)O] |
| 112 | 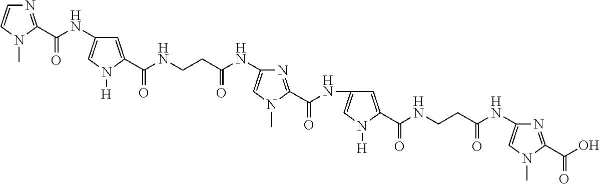 [SMILES: Cn1cc(NC(=O)CCNC(=O)c2cc(NC(=O)c3nc(NC(=O)CCNC(=O)c4cc(NC(=O)c5nccn5C)c[nH]4)cn3C)c[nH]2)nc1C(=O)O] |
| 113 | 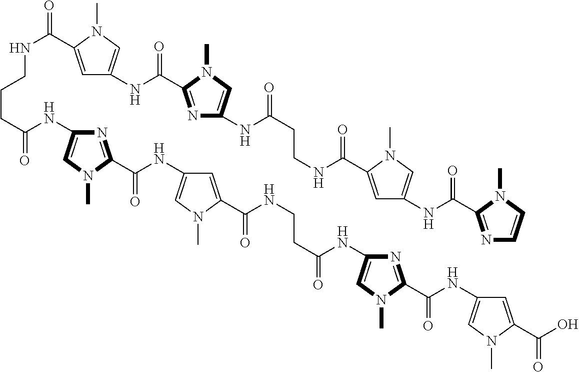 |
| 114 | 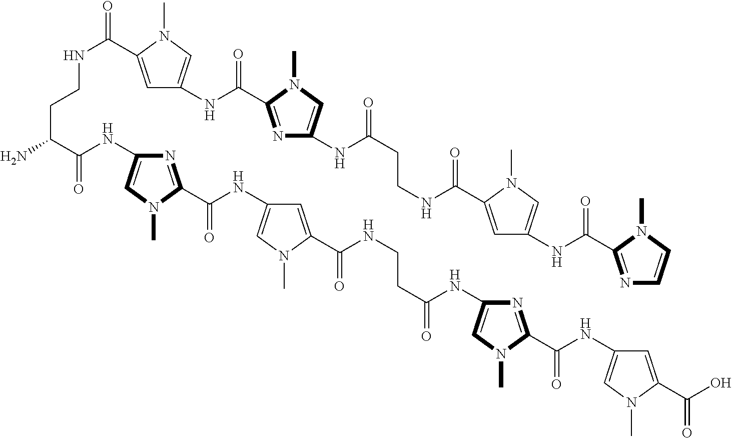 |
| 115 | 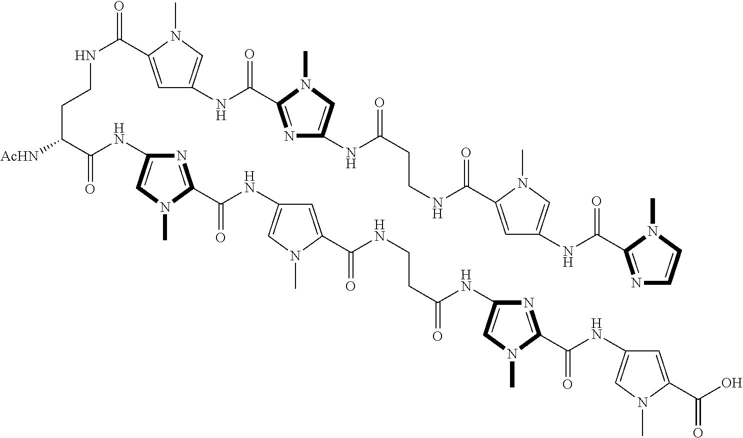 |
| 116 | 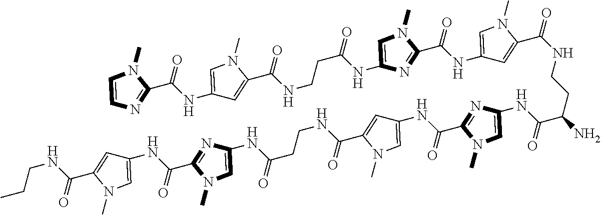 |
| 117 | 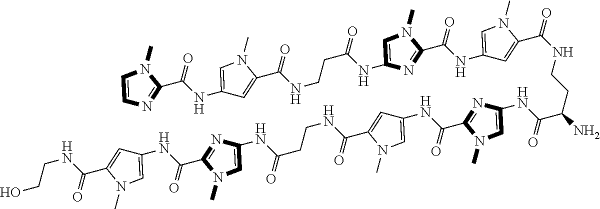 |
| 118 | 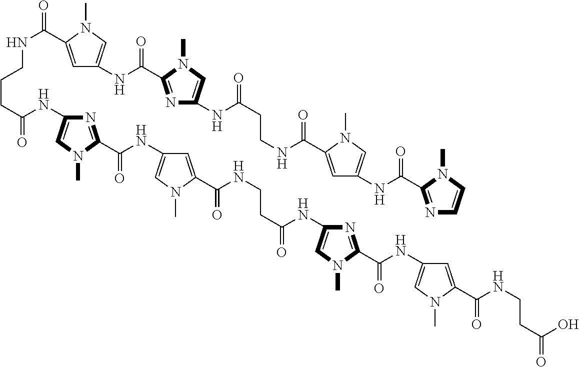 |
| 119 | 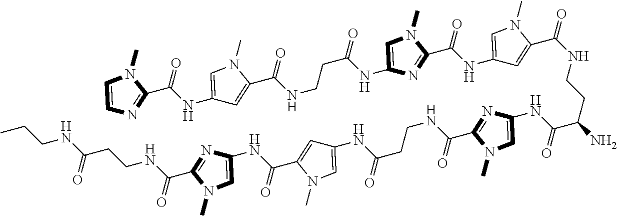 |
| 120 | 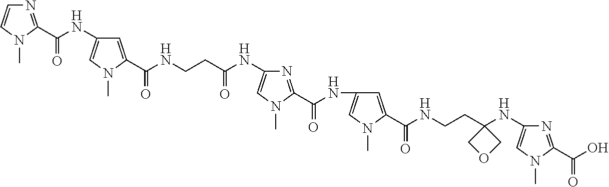 [SMILES: Cn1cc(NC(=O)c2nccn2C)cc1C(=O)NCCC(=O)Nc1cn(C)c(C(=O)Nc2cc(C(=O)NCCC3(Nc4cn(C)c(C(=O)O)n4)COC3)n(C)c2)n1] |
| 121 | 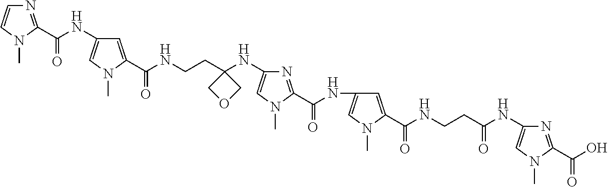 [SMILES: Cn1cc(NC(=O)c2nc(NC3(CCNC(=O)c4cc(NC(=O)c5nccn5C)cn4C)COC3)cn2C)cc1C(=O)NCCC(=O)Nc1cn(C)c(C(=O)O)n1] |
| 122 | 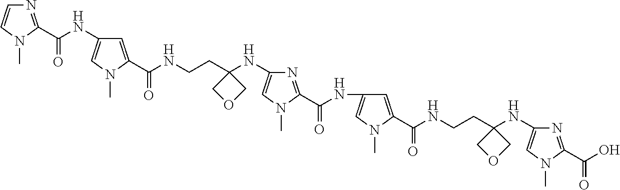 [SMILES: Cn1cc(NC(=O)c2nc(NC3(CCNC(=O)c4cc(NC(=O)c5nccn5C)cn4C)COC3)cn2C)cc1C(=O)NCCC1(Nc2cn(C)c(C(=O)O)n2)COC1] |
| 123 | 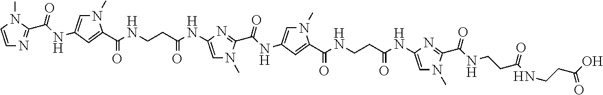 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCC(=O)O)n1] |
| 124 | 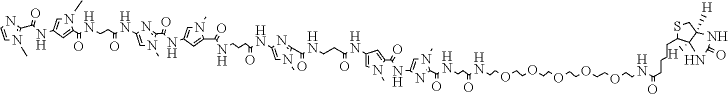 |
| 125 | 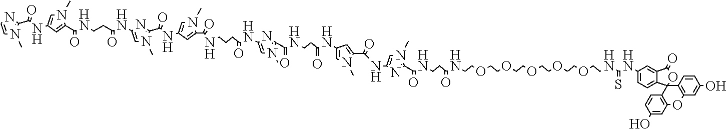 |
| 126 | 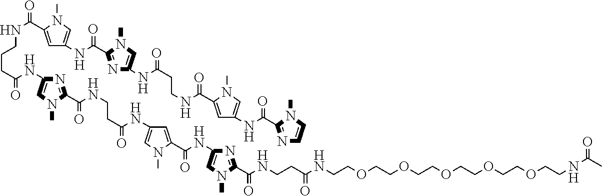 |
| 127 | 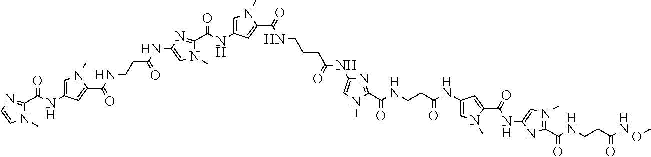 |

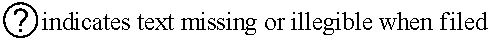

### Further Forms of the Polyamides and Pharmaceutical Compositions

Included in the present disclosure are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The polyamides presented herein, in some embodiments, exist as tautomers. In circumstances where tautomerization is possible, a chemical equilibrium of the tautomers will exist. The exact ratio of the tautomers depends on several factors, including physical state, temperature, solvent, and pH.

The polyamides disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include polyamides which differ only in the presence of one or more isotopically enriched atoms. For example, polyamides having the present structures except for the replacement of a hydrogen by a deuterium or tritium, or the replacement of a carbon by ¹³C- or ¹⁴C-enriched carbon are within the scope of the present disclosure.

The polyamides of the present disclosure optionally contain unnatural proportions of atomic isotopes at one or more atoms that constitute such polyamides. For example, the polyamides may be labeled with isotopes, such as for example, deuterium (²H), tritium (³H), iodine-125 (¹²⁵I) or carbon-14 (¹⁴C). Isotopic substitution with ²H, ¹¹C ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In some embodiments of the polyamides disclosed herein, one or more of R¹, R², R³, R⁴, R⁵, R^{A}, R^{B}, R^{C}, and R^{D} groups comprise deuterium at a percentage higher than the natural abundance of deuterium.

In some embodiments of the polyamides disclosed herein, one or more hydrogens are replaced with one or more deuteriums in one or more of the following groups R¹, R², R³, R⁴, R⁵, R^{A}, R^{B}, R^{C}, and R^{D}.

In some embodiments, the polyamides disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Also provided herein, are pharmaceutically acceptable compositions comprising a polyamide of Formula (I) or (II), or a pharmaceutically acceptable salt thereof, and a pharmaceutically acceptable carrier or excipient.

Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical composition comprising, for example, a pharmaceutical agent and a pharmaceutically acceptable carrier or excipient. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system, e.g., a skin patch.

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self-emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue; absorption through the oral mucosa, e.g., sublingually; anally, rectally or vaginally, for example, as a pessary, cream or foam; parenterally, including intramuscularly, intravenously, subcutaneously or intrathecally as, for example, a sterile solution or suspension; nasally; intraperitoneally; subcutaneously; transdermally, for example, as a patch applied to the skin; and topically, for example, as a cream, ointment or spray applied to the skin. The compound may also be formulated for inhalation. In certain embodiments, a compound may be simply dissolved or suspended in sterile water.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the subject, the type and severity of the subjects' disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

Pharmaceutical compositions comprising a pharmaceutical agent can be formulated in a manner appropriate for the delivery method by using techniques routinely practiced in the art. The composition may be in the form of a solid, e.g., tablet, capsule, semi-solid, e.g., gel, liquid, or gas, e.g., aerosol. In other embodiments, the pharmaceutical composition is administered as a bolus infusion.

### Methods of Use

In another aspect provide herein, the polyamides of Formula (I) or (II) or a pharmaceutically acceptable salt thereof, can bind to a nucleotide repeat. In some embodiments, the polyamide comprises a polymeric sequence of monomers, wherein each monomer in the polymer selectively binds to a certain DNA base pair. In some embodiments, the polyamides disclosed herein bind in the minor groove of DNA. In some embodiments, the polyamides bind non-covalently.

In some embodiments, the polyamide binds to a sequence comprising at least 1000 pentanucleotide repeats. In some embodiments, the polyamide binds to a sequence comprising at least 500 trinucleotide repeats. In some embodiments, the polyamide binds to a sequence comprising at least 200 trinucleotide repeats. In some embodiments, the polyamide binds to a sequence comprising at least 100 trinucleotide repeats. In some embodiments, the polyamide moiety binds to a sequence comprising at least 50 trinucleotide repeats. In some embodiments, the polyamide moiety binds to a sequence comprising at least 20 trinucleotide repeats.

In some embodiments, the nucleotide repeat sequence is selected from GAA, CAG, CTG, CGG, CCTG, TGGAA, or GGGGCC. In some embodiments, the nucleotide repeat is GAA. In some embodiments, the nucleotide repeat is CAG or CTG. In some embodiments, the nucleotide repeat is CAG. In some embodiments, the nucleotide repeat is CGG. In some embodiments, the nucleotide repeat is CCTG. In some embodiments, the nucleotide repeat is TGGAA, In some embodiments, the nucleotide repeat is GGGGCC.

The binding affinity between the polyamide and the target gene can be adjusted based on the composition of the polyamide. In some embodiments, the polyamide is capable of binding the DNA with an affinity of less than about 600 nM, about 500 nM, about 400 nM, about 300 nM, about 250 nM, about 200 nM, about 150 nM, about 100 nM, or about 50 nM. In some embodiments, the polyamide is capable of binding the DNA with an affinity of less than about 300 nM. In some embodiments, the polyamide is capable of binding the DNA with an affinity of less than about 200 nM. In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, or 100-300 nM.

The binding affinity between the polyamide and the target DNA can be determined using a quantitative footprint titration experiment. The experiment involve measuring the dissociation constant K_{d} of the polyamide for target sequence at either 24° C. or 37° C., and using either standard polyamide assay solution conditions or approximate intracellular solution conditions.

In some embodiments, the polyamides of described herein provide an effective treatment for a disease or disorder which is characterized by the presence of an excessive count of a nucleotide repeat (e.g. the trinucleotide repeat sequence CGG) in a target gene. In some embodiments, the pathology of the disease or disorder is due to the presence of mRNA containing an excessive count of the target nucleotide repeat sequence (e.g. CGG). In some embodiments, the pathology of the disease or disorder is due to the presence of a translation product containing an excessive count of glutamine amino acid residues. In some embodiments, the pathology of the disease or disorder is due to a loss of function in the translation product. In some embodiments, the pathology of the disease or disorder is due to a gain of function in the translation product. In some embodiments, the pathology of the disease or disorder can be alleviated by increasing the rate of transcription of the defective gene. In some embodiments, the pathology of the disease or disorder can be alleviated by decreasing the rate of transcription of the defective gene.

### Definitions

Unless defined otherwise, all technical and scientific terms used herein have the same meaning as is commonly understood by one of skill in the art to which this invention belongs.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkoxy” refers to a radical bonded through an oxygen atom of the formula —O-alkyl, where alkyl is an alkyl chain as defined above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₈ alkylene). Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃—C alkenylene). Unless stated otherwise specifically in the specification, an alkenylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenylene is optionally substituted with oxo, halogen, —CN, —COOH, COOMe, —OH, —OMe, —NH₂, or —NO₂.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₃-C₈ alkynylene). In other embodiments, an alkynylene comprises three to five carbon atoms (i.e., C₃-C₅ alkynylene). Unless stated otherwise specifically in the specification, an alkynylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynylene is optionally substituted with oxo, halogen, —CN, —COOH, COOMe, —OH, —OMe, —NH₂, or —NO₂.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R^{e} is an alkynylene chain as defined above.

“Carbocycle” refers to a saturated, unsaturated or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a fully saturated monocyclic or polycyclic hydrocarbon radical consisting solely of carbon and hydrogen atoms, which includes fused or bridged ring systems, and preferably having from three to twelve carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may also refer to a partially saturated, monocyclic or polycyclic carbocyclic ring such as a cycloalkenyl. A cycloalkyl includes fused (when fused with an aryl or a heteroaryl ring, the cycloalkyl is bonded through a non-aromatic ring atom), spiro, or bridged ring systems. Representative cycloalkyls include, but are not limited to, cycloalkyls having from three to fifteen carbon atoms (C₃-C₁₅ cycloalkyl, including C₃-C₁₅ cycloalkenyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 10-membered cycloalkyl, e.g., a 3- to 10-membered cycloalkenyl. In some embodiments, the cycloalkyl is a 3- to 6-membered cycloalkyl, e.g., a 3- to 6-membered cycloalkenyl. In some embodiments, the cycloalkyl is a 5- to 6-membered cycloalkyl, e.g., a 5-to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁s heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂.

“Heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. “Heterocyclene” refers to a divalent heterocycle linking the rest of the molecule to a radical group.

“Heteroaryl” or “aromatic heterocycle” refers to a radical derived from a heteroaromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, thiophene, benzthiazole, and imdazopyridine. An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

The term “substituted” refers to moieties having substituents replacing a hydrogen on one or more carbons or substitutable heteroatoms, e.g., NH₄, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds, In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene”, “heteroarylene.”

In some embodiments, substituents may include any substituents described herein, for example: halogen, hydroxy, oxo (=O), thioxo (=S), cyano (—CN), nitro (—NO₂), imino (=N—H), oximo (=N—OH), hydrazino (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (=O), thioxo (=S), cyano (—CN), nitro (—NO₂), imino (=N—H), oximo (=N—OH), hydrazine (=N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)OR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R is a straight or branched alkylene, alkenylene or alkynylene chain.

As used in the specification and claims, the singular form “a”, “an” and “the” includes plural references unless the context clearly dictates otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “polyamide” refers to polymers of linkable units chemically bound by amide (i.e., CONH) linkages; optionally, polyamides include chemical probes conjugated therewith. Polyamides may be synthesized by stepwise condensation of carboxylic acids (COOH) with amines (RR′NH) using methods known in the art. Alternatively, polyamides may be formed using enzymatic reactions in vitro, or by employing fermentation with microorganisms.

The term “linkable unit” refers to methylimidazoles, methylpyrroles, and straight and branched chain aliphatic functionalities (e.g., methylene, ethylene, propylene, butylene, and the like) which optionally contain nitrogen Substituents, and chemical derivatives thereof. The aliphatic functionalities of linkable units can be provided, for example, by condensation of B-alanine or dimethylaminopropylamine during synthesis of the polyamide by methods well known in the art.

The terms “nucleic acid and “nucleotide” refer to ribonucleotide and deoxyribonucleotide, and analogs thereof, well known in the art.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences.

The term “transcription,” well known in the art, refers to the synthesis of RNA (i.e., ribonucleic acid) by DNA-directed RNA polymerase. The term “modulate transcription” refers to a change in transcriptional level which can be measured by methods well known in the art, for example, assay of mRNA, the product of transcription. In certain embodiments, modulation is an increase in transcription. In other embodiments, modulation is a decrease in transcription.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

### Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

### List of Abbreviation

Ac₂O=acetic anhydride; AcCl=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(O); Pd₂(dba)₃=tris(dibenzylideneacetone)dipalladium(O); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)-tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TBS=TBDMS=tert-butyldimethylsilyl; TBSCl=TBDMSCl=tert-butyldimethylchlorosilane; TEA=Et₃N=triethylamine; TFA=trifluoroacetic acid; TFAA=trifluoroacetic anhydride; THF=tetrahydrofuran; Tol=toluene; TsCI=tosyl chloride; XPhos=2-dicyclohexylphosphino-2′,4′,6′-triisopropylbiphenyl.

### General Synthetic Methods for Preparing Compounds

In general, polyamides of the present disclosure may be synthesized by solid supported synthetic methods, using compounds such as Boc-protected straight chain aliphatic and heteroaromatic amino acids, and alkylated derivatives thereof, which are cleaved from the support by aminolysis, deprotected (e.g., with sodium thiophenoxide), and purified by reverse-phase HPLC, as well known in the art. The identity and purity of the polyamides may be verified using any of a variety of analytical techniques available to one skilled in the art such as ¹H-NMR, analytical HPLC, or mass spectrometry.

The following scheme can be used to practice the present disclosure:

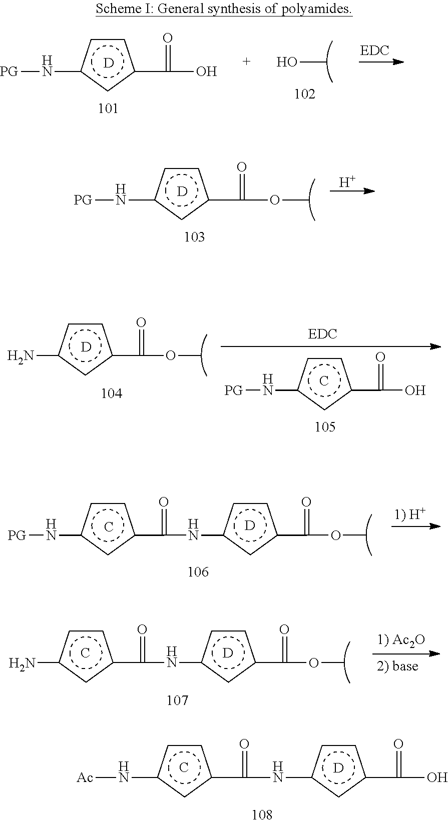

The compounds disclosed herein can be synthesized using Scheme I. For clarity and compactness, the scheme depicts the synthesis of a diamide comprising subunits “C” and “D”, both of which are represented as unspecified five-membered rings having amino and carboxy moieties. The amino group of subunit “D” is protected with a protecting group “PG” such as a Boc or CBz carbamate to give 101. The free)carboxylic acid is then reacted with a solid support, using a coupling reagent such as EDC, to give the supported compound 103. Removal of PG under acidic conditions gives the free amine 104, which is coupled with the nitrogen-protected carboxylic acid 105 to give amide 106. Removal of PG under acidic conditions gives the free amine 107. In this example, the free amine is reacted with acetic anhydride to form an acetamide (not shown. The molecule is then cleaved from the solid support under basic conditions to give carboxylic acid 108. Methods for attachment of the linker L and recruiting moiety X are disclosed below.

The person of skill will appreciate that many variations of the above scheme are available to provide a wide range of compounds:
1) The sequence 104-106-107 can be repeated as often as desired, in order to form longer polyamine sequences.
2) A variety of amino heterocycle carboxylic acids can be used, to form different subunits. Table 4, while not intended to be limiting, provides several heterocycle amino acids that are contemplated for the synthesis of the compounds in this disclosure. Carbamate protecting groups PG can be incorporated using techniques that are well established in the art.

TABLE 2
Heterocyclic amino acids.
Structure

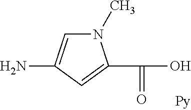

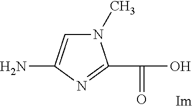

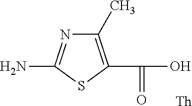

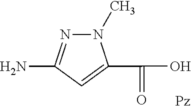

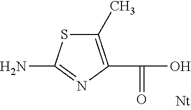

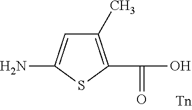

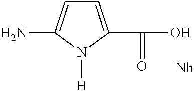

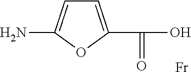

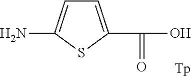

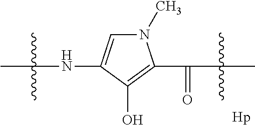 [SMILES: *Nc1cn(C)c(C(*)=O)c1O ; "Hp"]

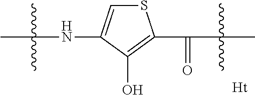 [SMILES: *Nc1csc(C(*)=O)c1O ; "Ht"]

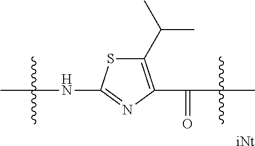 [SMILES: *Nc1nc(C(*)=O)c(C(C)C)s1 ; "iNt"]

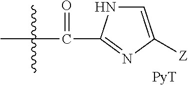

(Z is H, C₁₋₆ alkyl, amine, or halogen)

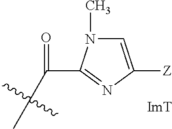

(Z is H, C₁₋₆ alkyl, amine, or halogen)

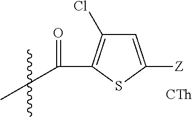

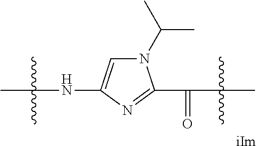 [SMILES: *Nc1cn(C(C)C)c(C(*)=O)n1 ; "iIm"]

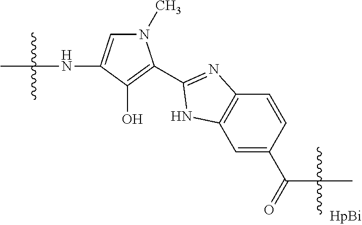 [SMILES: *Nc1cn(C)c(-c2nc3ccc(C(*)=O)cc3[nH]2)c1O]

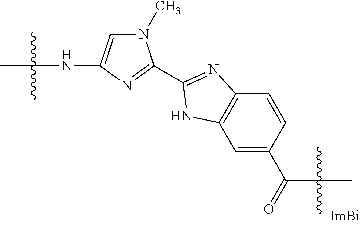 [SMILES: *Nc1cn(C)c(-c2nc3ccc(C(*)=O)cc3[nH]2)n1]

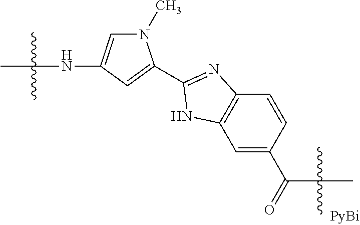 [SMILES: *Nc1cc(-c2nc3ccc(C(*)=O)cc3[nH]2)n(C)c1]

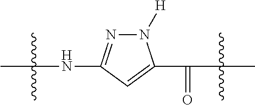 [SMILES: *Nc1cc(C(*)=O)[nH]n1]

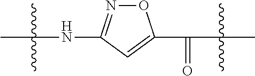 [SMILES: *Nc1cc(C(*)=O)on1]

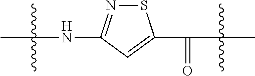 [SMILES: *Nc1cc(C(*)=O)sn1]

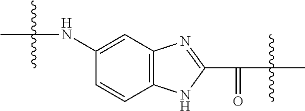 [SMILES: *Nc1ccc2[nH]c(C(*)=O)nc2c1]

3) Hydroxy-containing heterocyclic amino acids can be incorporated into Scheme I as their TBS ethers. While not intended to be limiting, Scheme II provides the synthesis of TBS-protected heterocyclic amino acids contemplated for the synthesis of the molecules in this disclosure.

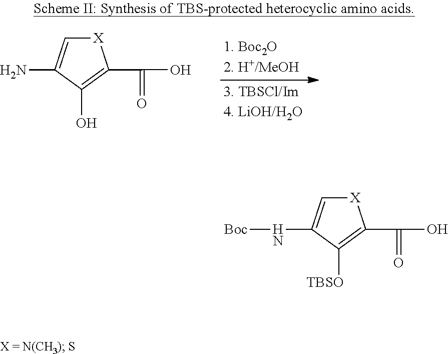

4) Aliphatic amino acids can be used in the above synthesis for the formation of spacer units “W” and subunits for recognition of DNA nucleotides. Table 5, while not intended to be limiting, provides several aliphatic amino acids contemplated for the synthesis of the or molecules in this disclosure.

TABLE 3
Aliphatic amino acids.

| Structure | |
|---|---|
| 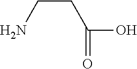 | beta-alanine (ß) |
| 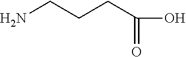 | gamma-aminobutyric acid (“gAB” or ß) |
| 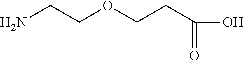 | 3-(2-aminoethoxy) propanoic acid |
| 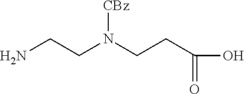 | 3-((2-aminoethyl)(2- oxo-2-phenyl-1ß²- ethyl)amino)propanoic acid |
| 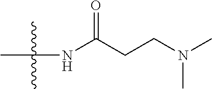 [SMILES: *NC(=O)CCN(C)C] | Dp |
| 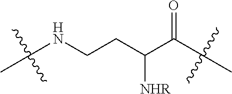 | (R is H, C₁₋₆ alkyl) |
| 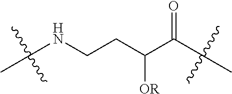 | (R is H, C₁₋₆ alkyl, aryl, or heteroaryl) |
| 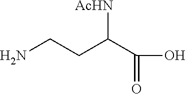 [SMILES: CC(=O)NC(CCN)C(=O)O] | |
| 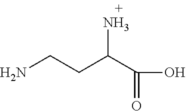 | |
| 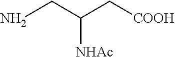 [SMILES: CC(=O)NC(CN)CC(=O)O] | |
| 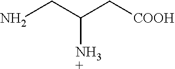 | |
| 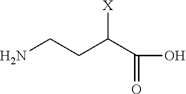 | X is F or OH |

### EXAMPLES

The following examples are given as examples for solution phase synthesis for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

### Example 1. Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (Compound 1)

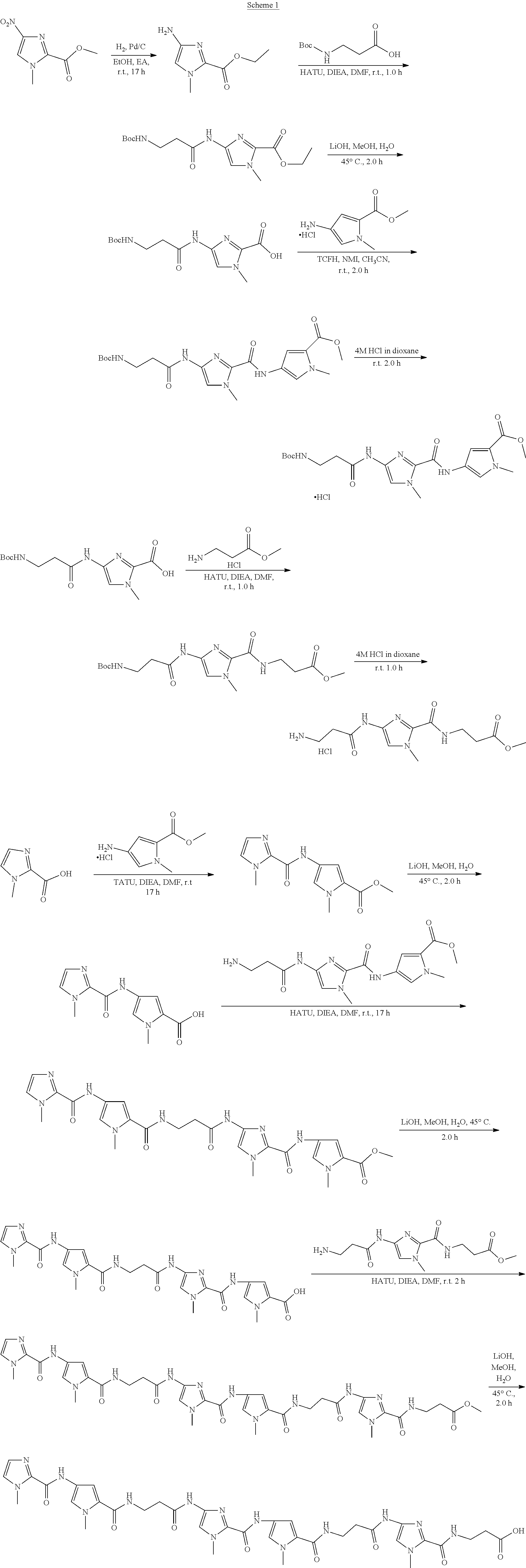

### Step 1: Synthesis of ethyl 4-amino-1-methylmidazole-2-carboxylate

To a solution of ethyl 1-methyl-4-nitroimidazole-2-carboxylate (30.00 g, 150.63 mmol, 1.00 equiv) in EtOH (120.00 mL) was added Pd/C (8.01 g, 27% w/w). Then the reaction was stirred for 17 h at room temperature under H₂ atmosphere. The solid was filtrated out and the filtrate was concentrated to afford ethyl 4-amino-1-methylimidazole-2-carboxylate (22.30 g, 75.20%) as yellow solid. LC/MS: mass calcd. For C₇H₁₁N₃O₂: 169.09, found: 170.10 [M+H]⁺. ¹H NMR (400 MHz, DMSO-d₆) δ: 7.37 (s, 1H), 4.29-4.34 (m, 2H), 3.94 (s, 3H), 1.31 (t, J=7.2 Hz, 3H).

### Step 2: Synthesis of ethyl 4-[3-[(tert-butoxycarbonyl)amino]propanamido]-1-methylimidazole-2-carboxylate

Into a 500 mL flask was added 3-[(tert-butoxycarbonyl) amino]propanoic acid (22.45 g, 118.65 mmol, 0.90 equiv), DMF (180.00 mL). The mixture was cooled to 0 degrees C., then HATU (75.18 g, 197.71 mmol, 1.50 equiv) and DIEA (51.11 g, 395.43 mmol, 3.00 equiv) were added, the mixture was stirred for 10 mins, then ethyl 4-amino-1-methylimidazole-2-carboxylate (22.30 g, 131.81 mmol, 1.00 equiv) was added in portions. The reaction was stirred at room temperature for 1 h. The reaction was quenched with ice water (600 mL), and the solution was stirred for 15 min. The precipitated solids were collected by filtration and washed with water (3×50 mL) and dried under vacuum. This resulted in ethyl 4-[3-[(tert-butoxycarbonyl)amino]propanamido]-1-methylimidazole-2-carboxylate (34.50 g, 76.90%) as light yellow solid. LC/MS: mass calcd. For C₁₅H₂₄N₄O₅: 340.17, found: 341.20 [M+H]⁺. ¹H NMR (400 MHz, DMSO-d₆) δ: 10.63 (s, 1H), 7.52 (s, 1H), 6.80 (t, J=5.6 Hz, 1H), 4.23-4.28 (m, 2H), 3.90 (s, 3H), 3.15-3.20 (m, 2H), 2.42 (t, J=7.2 Hz, 2H), 1.37 (s, 9H), 1.29 (t, J=7.2 Hz, 3H).

### Step 3: Synthesis of 4-[3-[(Tert-butoxycarbonyl)amino]propanamido]-1-methylimidazole-2-carboxylic acid

To a stirred solution of ethyl 4-[3-[(tert-butoxycarbonyl)amino]propanamido]-1-methylimidazole-2-carboxylate (34.50 g, 101.36 mmol, 1.00 equiv) in MeOH (200.00 mL) was added LiOH solution (2 M, 202.00 mL, 4.00 equiv) dropwise at room temperature. The resulting mixture was stirred for 2 h at 45 degree C. The resulting mixture was concentrated under reduced pressure. The residue was dissolved in H₂O (50 mL). The mixture was acidified to pH 3-5 with 2M HCl. The precipitated solids were collected by filtration and washed with H₂O (3×30 mL), dried under vacuum. 4-[3-[(Tert-butoxycarbonyl)amino]propanamido]-1-methylimidazole-2-carboxylic acid (30.00 g, 94.77%) was obtained as white solid. LC/MS: mass calcd. For C₁₃H₂₀N₄O₅: 312.14, found: 313.15 [M+H]⁺. ¹H NMR (300 MHz, DMSO-d₆) δ: 10.53 (s, 1H), 7.48 (s, 1H), 6.79 (t, J=5.4 Hz, 1H), 3.89 (s, 3H), 3.15-3.22 (m, 2H), 2.43 (t, J=7.2 Hz, 2H), 1.37 (s, 9H).

### Step 4: Synthesis of Methyl 4-(4-[3-[(tert-butoxycarbonyl)amino]propanamido]-1-methylimidazole-2-amido)-1-methylpyrrole-2-carboxylate

To a stirred solution of 4-[3-[(tert-butoxycarbonyl)amino]propanamido]-1-methylimidazole-2-carboxylic acid (16.00 g, 51.23 mmol, 1.00 equiv) in CH₃CN (150.00 mL) was added TCFH (21.56 g, 76.84 mmol, 1.50 equiv), NMI (12.62 g, 153.69 mmol, 3.00 equiv) and methyl 4-amino-1-methylpyrrole-2-carboxylate hydrochloride (10.74 g, 56.34 mmol, 1.10 equiv) in portions at 0 degrees C. The resulting mixture was stirred for 2.0 h at room temperature. The precipitated solids were collected by filtration and washed by CH₃CN (3×20 mL), dried under vacuum. Methyl 4-(4-[3-[(tert-butoxycarbonyl)amino]propanamido]-1-methylimidazole-2-amido)-1-methylpyrrole-2-carboxylate (19.00 g, 82.70%) was obtained as white solid. LC/MS: mass calcd. For C₂₀H₂₈N₆O₆: 448.21, found: 449.25 [M+H]. ¹H NMR (300 MHz, DMSO-d₆) δ: 10.24 (s, 1H), 10.11 (s, 1H), 7.52 (s, 1H), 7.33 (s, 1H), 6.99 (s, 1H), 6.82 (t, J=5.1 Hz, 1H), 3.94 (s, 3H), 3.85 (s, 3H), 3.74 (s, 3H), 3.16-3.23 (m, 2H), 2.47 (t, J=6.9 Hz, 2H), 1.38 (s, 9H).

### Step 5: Synthesis of Methyl 4-[4-(3-aminopropanamido)-1-methylimidazole-2-amido]-1-methylpyrrole-2-carboxylate hydrochloride

A solution of methyl 4-(4-[3-[(tert-butoxycarbonyl)amino]propanamido]-1-methylimidazole-2-amido)-1-methylpyrrole-2-carboxylate (19.00 g, 42.37 mmol, 1.00 equiv) in HCl/1,4-dioxane (4M, 200.00 mL) was stirred for 2 h at room temperature. The resulting mixture was concentrated under vacuum. Methyl 4-[4-(3-aminopropanamido)-1-methylimidazole-2-amido]-1-methylpyrrole-2-carboxylate hydrochloride (19.00 g crude) was obtained as yellow solid. LC/MS: mass calcd. For C₁₅H₂₁ClN₆O₄: 348.15, found: 349.05 [M+H]⁺. ¹H NMR (300 MHz, CD₃OD) δ: 7.37 (s, 2H), 6.91 (s, 1H), 4.03 (s, 3H), 3.88 (s, 3H), 3.79 (s, 3H), 3.09 (t, J=6.6 Hz, 2H), 2.64 (t, J=6.6 Hz, 2H).

### Step 6: Synthesis of methyl 3-[(4-[3-[(tert-butoxycarbonyl)amino]propanamido]-1-methylimidazol-2-yl)formamido]propanoate

Into a 1000 mL flask was added 4-[3-[(tert-butoxycarbonyl)amino] propanamido]-1-methylimidazole-2-carboxylic acid (11.00 g, 35.22 mmol, 1.00 equiv), DMF (300.00 mL), the mixture was cooled to 0 degrees C., then HATU (20.09 g, 52.83 mmol, 1.50 equiv), DIEA (18.21 g, 140.88 mmol, 4.00 equiv) was added dropwise, the mixture was stirred for 10 mins, methyl 3-aminopropanoate (3.63 g, 35.22 mmol, 1.00 equiv) was added in portions. The reaction was stirred at room temperature for 1 h. The reaction mixture was poured into ice/water (600 mL), the solid was filtered out and dried under vacuum. The aqueous phase was extracted by EA (3×200 mL), the organic phases were combined and washed by H₂O (1×200 mL) and NaCl (1×200 mL), dried over anhydrous Na₂SO₄. After filtration, the filtrate was concentrated under reduced pressure. The residue was purified by silica gel column, eluted with pure EA. The fractions were combined and concentrated. Methyl 3-[(4-[3-[(tert-butoxycarbonyl)amino]propanamido]-1-methylimidazol-2-yl)formamido]propanoate (13.00 g, 87.95%) was obtained as yellow solid. LC/MS: mass calcd. For C₁₇H₂₇N₅O₆: 397.20, found: 398.20 [M+H]⁺. ¹H NMR (400 MHz, DMSO-d₆) δ: 10.28 (s, 1H), 7.92 (t, J=6.0 Hz, 1H), 7.37 (s, 1H), 6.77 (t, J=6.0 Hz, 1H), 3.88 (s, 3H), 3.59 (s, 3H), 3.42-3.47 (m, 2H), 3.13-3.18 (m, 2H), 2.56 (t, J=6.0 Hz, 2H), 2.42 (t, J=6.0 Hz, 2H), 1.35 (s, 9H).

### Step 7: Synthesis of methyl 3-[[4-(3-aminopropanamido)-1-methylimidazol-2-yl]formamido]propanoate hydrochloride

A solution of methyl 3-[(4-[3-[(tert-butoxycarbonyl)amino]propanamido]-1-methylimidazol-2-yl)formamido]propanoate (11.00 g, 27.678 mmol, 1.00 equiv) in HCl/1,4-dioxane (4M, 110.00 mL) was stirred for 1.0 h at room temperature. The resulting mixture was concentrated under vacuum to afford methyl 3-[[4-(3-aminopropanamido)-1-methylimidazol-2-yl]formamido]propanoate hydrochloride (11.00 g, crude) as yellow oil. LC/MS: mass calcd. For C₁₂H₁₉N₅O₄: 297.14, found: 298.20 [M+H]⁺. ¹H NMR (400 MHz, DMSO-d₆) δ: 10.57 (s, 1H), 7.92 (t, J=6.0 Hz, 1H), 7.37 (s, 1H), 3.89 (s, 3H), 3.59 (s, 3H), 3.43-3.47 (m, 2H), 2.97-3.05 (m, 2H), 2.57-2.71 (m, 2H), 2.56 (t, J=6.0 Hz, 2H).

### Step 8: Synthesis of Methyl 1-methyl-4-(1-methylimidazole-2-amido)pyrrole-2-carboxylate

To a stirred solution of 1-methylimidazole-2-carboxylic acid (10.00 g, 79.29 mmol, 7.00 equiv) in DMF (150.00 mL) was added TBTU (38.19 g, 118.94 mmol, 1.50 equiv), methyl 4-amino-1-methylpyrrole-2-carboxylate hydrochloride (16.63 g, 87.24 mmol, 1.10 equiv) and DIEA (30.74 g, 237.88 mmol, 3.00 equiv) in portions at 0 degrees C. The resulting mixture was stirred for 17 h at room temperature. The reaction was poured into ice/water (450 mL). The precipitated solids were collected by filtration and washed with H₂O (3×50 mL), dried under vacuum. Methyl 1-methyl-4-(1-methylimidazole-2-amido)pyrrole-2-carboxylate (16.50 g, 78.37%) was obtained as white solid. LC/MS: mass calcd. For C₁₂H₁₄N₄O₃: 262.11, found: 263.15 [M+H]⁺. ¹H NMR (300 MHz, DMSO-d₆) δ: 10.54 (s, 1H), 7.54 (s, 1H), 7.40 (s, 1H), 7.04 (s, 2H), 3.99 (s, 3H), 3.85 (s, 3H), 3.74 (s, 3H).

### Step 9: Synthesis of 1-Methyl-4-(1-methylimidazole-2-amido)pyrrole-2-carboxylic acid

To a stirred solution of methyl 1-methyl-4-(1-methylimidazole-2-amido)pyrrole-2-carboxylate (16.50 g, 62.91 mmol, 1.00 equiv) in MeOH (100.00 mL) was added LiOH solution (2M, 158.00 mL, 5.00 equiv) dropwise at room temperature. The resulting mixture was stirred for 2 h at 45 degrees C. The resulting mixture was concentrated under reduced pressure. The residue was dissolved in H₂O (50 mL). The mixture was acidified to pH 3-5 with 2M HCl. The precipitated solids were collected by filtration and washed with H₂O (3×30 mL), dried under vacuum. 1-Methyl-4-(1-methylimidazole-2-amido)pyrrole-2-carboxylic acid (12.00 g, 76.84%) was obtained as a white solid. LC/MS: mass calcd. For C₁₁H₁₂N₄O₃: 248.09, found: 249.10 [M+H]⁺. ¹H NMR (300 MHz, DMSO-d₆) δ: 10.52 (s, 1H), 7.48 (s, 1H), 7.41 (s, 1H), 7.06 (s, 1H), 6.99 (s, 1H), 3.99 (s, 3H), 3.82 (s, 3H).

### Step 10: Synthesis of Methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate

To a stirred solution of 1-methyl-4-(1-methylimidazole-2-amido)pyrrole-2-carboxylic acid (9.00 g, 36.255 mmol, 1.00 equiv) in DMF (150.00 mL) was added HATU (20.68 g, 54.38 mmol, 1.50 equiv), DIEA (14.06 g, 108.77 mmol, 3.00 equiv) and methyl 4-[4-(3-aminopropanamido)-1-methylimidazole-2-amido]-1-methylpyrrole-2-carboxylate (13.89 g, 39.872 mmol, 1.10 equiv) in portions at 0 degrees C. The resulting mixture was stirred for 17 h at room temperature. The reaction was poured into water/ice (450 mL) at 0 degrees C. The precipitated solids were collected by filtration and washed with H₂O (3×50 mL), dried under vacuum. Methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate (14.00 g, 63.54%) was obtained as yellow solid. LC/MS: mass calcd. For C₂₆H₃₀N₁₀O₆: 578.23, found: 579.10 [M+H]⁺. ¹H NMR (300 MHz, DMSO-d₆) δ: 10.53 (s, 1H), 10.29 (s, 1H), 10.11 (s, 1H), 8.10 (t, J=5.4 Hz, 1H), 7.52 (s, 1H), 7.47 (s, 2H), 7.25 (s, 1H), 7.17 (s, 1H), 6.99 (s, 1H), 6.97 (s, 1H), 3.99 (s, 3H), 3.95 (s, 3H), 3.84 (s, 3H), 3.82 (s, 3H), 3.69 (s, 3H), 3.42-3.49 (m, 2H), 2.60 (t, J=7.2 Hz, 2H).

### Step 11: Synthesis of 1-methyl-4-[1-methyl-4-(3-[[1-methyl-4-(1-methylimidazole-2-amido)pyrrol-2-yl]formamido]propanamido)imidazole-2-affordamido] pyrrole-2-carboxylic acid

A solution of methyl 1-methyl-4-[1-methyl-4-(3-[[1-methyl-4-(1-methylimidazole-2-amido)pyrrol-2-yl]formamido]propanamido)imidazole-2-amido]pyrrole-2-yl]formamidocarboxylate (14.00 g, 24.20 mmol, 1.00 equiv) in MeOH (70.00 mL) was added LiOH (2M, 72.00 mL, 6.00 equiv). The mixture was stirred at 45 degrees C. for 2 h. The resulting mixture was concentrated under reduced pressure. The residue was dissolved in H₂O (50 mL). The mixture was acidified to pH 3-5 with 2M HCl. The precipitated solids were collected by filtration and washed with H₂O (3×20 mL), dried under vacuum. 1-methyl-4-[1-methyl-4-(3-[[1-methyl-4-(1-methylimidazole-2-amido)pyrrol-2-yl]formamido]propanamido)imidazole-2-affordamido] pyrrole-2-carboxylic acid (12.00 g, 81.49%) was obtained as yellow solid. LC/MS: mass calcd. For C₂₅H₂₈N₁₀O₆: 564.22, found: 565.15 [M+H]⁺. ¹H NMR (300 MHz, DMSO-d6) δ: 10.72 (s, 1H), 10.32 (s, 1H), 10.08 (s, 1H), 8.14 (t, J=6.0 Hz, 1H), 7.51 (s, 1H), 7.47 (s, 2H), 7.27 (s, 1H), 7.23 (s, 1H), 6.98 (s, 1H), 6.94 (s, 1H), 4.00 (s, 3H), 3.95 (s, 3H), 3.82 (s, 6H), 3.44-3.46 (m, 2H), 2.60 (t, J=6.6 Hz, 2H).

### Step 12: Synthesis of Methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate

To a stirred solution of 1-methyl-4-[1-methyl-4-(3-[[1-methyl-4-(1-methylimidazole-2-amido)pyrrol-2-yl]formamido]propanamido)imidazole-2-amido] pyrrole-2-carboxylic acid (12.00 g, 21.26 mmol, 1.00 equiv) in DMF (100.00 mL) was added HATU (12.12 g, 31.88 mmol, 1.50 equiv), DIEA (8.24 g, 63.77 mmol, 3.00 equiv) and methyl 3-[[4-(3-aminopropanamido)-1-methylimidazol-2-yl]formamido]propanoate (6.95 g, 23.38 mmol, 1.10 equiv) in portions at 0 degrees C. The resulting mixture was stirred for 2 h at room temperature. The reaction was poured into ice/water (300 mL) at 0 degrees C. The precipitated solids were collected by filtration and washed with H₂O (3×30 mL), dried under vacuum. Methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (13.00 g, 64.77%) was obtained as yellow solid. LC/MS: mass calcd. For C₃₇H₄₅N₁₅O₉: 843.35, found: 844.55 [M+H]⁺. ¹H NMR (300 MHz, DMSO-d6) δ: 10.41 (s, 1H), 10.37 (s, 1H), 10.32 (s, 1H), 9.96 (s, 1H), 8.08 (s, 2H), 7.96 (s, 1H), 7.46 (s, 1H), 7.42 (s, 1H), 7.38 (s, 1H), 7.24 (s, 2H), 7.03 (s, 1H), 6.98 (s, 1H), 6.93 (s, 1H), 4.13 (s, 3H), 3.98 (s, 3H), 3.95 (s, 3H), 3.81 (s, 9H), 3.60 (s, 6H), 2.57-2.69 (m, 6H).

### Step 13: Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid

A solution of methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (10.00 g, 10.59 mmol, 1.00 equiv) in MeOH (60.00 mL) was added 2M LiOH (21.20 mL, 42.40 mmol, 4.00 equiv), the resulting mixture was stirred for 2 h at 45 degrees C. The resulting mixture was concentrated under reduced pressure. The resulting mixture was diluted with water (60 mL). The mixture was acidified to pH 3-5 with 2M HCl. The precipitated solids were collected by filtration and washed with water (3×20 mL). The solid was dried under vacuum. This resulted in 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (8.70 g, 84.14%) as a brown solid. LC/MS: mass calcd. For C₃₆H₄₃N₅O₉: 829.34, found: 830.25 [M+H]⁺. ¹H NMR (300 MHz, DMSO-d₆) δ: 10.46 (s, 1H), 10.39 (s, 1H), 10.31 (s, 1H), 9.93 (s, 1H), 8.05-8.10 (m, 2H), 7.87 (t, J=6.0 Hz, 1H), 7.42-7.46 (m, 3H), 7.20-7.23 (m, 2H), 7.07 (s, 1H), 6.90-6.95 (m, 2H), 3.95 (s, 3H), 3.92 (s, 3H), 3.89 (s, 3H), 3.79 (s, 3H), 3.78 (s, 3H), 3.38-3.41 (m, 6H), 2.44-2.59 (m, 6H).

### Example 2. Synthesis of N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (Compound 127)

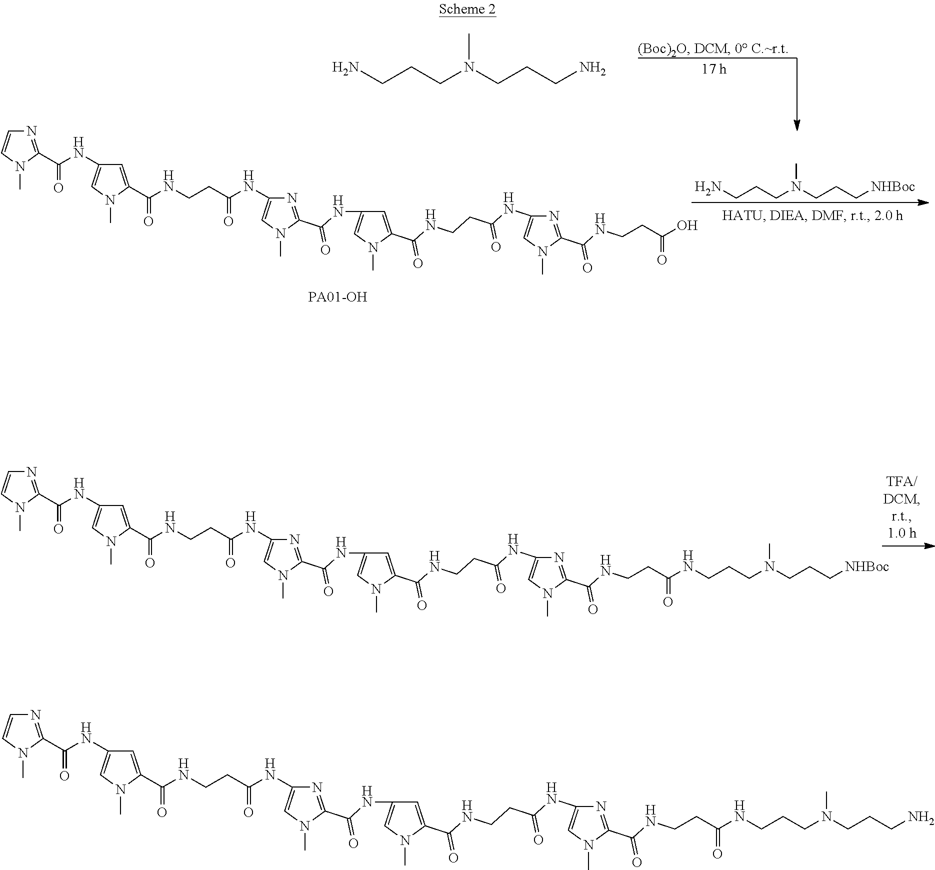

### Step 1: Synthesis of tert-butyl (3-((3-aminopropyl)(methyl)amino)propyl) carbamate

To a solution of bis(3-aminopropyl)(methyl)amine (30.00 g, 206.54 mmol, 2.00 equiv) in DCM (100.00 mL) was added dropwise a solution of Boc₂O (22.40 g, 102.64 mmol, 1.00 equiv) in DCM (100.00 mL) during 4.0 h at 0 degree C. Then the mixture was stirred at room temperature overnight. The solid was filtered out and the filtrate was concentrated. The crude was dissolved in CH₃CN (10 mL) and purified by reverse phase column: column, C18 column; mobile phase, CH₃CN in water (0.05% NH₄HCO₃), from 5% to 30% gradient in 2.0 h, detector UV 220 nm. The fractions were combined and concentrated. Tert-butyl (3-((3-aminopropyl)(methyl)amino)propyl)carbamate (18.00 g, 64.00%) was obtained as colorless oil. LC/MS: mass calcd. For C₁₂H₂₇N₃O₂: 245.21, found: 246.15 [M+H]⁺. ¹H NMR (400 MHz, DMSO) δ: 6.80 (t, J=5.2 Hz, 1H), 3.57-3.60 (m, 2H), 2.87-2.92 (m, 2H), 2.19-2.26 (m, 4H), 2.06 (s, 3H), 1.41-1.52 (m, 4H), 1.35 (s, 9H).

### Step 2: Synthesis of tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate

To a solution of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (2.50 g, 3.37 mmol, 1.00 equiv) in DMF (100.00 mL) was added tert-butyl N-[3-[(3-aminopropyl)(methyl)amino]propyl]carbamate (1.11 g, 4.52 mmol, 1.50 equiv) and HATU (1.72 g, 4.52 mmol, 1.50 equiv). Then DIEA (1.17 g, 9.04 mmol, 3.00 equiv) was added. The mixture was stirred at room temperature for 2.0 h. The mixture was poured into 200 mL ice/water, the solid was filtered out and the filter cake was washed by H₂O (20 mL), dried under high vacuum. Tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate (2.80 g, 79.12%) was obtained as yellow solid. LC/MS: mass calcd. For C₄₈H₆₈N₁₈O₁₀: 1056.54, found: 1057.80 [M+H]⁺.

### Step 3: Synthesis of N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA)

To a solution of tert-butyl N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate (2.80 g, 2.65 mmol, 1.00 equiv) in DCM (30.00 mL) was added TFA (10.00 mL). The reaction mixture was stirred at room temperature for 1.0 h. Then the solvent was removed and the residue was lyophilized. N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (2.80 g, 93.89%) was obtained as dark yellow solid. LC/MS: mass calcd. For C₄₃H₆₀N₁₈O₈: 956.48, found: 957.70 [M+H]⁺.

### Example 3. Synthesis of N-[5-[(2-[[2-([2-[(2-aminoethyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 128)

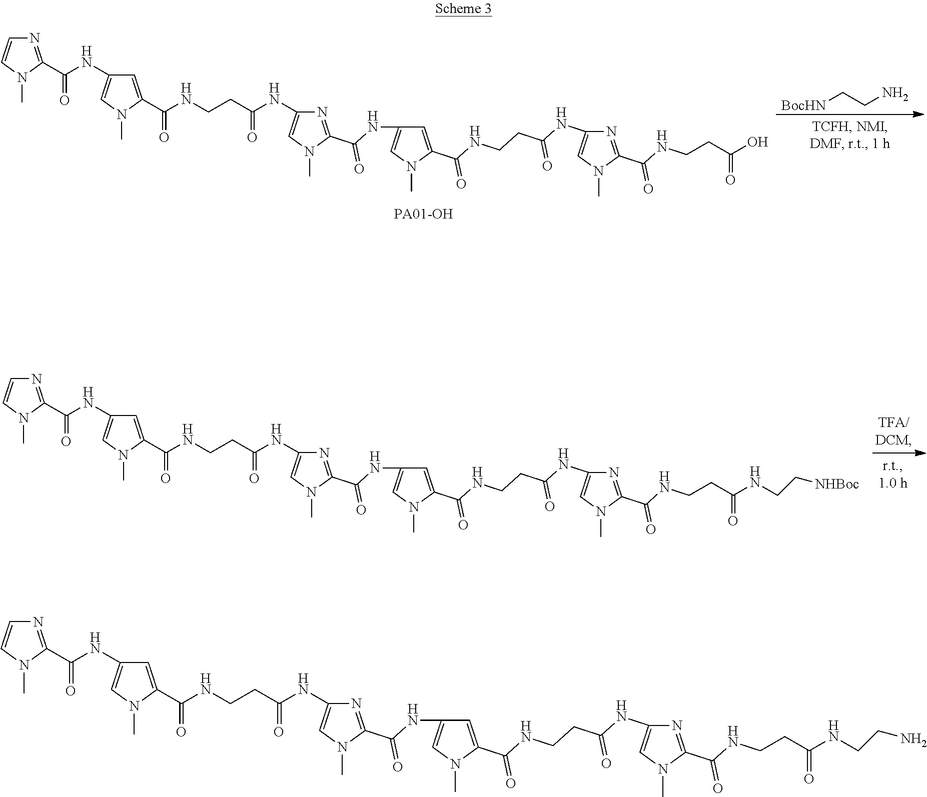 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1 ; CC(C)(C)OC(=O)NCCN ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCNC(=O)OC(C)(C)C)n1 ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCN)n1]

### Step 1: Synthesis of tert-butyl N-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido) propanamido]ethyl]carbamate

The procedure was the same as methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate. 150.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanoic acid was used, 120.00 mg of tert-butyl N-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethyl]carbamate was obtained as yellow oil (66.73% yield). LC/MS: mass calcd. For C₄₃H₅₇N₁₇O₁₀: 971.45, found: 487.05 [½M+H]⁺.

### Step 2: Synthesis of N-[5-[(2-[[2-([2-[(2-aminoethyl)carbamoyl]ethyl] carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl) amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide. 110.00 mg of tert-butyl N-[2-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]ethyl]carbamate was used, 100.00 mg of N-[5-[(2-[[2-([2-[(2-aminoethyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as yellow oil (81.08% yield). LC/MS: mass calcd. For C₃₈H₄₉N₁₇O₈: 871.40, found: 894.60 [M+Na]⁺.

### Example 4. Synthesis of N-[5-([2-[(2-[[2-([3-[(3-aminopropyl)(2,2,2-trifluoroethyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 129)

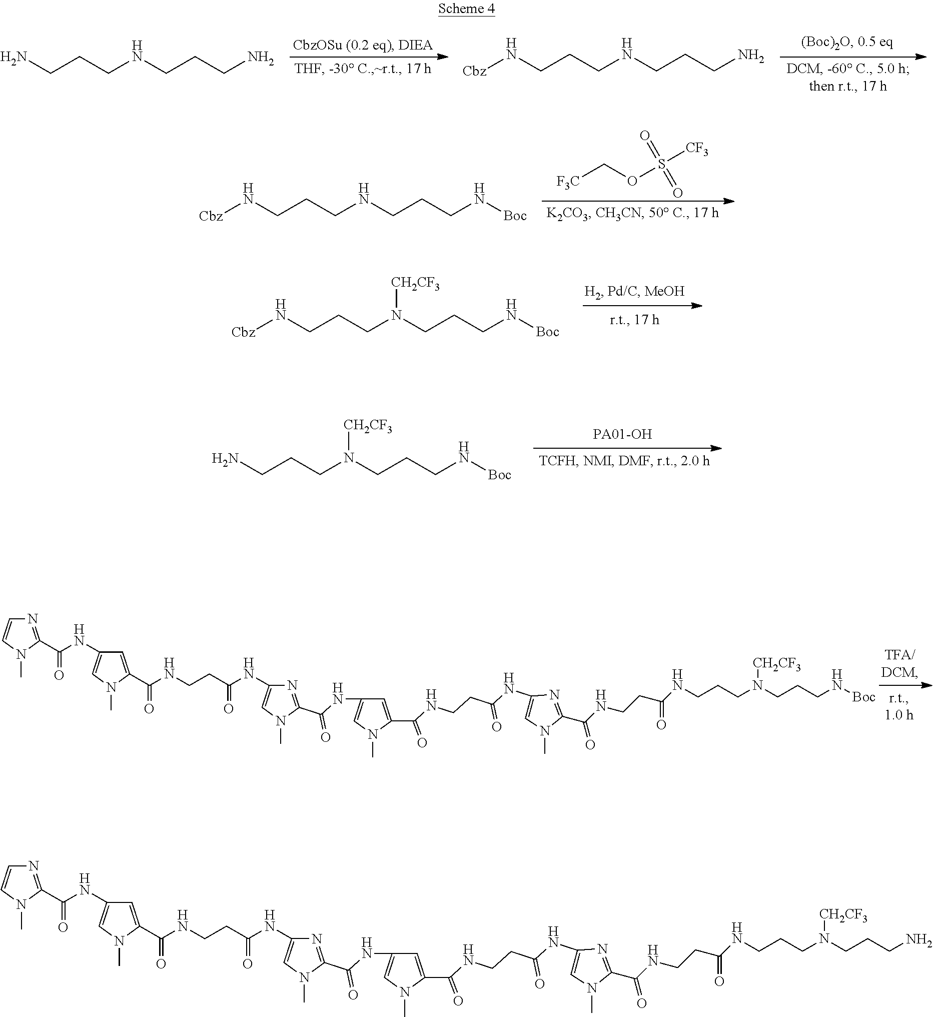

### Step 1: Synthesis of Benzyl N-[3-[(3-aminopropyl)amino]propyl]carbamate

To a stirred solution of norspermidine (6.00 g, 45.72 mmol, 1.00 equiv) in THF (50.00 mL) was added DIEA (5.91 g, 45.72 mmol, 1.00 equiv). Benzyl 2,5-dioxopyrrolidin-1-yl carbonate (2.28 g, 9.15 mmol, 0.20 equiv) in THF (30.00 mL) was added dropwise at −30 degrees C. The resulting mixture was stirred for 17 h at room temperature. The resulting mixture was filtered, the filter cake was washed with THF (3×10 mL). The filtrate was concentrated under reduced pressure. The residue was purified by reverse flash chromatography with the following conditions: column, C18 column; mobile phase, CH₃CN in water (0.05% TFA), 15% to 30% gradient in 10 min; detector, UV 254 nm. The fractions were combined and concentrated. Benzyl N-[3-[(3-aminopropyl)amino]propyl]carbamate (1.50 g, 60.00%) was obtained as yellow oil. LC/MS: mass calcd. For C₁₄H₂₃N₃O₂: 265.18, found: 266.15 [M+H]⁺.

### Step 2: Synthesis of Benzyl N-[3-([3-[(tert-butoxycarbonyl)amino]propyl]amino)propyl]carbamate

To a stirred solution of benzyl N-[3-[(3-aminopropyl)amino]propyl]carbamate (1.40 g, 5.28 mmol, 1.00 equiv) in DCM (20.00 mL) was added (Boc)₂O (0.58 g, 2.66 mmol, 0.50 equiv) in DCM (10.00 mL) solution dropwise at −60 degrees C. The resulting mixture was stirred for 5 h at −60 degrees C. and warmed to room temperature and stirred for additional 12 h at room temperature. The reaction mixture was concentrated under reduced pressure. The residue was purified by reverse flash chromatography with the following conditions: column, C18 column; mobile phase, CH₃CN in water (0.05% NH₄HCO₃), 30% to 50% gradient in 20 min; detector, UV 220 nm. The fractions were combined and concentrated. Benzyl N-[3-([3-[(tert-butoxycarbonyl)amino]propyl]amino)propyl]carbamate (500 mg, 23.63%) was obtained as yellow oil. LC/MS: mass calcd. For C₁₉H₃₁N₃O₄: 365.23, found: 366.10 [M+H]⁺.

### Step 3: Synthesis of Benzyl N-[3-([3-[(tert-butoxycarbonyl)amino]propyl] (2,2,2-trifluoroethyl)amino)propyl]carbamate

To a stirred solution of benzyl N-[3-([3-[(tert-butoxycarbonyl)amino]propyl]amino)propyl]carbamate (280.00 mg, 0.77 mmol, 1.00 equiv) in CH₃CN (8.00 mL) was added 2,2,2-trifluoroethyl trifluoromethanesulfonate (177.82 mg, 0.77 mmol, 1.00 equiv) and K₂CO₃ (317.65 mg, 2.30 mmol, 3.00 equiv) in portions at room temperature. The resulting mixture was stirred for 17 h at 50 degrees C. The solid was filtered out and the filtrate was concentrated. The residue was purified by reverse flash chromatography with the following conditions: column, C18 silica gel; mobile phase, ACN in water (0.05% NH₄HCO₃), 20% to 30% gradient in 100 min; detector, UV 254 nm. The fractions were combined and concentrated. Benzyl N-[3-([3-[(tert-butoxycarbonyl)amino]propyl](2,2,2-trifluoroethl)amino)propyl]carbamate (150 mg, 43.75%) was obtained as colorless oil. LC/MS: mass calcd. For C₂₁H₃₂F₃N₃O₄: 447.23, found: 448.20 [M+H]⁺.

### Step 4: Synthesis of Tert-butyl N-[3-[(3-aminopropyl)(2,2,2-trifluoroethyl) amino]propyl]carbamate

To a solution of benzyl N-[3-([3-[(tert-butoxycarbonyl)amino]propyl](2,2,2-trifluoroethyl)amino)propyl]carbamate (150.00 mg, 0.34 mmol, 1.00 equiv) in MeOH (8.00 mL) was added Pd/C (30.00 mg, 20% w/w). Then the reaction was stirred for 17 h at room temperature under H₂ atmosphere. The resulting mixture was filtered and the filter cake was washed with MeOH (3×5 mL). The filtrate was concentrated under reduced pressure. Tert-butyl N-[3-[(3-aminopropyl)(2,2,2-trifluoroethyl)amino] propyl]carbamate (90.00 mg, 85.68%) was obtained as colorless oil. LC/MS: mass calcd. For C₁₃H₂₆F₃N₃O₂: 313.20, found: 314.15 [M+H]⁺.

### Step 5: Synthesis of Tert-butyl N-[3-([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl](2,2,2-trifluoroethyl)amino)propyl]carbamate

The procedure was the same as ethyl 4-(3-[[4-(4-[3-[(tert-butoxycarbonyl)amino] propanamido]-1-methylimidazole-2-amido)-1-methylpyrrol-2-yl]formamido]propanamido)-1-methylimidazole-2-carboxylate. 212.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 150.00 mg of tert-butyl N-[3-([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl](2,2,2-trifluoroethyl)amino)propyl]carbamate was obtained as yellow solid (42.94% yield). LC/MS: mass calcd. For C₄₉H₆₇F₃N₁₈O: 1124.52, found: 1125.45 [M+H]⁺.

### Step 6: Synthesis of N-[5-([2-[(2-[[2-([3-[(3-aminopropyl) (2,2,2-trifluoroethyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl) amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 150.00 mg of tert-butyl N-[3-([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanamido]propyl](2,2,2-trifluoroethyl)amino)propyl]carbamate was used, 150.00 mg crude of N-[5-([2-[(2-[[2-([3-[(3-aminopropyl) (2,2,2-trifluoroethyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as yellow oil. LC/MS: mass calcd. For C₄₄H₅₉F₃N₁₈O₈: 1024.47, found: 1025.35 [M+H]⁺.

### Example 5. Synthesis of 1-methyl-4-(3-[[1-methyl-4-(1-methylimidazole-2-amido) pyrrol-2-yl]formamido]propanamido)-N-[1-methyl-5-[(2-[[1-methyl-2-([2-[(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide (Compound 130)

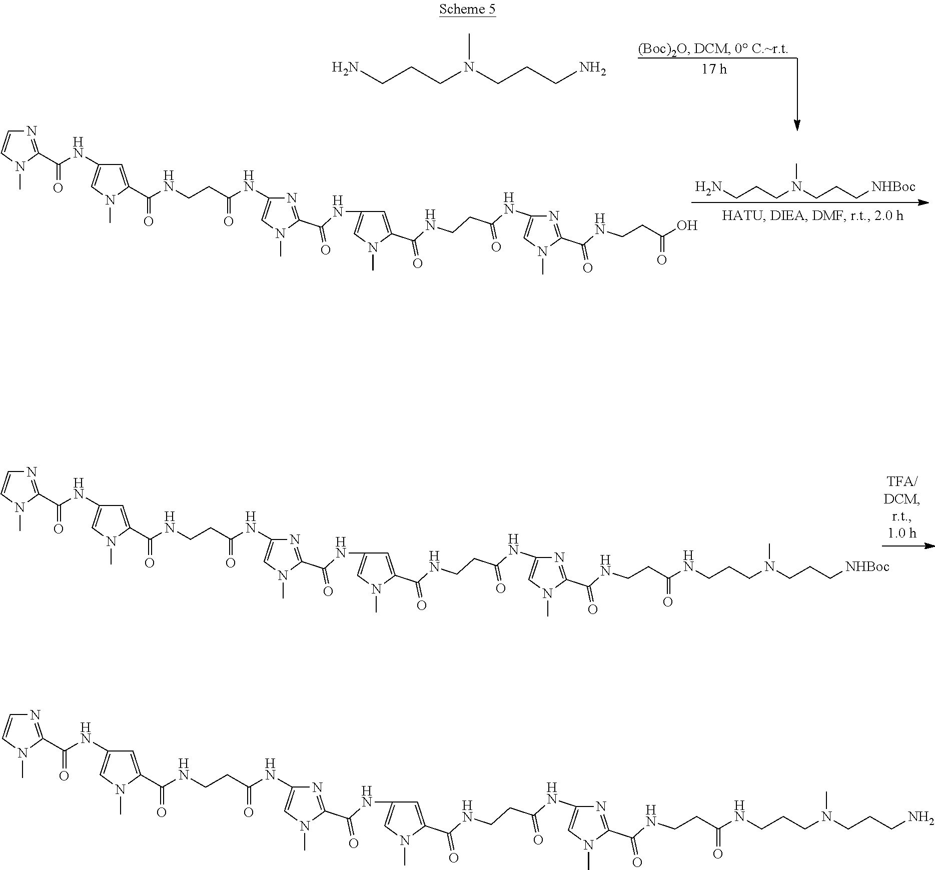

### Step 1: Synthesis of tert-butyl N-methyl-N-[3-(methylamino) propyl] carbamate

To a stirred solution of methyl [3-(methylamino)propyl]amine (2000.00 mg, 19.57 mmol, 1.00 equiv) in DCM (4.00 mL) was added (Boc) 20 (2135.89 mg, 9.79 mmol, 0.50 equiv) dropwise at 0 degrees C. The resulting mixture was stirred for 17 h at room temperature. After that, the reaction was quenched with water (30 mL) and extracted with DCM (3×50 mL). The organic layers were combined, dried over anhydrous sodium sulfate, filtered and concentrated. Tert-butyl N-methyl-N-[3-(methylamino) propyl] carbamate (2500.00 mg, 63.14%) was obtained as yellow oil. LC/MS: mass calcd. For C₁₀H₂₂N₂O₂:202.18, found: 203.20 [M+H]⁺.

### Step 2: Synthesis of tert-butyl N-(3-[[3-(1,3-dioxoisoindol-2-yl)propyl](methyl)amino]propyl)-N-methylcarbamate

To a stirred solution of tert-butyl N-methyl-N-[3-(methylamino) propyl] carbamate (2500.00 mg, 12.36 mmol, 1.00 equiv) in CH₃CN (100.00 mL) was added N-(3-bromopropyl) phthalimide (3313.31 mg, 12.36 mmol, 1.00 equiv) and K₂CO; (5123.83 mg, 37.07 mmol, 3.00 equiv) in portions at room temperature. The resulting mixture was stirred for 17 h at 70 degrees C. After cooling down to room temperature, the solid was filtered out and the filtrate was concentration under reduced pressure. The residue obtained was purified by silica gel chromatography (DCM:MeOH=10:1) to afford tert-butyl N-(3-[[3-(1,3-dioxoisoindol-2-yl) propyl] (methyl) amino] propyl)-N-methylcarbamate (2600.00 mg, 54.02%) as yellow oil. LC/MS: mass calcd. For C₂₁H₃N₃O₄: 389.23, found: 390.20 [M+H]⁺.

### Step 3: Synthesis of tert-butyl N-[3-[(3-aminopropyl)(methyl)amino]propyl]-N-methylcarbamate

To a stirred solution of tert-butyl N-(3-[[3-(1,3-dioxoisoindol-2-yl)propyl](methyl)amino]propyl)-N-methylcarbamate (2500.00 mg, 6.42 mmol, 1.00 equiv) in MeOH (30.00 mL) was added hydrazine (1028.44 mg, 32.09 mmol, 5.00 equiv) dropwise at room temperature. The resulting mixture was stirred for 3 h at 60 degrees C. After that, the reaction was quenched with water (40 mL) and extracted with ethyl acetate (3×60 mL). The organic layers were combined, dried over anhydrous sodium sulfate, filtered and concentrated. Tert-butyl N-[3-[(3-aminopropyl)(methyl)amino]propyl]-N-methylcarbamate (2.30 g, crude) was obtained as yellow oil. LC/MS: mass calcd. For C₁₃H₂₉N₃O₂:259.23, found: 260.20 [M+H]⁺.

### Step 4: Synthesis of tert-butyl N-methyl-N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate

The procedure was the same as tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 300.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanoic acid (PA01-OH) was used, 200.00 mg of tert-butyl N-methyl-N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was obtained as yellow oil (42.86% yield). LC/MS: mass calcd. For C₄₉H₇₀N₁₈O₁₀: 1070.55, found: 1071.75 [M+H]⁺.

### Step 5: Synthesis of 1-methyl-4-(3-[[1-methyl-4-(1-methylimidazole-2-amido)pyrrol-2-yl]formamido]propanamido)-N-[1-methyl-5-[(2-[[1-methyl-2-([2-[(3-[methyl[3-(methylamino)propyl]amino]propyl)carbamoyl]ethyl]carbamoyl) imidazol-4-yl]carbamoyl]ethyl)carbamoyl]pyrrol-3-yl]imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 90.00 mg of tert-butyl N-methyl-N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was used, 100.00 mg of 1-methyl-4-(3-[[1-methyl-4-(1-methylimidazole-2-amido)pyrrol-2-yl]formamido]propanamido)-N-[1-methyl-5-[(2-[[1-methyl-2-([2-[(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide was obtained as yellow oil. LC/MS: mass calcd. For C₄₄H₆₂N₁₈O₈:970.50, found: 971.75 [M+H]⁺.

### Example 6. Synthesis of 1-methyl-4-(3-[[1-methyl-4-(1-methylimidazole-2-amido) pyrrol-2-yl]formamido]propanamido)-N-[1-methyl-5-[(2-[[1-methyl-2-([2-[methyl(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide (Compound 131)

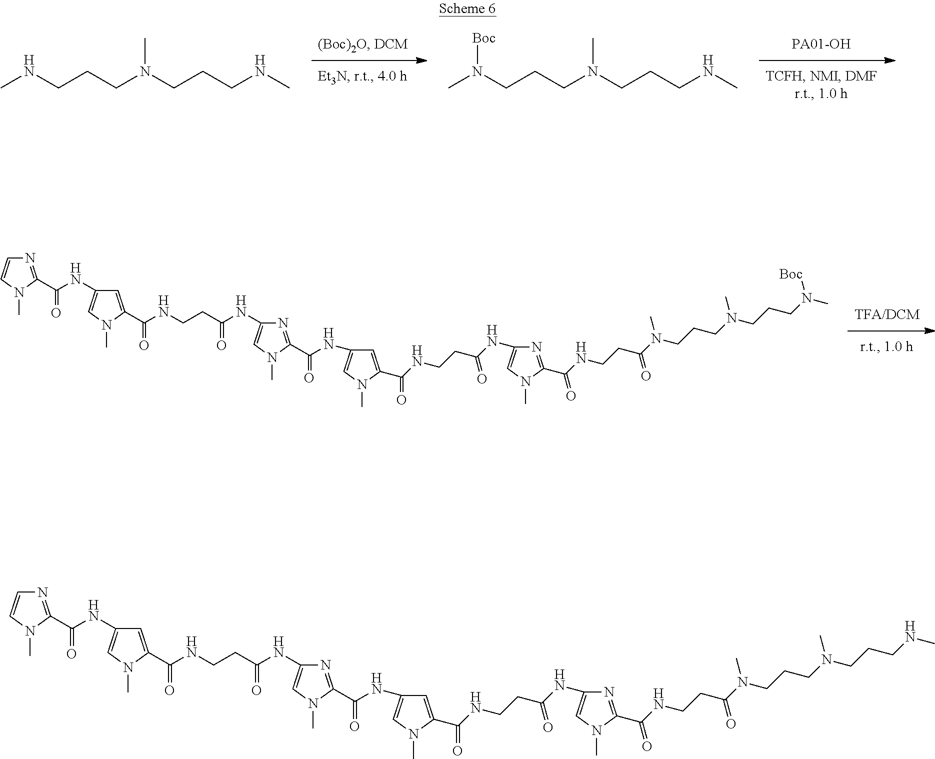

### Step 1: Synthesis of tert-butyl N-methyl-N-(3-[methyl[3-(methylamino)propyl] amino]propyl) carbamate

The procedure was the same as tert-butyl (3-((3-aminopropyl)(methyl)aminopropyl)carbamate. 1.40 g of methyl(3-[methyl[3-(methylamino)propyl]amino]propyl)amine was used, 350.00 mg of tert-butyl N-methyl-N-(3-[methyl[3-(methylamino)propyl]amino]propyl)carbamate was obtained as yellow oil (15.85% yield). LC/MS: mass calcd. For: C₁₄H₃₁N₃O₂: 273.24, found: 274.10 [M+H]⁺.

### Step 2: Synthesis of tert-butyl N-methyl-N-[3-[methyl([3-[N-methyl-3-([1-methyl-4-[3-([1-methyl-4-[1-methyl-4-(3-[[1-methyl-4-(1-methylimidazole-2-amido)pyrrol-2-yl]formamido])imidazole-2-amido]pyrrol-2-yl]formamido)propanamido]imidazol-2-yl]formamido)propanamido]propyl])amino]propyl]carbamate

The procedure was the same as ethyl 4-(3-[[4-(4-[3-[(tert-butoxycarbonyl)amino] propanamido]-1-methylimidazole-2-amido)-1-methylpyrrol-2-yl]formamido]propanamido)-1-methylimidazole-2-carboxylate. 300.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 150.00 mg of tert-butyl N-methyl-N-[3-[methyl([3-[N-methyl-3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was obtained as yellow solid (38.10% yield). LC/MS: mass calcd. For C₃₀H₇₂N₁₈O₁₀: 1084.57, found: 543.70 [M/2+H]⁺.

### Step 3: Synthesis of 1-methyl-4-(3-[[1-methyl-4-(1-methylimidazole-2-amido)pyrrol-2-yl]formamido]propanamido)-N-[1-methyl-5-[(2-[[1-methyl-2-([2-[methyl(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide. 150.00 mg of tert-butyl N-methyl-N-[3-[methyl([3-[N-methyl-3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was used, 150.00 mg crude of 1-methyl-4-(3-[[1-methyl-4-(1-methylimidazole-2-amido)pyrrol-2-yl]formamido]propanamido)-N-[1-methyl-5-[(2-[[1-methyl-2-([2-[methyl(3-[methyl[3-(methylamino)propyl]amino]propyl)carbamoyl]ethyl]carbamoyl)imidazol-4-yl]carbamoyl]ethyl)carbamoyl]pyrrol-3-yl]imidazole-2-carboxamide was obtained as yellow oil. LC/MS: mass calcd. For C₄₅H₆₄N₁₈O₈: 984.52, found: 985.40 [M+H]⁺.

### Example 7. Synthesis of N-(5-[[2-([2-[(2-[[3-(3-aminopropoxy)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide (Compound 132)

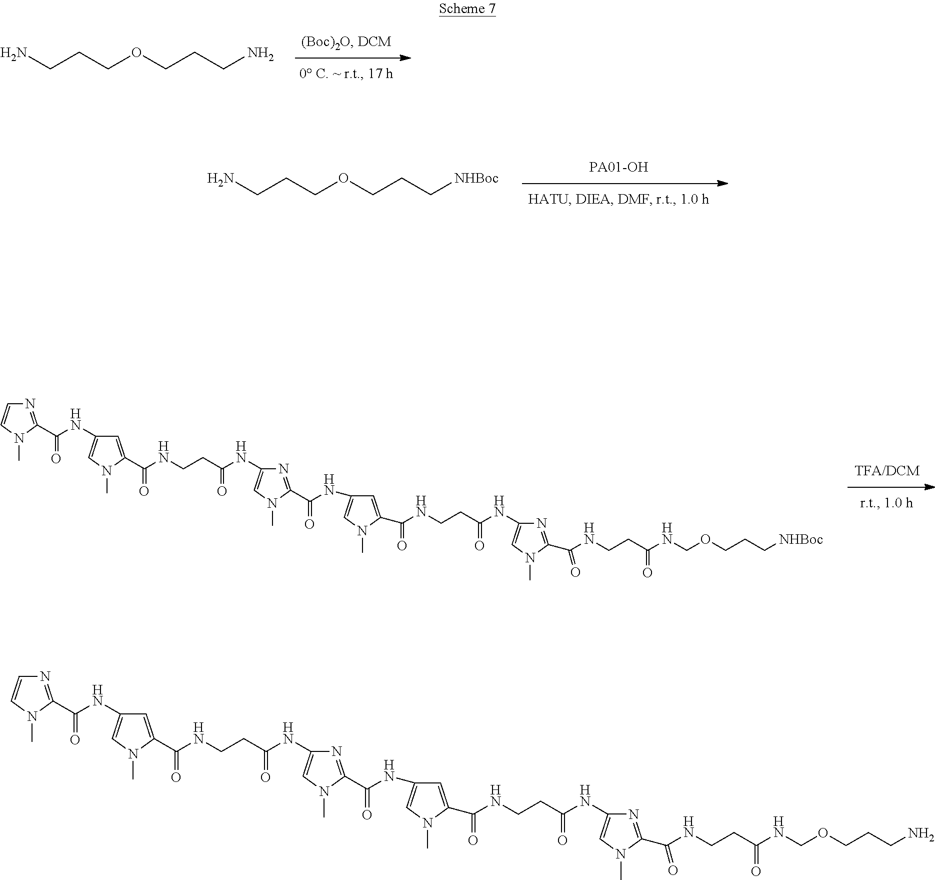 [SMILES: NCCCOCCCN ; CC(C)(C)OC(=O)NCCCOCCCN ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCOCCCNC(=O)OC(C)(C)C)n1 ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCOCCCN)n1]

### Step 1: Synthesis of tert-butyl N-[3-(3-aminopropoxy)propyl]carbamate

The procedure was the same as tert-butyl(3-((3-aminopropyl)(methyl)amino)propyl)carbamate. 1.00 g of 3-(3-aminopropoxy)propan-1-amine was used, 430.00 mg of tert-butyl N-[3-(3-aminopropoxy)propyl]carbamate was obtained as yellow oil (24.47% yield). LC/MS: mass calcd. For C₁₁H₂₄N₂O₃:232.18, found: 233.15 [M+H]⁺.

### Step 2: Synthesis of tert-butyl N-(3-[3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido] imidazol-2-yl]formamido)propanamido]propoxy]propyl)carbamate

The procedure was the same as tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 100.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 90.00 mg of tert-butyl N-(3-[3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propoxy]propyl)carbamate was obtained as yellow oil (64.37% yield). LC/MS: mass calcd. For C₄₇H₆₅N₁₇O₁₁: 1043.50, found: 1044.75 [M+H]⁺.

### Step 3: Synthesis of N-(5-[[2-([2-[(2-[[3-(3-aminopropoxy)propyl]carbamoyl] ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide. 90.00 mg of tert-butyl N-(3-[3-[3-([1-methyl-4-[3-([11-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propoxy]propyl)carbamate was used, 90.00 mg crude of N-(5-[[2-([2-[(2-[[3-(3-aminopropoxy)propyl]carbamoyl]ethyl)carbamoyl]-1-methylimidazol-4-yl]carbamoyl)ethyl]carbamoyl]-1-methylpyrrol-3-yl)-1-methyl-4-(3-[[1-methyl-4-(1-methylimidazole-2-amido)pyrrol-2-yl]formamido]propanamido)imidazole-2-carboxamide was obtained as yellow oil. LC/MS: mass calcd. For C₄₂H₅₇N₁₇O₉: 943.45, found: 944.70 [M+H]⁺.

### Example 8. Synthesis of N-[5-[(2-[[2-([2-[(7-aminoheptyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 133)

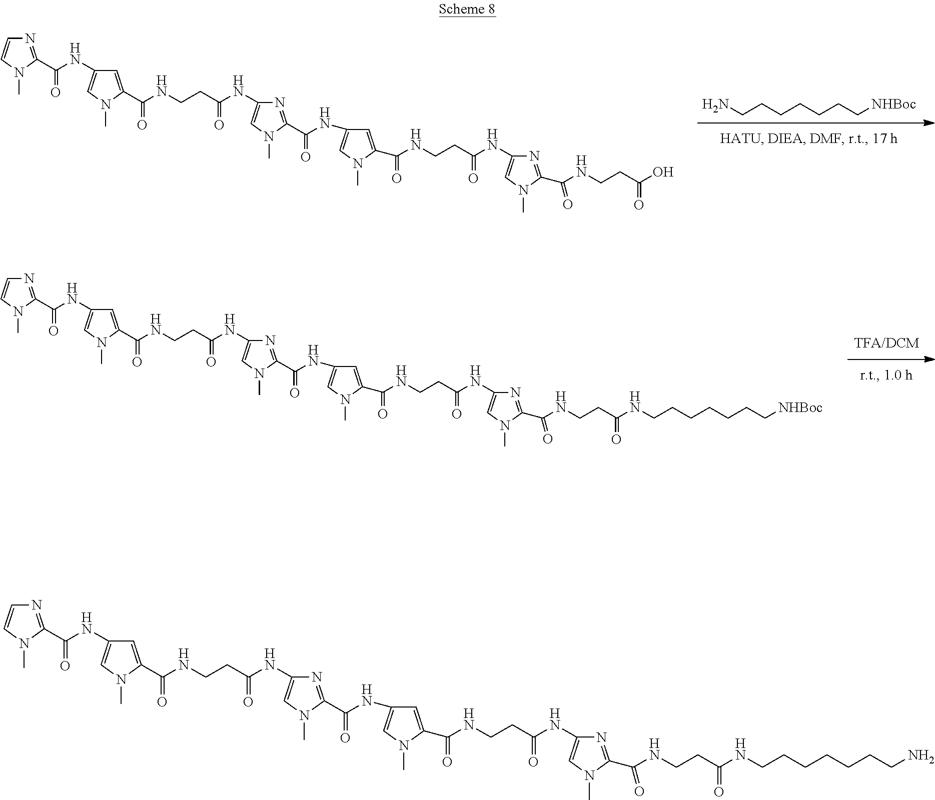 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1 ; CC(C)(C)OC(=O)NCCCCCCCN ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCCCCCCNC(=O)OC(C)(C)C)n1 ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCCCCCCN)n1]

### Step 1: Synthesis of tert-butyl N-[7-[3-([1-methyl-4-[3-([1-methyl-4-[1-methyl-4-(3-[[1-methyl-4-(1-methylimidazole-2- amido) pyrrol-2-yl]formamido]propanamido) imidazole-2-amido]pyrrol-2-yl]formamido)propanamido]imidazol-2-yl]formamido)propanamido]heptyl]carbamate

The procedure was the same as tert-butyl (3-(methyl(3-3-(1-methyl-4-(3-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 150.00 mg of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH) was used, 100.00 mg of tert-butyl N-[7-[3-([1-methyl-4-[3-([1-methyl-4-[1-methyl-4-(3-[[1-methyl-4-(1-methylimidazole-2-amido)pyrrol-2-yl] formamido]propanamido)imidazole-2-amido]pyrrol-2-yl]formamido)propanamido]imidazol-2-yl]formamido)propanamido]heptyl]carbamate was obtained as yellow oil (33.23% yield). LC/MS: mass calcd. For C₄₈H₆₇N₁₇O₁₀: 1041.53, found: 1042.85 [M+H]⁺.

### Step 2: Synthesis of N-[5-[(2-[[2-([2-[(7-aminoheptyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 90.00 mg of tert-butyl N-[7-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]heptyl]carbamate was obtained, 90.00 mg crude of N-[5-[(2-[[2-([2-[(7-aminoheptyl)carbamoyl]ethyl]carbamoyl)-1-methylimidazol-4-yl]carbamoyl]ethyl)carbamoyl]-1-methylpyrrol-3-yl]-1-methyl-4-(3-[[1-methyl-4-(1-methylimidazole-2-amido)pyrrol-2-yl]formamido]propanamido)imidazole-2-carboxamide was obtained as yellow oil. LC/MS: mass calcd. For C₄₃H₅₉N₁₇O₈: 941.47, found: 942.75 [M+H]⁺.

### Example 9. Synthesis of N-[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide (Compound 134)

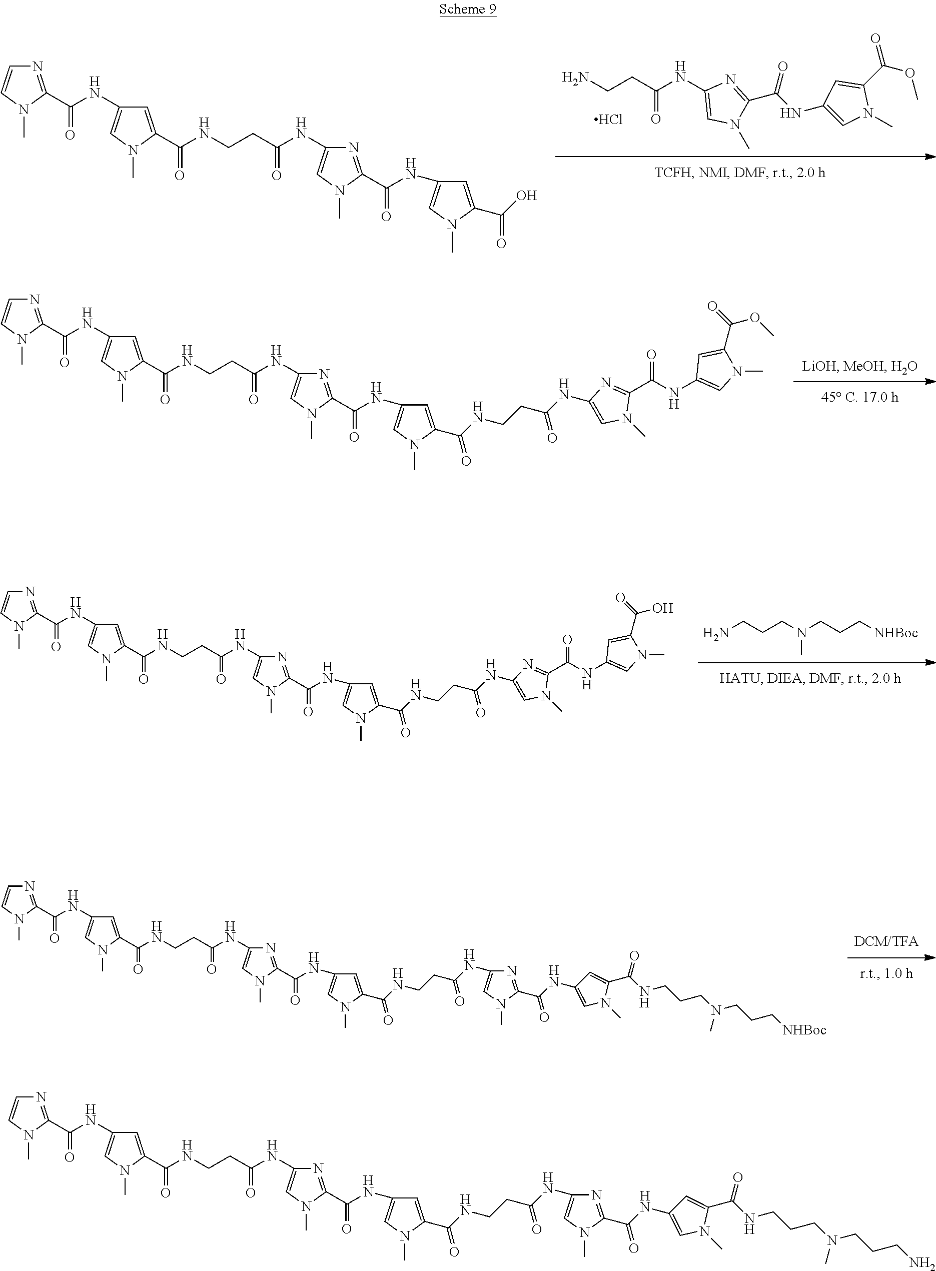

### Step 1 Synthesis of methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido] pyrrole-2-carboxylate

To a solution of 1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxylic acid (1.00 g, 1.77 mmol, 1.00 equiv) in DMF (10.00 mL) was added NMI (727.14 mg, 8.86 mmol, 5.00 equiv), TCFH (546.68 mg, 1.95 mmol, 1.10 equiv) and methyl 4-[4-(3-aminopropanamido)-1-methylimidazole-2-amido]-1-methylpyrrole-2-carboxylate (748.19 mg, 1.95 mmol, 1.10 equiv). Then the reaction was stirred at room temperature for 2 h. The reaction mixture was purified by reverse flash chromatography with the following conditions: column, C₁₈ column; mobile phase, MeOH in H₂O (0.05% TFA), 5% to 75% gradient in 70 min; detector, UV 254 nm. The fractions were combined and concentrated to afford methyl 1-methyl-4-[1-methyl-4-[13-([1-methyl-4-[1-methyl-4-(3-[[1-methyl-4-(1-methylimidazole-2-amido)pyrrol-2-yl]formamido]propanamido)imidazole-2-amido] pyrrol-2-yl]formamido)propanamido]imidazole-2-amido]pyrrole-2-carboxylate (1.00 g, 63.09% yield) as white solid. LC/MS: mass calcd. For C₄₀H₄₆N₁₆O₉: 894.36, found: 895.55 [M+H]⁺.

### Step 2: Synthesis of 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 1.00 g of methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate was used, 800.00 mg of 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylic acid was obtained as white solid(81.27% yield). LC/MS: mass calcd. For C₃₉H₄₄N₁₆O₉: 880.35, found: 881.45 [M+H]⁺.

### Step 3: Synthesis of tert-butyl N-[3-[methyl([3-[(1-methyl-4-[1-methyl-4-[3 ([1-methyl-4-[1-methyl-4-(3-[[1-methyl-4-(1-methylimidazole-2-amido)pyrrol-2-yl]formamido] propanamido)imidazole-2-amido]pyrrol-2-yl]formamido) propanamido]imidazole-2-amido]pyrrol-2-yl) formamido]propyl])amino]propyl] carbamate

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide. 800.00 mg of 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylic acid was used, 800.00 mg of tert-butyl N-[3-[methyl([3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propyl])amino]propyl]carbamate was obtained as white solid (79.49% yield). LC/MS: mass calcd. For C₁₁H₆₉N₁₉O₁₀: 1107.55 found: 1108.80 [M+H]⁺.

### Step 4: Synthesis of N-[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-[[1-methyl-4-(1-methylimidazole-2-amido)pyrrol-2-yl]formamido]propanamido)imidazole-2-carboxamide

The procedure was the same as N-(3-((3-((3-aminopropyl)(methyl)amino)propyl)amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide (PA01-TRA). 800.00 mg of tert-butyl N-[3-[methyl([3-[3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanamido]propyl])amino]propyl]carbamate was used, 800.00 mg of N-[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]-1-methyl-4-(3-([1-methyl-4-(1-methylimidazole-2-amido)pyrrol-2-yl]formamido]propanamido)imidazole-2-carboxamide was obtained as yellow solid. LC/MS: mass calcd. For C₄₆H₆₁N₁₉O₈: 1007.50 found: 1008.80 [M+H]⁺.

### Example 10. Synthesis of 3-[(1-Methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoic acid (Compound 18)

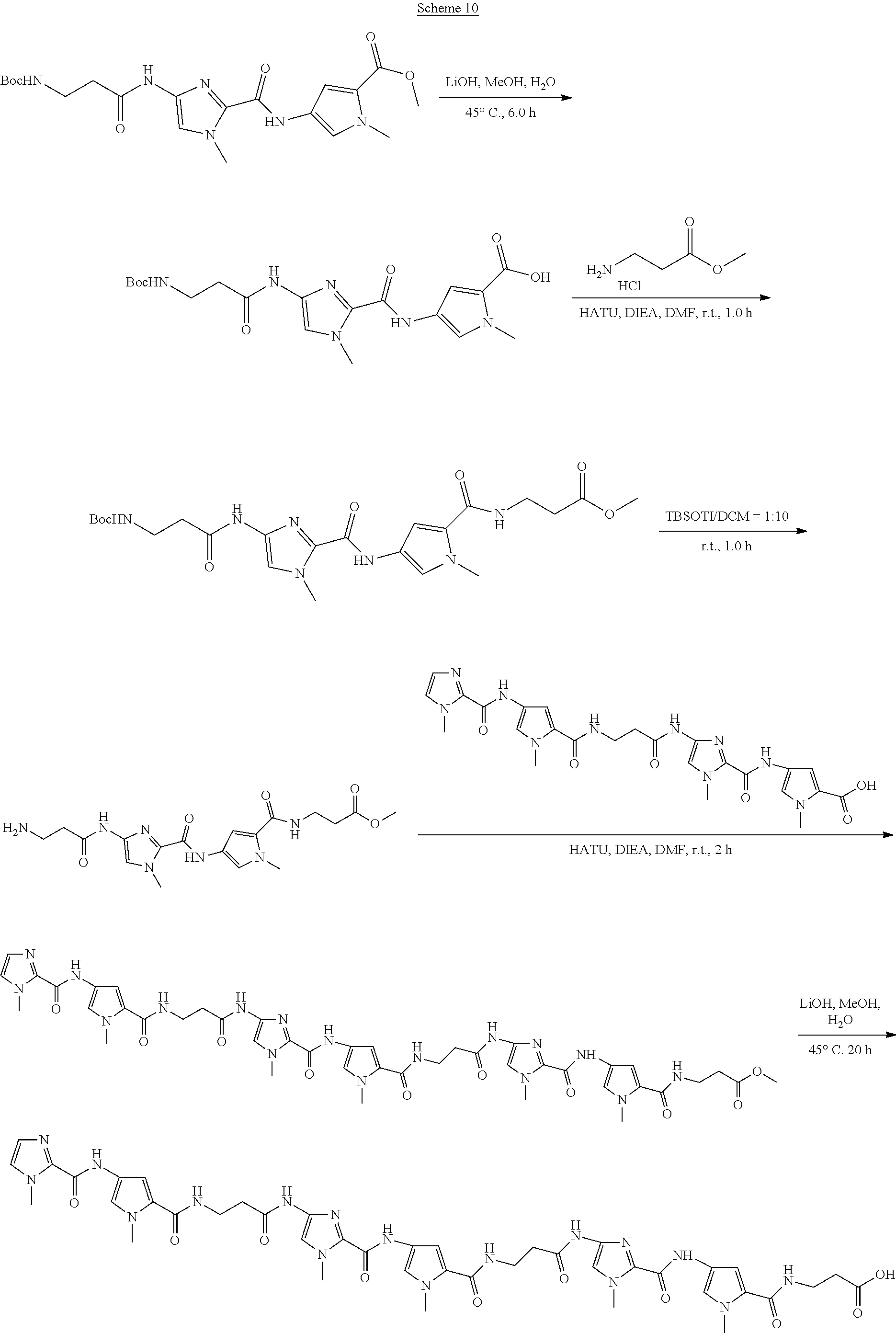

### Step 1: Synthesis of 4-(4-[3-[(Tert-butoxycarbonyl)amino]propanamido]-1-methylimidazole-2-amido)-1-methylpyrrole-2-carboxylic acid

The procedure was the same as 4-[3-[(Tert-butoxycarbonyl)amino]propanamido]-1-methylimidazole-2-carboxylic acid. 2.50 g methyl 4-(4-[3-[(tert-butoxycarbonyl)amino]propanamido]-1-methylimidazole-2-amido)-1-methylpyrrole-2-carboxylate was used, 2.12 g of 4-(4-[3-[(Tert-butoxycarbonyl)amino]propanamido]-1-methylimidazole-2-amido)-1-methylpyrrole-2-carboxylic acid was obtained as white solid (85.94% yield). LC/MS: mass calcd. For C₁₉H₂₆N₆O₆: 434.19, found: 435.20 [M+H]⁺.

### Step 2: Synthesis of methyl 3-[[4-(4-[3-[(tert-butoxycarbonyl)amino]propanamido]-1-methylimidazole-2-amido)-1-methylpyrrol-2-yl]formamido]propanoate

The procedure was the same as methyl 3-[(4-[3-[(tert-butoxycarbonyl)amino]propanamido]-1-methylimidazol-2-yl)formamido]propanoate. 2.12 g of 4-(4-[3-[(tert-butoxycarbonyl)amino]propanamido]-1-methylimidazole-2-amido)-1-methylpyrrole-2-carboxylic acid was used, 2.30 g of methyl 3-[[4-(4-[3-[(tert-butoxycarbonyl)amino]propanamido]-1-methylimidazole-2-amido)-1-methylpyrrol-2-yl]formamido]propanoate was obtained as yellow oil (87.33% yield). LC/MS: mass calcd. For C₂₃H₃₃N₇O₇: 519.24, found: 520.35 [M+H]⁺.

### Step 3: Synthesis of methyl 3-([4-[4-(3-aminopropan-amido)-1-methylimidazole-2-amido]-1-methylpyrrol-2-yl]formamido)propanoate

To a stirred solution of methyl 3-[[4-(4-[3-[(tert-butoxycarbonyl)amino]propanamido]-1-methylimidazole-2-amido)-1-methylpyrrol-2-yl]formamido]propanoate (1.50 g, 2.89 mmol, 1.00 equiv) in DCM (10.00 mL) was added TBSOTf (1.00 mL) dropwise at room temperature. The resulting mixture was stirred for 1 h at room temperature. The resulting mixture was concentrated under vacuum. Methyl 3-([4-[4-(3-aminopropan-amido)-1-methylimidazole-2-amido]-1-methylpyrrol-2-yl]formamido)propanoate (1.70 g, crude) was obtained as red oil. LC/MS: mass calcd. For C₁₈H₂₅N₇O₅: 419.19, found: 420.15 [M+H]⁺.

### Step 4: Synthesis of methyl 3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoate

The procedure was the same as methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate. 800.00 mg of 1-methyl-4-[1-methyl-4-(3-[[1-methyl-4-(1-methylimidazole-2-amido)pyrrol-2-yl]formamido]propanamido)imidazole-2-amido]pyrrole-2-carboxylic acid was used, 800.00 mg of methyl 3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoate was obtained as yellow solid (41.11% yield). LC/MS: mass calcd. For C₄₂H₄₉N₁₇O₁₀: 965.40, found: 966.40 [M+H]⁺.

### Step 5: Synthesis of 3-[(1-Methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido] pyrrol-2-yl)formamido]propanoic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 800.00 mg of methyl 3-[(1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoate was used, 650 mg of 3-[(1-Methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrol-2-yl)formamido]propanoic acid was obtained as yellow solid (68.67% yield). LC/MS: mass calcd. For C₄₂H₄₉N₁₇O₁₀: 951.38, found: 952.35 [M+H]⁺.

### Example 11. Synthesis of 4-(3-[[4-(4-amino-1-methylimidazole-2-amido)-1-methylpyrrol-2-yl]formamido]propanamido)-1-methyl-N-[1-methyl-5-([2-[(1-methyl-2-[[2-(propylcarbamoyl)ethyl]carbamoyl]imidazol-4-yl)carbamoyl]ethyl]carbamoyl)pyrrol-3-yl]imidazole-2-carboxamide (Compound 19)

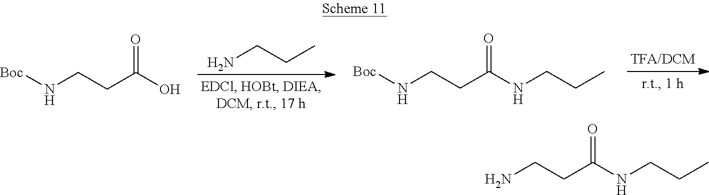

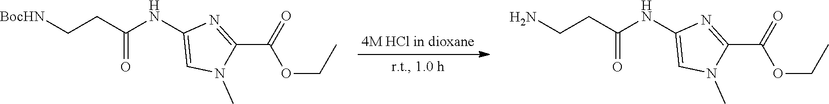 [SMILES: CCOC(=O)c1nc(NC(=O)CCNC(=O)OC(C)(C)C)cn1C ; CCOC(=O)c1nc(NC(=O)CCN)cn1C]

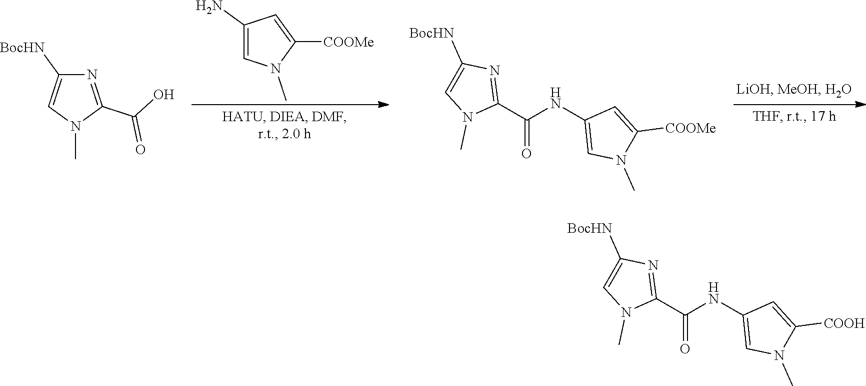

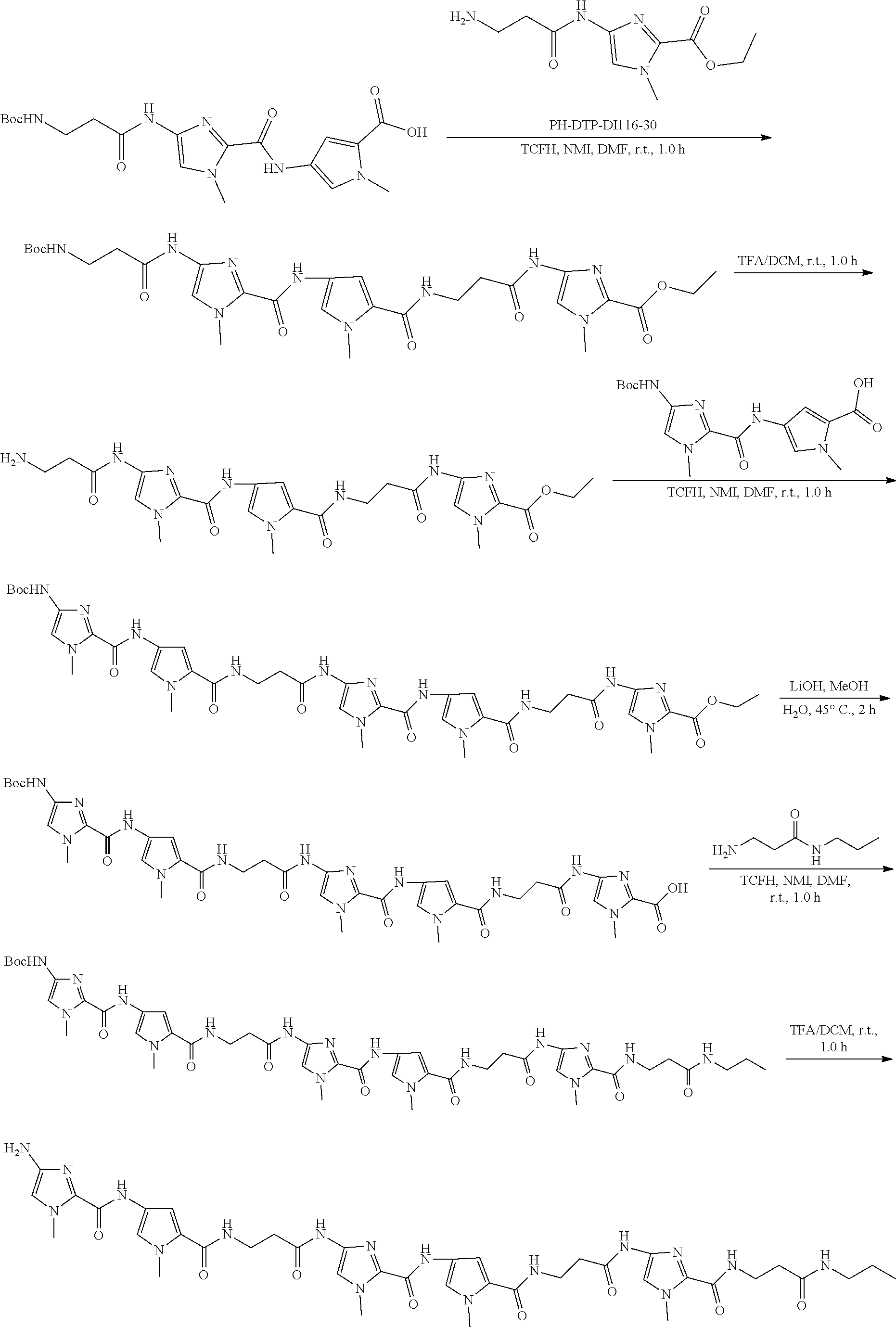

### Step 1: Synthesis of tert-butyl (3-oxo-3-(propylamino)propyl)carbamate

To a stirred solution of 3-[(tert-butoxycarbonyl) amino] propanoic acid (1000.00 mg, 5.29 mmol, 1.00 equiv) in DCM (30.00 mL) was added EDC (114.48 mg, 5.81 mmol, 1.10 equiv), HOBt (785.56 mg, 5.81 mmol, 1.10 equiv) and propylamine (312.41 mg, 5.23 mmol, 1.00 equiv) and DIEA (2732.26 mg, 21.14 mmol, 4.00 equiv) in portions at room temperature. The resulting mixture was stirred for 17 h at room temperature. The reaction was quenched with cool water (30 mL). The resulting mixture was extracted with dichloromethane (3×50 mL). The organic layers were combined, dried over anhydrous sodium sulfate, filtered and concentrated. Tert-butyl (3-oxo-3-(propylamino) propyl) carbamate (600.00 mg, crude) was obtained as white solid. LC/MS: mass calcd. For C₁₁H₂₂N₂O₃: 230.16, found: 231.15 [M+H]⁺.

### Step 2: Synthesis of 3-amino-N-propylpropanamide

To a stirred solution of tert-butyl N-[2-(propylcarbamoyl) ethyl] carbamate (500.00 mg, 2.17 mmol, 1.00 equiv) in DCM (10.00 mL) was added TFA (2.00 mL) dropwise at room temperature. The resulting mixture was stirred for 1 h at room temperature. The resulting mixture was concentrated under vacuum. 3-Amino-N-propylpropanamide (500.00 mg, crude) was obtained as yellow oil. LC/MS: mass calcd. For C₆H₁₄N₂O: 130.11, found: 131.20 [M+H]⁺.

### Step 3: Synthesis of ethyl 4-(3-aminopropanamido)-1-methylimidazole-2-carboxylate

The procedure was the same as methyl 4-[4-(3-aminopropanamido)-1-methylimidazole-2-amido]-1-methylpyrrole-2-carboxylate hydrochloride. 4.00 g of ethyl 4-[3-[(tert-butoxycarbonyl) amino] propanamido]-1-methylimidazole-2-carboxylate was used, 4.00 g crude of ethyl 4-(3-aminopropanamido)-1-methylimidazole-2-carboxylate was obtained as white solid. LC/MS: mass calcd. For C₁₀H₁₆N₄O₃: 240.12, found: 241.15 [M+H]⁺.

### Step 4: Synthesis of methyl 4-(4-((tert-butoxycarbonyl)amino)-1-methyl-1H-imidazole-2-carboxamido)-1-methyl-1H-pyrrole-2-carboxylate

The procedure was the same as ethyl 4-[3-[(tert-butoxycarbonyl)amino]propanamido]-1-methylimidazole-2-carboxylate. 1.00 g of 4-[(tert-butoxycarbonyl)amino]-1-methylimidazole-2-carboxylic acid was used, 1.34 g of methyl 4-(4-((tert-butoxycarbonyl)amino)-1-methyl-1H-imidazole-2-carboxamido)-1-methyl-1H-pyrrole-2-carboxylate was obtained as brown solid (85.00% yield). LC/MS: mass calcd. For C₁₇H₂₃N₅O₅: 377.17, found: 378.25 [M+H]⁺.

### Step 5: Synthesis of 4-(4-((tert-butoxycarbonyl)amino)-1-methyl-1H-imidazole-2-carboxamido)-1-methyl-1H-pyrrole-2-carboxylic acid

The procedure was the same as 4-[3-[(Tert-butoxycarbonyl)amino]propanamido]-1-methylimidazole-2-carboxylic acid. 1.33 g of methyl 4-(4-((tert-butoxycarbonyl)amino)-1-methyl-1H-imidazole-2-carboxamido)-1-methyl-1H-pyrrole-2-carboxylate was used, 946 mg of 4-(4-((tert-butoxycarbonyl)amino)-1-methyl-1H-imidazole-2-carboxamido)-1-methyl-1H-pyrrole-2-carboxylic acid was obtained as white solid (74.00% yield). LC/MS: mass calcd. For C₁₆H₂₁N₅O₅: 363.15, found: 364.25 [M+H]⁺.

### Step 6: Synthesis of Ethyl 4-(3-[[4-(4-[3-[(tert-butoxycarbonyl)amino]propanamido]-1-methylimidazole-2-amido)-1-methylpyrrol-2-yl]formamido] propanamido)-1-methylimidazole-2-carboxylate

To a stirred solution of 4-(4-[3-[(tert-butoxycarbonyl)amino]propanamido]-1-methylimidazole-2-amido)-1-methylpyrrole-2-carboxylic acid (3.60 g, 8.29 mmol, 1.00 equiv) in DMF (50.00 mL) was added NMI (2.04 g, 24.86 mmol, 3.00 equiv), TCFH (3.49 g, 12.43 mmol, 1.50 equiv) and ethyl 4-(3-aminopropanamido)-1-methylimidazole-2-carboxylate (2.19 g, 9.12 mmol, 1.10 equiv) in portions at 0 degrees C. The resulting mixture was stirred for 1 h at room temperature. The reaction was quenched with ice/water (150 mL) at 0 degrees C. The precipitated solids were collected by filtration and washed with H₂O (3×30 mL), dried under vacuum. Ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate (5.00 g, 90.08%) was obtained as yellow solid. LC/MS: mass calcd. For C₂₉H₄₀N₁₀O₈: 656.30, found: 657.50 [M+H]⁺.

### Step 7: Synthesis of Ethyl 4-[3-([4-[4-(3-aminopropanamido)-1-methylimidazole-2-amido]-1-methylpyrrol-2-yl]formamido)propanamido]-1-methylimidazole-2-carboxylate

The procedure was the same as 3-amino-N-propylpropanamide. 4.90 g of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was used, 4.90 g crude of ethyl 4-[3-([4-[4-(3-aminopropanamido)-1-methylimidazole-2-amido]-1-methylpyrrol-2-yl]formamido)propanamido]-1-methylimidazole-2-carboxylate was obtained as yellow oil. LC/MS: mass calcd. For C₂₄H₃₂N₁₀O₆: 556.25, found: 557.45 [M+H]⁺.

### Step 8: Synthesis of ethyl 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylate

The procedure was the same as ethyl 4-(3-[[4-(4-[3-[(tert-butoxycarbonyl)amino] propanamido]-1-methylimidazole-2-amido)-1-methylpyrrol-2-yl]formamido]propanamido)-1-methylimidazole-2-carboxylate. 2.80 g of 4-[4-[(tert-butoxycarbonyl)amino]-1-methylimidazole-2-amido]-1-methylpyrrole-2-carboxylic acid was used, 5.00 g of ethyl 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylate was obtained as yellow solid (71.94% yield). LC/MS: mass calcd. For C₄₀H₅₁N₁₅O₁₀: 901.39, found: 902.70 [M+H]⁺.

### Step 9: 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid. 2.00 of ethyl 4-(3-[[4-(4-[3-[(4-[4-[(tert-butoxycarbonyl) amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylate was used, 1.60 g of 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylic acid was obtained as yellow solid (71.60% yield). LC/MS: mass calcd. For C₃₈H₄₇N₁₅O₁₀: 873.36, found: 874.55 [M+H]⁺.

### Step 10: Synthesis of tert-butyl N-(1-methyl-2-[[1-methyl-5-([2-[(1-methyl-2-[[1-methyl-5-([2-[(1-methyl-2-[[2-(propylcarbamoyl)ethyl]carbamoyl]imidazol-4-yl)carbamoyl]ethyl]carbamoyl)pyrrol-3-yl]carbamoyl]imidazol-4-yl)carbamoyl]ethyl]carbamoyl)pyrrol-3-yl]carbamoyl]imidazol-4-yl)carbamate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 550.00 mg of 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl]formamido]propanamido)-1-methylimidazole-2-carboxylic acid was used, 310.00 mg of tert-butyl N-(1-methyl-2-[[1-methyl-5-([2-[(1-methyl-2-[[1-methyl-5-([2-[(1-methyl-2-[[2-(propylcarbamoyl)ethyl]carbamoyl]imidazol-4-yl)carbamoyl] ethyl]carbamoyl)pyrrol-3-yl]carbamoyl]imidazol-4-yl)carbamoyl]ethyl]carbamoyl)pyrrol-3-yl]carbamoyl]imidazol-4-yl)carbamate was obtained as yellow solid (44.96% yield). LC/MS: mass calcd. For C₄₄H₅₉N₁₇O₁₀: 985.46, found: 986.35 [M+H]⁺.

### Step 11: Synthesis of 4-(3-[[4-(4-amino-1-methylimidazole-2-amido)-1-methylpyrrol-2-yl]formamido]propanamido)-1-methyl-N-[1-methyl-5-([2-[(1-methyl-2-[[2-(propylcarbamoyl)ethyl]carbamoyl]imidazol-4-yl)carbamoyl]ethyl] carbamoyl)pyrrol-3-yl]imidazole-2-carboxamide

The procedure was the same as 3-amino-N-propylpropanamide. 140.00 mg of tert-butyl N-(1-methyl-2-[[1-methyl-5-([2-[(1-methyl-2-[[1-methyl-5-([2-[(1-methyl-2-[[2-(propylcarbamoyl)ethyl]carbamoyl]imidazol-4-yl)carbamoyl]ethyl]carbamoyl)pyrrol-3-yl]carbamoyl]imidazole-4-yl)carbamoyl]ethyl]carbamoyl)pyrrol-3-yl]carbamoyl]imidazol-4-yl)carbamate was used, 140.00 mg crude of 4-(3-[[4-(4-amino-1-methylimidazole-2-amido)-1-methylpyrrol-2-yl]formamido]propanamido)-1-methyl-N-[1-methyl-5-([2-[(1-methyl-2-[[2-(propylcarbamoyl)ethyl]carbamoyl]imidazol-4-yl)carbamoyl]ethyl]carbamoyl)pyrrol-3-yl]imidazole-2-carboxamide was obtained as yellow oil. LC/MS: mass calcd. For C₃₉H₅₁N₁₇O₈: 885.41, found: 886.60 [M+H]⁺.

### Example 12. Synthesis of 4-[3-[(4-[4-[3-([4-[4-(3-aminopropanamido)-1-methylimidazole-2-amido]-1-methylpyrrol-2-yl]formamido)propanamido]-1-methylimidazole-2-amido]-1-methylpyrrol-2-yl)formamido]propanamido]-1-methyl-N-propylimidazole-2-carboxamide (Compound 135)

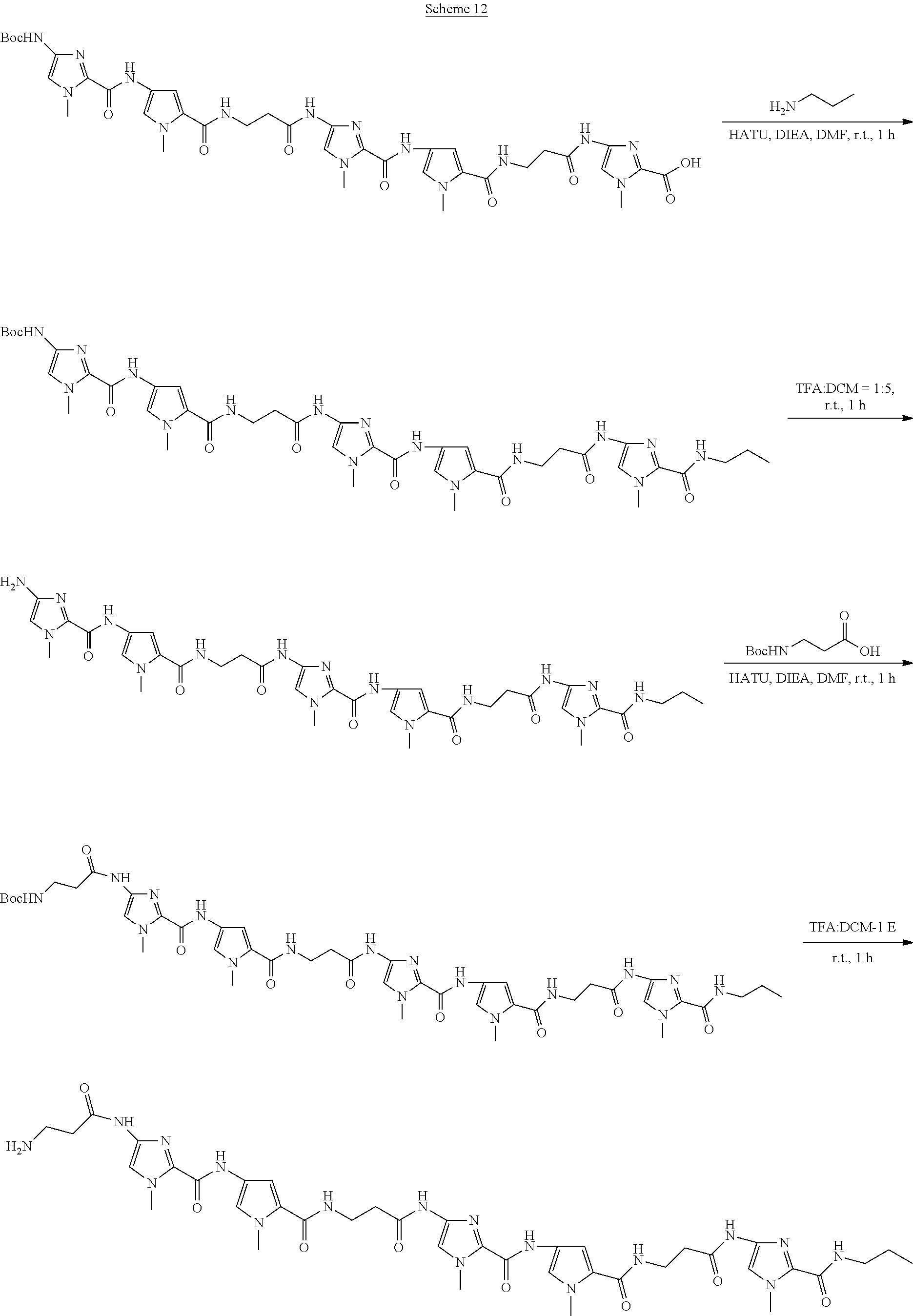

### Step 1: Synthesis of tert-butyl N-[1-methyl-2-([1-methyl-5-[(2-[[1-methyl-2-([1-methyl-5-[(2-[[1-methyl-2-(propylcarbamoyl)imidazol-4-yl]carbamoyl]ethyl) carbamoyl]pyrrol-3-yl]carbamoyl)imidazol-4-yl]carbamoyl]ethyl)carbamoyl]pyrrol-3-yl]carbamoyl) imidazol-4-yl]carbamate

The procedure was the same as ethyl 4-[3-[(tert-butoxycarbonyl)amino] propanamido]-1-methylimidazole-2-carboxylate. 1.00 g of 4-(3-[[4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrol-2-yl] formamido]propanamido)-1-methylimidazole-2-carboxylic acid was used, 680.00 mg of tert-butyl N-[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamate was obtained as brown solid (57.86% yield). LC/MS: mass calcd. For C₄₁H₅₄N₁₆O₉: 914.43, found: 915.65 [M+H]⁺.

### Step 2: Synthesis of 4-[3-([4-[4-(3-[[4-(4-amino-1-methylimidazole-2-amido)-1-methylpyrrol-2-yl]formamido]propanamido)-1-methylimidazole-2-amido]-1-methylpyrrol-2-yl]formamido)propanamido]-1-methyl-N-propylimidazole-2-carboxamide

The procedure was the same as 3-amino-N-propylpropanamide. 657.00 mg of tert-butyl N-[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamate was used, 630.00 mg crude of 4-[3-([4-[4-(3-[[4-(4-amino-1-methylimidazole-2-amido)-1-methylpyrrol-2-yl]formamido]propanamido)-1-methylimidazole-2-amido]-1-methylpyrrol-2-yl] formamido)propanamido]-1-methyl-N-propylimidazole-2-carboxamide was obtained as yellow oil. LC/MS: mass calcd. For C₃₆H₄₆N₁₆O₇: 814.37, found: 815.60 [M+H]⁺.

### Step 3: Synthesis of tert-butyl N-(2-[[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamoyl]ethyl)carbamate

The procedure was the same as ethyl 4-[3-[(tert-butoxycarbonyl)amino]propanamido]-1-methylimidazole-2-carboxylate, but the product was purified by reverse phase column under NH₃·H₂O system. 630.00 mg of 4-[3-([4-[4-(3-[[4-(4-amino-1-methylimidazole-2-amido)-1-methylpyrrol-2-yl]formamido]propanamido)-1-methylimidazole-2-amido]-1-methylpyrrol-2-yl]formamido)propanamido]-N-ethyl-1-methylimidazole-2-carboxamide was used, 200.00 mg of tert-butyl N-(2-[[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl] carbamoyl)imidazol-4-yl]carbamoyl]ethyl)carbamoyl]pyrrol-3-yl]carbamoyl)imidazol-4-yl]carbamoyl]ethyl)carbamate was obtained as yellow solid (25.80% yield). LC/MS: mass calcd. For C₄₄H₅₉N₁₇O₁₀: 985.46, found: 986.70 [M+H]⁺.

### Step 4: Synthesis of 4-[3-[(4-[4-[3-([4-[4-(3-aminopropanamido)-1-methylimidazole-2-amido]-1-methylpyrrol-2-yl]formamido)propanamido]-1-methylimidazole-2-amido]-1-methylpyrrol-2-yl)formamido]propanamido]-1-methyl-N-propylimidazole-2-carboxamide

The procedure was the same as 3-amino-N-propylpropanamide. 100.00 mg of tert-butyl N-(2-[[1-methyl-2-([1-methyl-5-[(2-[[1-methyl-2-([1-methyl-5-[(2-[[1-methyl-2-(propylcarbamoyl)imidazol-4-yl]carbamoyl]ethyl)carbamoyl]pyrrol-3-yl]carbamoyl)imidazol-4-yl]carbamoyl]ethyl)carbamoyl]pyrrol-3-yl]carbamoyl)imidazol-4-yl]carbamoyl]ethyl)carbamate was used, 100.00 mg crude of 4-[3-[(4-[4-[3-([4-[4-(3-aminopropanamido)-1-methylimidazole-2-amido]-1-methylpyrrol-2-yl]formamido)propanamido]-1-methylimidazole-2-amido]-1-methylpyrrol-2-yl)formamido]propanamido]-1-methyl-N-propylimidazole-2-carboxamide was obtained as yellow oil. LC/MS: mass calcd. For C₃₉H₅₁N₁₇O₈: 885.41, found: 886.70 [M+H]⁺.

### Example 13. Synthesis of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid (Compound 13)

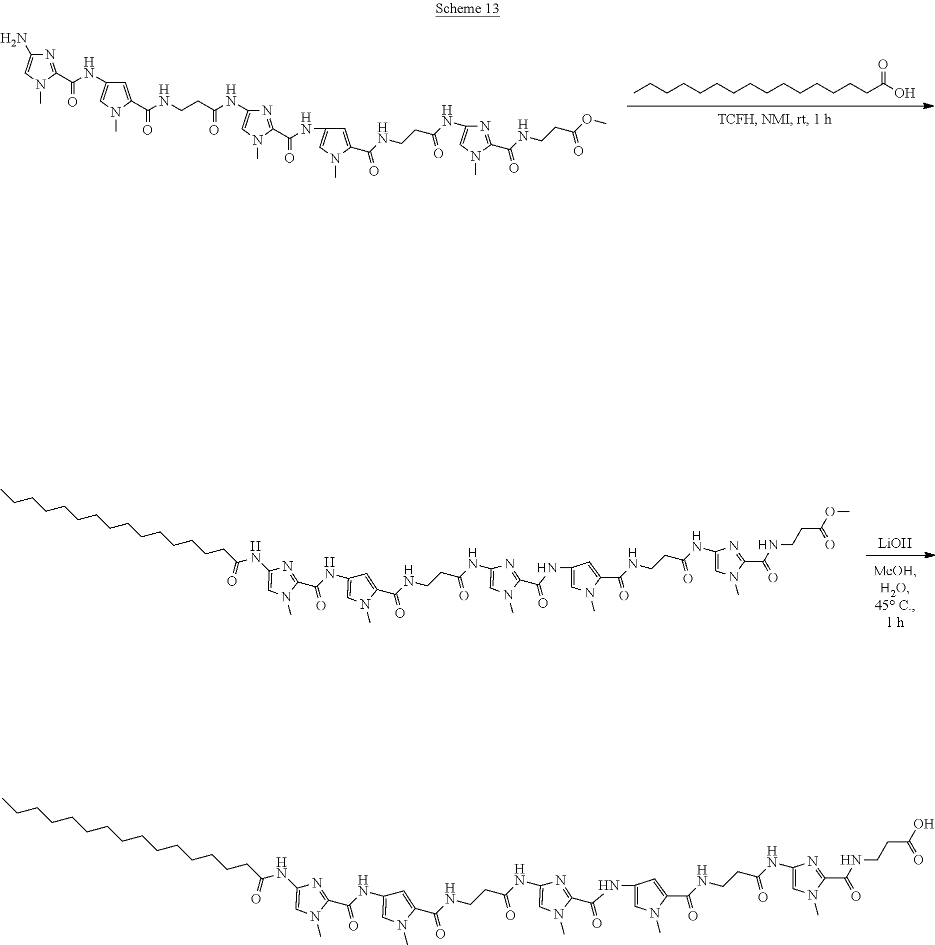

### Step 1: Synthesis of methyl 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido) propanamido]-1-methylimidazol-2-yl]formamido)propanoate

The procedure was the same as ethyl 4-(3-[[4-(4-[3-[(tert-butoxycarbonyl)amino]propanamido]-1-methylimidazole-2-amido)-1-methylpyrrol-2-yl] formamido] propanamido)-1-methylimidazole-2-carboxylate. 500.00 mg of methyl 3-([4-[3-([4-[4-(3-[[4-(4-amino-1-methylimidazole-2-amido)-1-methylpyrrol-2-yl] formamido] propanamido)-1-methylimidazole-2-amido]-1-methylpyrrol-2-yl]formamido)propanamido]-1-methylimidazol-2-yl]formamido)propanoate was used, 510.00 mg of methyl 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoate was obtained as brown solid (79.84% yield). LC/MS: mass calcd. For C₅₃H₇₆N₁₆O₁₀: 1096.59, found: 1097.50 [M+H]⁺.

### Step 2: Synthesis of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid

The procedure was the same as 3-([1-methyl-4-[3-([1-methyl-4-[1-methyl-4-(3-[[1-methyl-4-(1-methylimidazole-2-amido)pyrrol-2-yl] formamido]propanamido)imidazole-2-amido]pyrrol-2-yl]formamido)propanamido]imidazol-2-yl]formamido)propanoic acid. 270.00 mg of methyl 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoate was used, 260.00 mg crude of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid was obtained as brown solid. LC/MS: mass calcd. For C₅₂H₇₄N₁₆O₁₀: 1082.57, found: 1083.90 [M+H]⁺.

### Example 14. Synthesis of N-[5-([2-[(2-[[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]-4-hexadecanamido-1-methylimidazole-2-carboxamide (Compound 136)

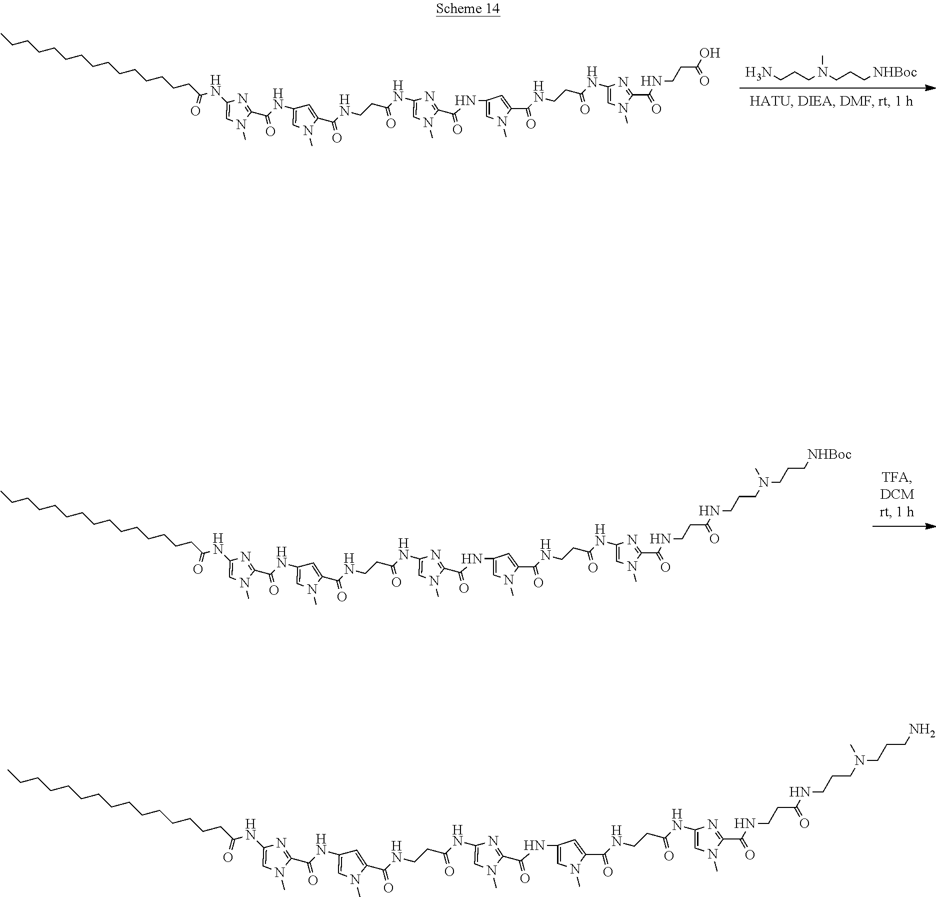

### Step 1: Synthesis of tert-butyl N-[3-([3-[3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanamido]propyl](methyl)amino)propyl]carbamate

The procedure was the same as tert-butyl (3-(methyl(3-(3-(1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamido)propanamido)propyl)amino)propyl)carbamate. 70.00 mg of 3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanoic acid was used, 50.00 mg of tert-butyl N-[3-([3-[3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanamido]propyl](methyl)amino)propyl]carbamate was obtained as yellow solid (59.04% yield). LC/MS: mass calcd. For C₆₄H₉₉N₁₉O₁₁: 1309.78, found: 1311.20 [M+H]⁺.

### Step 2: Synthesis of N-[5-([2-[(2-[[5-([2-[(2-[[2-([3-[(3-aminopropyl)(methyl) amino]propyl]carbamoyl)ethyl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]-4-hexadecanamido-1-methylimidazole-2-carboxamide

The procedure was the same as N-(3-((3-((3-aminopropyl)(methyl)amino)propyl) amino)-3-oxopropyl)-1-methyl-4-(3-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxamide. 50.00 mg of tert-butyl N-[3-([3-[3-([4-[3-([4-[4-(3-[[4-(4-hexadecanamido-1-methylimidazole-2-amido)-1-methylpyrrol-2-yl]formamido]propanamido)-1-methylimidazole-2-amido]-1-methylpyrrol-2-yl]formamido)propanamido]-1-methylimidazol-2-yl]formamido)propanamido]propyl](methyl)amino)propyl]carbamate was used, 40.00 mg crude of N-[5-([2-[(2-[[5-([2-[(2-[[2-([3-[(3-aminopropyl)(methyl)amino]propyl]carbamoyl)ethyl]carbamoyl]-1-methylimidazol-4-yl)carbamoyl]ethyl]carbamoyl)-1-methylpyrrol-3-yl]carbamoyl]-1-methylimidazol-4-yl)carbamoyl]ethyl]carbamoyl)-1-methylpyrrol-3-yl]-4-hexadecanamido-1-methylimidazole-2-carboxamide was obtained as yellow oil. LCMS: mass calcd. For C₅₉H₉₁N₁₉O₉: 1209.72, found: 1211.05 [M+H]⁺.

### Example 15. Synthesis of 4-[4-(3-[[4-(4-[3-[(tert-butoxycarbonyl)amino]]propanamido]-1-methylimidazole-2-amido)-1-methylpyrrol-2-yl]formamido]propanamido)-1-methylimidazole-2-amido]-1-methylpyrrole-2-carboxylic acid (Compound 28)

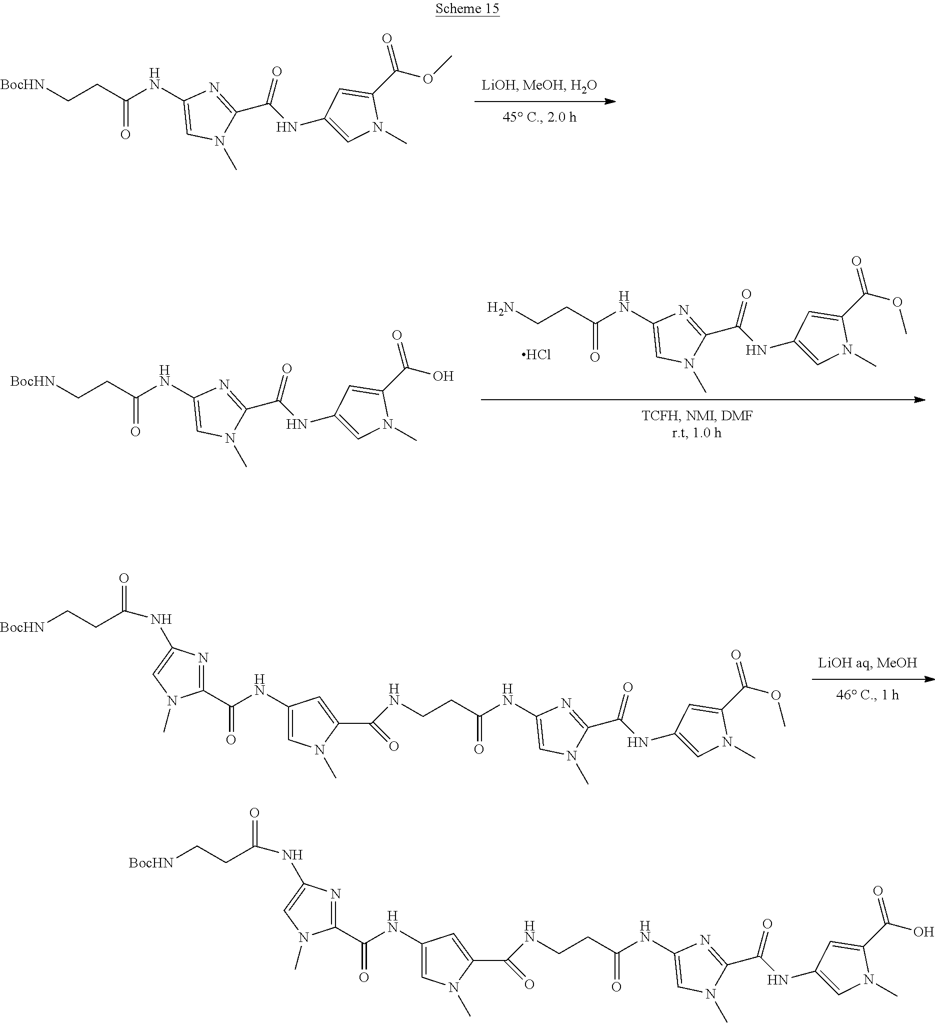 [SMILES: COC(=O)c1cc(NC(=O)c2nc(NC(=O)CCNC(=O)OC(C)(C)C)cn2C)cn1C ; Cn1cc(NC(=O)c2nc(NC(=O)CCNC(=O)OC(C)(C)C)cn2C)cc1C(=O)O ; COC(=O)c1cc(NC(=O)c2nc(NC(=O)CCN)cn2C)cn1C ; Cl ; COC(=O)c1cc(NC(=O)c2nc(NC(=O)CCNC(=O)c3cc(NC(=O)c4nc(NC(=O)CCNC(=O)OC(C)(C)C)cn4C)cn3C)cn2C)cn1C ; Cn1cc(NC(=O)c2nc(NC(=O)CCNC(=O)c3cc(NC(=O)c4nc(NC(=O)CCNC(=O)OC(C)(C)C)cn4C)cn3C)cn2C)cc1C(=O)O]

### Step 1: Synthesis of 4-(4-[3-[(tert-butoxycarbonyl)amino]propanamido]-1-methylinidazole-2-amido)-1-methylpyrrole-2-carboxylic acid

The procedure was the same as 4-[3-[(tert-butoxycarbonyl)amino]propanamido]-1-methylimidazole-2-carboxylic acid. 10.00 g of methyl 4-(4-[3-[(tert-butoxy carbonyl)amino]propanamido]-1-methylimidazole-2-amido)-1-methylpyrrole-2-carboxy late was used. 8.50 g of 4-(4-[3-[(tert-butoxycarbonyl)amino]propanamido]-1-methylimidazole-2-amido)-1-methylpyrrole-2-carboxylic acid was obtained as white solid (87.74% yield) LC/MS: mass calcd. for C₁₉H₂₆N₆O₆:434.19. found: 435.20 [M+H]⁺.

### Step 2: Synthesis of Methyl 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylate

The procedure was the same as methyl 4-(4-[3-[(tert-butoxycarbonyl)amino]propanamido]-1-methylimidazole-2-amido)-1-methylpyrrole-2-carboxylate. 1.00 g of 4-(4-[3-[(tert-butoxycarbonyl)amino]propanamido]-1-methylimidazole-2-amido)-1-methylpyrrole-2-carboxylic acid was used, 1.65 g of methyl 4-[4-(3-[[4-[[4-[3-[(tert-butoxy carbonyl)amino]propanamido]-1-methylimidazole-2 amido)-1-methylpyrrol-2-yl]formamido]propanamido)-1-methylimidazole-2-amido]-1-methylpyrrole-2-carboxylate was obtained as white solid (30.11% yield). LCMS: mass calcd. for C₃₄H₄N₁₂O₉: 764.34, found: 765.50 [M+H]⁺.

### Step 3: Synthesis of 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylic acid

The procedure was the same as 4-[3-[(tert-butoxycarbonyl)amino]propanamido]-1-methylimidazole-2-carboxylic acid. 100.00 mg of 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylate was used, 80.00 mg of 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylic acid was obtained as yellow solid (77.42% yield). LC/MS: mass calcd. for C₃₃H₄₂N₁₂O₉: 750.32, found: 751.30 [M+H]⁺.

### Example 16. Synthesis of Methyl 4-[4-[3-([4-[4-(3-aminopropanamido)-1-methylimidazole-2-amido]-1-methylpyrrol-2-yl]formamido)propanamido]-1-methylimidazole-2-amido]-1-methylpyrrole-2-carboxylate (Compound 137)

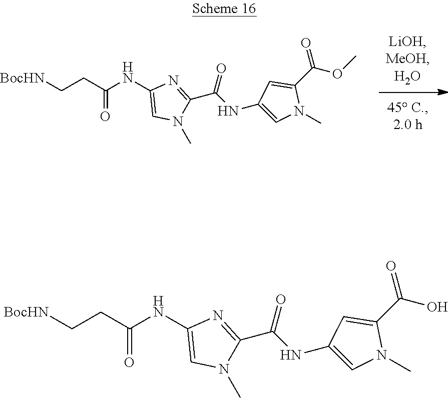 [SMILES: COC(=O)c1cc(NC(=O)c2nc(NC(=O)CCNC(=O)OC(C)(C)C)cn2C)cn1C ; Cn1cc(NC(=O)c2nc(NC(=O)CCNC(=O)OC(C)(C)C)cn2C)cc1C(=O)O]

The procedure was the same as 3-amino-N-propylpropanamide, but the reaction time was 2.0 h. 500.00 mg of methyl 4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrole-2-carboxylate was used, 500.00 mg of methyl 4-[4-[3-([4-[4-(3-aminopropanamido)-1-methylimidazole-2-amido]-1-methylpyrrol-2-yl]formamido)propanamido]-1-methylimidazole-2-amido]-1-methylpyrrole-2-carboxylate was obtained as yellow solid. HRMS: mass calcd. for C₂₉H₃₆N₁₂O₇: 664.2830, found: 665.2891 [M+H]⁺.

### Example 17. Synthesis of 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylic acid (Compound 138)

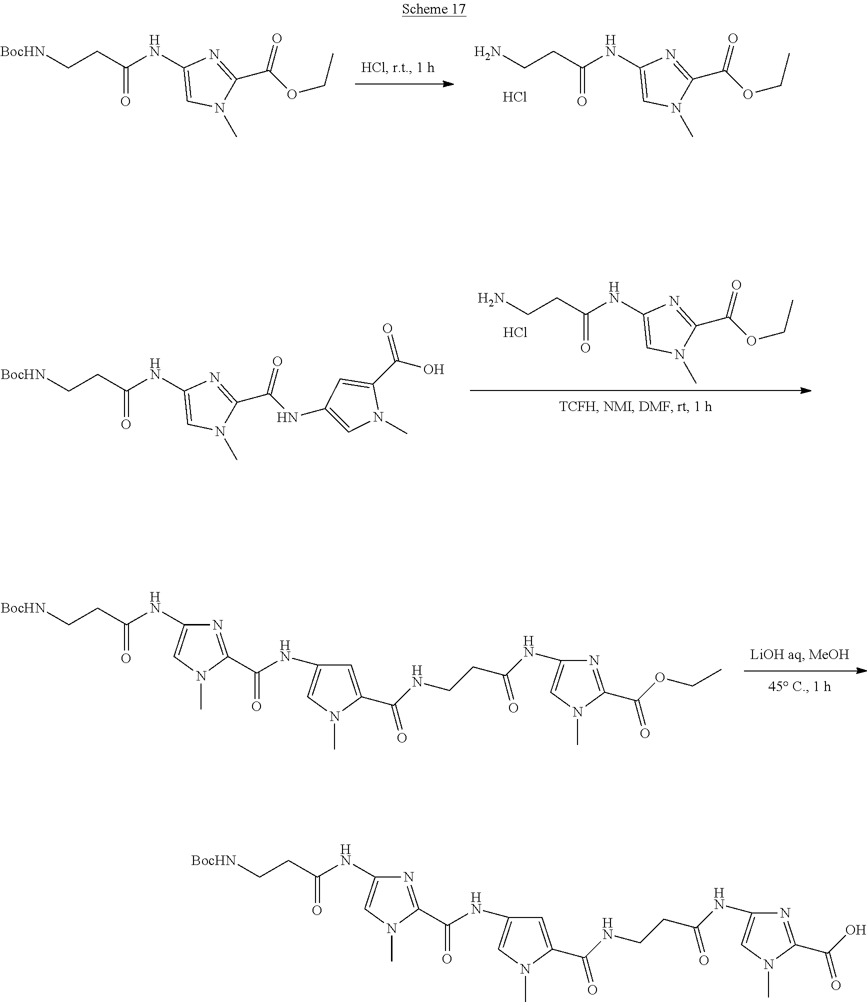

### Step 1: Synthesis of ethyl 4-(3-aminopropanamido)-1-methylimidazole-2-carboxylate

The procedure was the same as methyl 4-[4-(3-aminopropanamido)-1-methylimidazole-2-amido]-1-methylpyrrole-2-carboxylate hydrochloride. 2.00 g of ethyl 4-[3-[(tert-butoxycarbonyl)amino]propanamido]-1-methylimidazole-2-carboxylate was used, 2.00 g crude of ethyl 4-(3-aminopropanamido)-1-methylimidazole-2-carboxylate was obtained as off-white solid. LCMS: mass calcd. For C₁₀H₁₆N₄O₃: 240.12, found: 241.10 [M+H]⁺.

### Step 2: Synthesis of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate

The procedure was the same as ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate. 900.00 mg of 4-(4-[3-[(tert-butoxycarbonyl)amino]propanamido]-1-methylimidazole-2-amido)-1-methylpyrrole-2-carboxylic acid was used, 1.10 g of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was obtained as off-white solid (75.80% yield). LCMS: mass calcd. For C₂₉H₄₀N₁₀O₈: 656.30, found: 657.50 [M+H]⁺.

### Step 3: Synthesis of 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid. 600 mg of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was used, 500.00 mg of 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylic acid was obtained as yellow solid (78.35% yield). LCMS: mass calcd. For C₂₇H₃₆N₁₀O₈: 628.27, found: 629.45 [M+H]⁺.

### Example 18. Synthesis of Ethyl 4-(3-(4-(4-(3-aminopropanamido)-1-methyl-1H-imidazole-2-carboxamido)-1-methyl-1H-pyrrole-2-carboxamido)propanamido)-1-methyl-1H-imidazole-2-carboxylate (Compound 139)

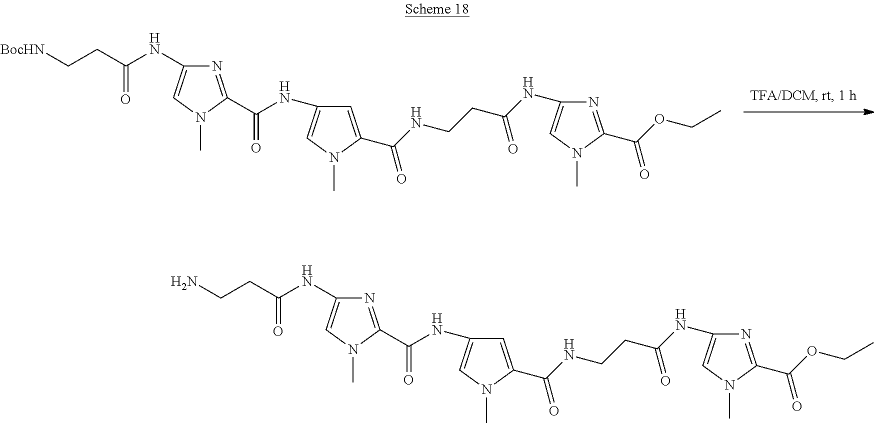 [SMILES: CCOC(=O)c1nc(NC(=O)CCNC(=O)c2cc(NC(=O)c3nc(NC(=O)CCNC(=O)OC(C)(C)C)cn3C)cn2C)cn1C ; CCOC(=O)c1nc(NC(=O)CCNC(=O)c2cc(NC(=O)c3nc(NC(=O)CCN)cn3C)cn2C)cn1C]

The procedure was the same as 3-amino-N-propylpropanamide. 900.00 mg of ethyl 4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-carboxylate was used, 900.00 mg crude of ethyl 4-[3-([4-[4-(3-aminopropanamido)-1-methylimidazole-2-amido]-1-methylpyrrol-2-yl]formamido)propanamido]-1-methylimidazole-2-carboxylate was obtained as white solid. LCMS: mass calcd. For C₂₄H₃₂N₁₀O₆: 556.25, found: 557.50 [M+H]⁺.

### Example 19. Synthesis of 4-(3-(4-(4-(3-(4-(4-(3-((tert-butoxycarbonyl)amino)propanamido)-1-methyl-1H-imidazole-2-carboxamido)-1-methyl-1H-pyrrole-2-carboxamido)propanamido)-1-methyl-1H-imidazole-2-carboxamido)-1-methyl-1H-pyrrole-2-carboxamido)propanamido)-1-methyl-1H-imidazole-2-carboxylic acid (PA16-OH)

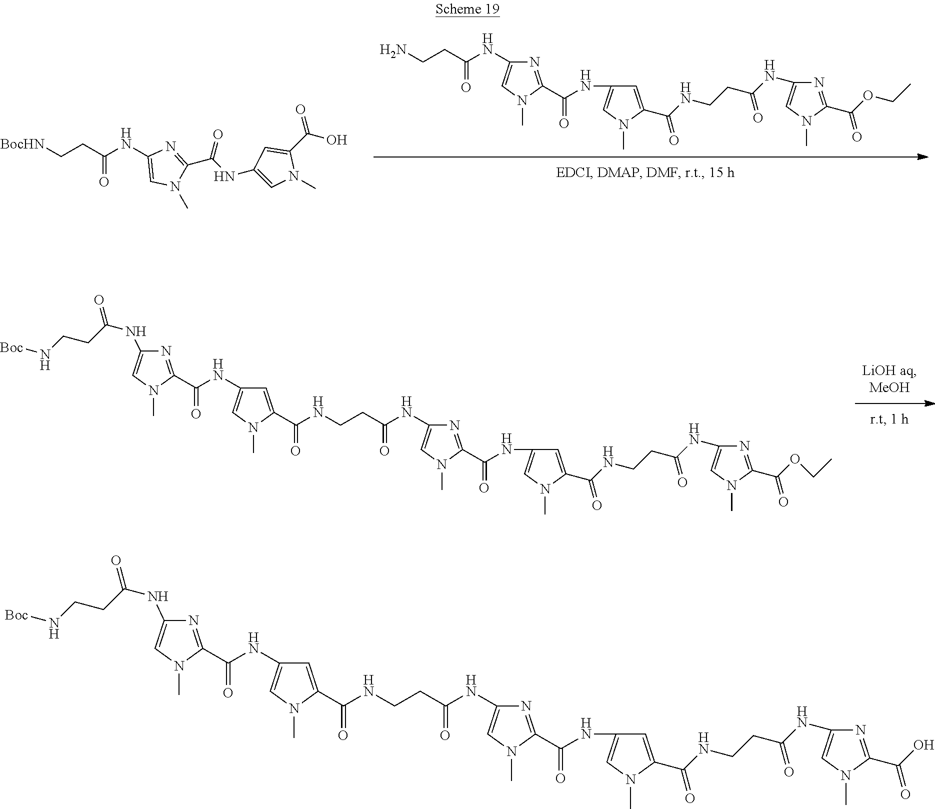 [SMILES: Cn1cc(NC(=O)c2nc(NC(=O)CCNC(=O)OC(C)(C)C)cn2C)cc1C(=O)O ; CCOC(=O)c1nc(NC(=O)CCNC(=O)c2cc(NC(=O)c3nc(NC(=O)CCN)cn3C)cn2C)cn1C ; CCOC(=O)c1nc(NC(=O)CCNC(=O)c2cc(NC(=O)c3nc(NC(=O)CCNC(=O)c4cc(NC(=O)c5nc(NC(=O)CCNC(=O)OC(C)(C)C)cn5C)cn4C)cn3C)cn2C)cn1C ; Cn1cc(NC(=O)c2nc(NC(=O)CCNC(=O)c3cc(NC(=O)c4nc(NC(=O)CCNC(=O)OC(C)(C)C)cn4C)cn3C)cn2C)cc1C(=O)NCCC(=O)Nc1cn(C)c(C(=O)O)n1]

### Step 1: Synthesis of ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate

To a stirred solution of 4-(4-[3-[(tert-butoxycarbonyl)amino]propanamido]-1-methylimidazole-2-amido)-1-methylpyrrole-2-carboxylic acid (120.00 mg, 0.276 mmol, 1.00 equiv) and EDCI (134.00 mg, 0.70 mmol, 2.53 equiv), DMAP (86.00 mg, 0.70 mmol, 2.55 equiv) in DMF (5.00 mL) was added ethyl 4-[3-([4-[4-(3-aminopropanamido)-1-methylimidazole-2-amido]-1-methylpyrrol-2-yl]formamido)propanamido]-1-methylimidazole-2-carboxylate (199.85 mg, 0.36 mmol, 1.30 equiv). The resulting solution was stirred for 16 h at room temperature. The resulting mixture was pour into ice/water (20 mL), the precipitated solids were collected by filtration and washed with water (3×10 mL). The solid was dried under reduced pressure to afford the ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate (240.00 mg, 80.37%) as yellow solid. LCMS: mass calcd. For C₄₃H₅₆N₁₆O₁₁: 972.43, found: 973.45 [M+H]⁺.

### Step 2: Synthesis of 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylic acid

The procedure was the same with 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA01-OH). 400.00 mg of ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate was used, 260.00 mg of 4-[3-([4-[4-(3-[[4-(4-[3-[(tert-butoxycarbonyl) amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylic acid was obtained as white solid (66.93% yield). LC/MS: mass calcd. For C₄₁H₅₂N₁₆O₁₁: 944.40, found: 945.40[M+H]⁺.

### Example 20. Synthesis of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylic acid (Compound 41)

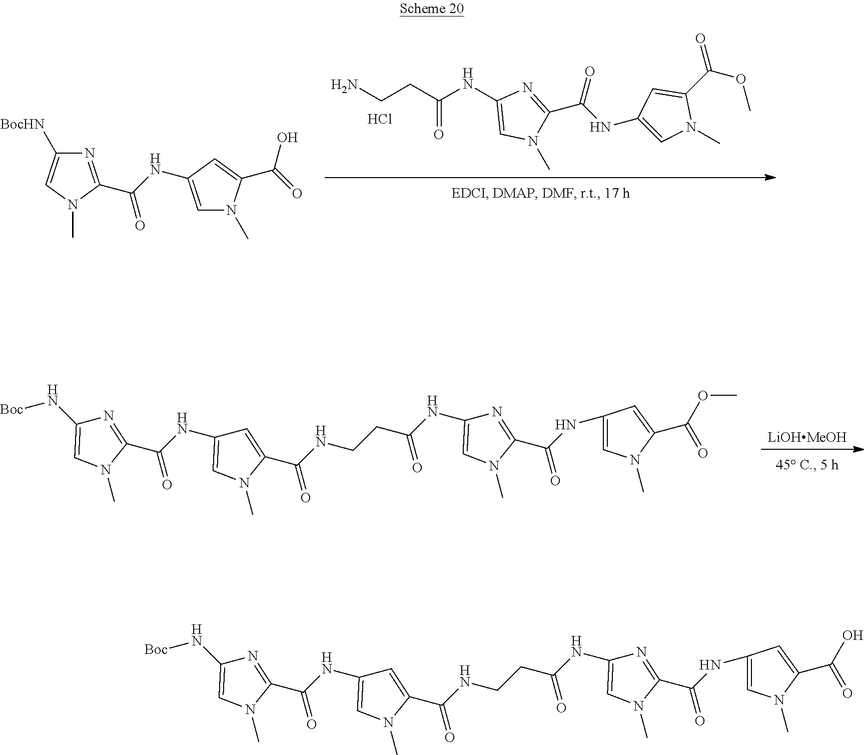 [SMILES: Cn1cc(NC(=O)c2nc(NC(=O)OC(C)(C)C)cn2C)cc1C(=O)O ; COC(=O)c1cc(NC(=O)c2nc(NC(=O)CCN)cn2C)cn1C ; Cl ; COC(=O)c1cc(NC(=O)c2nc(NC(=O)CCNC(=O)c3cc(NC(=O)c4nc(NC(=O)OC(C)(C)C)cn4C)cn3C)cn2C)cn1C ; "LiOH·MeOH" ; Cn1cc(NC(=O)c2nc(NC(=O)CCNC(=O)c3cc(NC(=O)c4nc(NC(=O)OC(C)(C)C)cn4C)cn3C)cn2C)cc1C(=O)O]

### Step 1: Synthesis of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylate

The procedure was the same as ethyl 4-[3-([4-[4-(3-[[4-(4-[3-[(tert-butoxycarbonyl)amino]propanamido]-1-methylimidazole-2-amido)-1-methylpyrrol-2-yl]formamido]propanamido)-1-methylimidazole-2-amido]-1-methylpyrrol-2-yl]formamido)propanamido]-1-methylimidazole-2-carboxylate. 1.00 g of 4-[4-[(tert-butoxycarbonyl)amino]-1-methylimidazole-2-amido]-1-methylpyrrole-2-carboxylic acid was used, 1.10 g of methyl 4-(4-[3-[(4-[4-[(tert-butoxycarbonyl) amino]-1-methylimidazole-2-amido]-1-methylpyrrol-2-yl)formamido]propanamido]-1-methylimidazole-2-amido)-1-methylpyrrole-2-carboxylate was obtained as dark yellow solid (51.28% yield). LC/MS: mass calcd. For C₃₁H₃₉NO₈: 693.30, found: 694.15 [M+H]⁺.

### Step 2: Synthesis of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylic acid

The procedure was the same as 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid. 500.00 mg of methyl 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylate was used, 400.00 mg of 4-(4-[3-[(4-[4-[(tert-butoxycarbonyl)amino]-1-methylimidazole-2-amido]-1-methylpyrrol-2-yl)formamido]propanamido]-1-methylimidazole-2-amido)-1-methylpyrrole-2-carboxylic acid was obtained as dark yellow solid (81.65% yield). LCMS: mass calcd. For C₃₀H₃₇N₁₁O: 679.28, found: 680.25 [M+H]⁺.

### Example 21. Synthesis of 3-[(1-methyl-4-[1-methyl-4-[3-([1-methyl-4-[1-methyl-4-(3-[[1-methyl-4-(1-methylimidazole-2-amido)imidazol-2-yl]formamido]propanamido)imidazole-2-amido]imidazol-2-yl]formamido)propanamido]imidazole-2-amido]imidazol-2-yl)formamido]propanoic acid (Compound 3)

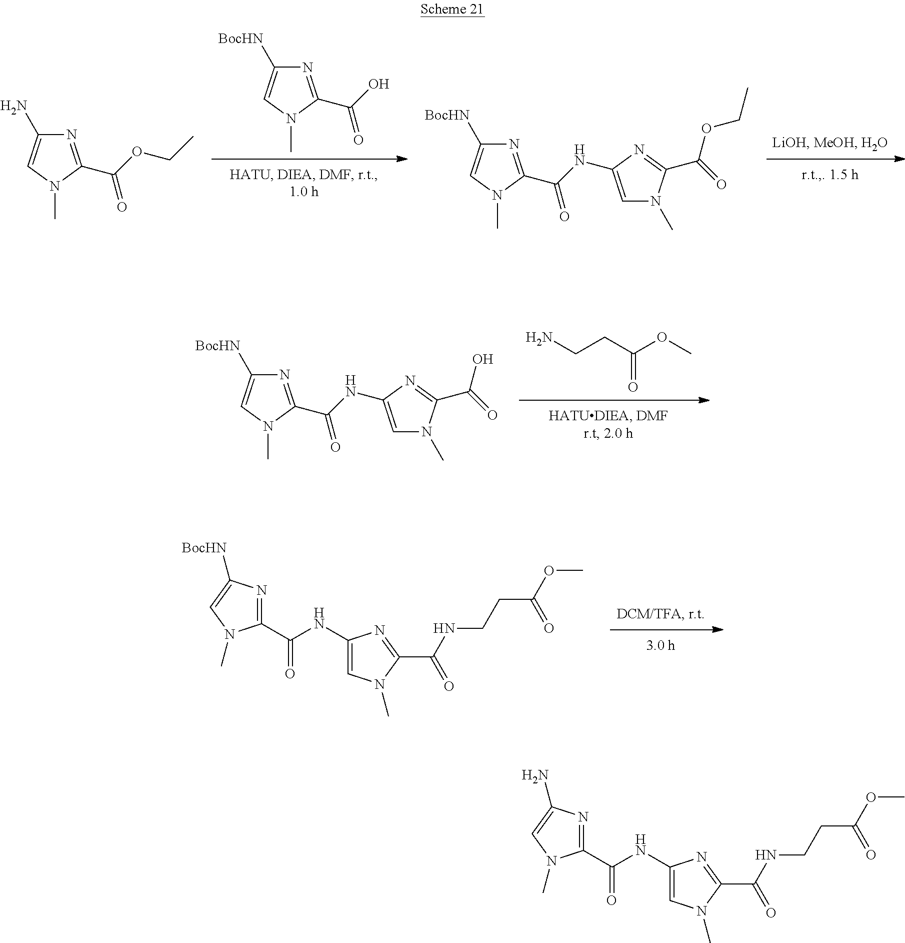

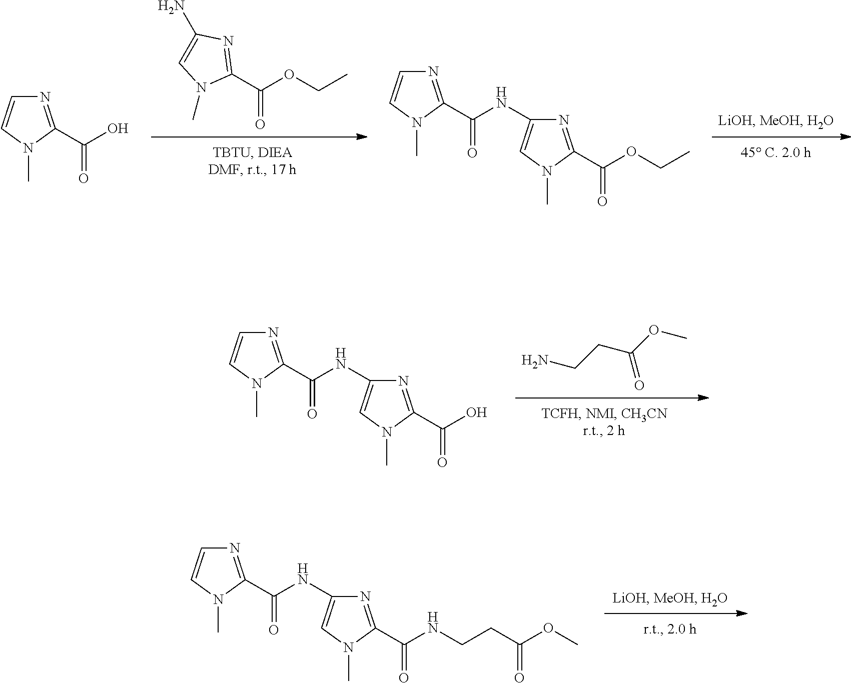

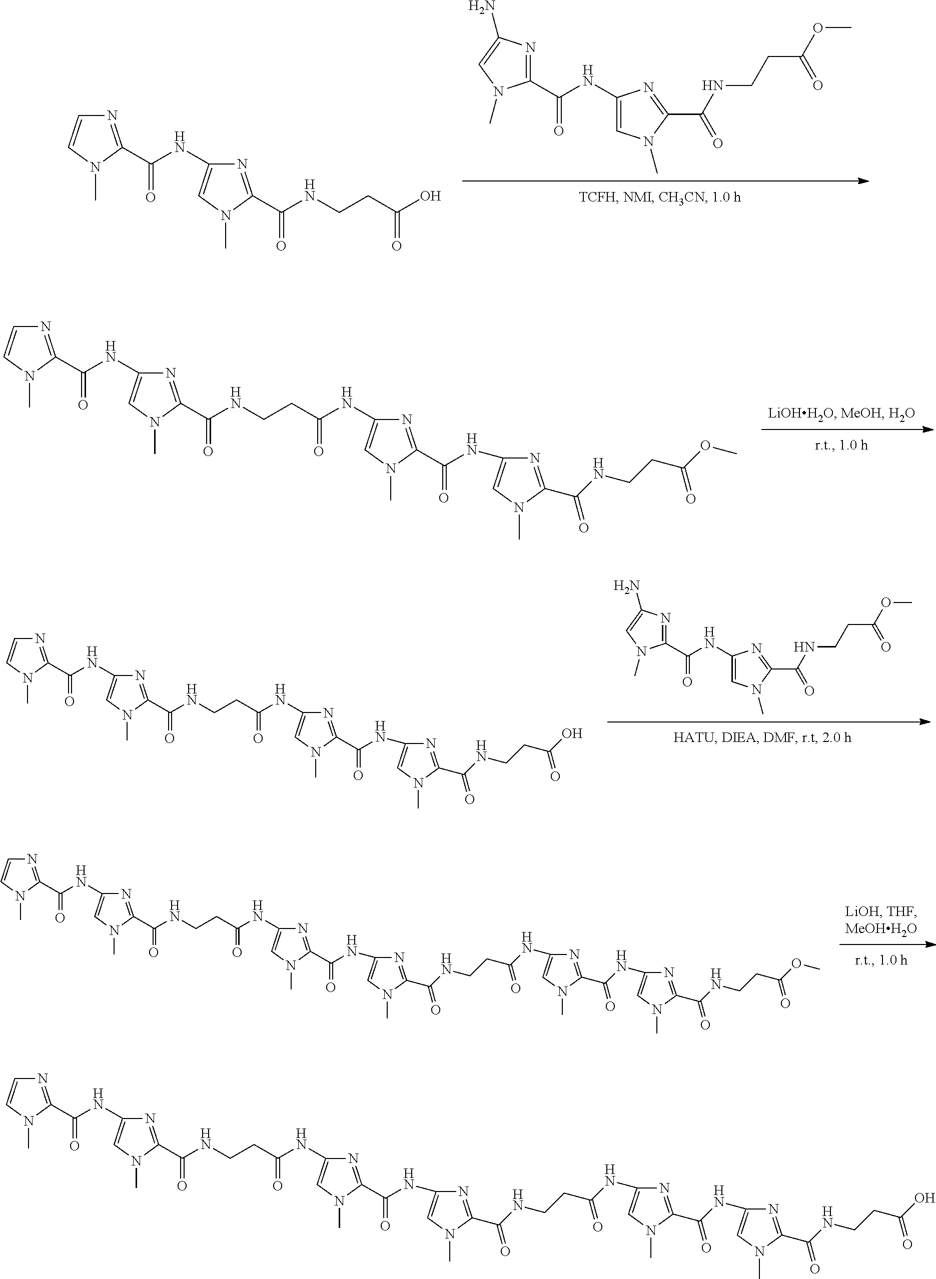

### Step 2: Synthesis of 4-[4-[(tert-butoxycarbonyl)amino]-1-methylimidazole-2-amido]-1-methylimidazole-2-carboxylic acid

The procedure was the same as 4-[3-[(tert-butoxycarbonyl)amino] propanamido]-1-methylimidazole-2-carboxylic acid, but the reaction temperature was r.t. and the reaction time was 1.5 h. 1.40 g of ethyl 4-[4-[(tert-butoxycarbonyl)amino]-1-methylimidazole-2-amido]-1-methylimidazole-2-carboxylate was used, 1.10 g of 4-[4-[(tert-butoxycarbonyl)amino]-1-methylimidazole-2-amido]-1-methylimidazole-2-carboxylic acid was obtained as white solid (84.00% yield). LC/MS: mass calcd. For C₁₅H₂₀N₆O₅: 364.15, found: 365.10 [M+H]⁺.

### Step 3: Synthesis of methyl 3-[(4-[4-[(tert-butoxycarbonyl)amino]-1-methylimidazole-2-amido]-1-methylimidazol-2-yl)formamido]propanoate

The procedure was the same as ethyl 4-[3-[(tert-butoxycarbonyl)amino]propanamido]-1-methylimidazole-2-carboxylate. 1.00 g of 4-[4-[(tert-butoxycarbonyl)amino]-1-methylimidazole-2-amido]-1-methylimidazole-2-carboxylic acid was used, 1.20 g of desired product was obtained as white solid (91.00% yield). LC/MS: mass calcd. For C₁₉H₂₇N₇O₆: 449.20, found: 450.30 [M+H]⁺.

### Step 4: Synthesis of methyl 3-[[4-(4-amino-1-methylimidazole-2-amido)-1-methylimidazol-2-yl]formamido]propanoate

The procedure was the same as 3-amino-N-propylpropanamide. 370.00 mg of methyl 3-[(4-[4-[(tert-butoxycarbonyl)amino]-1-methylimidazole-2-amido]-1-methylimidazol-2-yl)formamido]propanoate was used, 300.00 mg of methyl 3-[[4-(4-amino-1-methylimidazole-2-amido)-1-methylimidazol-2-yl]formamido]propanoate was obtained as a yellow oil (89.71% yield). LC/MS: mass calcd. For C₁₄H₁₉N₇O₄: 349.15, found: 350.15 [M+H]⁺.

### Step 5: Synthesis of ethyl 1-methyl-4-(1-methylimidazole-2-amido)imidazole-2-carboxylate

To a solution of 1-methylimidazole-2-carboxylic acid (500.00 mg, 3.97 mmol, 1.00 equiv) in DMF (12.00 mL) was added TBTU (1.80 g, 4.76 mmol, 1.20 equiv), ethyl4-amino-1-methylimidazole-2-carboxylate (737.83 mg, 4.36 mmol, 1.10 equiv) and DIPEA (1.02 g, 7.93 mmol, 2.00 equiv). The reaction was stirred at rt overnight. The mixture was poured into 30 mL ice water and stirred for 10 min. The precipitated solids were collected by filtration and washed with water (8 mL) to afford ethyl 1-methyl-4-(1-methylimidazole-2-amido)imidazole-2-carboxylate (920.00 mg, 79.50% yield) as white solid. LC/MS: mass calcd. For C₁₂H₁₅N₅O₃: 277.12, found: 278.05 [M+H]⁺.

### Step 6: Synthesis of 1-methyl-4-(1-methylimidazole-2-amido) imidazole-2-carboxylic acid

The procedure was the same as 4-[3-[(tert-butoxycarbonyl)amino]propanamido]-1-methylimidazole-2-carboxylic acid. 0.92 g of ethyl 1-methyl-4-(1-methylimidazole-2-amido)imidazole-2-carboxylate was used, 1.50 g crude of desired product was obtained as white solid. LC/MS: mass calcd. For C₁₀H₁₁N₅O₃: 249.09, found: 250.00 [M+H]⁺.

### Step 7: Synthesis of methyl 3-[[1-methyl-4-(1-methylimidazole-2-amido) imidazole-2-yl]formamido]propanoate

To a solution of 1-methyl-4-(1-methylimidazole-2-amido)imidazole-2-carboxylic acid (1.10 g, 4.40 mmol, 1.00 eq.) in MeCN (10.00 mL) was added NMI (1.12 g, 13.20 mmol, 3.10 eq.), TCFH (1.36 g, 5.00 mmol, 1.10 eq.) and methyl 3-aminopropanoate (0.50 g, 5.00 mmol, 1.10 eq.). Then the reaction was stirred at r.t. for 2.0 h. The precipitated solids was filtered out to give the desired product (900.00 mg) as white solid and the filtration was concentrated and the residue was purified by reverse flash chromatography with the following conditions: column, C18; mobile phase: MeCN in water (0.05% TFA), 5% to 50% gradient in 50 min; detector, UV 254 nm. The fractions were combined and concentrated. Another 500.00 mg of desired product was obtained as white solid. Totally 1.40 g desired product was obtained as light yellow solid (95.3% yield). LC/MS: mass calcd. For C₁₄H₁₈N₆O₄: 334.14, found: 335.05[M+H]⁺.

### Step 8: Synthesis of 3-[[1-methyl-4-(1-methylimidazole-2-amido) imidazol-2-yl]formamido]propanoic acid

To a solution of methyl 3-[[1-methyl-4-(1-methylimidazole-2-amido) imidazole-2-yl]formamido]propanoate (1.00 g, 2.99 mmol, 1.00 equiv) in MeOH (20.00 mL) was added LiOH solution (2M, 10.00 mmol, 3.34 equiv). The mixture was stirred at room temperature for 2.0 h. The resulting mixture was concentrated under the reduce pressure. The resulting mixture was diluted with water (5 mL) and cool to 0 degree C. Then the mixture was acidified to pH=5 with 2M HCl (aq.). The solid was filtered out and dried under vacuum. Then the aqueous phase was concentrated and the residue was purified by reverse phase column under the condition: column, C₁₈ silica gel; mobile phase, MeCN in water (0.05% TFA), 10% to 70% in 50 min; detector, UV 254 nm and 220 nm. The fractions were combined and concentrated. The result solid was combined with the filter cake. Totally 790.00 mg of 3-[[1-methyl-4-(1-methylimidazole-2-amido)imidazol-2-yl]formamido]propanoic acid as white solid(80.56% yield). LC/MS: mass calcd. For C₁₃H₁₆N₆O₄: 320.12, found: 321.05 [M+H]⁺.

### Step 9: Synthesis of methyl 3-([1-methyl-4-[1-methyl-4-(3-[[1-methyl-4-(1-methylimidazole-2-amido)imidazol-2-yl]formamido]propanamido)imidazole-2-amido]imidazol-2-yl]formamido)propanoate

The procedure was the same as methyl 4-(4-[3-[(tert-butoxycarbonyl)amino] propanamido]-1-methylimidazole-2-amido)-1-methylpyrrole-2-carboxylate. 630.00 mg of 3-[[1-methyl-4-(1-methylimidazole-2-amido)imidazol-2-yl]formamido]propanoic acid was used, 900.00 mg of desired product was obtained as off-white solid (70.31% yield). LC/MS: mass calcd. For C₂₇H₃₃N₁₃O₇: 651.26, found: 652.35 [M+H]⁺.

### Step 10: Synthesis of 3-([1-methyl-4-[1-methyl-4-(3-[[1-methyl-4-(1-methylimidazole-2-amido)imidazol-2-yl]formamido]propanamido)imidazole-2-amido]imidazol-2-yl]formamido)propanoic acid

The procedure was the same as 4-[3-[(tert-butoxycarbonyl)amino]propanamido]-1-methylimidazole-2-carboxylic acid. 1.00 g of methyl 3-([1-methyl-4-[1-methyl-4-(3-[[1-methyl-4-(1-methylimidazole-2-amido) imidazol-2-yl]formamido]propanamido)imidazole-2-amido]imidazol-2-yl]formamido) propanoate was used, 740.00 mg of desired product was obtained as white solid (74.12% yield). LC/MS: mass calcd. For C₂₆H₃₁N₁₃O₇: 637.25, found: 638.35 [M+H]⁺.

### Step 11: Synthesis of methyl 3-(1-methyl-4-(1-methyl-4-(3-(1-methyl-4-(1-methyl-4-(3-(1-methyl-4-(1-methyl-1H-imidazole-2-carboxamido)-1H-imidazole-2-carboxamido)propanamido)-1H-imidazole-2-carboxamido)-1H-imidazole-2-carboxamido)propanamido)-1H-imidazole-2-carboxamido)-1H-imidazole-2-carboxamido)propanoate

Into a 100 mL flask was added 3-([1-methyl-4-[1-methyl-4-(3-[[1-methyl-4-(1-methylimidazole-2-amido)imidazol-2-yl]formamido]propanamido)imidazole-2-amido]imidazol-2-1]formamido)propanoic acid (350.00 mg, 0.55 mmol, 1.00 equiv), DMF (20.00 mL). The mixture was cooled to 0 degrees C., HATU (313.07 mg, 0.82 mmol, 1.50 equiv) and DIEA (354.72 mg, 2.75 mmol, 5.00 equiv) were added. The mixture was stirred for 5 min, then methyl 3-[[4-(4-amino-1-methylimidazole-2-amido)-1-methylimidazol-2-yl]formamido]propanoate (230.12 mg, 0.66 mmol, 1.20 equiv) was added. The reaction was stirred at r.t. for 2.0 h. The reaction was concentrated, the residue was purified by reverse flash chromatography with the following conditions: column, C₁₈ silica gel; mobile phase, MeCN in water (0.05% TFA), 10% to 50% gradient in 50 min; detector, UV 254 nm. The fractions were combined and concentrated. 440.00 mg of desired product was obtained as yellow solid (62.87% yield). LC/MS: mass calcd. For C₄₀H₄₈N₂₀O₁₀: 968.39, found: 969.40 [M+H]⁺.

### Step 12: Synthesis of 3-[(1-methyl-4-[1-methyl-4-[3-([1-methyl-4-[1-methyl-4-(3-[[1-methyl-4-(1-methylimidazole-2-amido)imidazol-2-yl]formamido]propanamido)imidazole-2-amido]imidazol-2-yl]formamido)propanamido]imidazole-2-amido]imidazol-2-yl)formamido]propanoic acid

Into a 100 mL flask was added methyl 3-(1-methyl-4-(1-methyl-4-(3-(1-methyl-4-(1-methyl-4-(3-(1-methyl-4-(1-methyl-1H-imidazole-2-carboxamido)-1H-imidazole-2-carboxamido)propanamido)-1H-imidazole-2-carboxamido)-1H-imidazole-2-carboxamido)propanamido)-1H-imidazole-2-carboxamido)-1H-imidazole-2-carboxamido)propanoate (570.00 mg, 0.59 mmol, 1.00 equiv), MeOH (6.00 mL), THF (6.00 ml), LiOH (2.0 M, 1.50 mL, 16.95 mmol, 5.00 equiv). The reaction was stirred at r.t. for 1.0 h. The mixture was concentrated, the residue was dissolved with 10 mL water, cooled to 0 degrees C., adjust pH to 3-5 by 2 M HCl, the product did not precipitated out, so the water phase was concentrated, and water (5 mL) was added, when a few MeCN was added, some white solid was precipitated out and filtered. The filter cake was dried under vacuum. This result in 320.00 mg of 3-[(1-methyl-4-[1-methyl-4-[3-([1-methyl-4-[1-methyl-4-(3-[[1-methyl-4-(1-methylimidazole-2-amido)imidazol-2-yl]formamido]propanamido)imidazole-2-amido]imidazol-2-yl]formamido)propanamido]imidazole-2-amido]imidazol-2-yl)formamido]propanoic acid as dark yellow solid (51.27% yield). LC/MS: mass calcd. For C₃₉H₄₆N₂₀O₁₀: 954.37, found: 977.25 [M+Na]⁺.

### Example 22. Synthesis of 1-methyl-4-(3-(1-methyl-4-(1-methyl-4-(3-(1-methyl-4-(1-methyl-4-(3-(1-methyl-1H-pyrrole-2-carboxamido)propanamido)-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxylicacid (Compound 6)

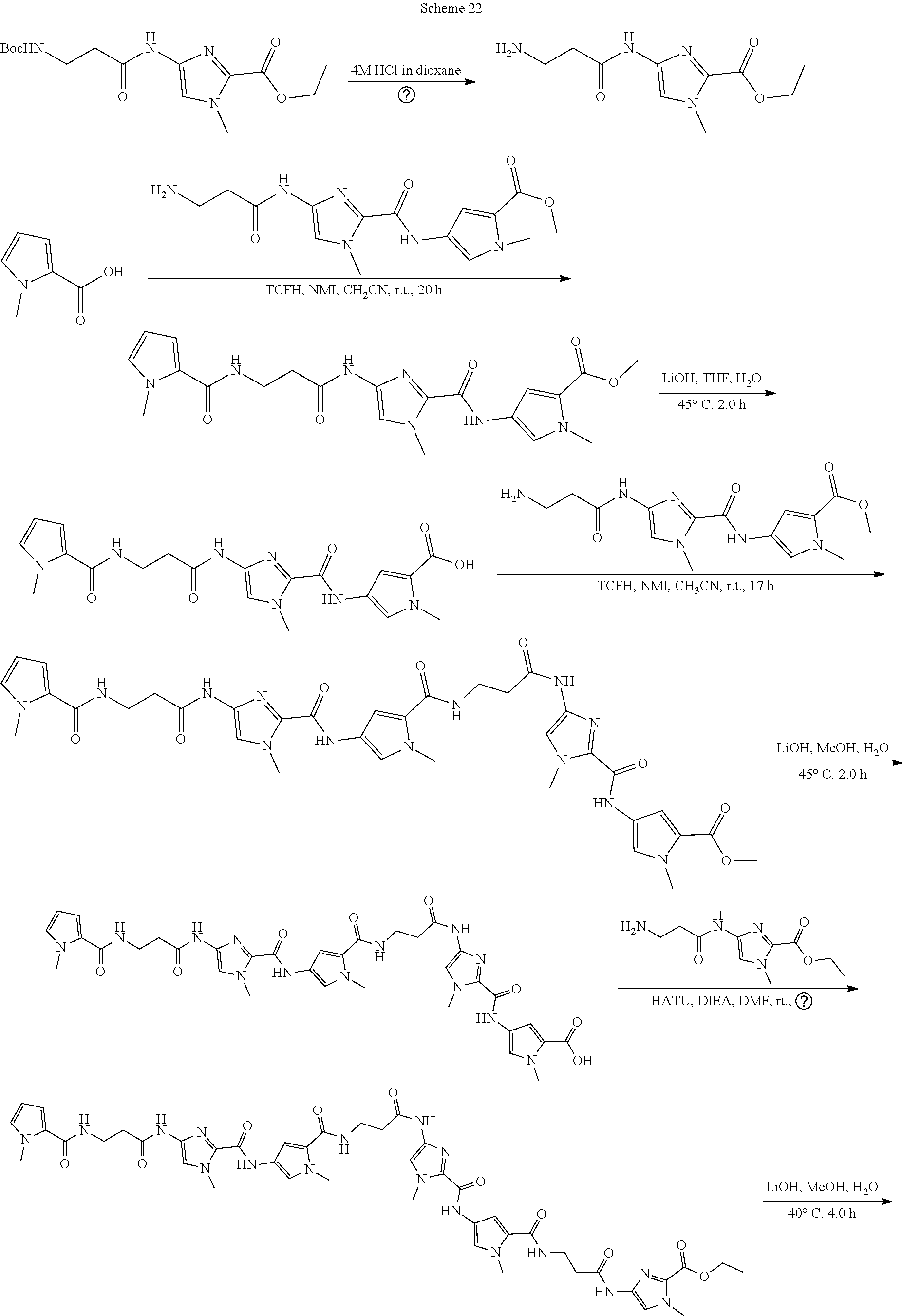

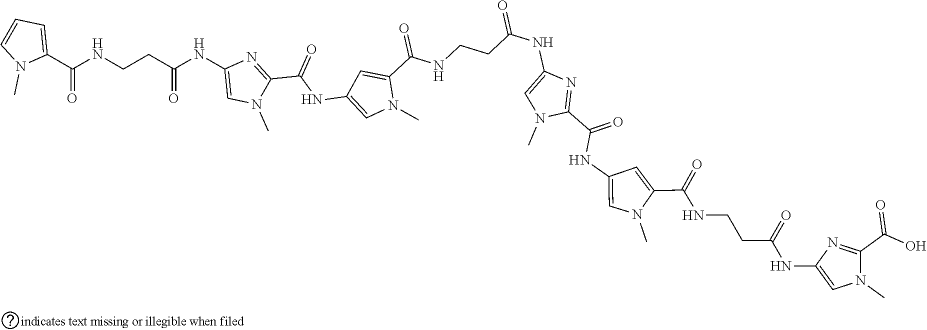 [SMILES: Cn1cc(NC(=O)c2nc(NC(=O)CCNC(=O)c3cc(NC(=O)c4nc(NC(=O)CCNC(=O)c5cccn5C)cn4C)cn3C)cn2C)cc1C(=O)NCCC(=O)Nc1cn(C)c(C(=O)O)n1]

### Step 1: Synthesis of ethyl 4-(3-aminopropanamido)-1-methyl-1H-imidazole-2-carboxylate

The procedure was the same as methyl 4-[4-(3-aminopropanamido)-1-methylimidazole-2-amido]-1-methylpyrrole-2-carboxylate hydrochloride. 2.00 g of ethyl 4-[13-[(tert-butoxycarbonyl)amino]propanamido]-1-methylimidazole-2-carboxylate was used, 2.00 g crude of ethyl 4-(3-aminopropanamido)-1-methyl-1H-imidazole-2-carboxylate was obtained as off-white solid. LC/MS: mass calcd. For C₁₀H₁₆N₄O₃: 240.12, found: 241.10 [M+H]⁺.

### Step 2: Synthesis of methyl 1-methyl-4-(1-methyl-4-[3-[(1-methylpyrrol-2-yl) formamido]propanamido]imidazole-2-amido)pyrrole-2-carboxylate

The procedure was the same as methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate. 600.00 mg of 1-methylpyrrole-2-carboxylic acid was used, 1.30 g of desired product was obtained ad white solid (56.00% yield). LC/MS: mass calcd. For C₂₁H₂₅N₇O₅: 455.19, found: 456.30 [M+H]⁺.

### Step 3: Synthesis of 1-methyl-4-(1-methyl-4-[3-[(1-methylpyrrol-2-yl)formamido]propanamido]imidazole-2-amido)pyrrole-2-carboxylic acid

The procedure was the same as 4-[3-[(tert-butoxycarbonyl)amino]propanamido]-1-methylimidazole-2-carboxylic acid. 2.00 g of methyl 1-methyl-4-(1-methyl-4-[3-[(1-methylpyrrol-2-yl)formamido]propanamido]imidazole-2-amido)pyrrole-2-carboxylate was used, 1.90 g of 1-methyl-4-(1-methyl-4-[3-[(1-methylpyrrol-2-yl)formamido]propanamido]imidazole-2-amido)pyrrole-2-carboxylic acid was obtained as white solid (92.00% yield). LC/MS: mass calcd. For C₂₀H₂₃N₇O₅: 441.18, found: 442.25 [M+H]⁺.

### Step 4: Synthesis of methyl 1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrole-2-carboxylate

The procedure was the same as methyl 3-[[1-methyl-4-(1-methylimidazole-2-amido)imidazole-2-yl]formamido]propanoate. 1.90 g of 1-methyl-4-(1-methyl-4-[3-[(1-methylpyrrol-2-yl)formamido]propanamido]imidazole-2-amido)pyrrole-2-carboxylic acid was used, 2.70 g of desired product was obtained as whit solid (71.00% yield). LC/MS: mass calcd. For C₃₅H₄₁N₁₃O₈: 771.32, found: 772.35 [M+H]⁺.

### Step 5: Synthesis of 1-methyl-4-[1-methyl-4-(3-[[1-methyl-4-(1-methyl-4-[3-[(1-methylpyrrol-2-1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrole-2-carboxylic acid

The procedure was the same as 4-[3-[(tert-butoxycarbonyl)amino]propanamido]-1-methylimidazole-2-carboxylic acid. 2.70 g of methyl 1-methyl-4-[1-methyl-4-(3-[[1-methyl-4-(1-methyl-4-[3-[(1-methylpyrrol-2-yl) formamido]propanamido]imidazole-2-amido)pyrrol-2-yl]formamido]propanamido)imidazole-2-amido]pyrrole-2-carboxylate was used, 2.80 g desired product was obtained as white solid. (78.00% yield). LC/MS: mass calcd. For C₃₄H₃₉N₁₃O₈: 757.30, found: 758.50 [M+H]⁺.

### Step 6: Synthesis of ethyl 1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-carboxylate

The procedure was the same as ethyl 4-[3-[(tert-butoxycarbonyl)amino]propanamido]-1-methylimidazole-2-carboxylate, but the obtained solid was purified by silica gel column with (DCM/MeOH=10/1). 2.50 g desired product was obtained as white solid (66.00% yield). LC/MS: mass calcd. For C₄₄H₅₃N₁₇O₁₀: 979.42, found: 980.80 [M+H]⁺.

### Step 7: Synthesis of 1-methyl-4-(3-(1-methyl-4-(1-methyl-4-(3-(1-methyl-4-(1-methyl-4-(3-(1-methyl-1H-pyrrole-2-carboxamido)propanamido)-1H-imidazole-2-carboxamido)-1H-pyrrole-2-carboxamido)propanamido)-1H-imidazole-2-carboxamido)-1H-pyrrole-2-carboxamido)propanamido)-1H-imidazole-2-carboxylic acid

The procedure was the same as 4-[3-[(tert-butoxycarbonyl)amino] propanamido]-1-methylimidazole-2-carboxylic acid, but the reaction temperature was 40° C. and reaction time is 4.0 h. 2.50 g of ethyl 1-methyl-4-[3-([1-methyl-4-[1-methyl-4-(3-[[1-methyl-4-(1-methyl-4-[3-[(1-methylpyrrol-2-yl)formamido]propanamido]imidazole-2-amido)pyrrol-2-yl]formamido]propanamido)imidazole-2-amido]pyrrol-2-yl]formamido)propanamido]imidazole-2-carboxylate was used, 1.90 g of desired product was observed as white solid (78.00% yield). LC/MS: mass calcd. For C₄₂H₄₉N₁₇O₁₀: 951.38, found: 952.65 [M+H]⁺.

### Example 23. Route 1 synthesis of 3-[(1-methyl-4-[1-methyl-4-[3-([l-methyl-4-[4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanamido]imidazol-2-yl]form amido)propanamido]pyrrole-2-amido]imidazol-2-yl)formamido]propanoic acid (Compound 8)

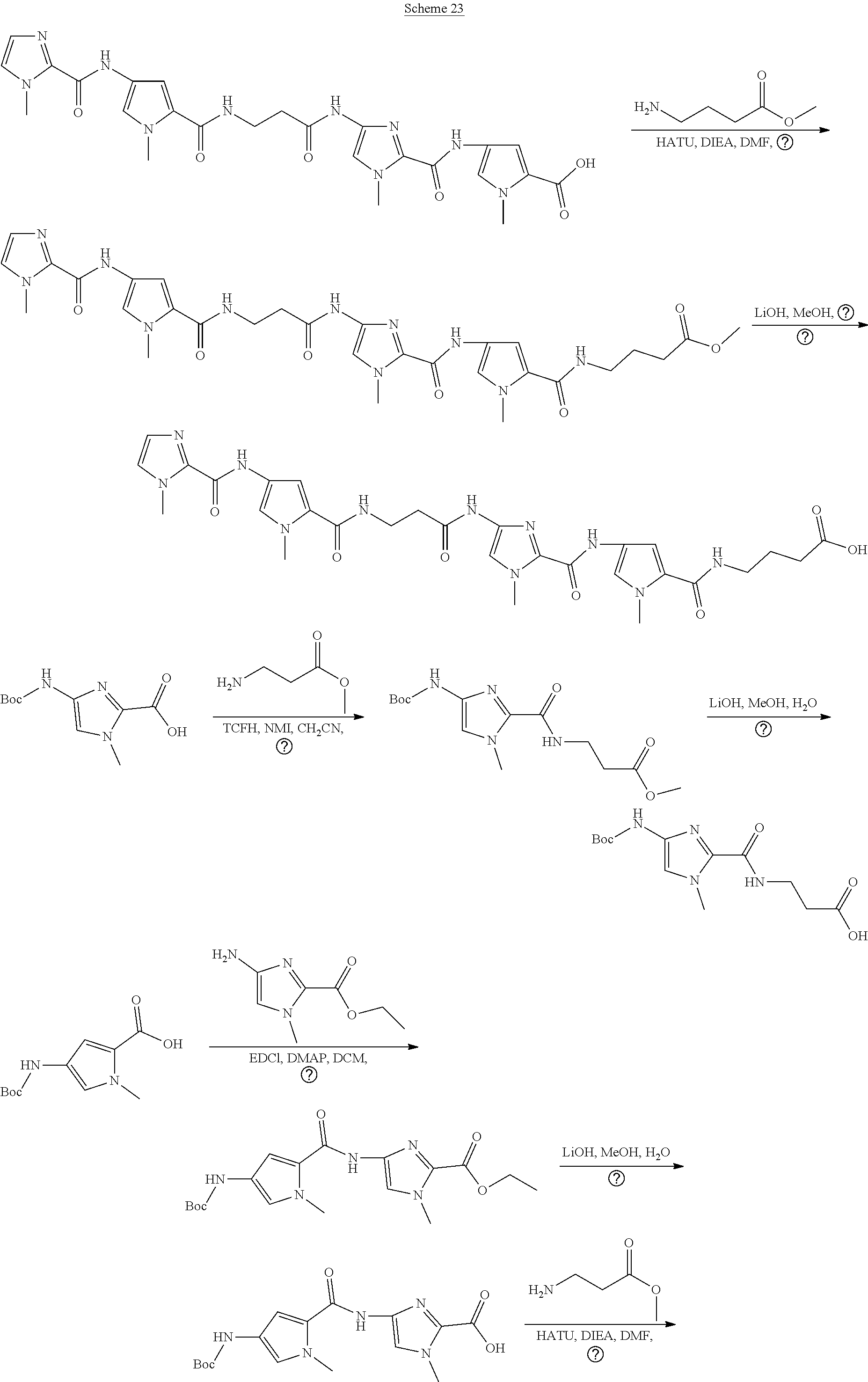

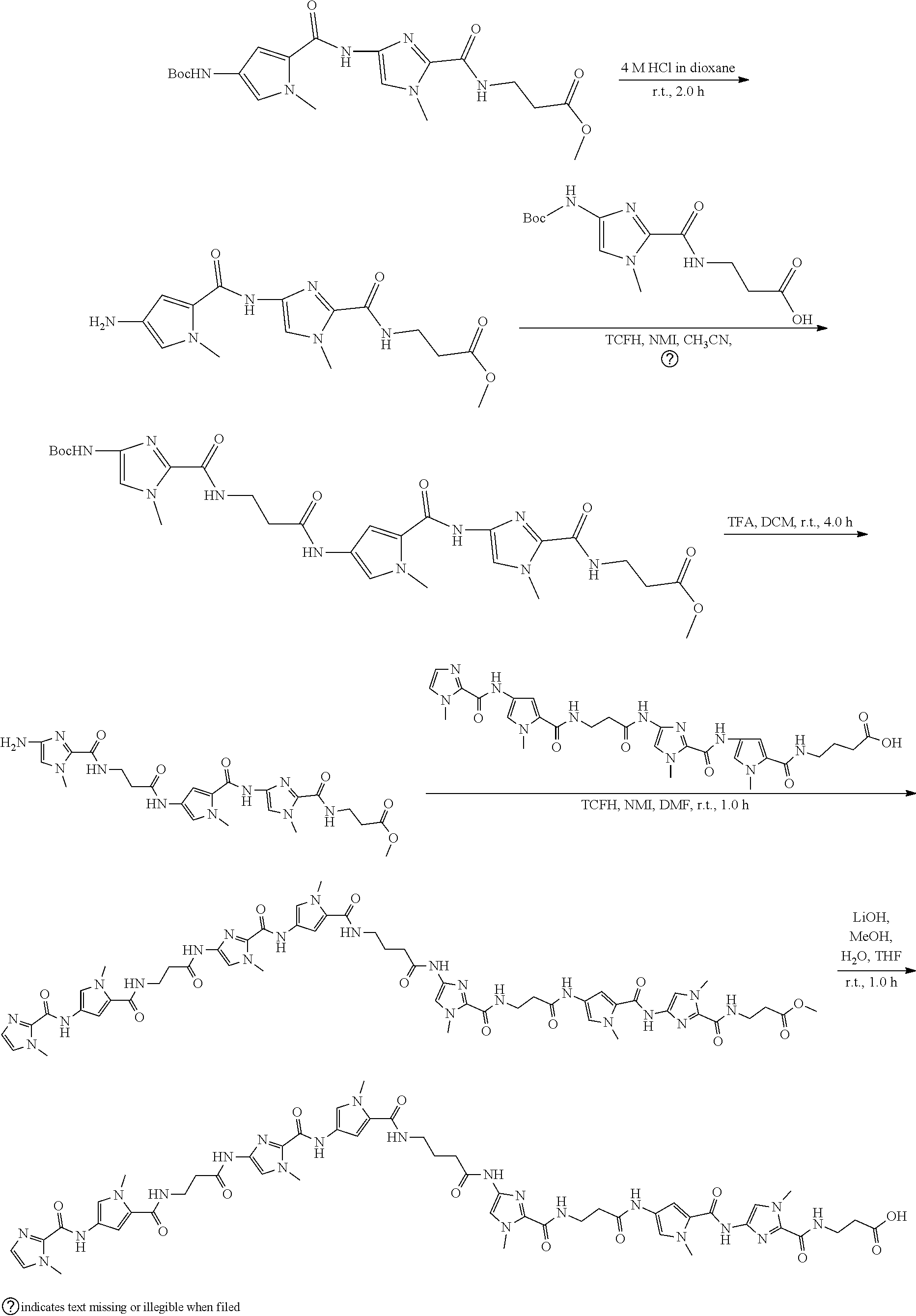

### Step 1: Synthesis of methyl 4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanoate

The procedure was the same as ethyl 4-[3-[(tert-butoxycarbonyl)amino]propanamido]-1-methylimidazole-2-carboxylate. 650.00 mg of 1-methyl-4-[1-methyl-4-(3-[[1-methyl-4-(1-methylimidazole-2-amido)pyrrol-2-yl]formamido]propanamido)imidazole-2-amido]pyrrole-2-carboxylic acid was used, 760.00 mg of methyl 4-([1-methyl-4-[1-methyl-4-(3-[[1-methyl-4-(1-methylimidazole-2-amido)pyrrol-2-yl]formamido]propanamido)imidazole-2-amido]pyrrol-2-yl] formamido)butanoate was obtained as yellow solid (95.23% yield). LC/MS: mass calcd. For C₃₀H₃₇N₁₁O₇: 663.29, found: 664.40 [M+H]⁺.

### Step 2: Synthesis of 4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanoic acid

The procedure was the same as 4-[3-[(tert-butoxycarbonyl)amino] propanamido]-1-methylimidazole-2-carboxylic acid, but the reaction temperature was r.t., the reaction time was 1.0 h. 760.00 of methyl 4-([1-methyl-4-[1-methyl-4-(3-[[1-methyl-4-(1-methylimidazole-2-amido) pyrrol-2-yl]formamido]propanamido)imidazole-2-amido]pyrrol-2-yl]formamido) butanoate was used, 750.00 mg of in 4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanoic acid was obtained as yellow solid (99.49% yield). LC/MS: mass calcd. For C₂₉H₃₅N₁₁O₇: 649.27, found: 650.20 [M+H]⁺.

### Step 3: Synthesis of methyl 3-([4-[(tert-butoxycarbonyl)amino]-1-methylimidazol-2-yl]formamido)propanoate

The procedure was the same as methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate. 1.00 g of 4-[(tert-butoxycarbonyl)amino]-1-methylimidazole-2-carboxylic acid was obtained, 1.00 g of methyl 3-([4-[(tert-butoxycarbonyl)amino]-1-methylimidazol-2-yl]formamido)propanoate was obtained as yellow oil (64.31% yield). LC/MS: mass calcd. For C₁₄H₂₂N₄O₈: 326.16, found: 327.15 [M+H]⁺.

### Step 4: Synthesis of 3-([4-[(tert-butoxycarbonyl)amino]-1-methylimidazol-2-yl]formamido)propanoic acid

The procedure was the same as 3-[[1-methyl-4-(1-methylimidazole-2-amido) imidazol-2-yl]formamido]propanoic acid, but the reaction time was 1.0 h. 1.00 g of methyl 3-([4-[(tert-butoxycarbonyl)amino]-1-methylimidazol-2-yl]formamido)propanoate was used, 600.00 mg of 3-([4-[(tert-butoxycarbonyl)amino]-1-methylimidazol-2-yl]formamido)propanoic acid was obtained as white solid (62.80% yield). LC/MS: mass calcd. For C₁₃H₂₀N₄O₈: 312.14, found: 313.10 [M+H]⁺.

### Step 5: Synthesis of ethyl 4-[4-[(tert-butoxycarbonyl)amino]-1-methylpyrrole-2-amido]-1-methylimidazole-2-carboxylate

Into a 250 ml flask was added 4-[(tert-butoxycarbonyl)amino]-1-methylpyrrole-2-carboxylic acid (1.00 g, 4.16 mmol, 1.00 equiv), DCM (40.00 mL), ethyl 4-amino-1-methylimidazole-2-carboxylate (0.70 g, 4.16 mmol, 1.00 equiv), EDCI (1.60 g, 8.32 mmol, 2.00 equiv), the mixture was stirred at 0 degrees C. for 10 mins, DMAP (1.27 g, 10.41 mmol, 2.50 equiv) was added in portions. The reaction mixture was stirred at r.t. for 24 h. The reaction was concentrated, the residue was purified by reverse flash chromatography with the following conditions: column, C₁₈ column; mobile phase A: 0.05% TFA water; mobile phase B: MeCN, 30% to 50% gradient in 40 min; detector, UV 254 nm. The fractions were combined and concentrated. This resulted in ethyl 4-[4-[(tert-butoxycarbonyl) amino]-1-methylpyrrole-2-amido]-1-methylimidazole-2-carboxylate (970.00 mg, 54.72%) as a yellow solid. LC/MS: mass calcd. For C₁₈H₂₅N₅O₅: 391.19, found: 392.30 [M+H]⁺.

### Step 6: Synthesis of 4-[4-[(tert-butoxycarbonyl)amino]-1-methylpyrrole-2-amido]-1-methylimidazole-2-carboxylic acid

The procedure was the same as 4-[3-[(tert-butoxycarbonyl)amino] propanamido]-1-methylimidazole-2-carboxylic acid, but the reaction temperature was r.t., the reaction time was 1.0 h. 970.00 mg of ethyl 4-[4-[(tert-butoxycarbonyl)amino]-1-methylpyrrole-2-amido]-1-methylimidazole-2-carboxylate was used, 638.00 mg of 4-[4-[(tert-butoxycarbonyl)amino]-1-methylpyrrole-2-amido]-1-methylimidazole-2-carboxylic acid was obtained as yellow solid (64.36% yield). LC/MS: mass calcd. For C₁₆H₂₁N₅O₅: 363.15, found: 364.15[M+H]⁺.

### Step 7: Synthesis of methyl 3-[(4-[4-[(tert-butoxycarbonyl)amino]-1-methylpyrrole-2-amido]-1-methylimidazol-2-yl)formamido]propanoate

The procedure was the same as methyl 3-[(4-[3-[(tert-butoxycarbonyl)amino] propanamido]-1-methylimidazol-2-yl)formamido]propanoate. 638.00 mg of 4-[4-[(tert-butoxycarbonyl)amino]-1-methylpyrrole-2-amido]-1-methylimidazole-2-carboxylic acid was used, 500.00 mg of methyl 3-[(4-[4-[(tert-butoxycarbonyl)amino]-1-methylpyrrole-2-amido]-1-methylimidazol-2-yl)formamido]propanoate was obtained as yellow oil (54.73% yield). LC/MS: mass calcd. For C₂₀H₂₅N₆O₆: 448.21, found: 449.25[M+H]⁺.

### Step 8: Synthesis of methyl 3-[[4-(4-amino-1-methylpyrrole-2-amido)-1-methylimidazol-2-yl]formamido]propanoate

The procedure was the same as methyl 4-[4-(3-aminopropanamido)-1-methylimidazole-2-amido]-1-methylpyrrole-2-carboxylate hydrochloride. 1.00 g of methyl 3-[(4-[4-[(tert-butoxycarbonyl)amino]-1-methylpyrrole-2-amido]-1-methylimidazol-2-yl)formamido]propanoate was used, 850.00 mg of methyl 3-[[4-(4-amino-1-methylpyrrole-2-amido)-1-methylimidazol-2-yl]formamido]propanoate was obtained as yellow oil (68.61% yield). LC/MS: mass calcd. For C₁₅H₂₀N₆O₄: 348.15, found: 371.05[M+Na]⁺.

### Step 9: Synthesis of methyl 3-[(4-[4-[3-([4-[(tert-butoxycarbonyl)amino]-1-methylimidazol-2-yl]formamido)propanamido]-1-methylpyrrole-2-amido]-1-methylimidazol-2-yl)formamido]propanoate

The procedure was the same as methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate. 753.00 mg of 3-([4-[(tert-butoxycarbonyl)amino]-1-methylimidazol-2-yl]formamido)propanoic acid was used, 700.00 mg of methyl 3-[(4-[4-[3-([4-[(tert-butoxycarbonyl)amino]-1-methylimidazol-2-yl]formamido)propanamido]-1-methylpyrrole-2-amido]-1-methylimidazol-2-yl)formamido]propanoate was obtained as yellow solid (38.22% yield). LC/MS: mass calcd. For C₂₈H₃₈N₁₀O₈: 642.29, found: 643.40[M+H]⁺.

### Step 10: Synthesis of methyl 3-[[4-(4-[3-[(4-amino-1-methylimidazol-2-yl) formamido]propanamido]-1-methylpyrrole-2-amido)-1-methylimidazol-2-yl]formamido]propanoate

The procedure was the same as 3-amino-N-propylpropanamide. 700.00 mg of methyl 3-[(4-[4-[3-([4-[(tert-butoxycarbonyl)amino]-1-methylimidazol-2-yl]formamido)propanamido]-1-methylpyrrole-2-amido]-1-methylimidazol-2-yl)formamido]propanoate was used, 700.00 mg crude of methyl 3-[[4-(4-[3-[(4-amino-1-methylimidazol-2-yl)formamido] propanamido]-1-methylpyrrole-2-amido)-1-methylimidazol-2-yl]formamido]propanoate was obtained as yellow oil. LC/MS: mass calcd. For C₂₃H₃₀N₁₀O₆: 542.23, found: 543.35[M+H]⁺.

### Step 11: Synthesis of methyl 3-[(1-methyl-4-[1-methyl-4-[3-([1-methyl-4-[4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido) butanamido] imidazol-2-yl]formamido)propanamido]pyrrole-2-amido]imidazole-2-yl)formamido]propanoate

The procedure was the same as methyl 1-methyl-4-[1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazole-2-amido]pyrrole-2-carboxylate. 541.23 mg of 4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanoic acid was used, 900.00 mg of methyl 3-[(1-methyl-4-[1-methyl-4-[3-([1-methyl-4-[4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanamido]imidazol-2-yl]formamido)propanamido]pyrrole-2-amido]imidazole-2-yl)formamido]propanoate was obtained as yellow solid (68.96% yield). LC/MS: mass calcd. For C₅₂H₆₃N₂₁O₁₂: 1173.50, found: 588.15[M/2+H]⁺.

### Step 12: Synthesis of 3-[(1-methyl-4-[1-methyl-4-[3-([1-methyl-4-[4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanamido] imidazol-2-yl]formamido)propanamido]pyrrole-2-amidofimidazo]-2-yl) formamido]propanoic acid

The procedure was the same as 4-[3-[(tert-butoxycarbonyl)amino]propanamido]-1-methylimidazole-2-carboxylic acid, but the reaction temperature was r.t., the reaction time was 1.0 h. 900.00 mg of methyl 3-[(1-methyl-4-[1-methyl-4-[3-([1-methyl-4-[4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanamido]imidazol-2-yl]formamido) propanamido]pyrrole-2-amido]imidazole-2-yl)formamido]propanoate was used, 670.00 mg of 3-[(1-methyl-4-[1-methyl-4-[3-([1-methyl-4-[4-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanamido]imidazol-2-yl]formamido)propanamido]pyrrole-2-amido]imidazol-2-yl)formamido]propanoic acid was obtained as yellow solid (52.15% yield). LC/MS: mass calcd. For C₅₂H₆₃N₂₁O₁₂: 1159.48, found: 580.95[M/2+H]⁺.

### Example 24. Route 2 synthesis of 3-[(1-methyl-4-[1-methyl-4-[3-([1-methyl-4-[4-([l-methyl-4-[1-methyl-4-(3-[[1-methyl-4-(1-methylimidazole-2-amido)pyrrol-2-yl]formamido]propanamido)imidazole-2-amido]pyrrol-2-yl]formamido)butanamido]imidazol-2-yl]formamido)propanamido]pyrrole-2-amido]imidazol-2-VI)formamido]propanoic acid (Compound 8)

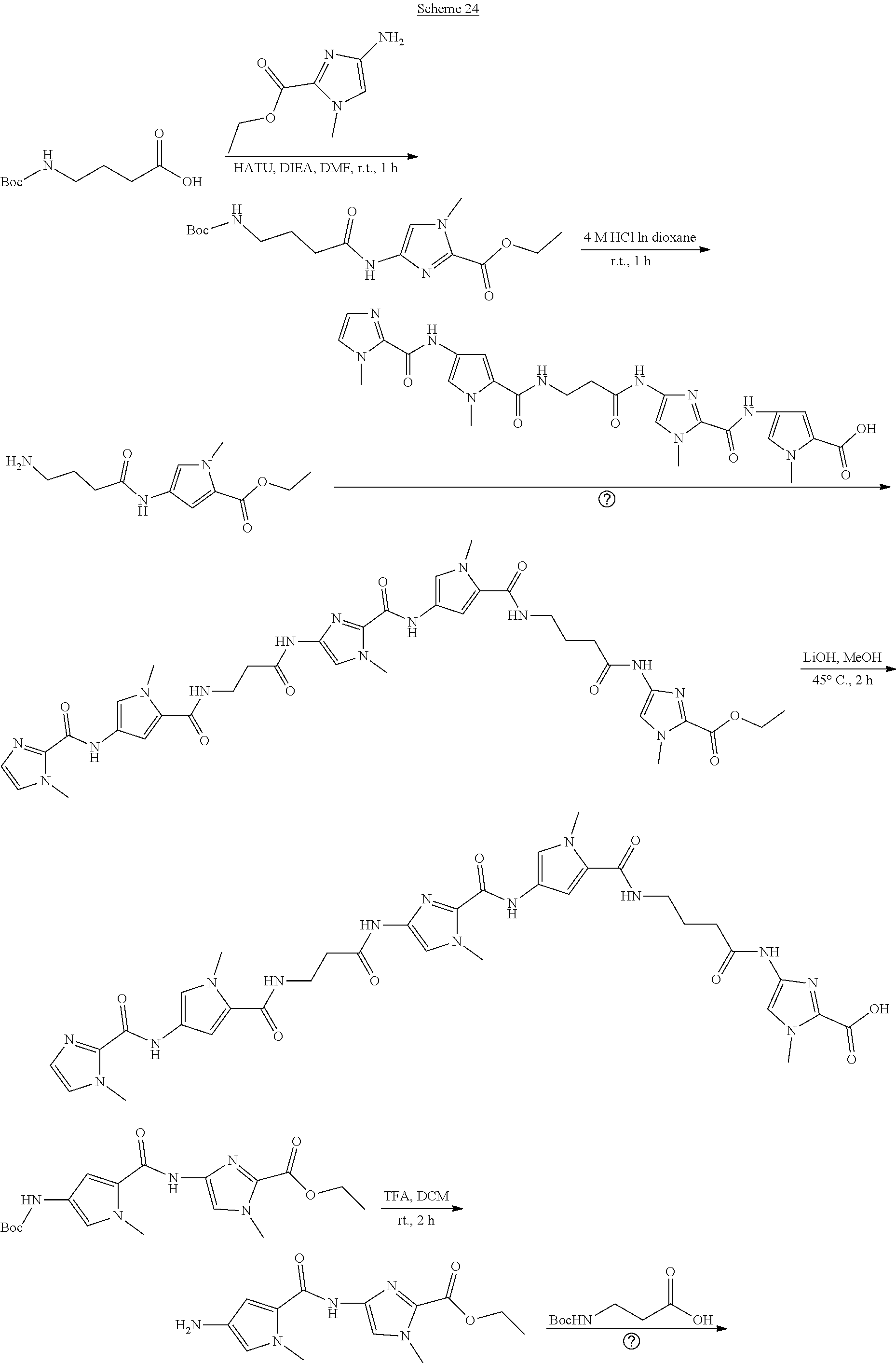

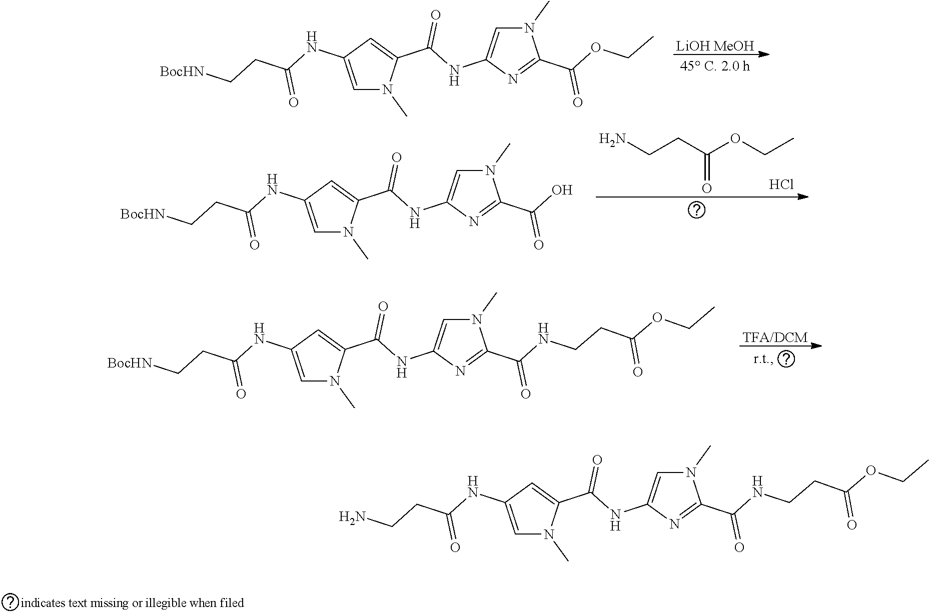

### Step 1: Synthesis of ethyl 4-[4-[(tert-butoxycarbonyl)amino]butanamido]-1-methylimidazole-2-carboxylate

The procedure was the same as ethyl 4-[3-[(tert-butoxycarbonyl)amino] propanamido]-1-methylimidazole-2-carboxylate. 7.80 g of 4-[(tert-butoxycarbonyl)amino]butanoic acid was obtained, 11.00 g of ethyl 4-{4-[(tert-butoxycarbonyl)amino]butanamido}-1-methylimidazole-2-carboxylate was obtained as little pink solid (80.70% yield). LC/MS: mass calcd. For C₁₆H₂₆N₄O₈: 354.19, found: 355.15[M+H]⁺.

### Step 2: Synthesis of ethyl 4-(4-aminobutanamido)-1-methylimidazole-2-carboxylate

The procedure was the same as methyl 4-[4-(3-aminopropanamido)-1-methylimidazole-2-amido]-1-methylpyrrole-2-carboxylate hydrochloride. 9.40 g of ethyl 4-{4-[(tert-butoxycarbonyl)amino]butanamido}-1-methylimidazole-2-carboxylate was used, 6.20 g of ethyl 4-(4-aminobutanamido)-1-methylimidazole-2-carboxylate was obtained as a white solid (90.89% yield). LCMS: mass calcd. For C₁₁H₁₈N₄O₃: 254.14, found: 255.15[M+H]⁺.

### Step 3: Synthesis of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate

To a stirred solution of 1-methyl-4-[1-methyl-4-(3-{[1-methyl-4-(1-methylimidazole-2-amido)pyrrol-2-yl]formamido}propanamido)imidazole-2-amido]pyrrole-2-carboxylic acid (18.20 g, 32.24 mmol, 1.00 equiv) in DMF (250.00 mL) was added DIEA (12.50 g, 96.71 mmol, 3.00 equiv), ethyl 4-(4-aminobutanamido)-1-methylimidazole-2-carboxylate (9.02 g, 35.46 mmol, 1.10 equiv) and PyBOP (20.13 g, 38.68 mmol, 1.20 equiv) at 0 degrees C. The resulting mixture was stirred for 1.0 h at room temperature. The reaction was poured into ice/water (800 mL). The precipitated solids were collected by filtration and washed with H₂O (3×200 mL), dried under vacuum. 24.70 g of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate was obtained as a yellow solid (95.74% yield). LC/MS: mass calcd. For C₃₁H₄₄N₁₄O₈: 800.35, found: 801.30[M+H]⁺.

### Step 4: Synthesis of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid

The procedure was the same as 4-[3-[(tert-butoxycarbonyl)amino]propanamido]-1-methylimidazole-2-carboxylic acid. 24.00 g of ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate was used, 23.10 g of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was obtained as a yellow solid (99.36% yield). LC/MS: mass calcd. For C₃₄H₄₀N₁₄O₈: 772.32, found: 773.30[M+H]⁺.

### Step 5: Synthesis of ethyl 4-(4-amino-1-methylpyrrole-2-amido)-1-methylimidazole-2-carboxylate

The procedure was the same as 3-amino-N-propylpropanamide, and the obtained crude was washed by Et₂O, dried under vacuum. 16.00 g of ethyl 4-{4-[(tert-butoxycarbonyl)amino]-1-methylpyrrole-2-amido}-1-methylimidazole-2-carboxylate was used, 16.00 g crude of ethyl 4-(4-amino-1-methylpyrrole-2-amido)-1-methylimidazole-2-carboxylate was obtained as brown solid. LC/MS: mass calcd. For C₁₃H₁₇N₅O₃: 291.13, found: 292.15[M+H]⁺.

### Step 6: Synthesis of ethyl 4-(4-{3-[(tert-butoxycarbonyl)amino]propanamido}-1-methylpyrrole-2-amido)-1-methylimidazole-2-carboxylate

A solution of ethyl 4-(4-amino-1-methylpyrrole-2-amido)-1-methylimidazole-2-carboxylate (12.00 g, 41.19 mmol, 1.00 equiv) and 3-[(tert-butoxycarbonyl)amino]propanoic acid (7.50 g, 39.64 mmol, 0.96 equiv), PyBOP (22.00 g, 42.28 mmol, 1.03 equiv), DIEA (45.00 g, 348.18 mmol, 8.45 equiv) in DMF (120.00 mL) was stirred for 1h at room temperature. The reaction was poured into ice water (400 mL), and the mixture was stirred for 15 min. The precipitated solids were collected by filtration and washed with water (3×150 mL) and dried under vacuum. The aqueous phase was extracted by EA (3×150 mL), the combined organic phases were combined and washed by H₂O (200 mL), dried over anhydrous Na₂SO₄. The solid was filtered out and the filtrate was concentrated. The residue was purified by silica gel column chromatography, eluted with PE/EA (1:8). Totally 17.00 g of ethyl 4-(4-{3-[(tert-butoxycarbonyl)amino]propanamido}-1-methylpyrrole-2-amido)-1-methylimidazole-2-carboxylate was obtained as a yellow solid (89.28%). LC/MS: mass calcd. For C₂₁H₃₀N₆O₆: 462.22, found: 463.35[M+H]⁺.

### Step 7: Synthesis of 4-(4-{3-[(tert-butoxycarbonyl)amino]propanamido}-1-methylpyrrole-2-amido)-1-methylimidazole-2-carboxylic acid

The procedure was the same as 4-[3-[(tert-butoxycarbonyl)amino]propanamido]-1-methylimidazole-2-carboxylic acid. 12.00 g of ethyl 4-(4-{3-[(tert-butoxycarbonyl)amino]propanamido}-1-methylpyrrole-2-amido)-1-methylimidazole-2-carboxylate was used, 10.00 g of 4-(4-{3-[(tert-butoxycarbonyl)amino]propanamido}-1-methylpyrrole-2-amido)-1-methylimidazole-2-carboxylic acid was obtained as white solid (88.81% yield). LC/MS: mass calcd. For C₁₉H₂₆N₆O₆: 434.19, found: 435.25[M+H]⁺.

### Step 8: Synthesis of ethyl 3-{[4-(4-{3-[(tert-butoxycarbonyl)amino]propanamido}-1-methylpyrrole-2-amido)-1-methylimidazol-2-yl]formamido}propanoate

A solution of 4-(4-{3-[(tert-butoxycarbonyl)amino]propanamido}-1-methylpyrrole-2-amido)-1-methylimidazole-2-carboxylic acid (10.00 g, 23.02 mmol, 1.00 equiv) and P-alanine ethyl ester hydrochloride (4.90 g, 31.90 mmol, 1.39 equiv), PyBOP (12.50 g, 24.02 mmol, 1.04 equiv), DIEA (9.00 g, 69.64 mmol, 3.03 equiv) in DMF (120.00 mL) was stirred for 1h at room temperature. The reaction was quenched by the addition of water (500 mL) at room temperature. The resulting mixture was extracted with EtOAc (3×400 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EA (1:8) to afford ethyl 3-{[4-(4-{3-[(tert-butoxycarbonyl)amino]propanamido}-1-methylpyrrole-2-amido)-1-methylimidazol-2-yl]formamido}propanoate (12.00 g, 93.80%) as a yellow solid. LC/MS: mass calcd. For C₂₄H₃₅N₇O₇: 533.26, found: 534.30[M+H]⁺.

### Step 9: Synthesis of ethyl 3-({4-[4-(3-aminopropanamido)-1-methylpyrrole-2-amido]-1-methylimidazol-2-yl}formamido)propanoate

The procedure was the same as 3-amino-N-propylpropanamide. 12.00 g of ethyl 3-{[4-(4-{3-[(tert-butoxycarbonyl)amino]propanamido}-1-methylpyrrole-2-amido)-1-methylimidazol-2-yl]formamido}propanoate was used, 12.00 g crude of ethyl 3-({4-[4-(3-aminopropanamido)-1-methylpyrrole-2-amido]-1-methylimidazol-2-yl}formamido)propanoate was obtained as white solid. LC/MS: mass calcd. For C₁₉H₂₇N₇O₅: 433.21, found: 434.25[M+H]⁺.

### Step 10: Synthesis of ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 10.00 g of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was used, 13.60 g of ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate was obtained as yellow solid (88.61% yield). LC/MS: mass calcd. For C₅₃H₆₅N₂₁O₁₂: 1187.51, found: 1211.05[M+Na]⁺.

### Step 11: Synthesis of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid

The procedure was the same as 4-[3-[(tert-butoxycarbonyl)amino]propanamido]-1-methylimidazole-2-carboxylic acid, but the reaction temperature was 35 degree C. 10.60 g of ethyl 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoate was used, 10.00 g crude of 3-[(1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazol-2-yl)formamido]propanoic acid was obtained as yellow solid. LC/MS: mass calcd. For C₅₁H₆₁N₂₁O₁₂: 1159.48, found: 581.25[M/2+H]⁺.

### Example 25. Synthesis of 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazole-2-carboxylic acid (Compound 86)

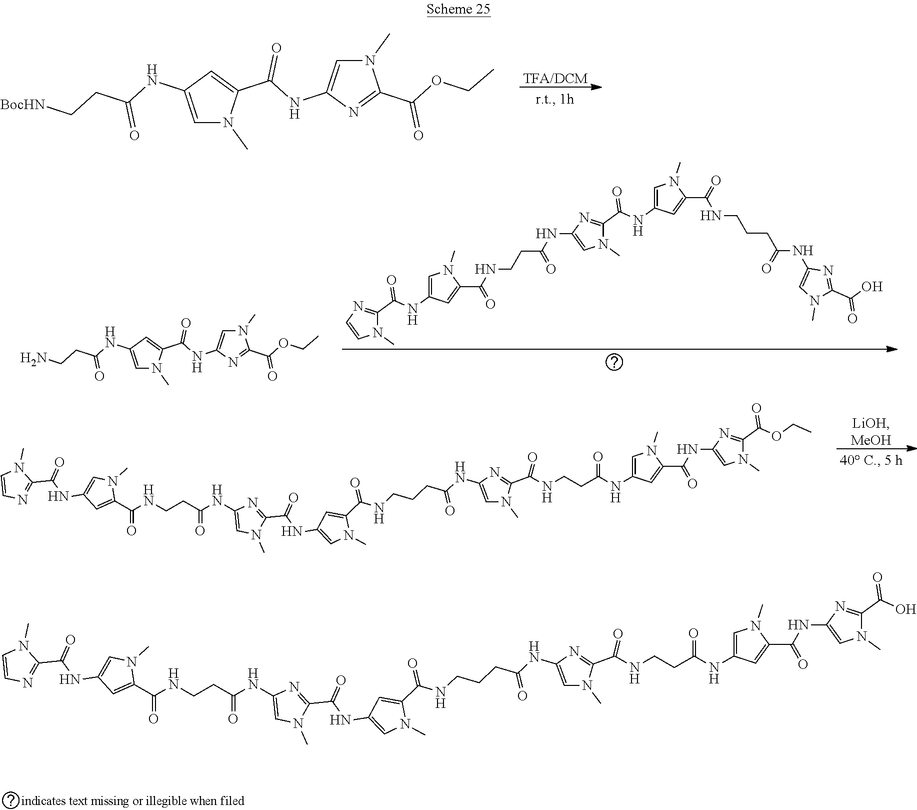

### Step 1: Synthesis of ethyl 4-[4-(3-aminopropanamido)-1methylpyrrole-2-amido]-methylimidazole-2-carboxylate

The procedure was the same as 3-amino-N-propylpropanamide, and the obtained residue was washed with Et₂. 2.00 g of ethyl 4-(4-{3-[(tert-butoxycarbonyl)amino]propanamido}-1-methylpyrrole-2-amido)-1-methylimidazole-2-carboxylate was used, 2.00 g crude of ethyl 4-[4-(3-aminopropanamido)-1-methylpyrrole-2-amido]-1-methylimidazole-2-carboxylate was obtained as white solid. LC/MS: mass calcd. For C₁₆H₂₂N₆O₄: 362.17, found: 363.25[M+H]⁺.

### Step 2: Synthesis of tert-butyl N-(3-[[3-(1,3-dioxoisoindol-2 yl)propyl](methyl)amino]propyl)-N-methylcarbamate

The procedure was the same as ethyl 1-methyl-4-[4-(1-methyl-4-{1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 3.00 g of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was used, 4.30 g of ethyl 1-methyl-4-{11-methyl-4-[3-({1-methyl-4-[4-({1-methyl-4-[1-methyl-4-(3-1 {[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazole-2-carboxylate was obtained as yellow solid (96.84% yield). LC/MS: mass calcd. For C₅₀H₆₀N₂₀O₁₁: 1116.48, found: 1117.60[M+H]⁺.

### Step 3: Synthesis of 1-methyl-4-[1-methyl-4-[3-({1-methyl-4-[4-({1-methyl-4-[1-methyl-4-(3-{[1-methyl-4-(1-methylimidazole-2-amido)pyrrol-2-yl]formamido]propanamido)imidazole-2-amido]pyrrol-2-yl}formamido)butanamido]imidazol-2-yl}formamido)propanamido]pyrrole-2-amido}imidazole-2-carboxylic acid

The procedure was the same as 4-[3-[(tert-butoxycarbonyl)amino]propanamido]-1-methylimidazole-2-carboxylic acid, but the reaction temperature was 40 degree c., the reaction time was 5.0 h. 4.20 g of ethyl 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazole-2-carboxylate was used, 4.00 g of 1-methyl-4-{1-methyl-4-[3-({1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)propanamido]pyrrole-2-amido}imidazole-2-carboxylic acid was obtained as yellow solid (97.97% yield). LC/MS: mass calcd. For C₄₈H₅₆N₂₀O₁₁: 1088.44, found: 1089.55[M+H]⁺.

### Example 26. Synthesis of 3-[(1-methyl-4-{3-[(1-methyl-4-{1-methyl-4-[4-({l-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazol-2-yl)formamido]propanoic acid (Compound

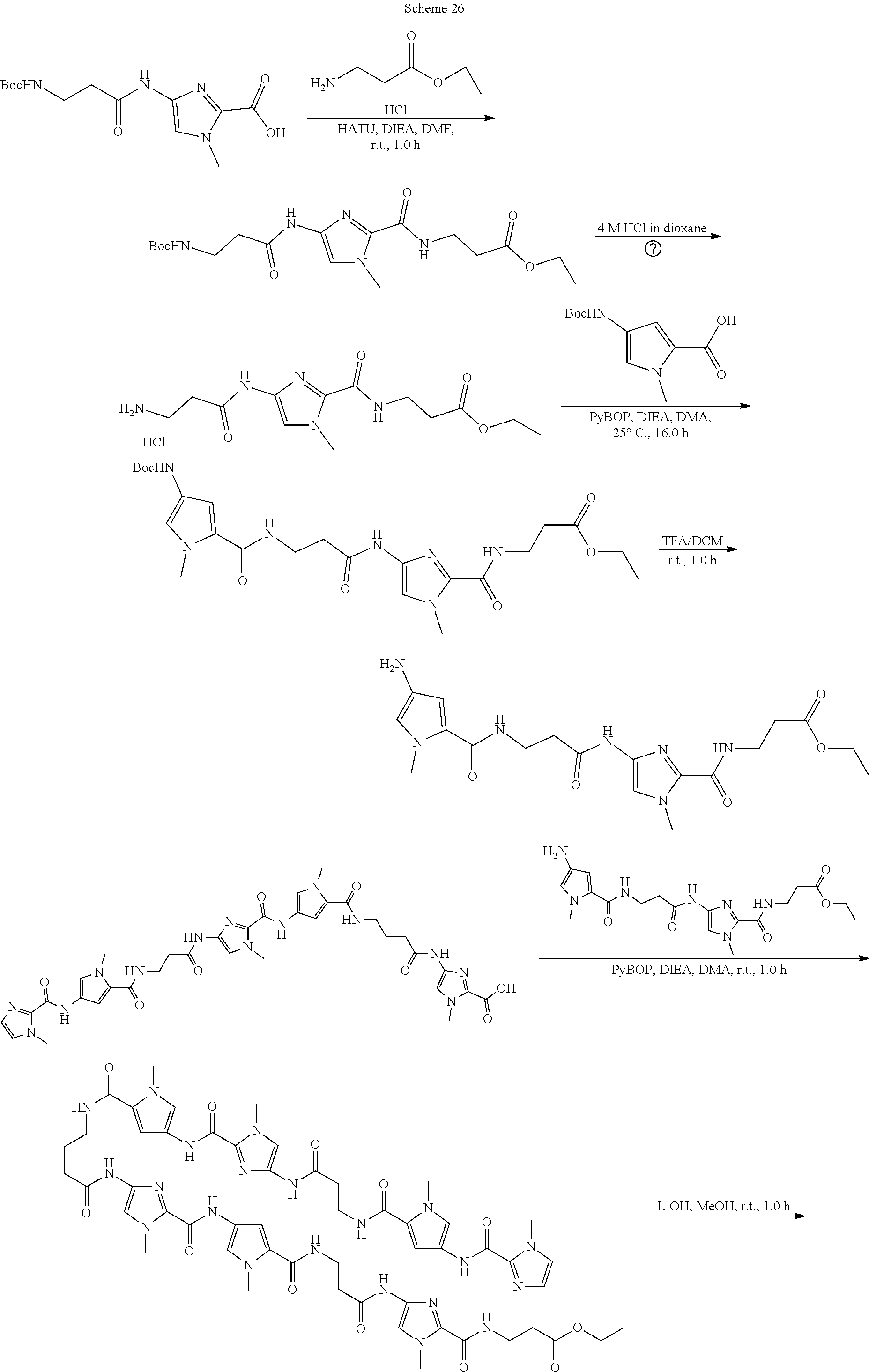

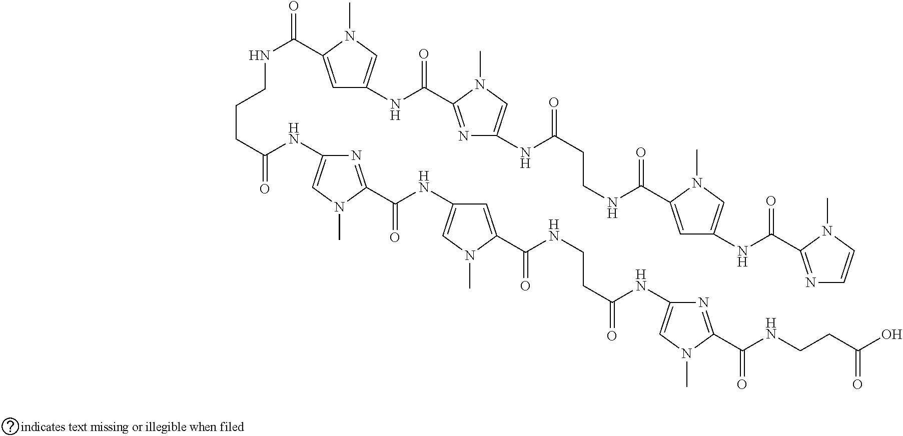 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)Nc2cc(C(=O)NCCC(=O)Nc3cn(C)c(C(=O)NCCC(=O)O)n3)n(C)c2)n1]

### Step 1: Synthesis of Ethyl 3-[(4-{3-[(tert-butoxycarbonyl)amino]propanamido}-1-methylimidazol-2-yl)formamido]propanoate

The procedure was the same as ethyl 4-[3-[(tert-butoxycarbonyl)amino]propanamido]-1-methylimidazole-2-carboxylate. 2.00 g of 4-{3-[(tert-butoxycarbonyl)amino]propanamido}-1-methylimidazole-2-carboxylic acid was used, 2.60 g of ethyl 3-[(4-{3-[(tert-butoxycarbonyl)amino]propanamido}-1-methylimidazol-2-yl)formamido]propanoate was obtained as off-white solid (95.23% yield). LC/MS: mass calcd. For C₁₈H₂₉N₅O₆: 411.21, found: 412.25[M+H]⁺.

### Step 2: Synthesis of ethyl 3-{[4-(3-aminopropanamido)-1-methylimidazol-2-yl]formamido}propanoate

The procedure was the same as methyl 4-[4-(3-aminopropanamido)-1-methylimidazole-2-amido]-1-methylpyrrole-2-carboxylate hydrochloride. 1.00 g of ethyl 3-[(4-{3-[(tert-butoxycarbonyl)amino]propanamido}-1-methylimidazol-2-yl)formamido]propanoate was used, 1.00 g of ethyl 3-{[4-(3-aminopropanamido)-1-methylimidazol-2-yl]formamido}propanoate was obtained as off-white solid. LC/MS: mass calcd. For C₁₃H₂₁N₅O₄: 311.16, found: 312.15[M+H]⁺.

### Step 3: Synthesis of ethyl 3-({4-[3-({4-[(tert-butoxycarbonyl)amino]-1-methylpyrrol-2-yl}formamido)propanamido]-1-methylimidazol-2-yl}formamido)propanoate

The procedure was the same as ethyl 3-{[4-(4-{3-[(tert-butoxycarbonyl)amino] propanamido}-1-methylpyrrole-2-amido)-1-methylimidazol-2-yl]formamido}propanoate, but the solvent was DMA and the reaction time was 16 h. 1.00 g of ethyl 3-{[4-(3-aminopropanamido)-1-methylimidazol-2-yl]formamido}propanoate was used, 1.00 g of ethyl 3-({4-[3-({4-[(tert-butoxycarbonyl)amino]-1-methylpyrrol-2-yl}formamido)propanamido]-1-methylimidazol-2-yl}formamido)propanoate was obtained as white solid (58.35% yield). LC/MS: mass calcd. For C₂₄H₃₅N₇O₇: 533.25, found: 534.45 [M+H]⁺.

### Step 4: Synthesis of ethyl 3-[(4-{3-[(4-amino-1-methylpyrrol-2-yl)formamido]propanamido}-1-methylimidazol-2-yl)formamido]propanoate

The procedure was the same as 3-amino-N-propylpropanamide. 1.00 g of ethyl 3-({4-[3-({4-[(tert-butoxycarbonyl)amino]-1-methylpyrrol-2-yl}formamido)propanamido]-1-methylimidazol-2-yl}formamido)propanoate was used, 800.00 mg crude of ethyl 3-[(4-{3-[(4-amino-1-methylpyrrol-2-yl)formamido]propanamido}-1-methylimidazol-2-yl)formamido]propanoate was obtained as yellow oil. LC/MS: mass calcd. For C₁₉H₂₇N₇O₅: 433.20, found: 434.35 [M+H]⁺.

### Step 5: Synthesis of ethyl 3-[(4-{3-[(4-amino-1-methylpyrrol-2-yl)formamido]propanamido}-1-methylimidazol-2-yl)formamido]propanoate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate, but the solvent was DMA. 1.44 g of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was used, 2.20 g of ethyl 3-[(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido] propanamido}imidazol-2-yl)formamido]propanoate was obtained as yellow oil (99.64% yield). LC/MS: mass calcd. For C₅₃H₆₅N₂₁O₁₂: 1187.51, found: 595.30 [M/2+H]⁺.

### Step 6: Synthesis of 3-[(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazol-2-yl)formamido]propanoic acid

The procedure was the same as 4-[3-[(tert-butoxycarbonyl)amino] propanamido]-1-methylimidazole-2-carboxylic acid, but the reaction temperature was r.t., the reaction time was 1.0 h. 2.20 g of ethyl 3-[(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazol-2-yl)formamido]propanoate was used, 2.00 g of 3-[(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazol-2-yl)formamido]propanoic acid was obtained as yellow solid. LC/MS: mass calcd. For C₅₁H₆₁N₂₁O₁₂: 1159.48, found: 581.25 [M/2+H]⁺.

### Example 27. Synthesis of 3-[(4-{4-[3-({4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoic acid (Compound 141)

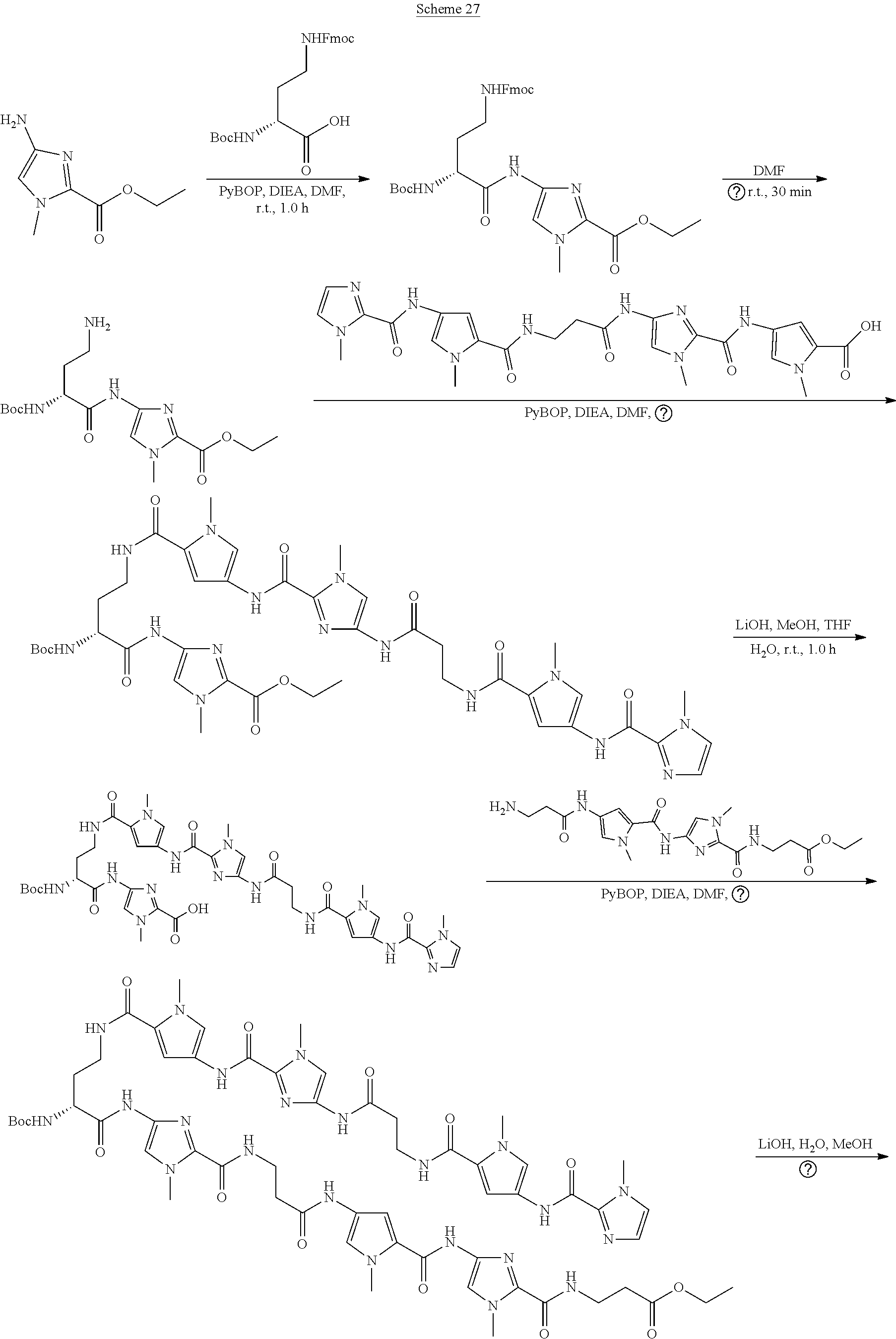

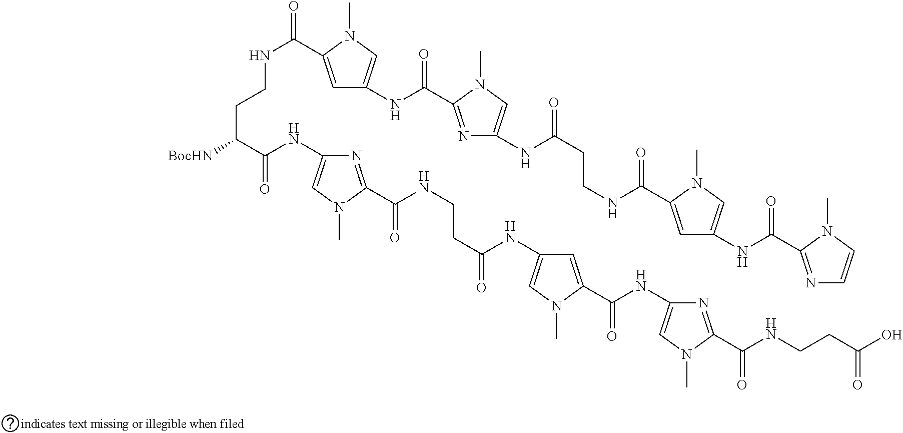 [SMILES: Cn1cc(NC(=O)c2nccn2C)cc1C(=O)NCCC(=O)Nc1cn(C)c(C(=O)Nc2cc(C(=O)NCC[C@@H](NC(=O)OC(C)(C)C)C(=O)Nc3cn(C)c(C(=O)NCCC(=O)Nc4cc(C(=O)Nc5cn(C)c(C(=O)NCCC(=O)O)n5)n(C)c4)n3)n(C)c2)n1]

### Step 1: Synthesis of ethyl 4-[(2R)-2-[(tert-butoxycarbonyl)amino-4-{[(9H-fluoren-9-ylmethoxy)carbonyl]amino}butanamido]-1-methylimidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 3.00 g of(2R)-2-[(tert-butoxycarbonyl)amino]-4-{[(9H-fluoren-9-ylmethoxy)carbonyl] amino}butanoic acid was used, 3.50 g of ethyl 4-[(2R)-2-[(tert-butoxycarbonyl)amino]-4-{[(9H-fluoren-9-ylmethoxy)carbonyl]amino}butanamido]-1-methylimidazole-2-carboxylate was obtained as light yellow solid. LC/MS: mass calcd. For C₃H₃₇N₅O₇: 591.27, found: 592.25 [M+H]⁺.

### Step 2: Synthesis of ethyl 4-[(2R)-4-amino-2-[(tert-butoxycarbonyl)amino] butanamido]-1-methylimidazole-2-carboxylate

Into a 50 ml flask was added ethyl 4-[(2R)-2-[(tert-butoxycarbonyl)amino]-4-{[(9H-fluoren-9-ylmethoxy)carbonyl]amino}butanamido]-1-methylimidazole-2-carboxylate (500.00 mg, 0.85 mmol, 1.00 equiv), DMF (5.00 mL), piperidine (1.00 mL). The reaction was stirred at r.t. for 30 mins, the piperidine was removed, the reaction mixture was purified by reverse flash chromatography with the following conditions: column, C18 column; mobile phase, MeCN in water (0.5% NH₄HCO₃), 100% to 50% gradient in 30 min; detector, UV 254 nm. The fractions were combined and concentrated. This resulted in ethyl 4-[(2R)-4-amino-2-[(tert-butoxycarbonyl)amino]butanamido]-1-methylimidazole-2-carboxylate (250.00 mg, 96.10%) as a yellow oil. LC/MS: mass calcd. For C₁₆H₂₇N₅O₅: 369.20, found: 370.35[M+H]⁺.

### Step 3: Synthesis of ethyl 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 230.00 mg of ethyl 4-[(2R)-4-amino-2-[(tert-butoxycarbonyl)amino]butanamido]-1-methylimidazole-2-carboxylate was used, 200.00 mg of ethyl 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylate was obtained as yellow solid (68.39% yield). LC/MS: mass calcd. For C₄₁H₅₃N₁₅O₁₀: 915.41, found: 916.75[M+H]⁺.

### Step 4: Synthesis of 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid

The procedure was the same as 4-[3-[(tert-butoxycarbonyl)amino] propanamido]-1-methylimidazole-2-carboxylic acid, but the reaction solvent was the mixture of MeOH/THF(5:3), the reaction temperature was r.t., the reaction time was 1.0 h. 570.00 mg of ethyl 4-[(2R)-2-[(tert-butoxycarbonyl) amino]-4-({1-methyl-4-[1-methyl-4-(3-{[1-methyl-4-(1-methylimidazole-2-amido) pyrrol-2-yl]formamido}propanamido)imidazole-2-amido]pyrrol-2-yl}formamido)butanamido]-1-methylimidazole-2-carboxylate was used, 370.00 mg of 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid was obtained as yellow solid (66.90% yield). LC/MS: mass calcd. For C₃₉H₄₉N₁₅O₁₀: 887.38, found: 888.85[M+H]⁺.

### Step 5: Synthesis of ethyl 3-[(4-{4-[3-({4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 380.00 mg of 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid was used, 521.00 mg of ethyl 3-[(4-{4-[3-({4-[(2R)—2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate was obtained as white solid (91.75% yield). LC/MS: mass calcd. For C₅₈H₇₄N₂₂O₁₄: 1302.58, found: 652.80[M/2+H]⁺.

### Step 6: Synthesis of 3-[(4-{4-[3-({4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoic acid

The procedure was the same as 4-[3-[(tert-butoxycarbonyl)amino]propanamido]-1-methylimidazole-2-carboxylic acid, but the reaction temperature was r.t. 150.00 mg of ethyl 3-[(4-{4-[3-({4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate was used, 146.00 mg crude of 3-[(4-{4-[3-({4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoic acid was obtained as white solid. LC/MS: mass calcd. For C₅₆H₇₀N₂₂O₁₄: 1274.54 found: 638.85[M/2+H]⁺.

### Example 28. Synthesis of 3-[(4-{4-[3-({4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoic acid (Compound 142)

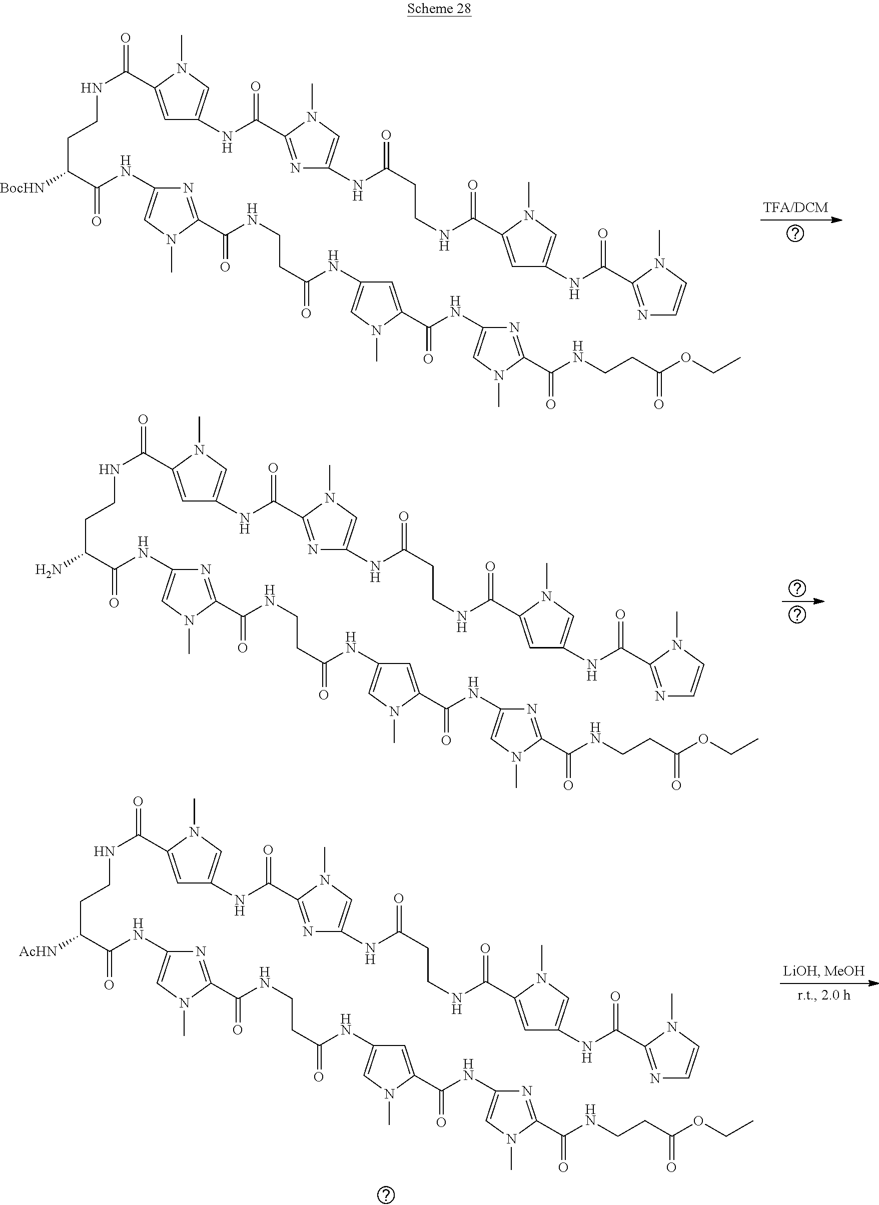 [SMILES: CCOC(=O)CCNC(=O)c1nc(NC(=O)c2cc(NC(=O)CCNC(=O)c3nc(NC(=O)[C@@H](CCNC(=O)c4cc(NC(=O)c5nc(NC(=O)CCNC(=O)c6cc(NC(=O)c7nccn7C)cn6C)cn5C)cn4C)NC(=O)OC(C)(C)C)cn3C)cn2C)cn1C ; CCOC(=O)CCNC(=O)c1nc(NC(=O)c2cc(NC(=O)CCNC(=O)c3nc(NC(=O)[C@H](N)CCNC(=O)c4cc(NC(=O)c5nc(NC(=O)CCNC(=O)c6cc(NC(=O)c7nccn7C)cn6C)cn5C)cn4C)cn3C)cn2C)cn1C ; CCOC(=O)CCNC(=O)c1nc(NC(=O)c2cc(NC(=O)CCNC(=O)c3nc(NC(=O)[C@@H](CCNC(=O)c4cc(NC(=O)c5nc(NC(=O)CCNC(=O)c6cc(NC(=O)c7nccn7C)cn6C)cn5C)cn4C)NC(C)=O)cn3C)cn2C)cn1C]

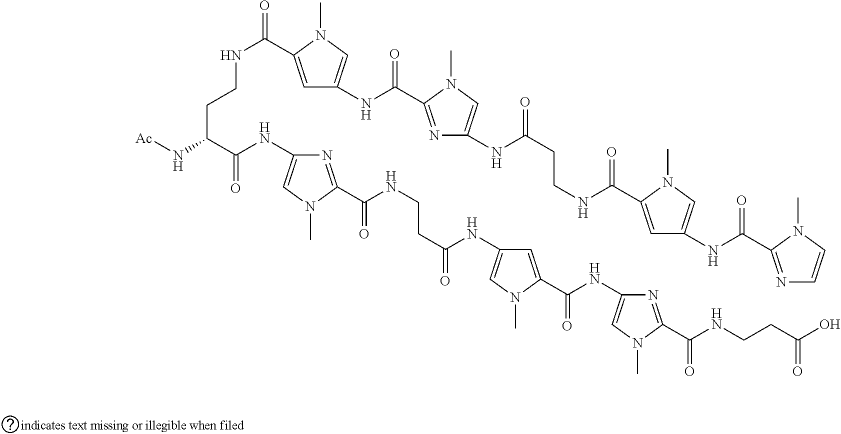 [SMILES: CC(=O)N[C@H](CCNC(=O)c1cc(NC(=O)c2nc(NC(=O)CCNC(=O)c3cc(NC(=O)c4nccn4C)cn3C)cn2C)cn1C)C(=O)Nc1cn(C)c(C(=O)NCCC(=O)Nc2cc(C(=O)Nc3cn(C)c(C(=O)NCCC(=O)O)n3)n(C)c2)n1]

### Step 1: Synthesis of ethyl 3-[(4-[4-[3-([{4-[(2R)-2-acetamido-4-({1-methyl-4-[1-methyl-4-(3-{[1-methyl-4-(1-methylimidazole-2-amido)pyrrol-2-yl]formamido] propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate

The procedure was the same as 3-amino-N-propylpropanamide. 319.00 mg of ethyl 3-[(4-{4-[3-({4-[(2R)-2-amino-4-({1-methyl-4-[1-methyl-4-(3-{[1-methyl-4-(1-methylimidazole-2-amido)pyrrol-2-yl]formamido} propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate was used, 300.00 mg of ethyl 3-[(4-{4-[3-({4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate was obtained as brown solid. LC/MS: mass calcd. For C₅₃H₆₆N₂₂O₁₂: 1202.52, found: 602.75[M/2+H]⁺.

### Step 2: Synthesis of ethyl 3-[(4-{4-[3-({4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate

To a stirred solution of ethyl 3-[(4-{4-[3-({4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate (294.50 mg, 0.25 mmol, 1.00 equiv) in DCM (6.00 mL) was added Ac₂O (0.23 mL, 2.45 mmol, 10.00 equiv) and Et₃N (0.34 mL, 2.45 mmol, 10.00 equiv) in portions at 0° C. The resulting mixture was stirred for 1 h at room temperature. The resulting mixture was concentrated under vacuum. The crude product was purified by reverse phase column with the following condition: column, C18; mobile phase, ACN in water (0.5% TFA), 10% to 50% gradient in 30 min; detector, UV 254 nm. The factions were combined and lyophilized directly. This resulted in ethyl 3-[(4-{4-[3-({4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoate (200.00 mg, 64.31%) as a white solid. LC/MS: mass calcd. For C₅₅H₆₈N₂₂O₁₃: 1244.53 found: 623.75[M/2+H]⁺.

### Step 3: Synthesis of 3-[(4-{4-[3-({4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoic acid

The procedure was the same as 4-[3-[(tert-butoxycarbonyl)amino] propanamido]-1-methylimidazole-2-carboxylic acid, but the reaction temperature was r.t. 190.00 mg of ethyl (R)-3-(4-(4-(3-(4-(2-acetamido-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxamido)propanamido)-1-methyl-1H-pyrrole-2-carboxamido)-1-methyl-1H-imidazole-2-carboxamido) propanoate was used, 91.50 mg of 3-[(4-{4-[3-({4-[(2R)-2-acetamido-4-({1-methyl-4-[1-methyl-4-(3-{[1-methyl-4-(1-methylimidazole-2-amido)pyrrol-2-yl]formamido} propanamido)imidazole-2-amido]pyrrol-2-yl}formamido)butanamido]-1-methylimidazol-2-yl}formamido)propanamido]-1-methylpyrrole-2-amido}-1-methylimidazol-2-yl) formamido]propanoic acid was obtained as white solid (50.12% yield). LC/MS: mass calcd. For C₅₃H₆₄N₂₂O₁₃: 1216.50, found: 609.80 [M/2+H]⁺.

### Example 29. Synthesis of 1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrole-2-carboxylic acid (Compound 113)

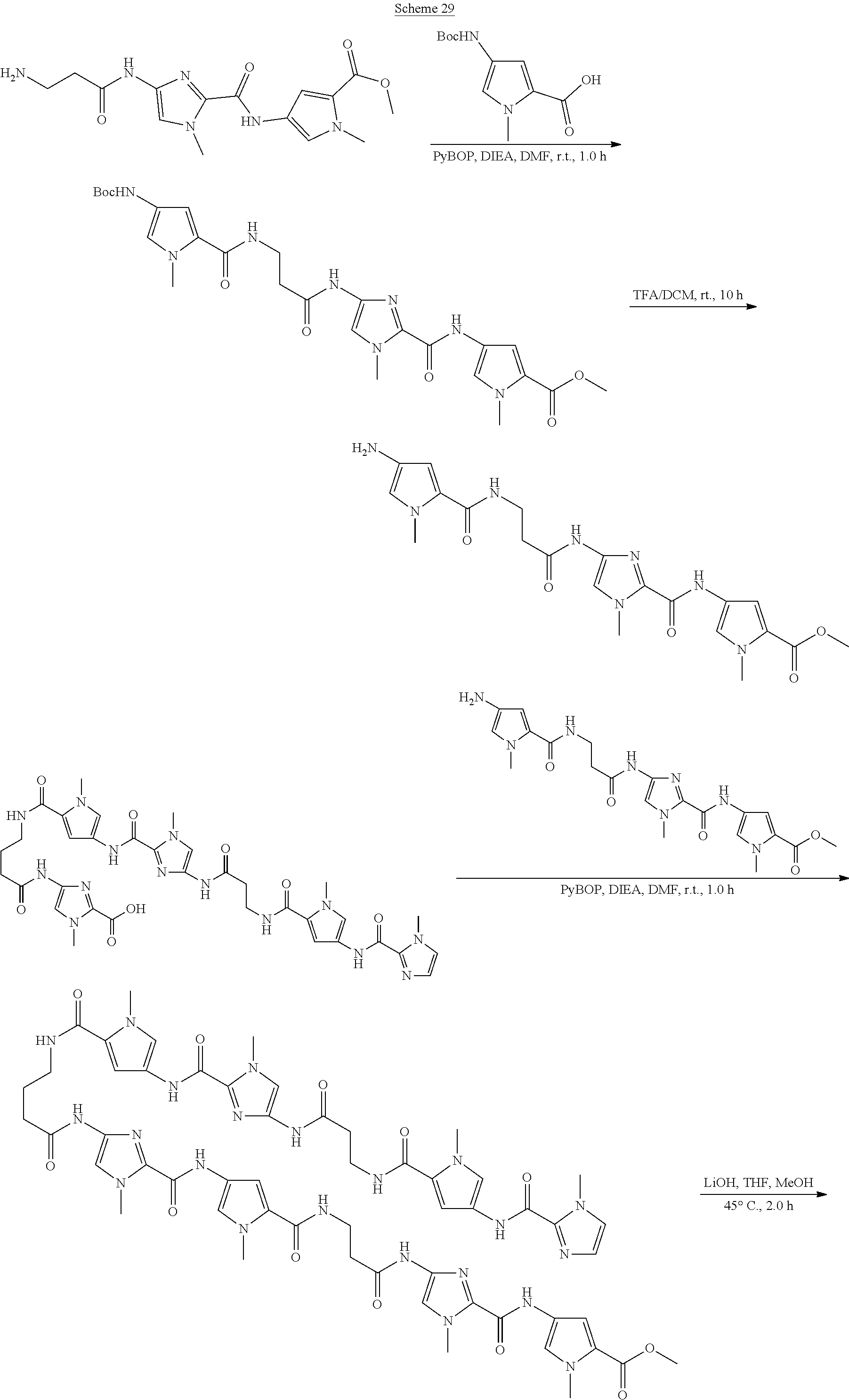

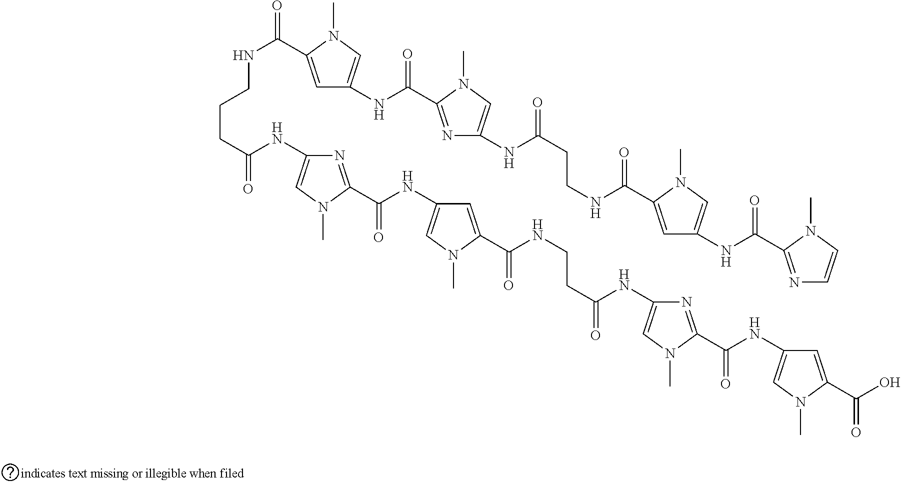 [SMILES: Cn1cc(NC(=O)c2nc(NC(=O)CCNC(=O)c3cc(NC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cc1C(=O)O]

### Step 1: Synthesis of methyl 4-{4-[3-({4-[(tert-butoxycarbonyl)amino]-1-methylpyrrol-2-yl}formamido)propanamido]-1-methylimidazole-2-amido}-1-methylpyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate. 2.07 g of 4-[(tert-butoxycarbonyl)amino]-1-methylpyrrole-2-carboxylic acid was used, 3.00 g of Methyl 4-{4-[3-({4-[(tert-butoxycarbonyl) amino]-1-methylpyrrol-2-yl}formamido)propanamido]-1-methylimidazole-2-amido}-1-methylpyrrole-2-carboxylate was obtained as a light yellow solid. LC/MS: mass calcd. For C₂₆H₃₄N₈O₇: 570.26, found: 571.30 [M+H]⁺.

### Step 2: Synthesis of methyl 4-(4-{3-[(4-amino-1-methylpyrrol-2-yl)formamido] propanamido}-1-methylimidazole-2-amido)-1-methylpyrrole-2-carboxylate

The procedure was the same as 3-amino-N-propylpropanamide. 355.00 mg of methyl 4-{4-[3-({4-[(tert-butoxycarbonyl)amino]-1-methylpyrrol-2-yl}formamido)propanamido]-1-methylimidazole-2-amido}-1-methylpyrrole-2-carboxylate was used, 350.00 mg crude of methyl 4-(4-{3-[(4-amino-1-methylpyrrol-2-yl)formamido]propanamido}-1-methylimidazole-2-amido)-1-methylpyrrole-2-carboxylate was obtained as a brown yellow oil. LC/MS: mass calcd. For C₂₁H₂₆N₈O₅:470.20, found: 471.45[M+H]⁺.

### Step 3: Synthesis of Methyl 1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate. 450.00 mg of 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylic acid was used, 790.00 mg of methyl 1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrole-2-carboxylate was obtained as white solid (95.77% yield). LC/MS: mass calcd. For C₅₅H₆₄N₂₂O₁₂:1224.51, found: 1225.85 [M+H]⁺.

### Step 4: Synthesis of 1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrole-2-carboxylic acid

The procedure was the same as 4-[3-[(tert-butoxycarbonyl)amino]propanamido]-1-methylimidazole-2-carboxylic acid, but the reaction solvent was MeOH/THF=1:1. 300.00 mg of methyl 1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido} imidazole-2-amido)pyrrole-2-carboxylate was used, 290.00 mg of 1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrole-2-carboxylic acid was obtained as white solid (68.45% yield). LC/MS: mass calcd. For C₅₄H₆₂N₂₂O₁₂:1210.49, found: 1211.80 [M+H]⁺.

### Scheme 30. Synthesis of 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylic acid (Compound 143)

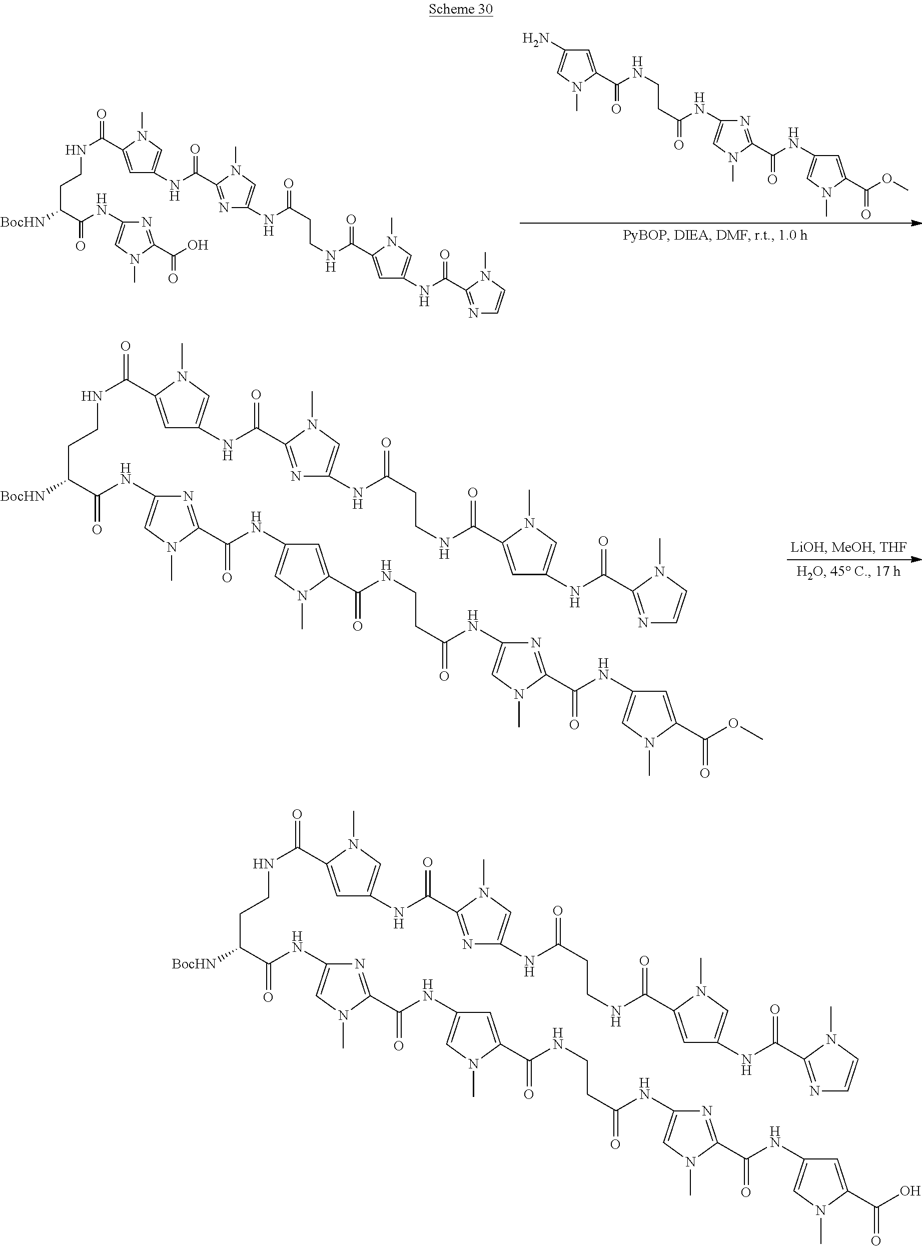

### Step 1: Synthesis of Methyl 4-(4-{3-[(4-{4-[(2R)-2-[(tert-butoxycarbonyl)amino]-4-({1-methyl-4-[1-methyl-4-(3-{[[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate

To a stirred solution of 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid (600.00 mg, 0.68 mmol, 1.00 equiv) in DMF (6.00 mL) was added PyBOP (527.47 mg, 1.01 mmol, 1.50 equiv), DIEA (262.00 mg, 2.03 mmol, 3.00 equiv) and methyl 4-(4-{3-[(4-amino-1-methylpyrrol-2-yl)formamido] propanamido}-1-methylimidazole-2-amido)-1-methylpyrrole-2-carboxylate (317.93 mg, 0.68 mmol, 1.00 equiv) in portions at 0 degrees C. The resulting mixture was stirred for 1.0 h at room temperature. The reaction mixture was purified by reverse flash chromatography with the following conditions: column, C18 column; mobile phase, ACN in water (0.05% TFA), 30% to 40% gradient in 20 min; detector, UV 254 nm. The fractions were combined and concentrated. Methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido-}-methylpyrrol-2-yl) formamido]propanamido}-1-methylimidazole-2-amido)-1-methylpyrrole-2-carboxylate (740.00 mg, 81.70%) was obtained as yellow solid. LC/MS: mass calcd. For C₆₀H₇₃N₂₃O₁₄: 1339.57, found: 671.35 [M/2+H]⁺.

### Step 2: Synthesis of 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)imidazole-2-amido)butanamido]}-1-methylpyrrol-2-yl)formamido]propanamido}-1-methylimidazole-2-amido)-1-methylpyrrole-2-carboxylic acid

The procedure was the same as 4-[3-[(tert-butoxycarbonyl)amino]propanamido]-1-methylimidazole-2-carboxylic acid, but the reaction solvent was MeOH/THF (1:1), the reaction time was 17.0 h. 630.00 mg of methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate was used, 460.00 mg of 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylic acid was obtained as white solid (73.79% yield). LC/MS: mass calcd. For C₅₉H₇N₂₃O₁₄: 1325.56, found: 1326.65 [M+H]⁺.

### Example 31. Synthesis of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-N-{5-[(2-{[2-({5-[(2-hydroxyethyl)carbamoyl]-1-methylpyrrol-3-yl}carbamoyl)-1-methylimidazol-4-yl]carbamoyl}ethyl)carbamoyl]-1-methylpyrrol-3-yl}-1-methylimidazole-2-carboxamide (Compound 144)

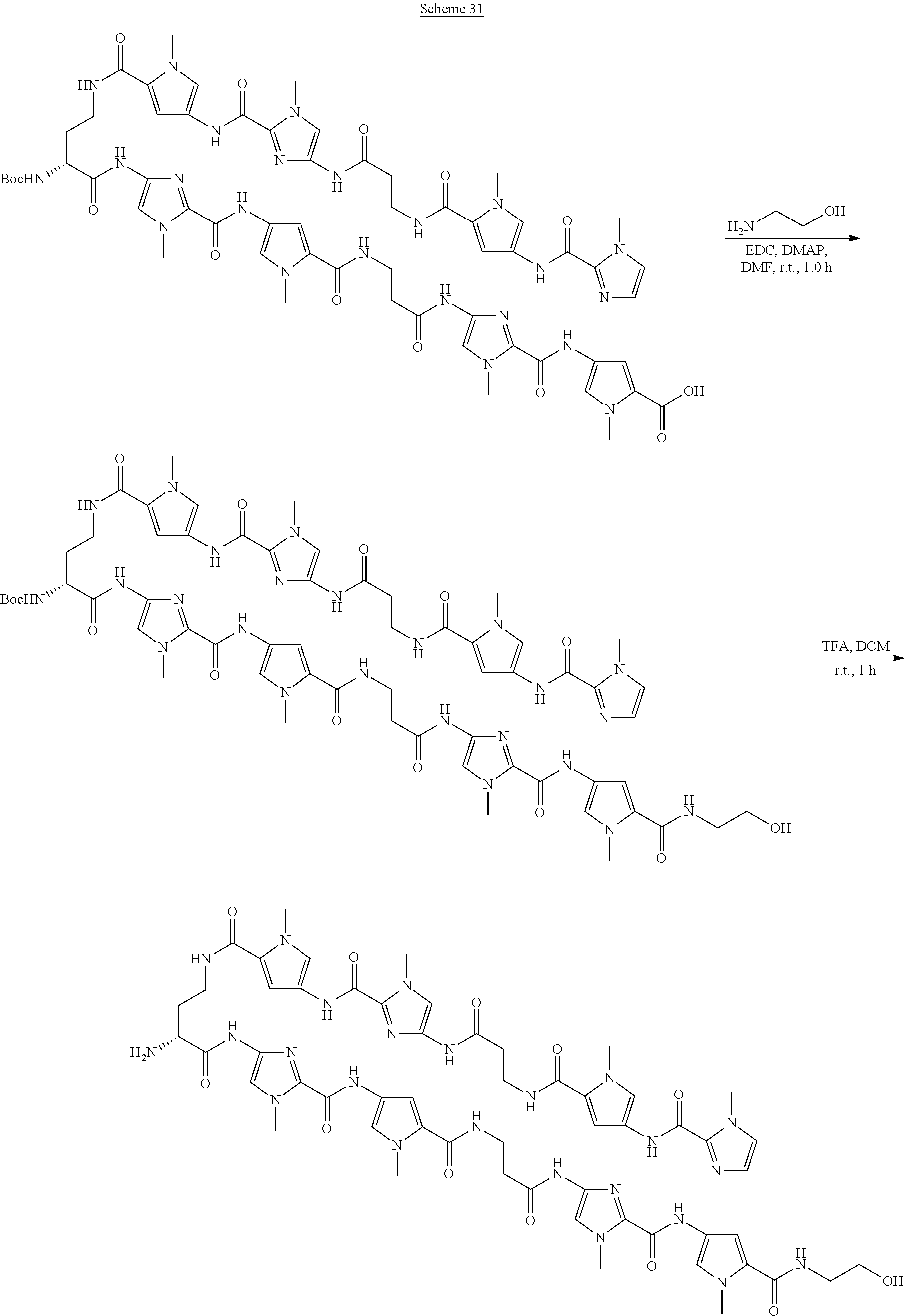

### Step 1: Synthesis of tert-butyl N—[(R)-1-{[2-({5-[(2-{[2-({5-[(2-hydroxyethyl) carbamoyl]-1-methylpyrrol-3-yl}carbamoyl)-1-methylimidazol-4-yl]carbamoyl}ethyl)carbamoyl]-1-methylpyrrol-3-yl}carbamoyl)-1-methylimidazol-4-yl]carbamoyl}-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propyl]carbamate

The procedure was the same as ethyl 4-[4-[(tert-butoxycarbonyl)amino]-1-methylpyrrole-2-amido]-1-methylimidazole-2-carboxylate, but the reaction time was 1.0 h. 60.00 mg of 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylic acid was used, 56.00 mg of tert-butyl N-[(1R)-1-{[2-({5-[(2-{[2-({5-[(2-hydroxyethyl) carbamoyl]-1-methylpyrrol-3-yl}carbamoyl)-1-methylimidazol-4-yl]carbamoyl}ethyl)carbamoyl]-1-methylpyrrol-3-yl}carbamoyl)-1-methylimidazol-4-yl]carbamoyl}-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propyl]carbamate was obtained as brown oil (81.36% yield). LC/MS: mass calcd. For C₆₁H₇₆N₂₄O₁₄:1368.60, found: 685.95 [M/2+H]⁺.

### Step 2: Synthesis of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-N-{5-[(2-{[2-({5-[(2-hydroxyethyl)carbamoyl]-1-methylpyrrol-3-yl}carbamoyl)-1-methylimidazol-4-yl]carbamoyl}ethyl)carbamoyl]-1-methylpyrrol-3-yl}-1-methylimidazole-2-carboxamide

The procedure was the same as 3-amino-N-propylpropanamide. 46.00 mg of tert-butyl N-[(1R)-1-{[2-({5-[(2-{[2-({5-[(2-hydroxyethyl)carbamoyl]-1-methylpyrrol-3-yl}carbamoyl)-1-methylimidazol-4-yl]carbamoyl}ethyl)carbamoyl]-1-methylpyrrol-3-yl}carbamoyl)-1-methylimidazol-4-yl]carbamoyl}-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propyl]carbamate was used, 46.00 mg crude of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-N-{5-[(2-{[2-({5-[(2-hydroxyethyl)carbamoyl]-1-methylpyrrol-3-yl}carbamoyl)-1-methylimidazol-4-yl]carbamoyl}ethyl)carbamoyl]-1-methylpyrrol-3-yl}-1-methylimidazole-2-carboxamide was obtained as brown oil. LC/MS: mass calcd. For C₅₆H₆₈N₂₄O₁₂: 1268.54, found: 635.75 [M/2+H]⁺.

### Example 32. Synthesis of N-(5-{[(3R)-3-amino-3-[(1-methyl-2-{[2-({1-methyl-5-[(1-methyl-2-{[2-(propylcarbamoyl)ethyl]carbamoyl}imidazol-4-yl)carbamoyl]pyrrol-3-yl}carbamoyl) ethyl]carbamoyl}imidazol-4-yl)carbamoyl]propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Compound 145)

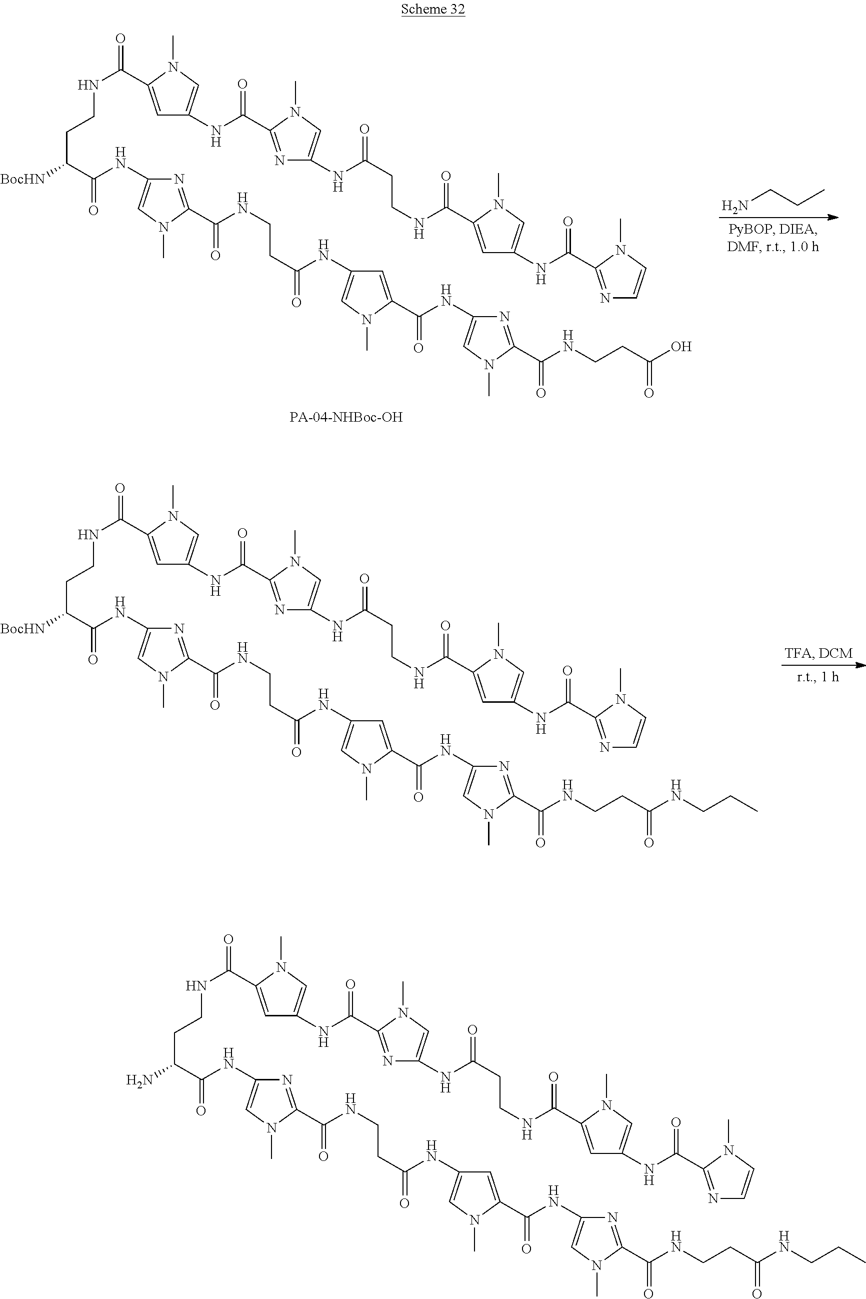

### Step 1: Synthesis of tert-butyl N-[(1R)-1-[(1-methyl-2-{[2-({1-methyl-5-[(1-methyl-2-{[2-(propylcarbamoyl)ethyl]carbamoyl}imidazol-4-yl)carbamoyl]pyrrol-3-yl}carbamoyl)ethyl]carbamoyl}imidazol-4-yl)carbamoyl]-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propyl]carbamate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate (PH-DTP-DT469-2). 50.00 mg of 3-[(4-{4-[3-({4-[(2R)-2-[(tert-butoxycarbonyl) amino]-4-({1-methyl-4-[1-methyl-4-(3-{[1-methyl-4-(1-methylimidazole-2-amido) pyrrol-2-yl]formamido}propanamido)imidazole-2-amido]pyrrol-2-yl}formamido)butanamido]-1-methylimidazol-2-yl}formamido)propanamido]-1-methylpyrrole-2-amido}-1-methylimidazol-2-yl)formamido]propanoic acid was used, 50.00 mg of tert-butyl N-[(1R)-1-[(1-methyl-2-{[2-({1-methyl-5-[(1-methyl-2-{[2-(propylcarbamoyl)ethyl] carbamoyl}imidazol-4-yl)carbamoyl]pyrrol-3-yl}carbamoyl)ethyl]carbamoyl}imidazol-4-yl)carbamoyl]-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propyl]carbamate was obtained as white solid (90.96% yield). LC/MS: mass calcd. For C₅₉H₇₇N₂₂O₁₃: 1315.61, found: 659.35 [M/2+H]⁺.

### Step 2: Synthesis of N-(5-{[(3R)-3-amino-3-[(1-methyl-2-{[2-({1-methyl-5-[(1-methyl-2-{[2-(propylcarbamoyl)ethyl]carbamoyl}imidazol-4-yl)carbamoyl] pyrrol-3-yl}carbamoyl)ethyl]carbamoyl}imidazol-4-yl)carbamoyl]propyl] carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide

The procedure was the same as 3-amino-N-propylpropanamide. 45.00 mg of tert-butyl N-[(1R)-1-[(1-methyl-2-{[2-({1-methyl-5-[(1-methyl-2-{[2-(propylcarbamoyl)ethyl]carbamoyl}imidazol-4-yl)carbamoyl]pyrrol-3-yl}carbamoyl)ethyl]carbamoyl}imidazol-4-yl)carbamoyl]-3-({1-methyl-4-[1-methyl-4-(3-{[1-methyl-4-(1-methylimidazole-2-amido)pyrrol-2-yl]formamido} propanamido)imidazole-2-amido]pyrrol-2-yl}formamido)propyl]carbamate was obtained, 50.00 mg crude of N-(5-{[(3R)-3-amino-3-[(1-methyl-2-{[2-({1-methyl-5-[(1-methyl-2-{[2-(propylcarbamoyl)ethyl]carbamoyl}imidazol-4-yl)carbamoyl]pyrrol-3-yl}carbamoyl)ethyl]carbamoyl}imidazol-4-yl)carbamoyl]propyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was obtained as brown yellow oil. LC/MS: mass calcd. For C₅₄H₆₉N₂₃O₁₁: 1215.55, found: 1217.15 [M+H]⁺.

### Example 33. Synthesis of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methyl-N-[1-methyl-5-({2-[(1-methyl-2-{[1-methyl-5-(propylcarbamoyl)pyrrol-3-yl]carbamoyl}imidazol-4-yl)carbamoyl]ethyl}carbamoyl)pyrrol-3-yl]imidazole-2-carboxamide (Compound 146)

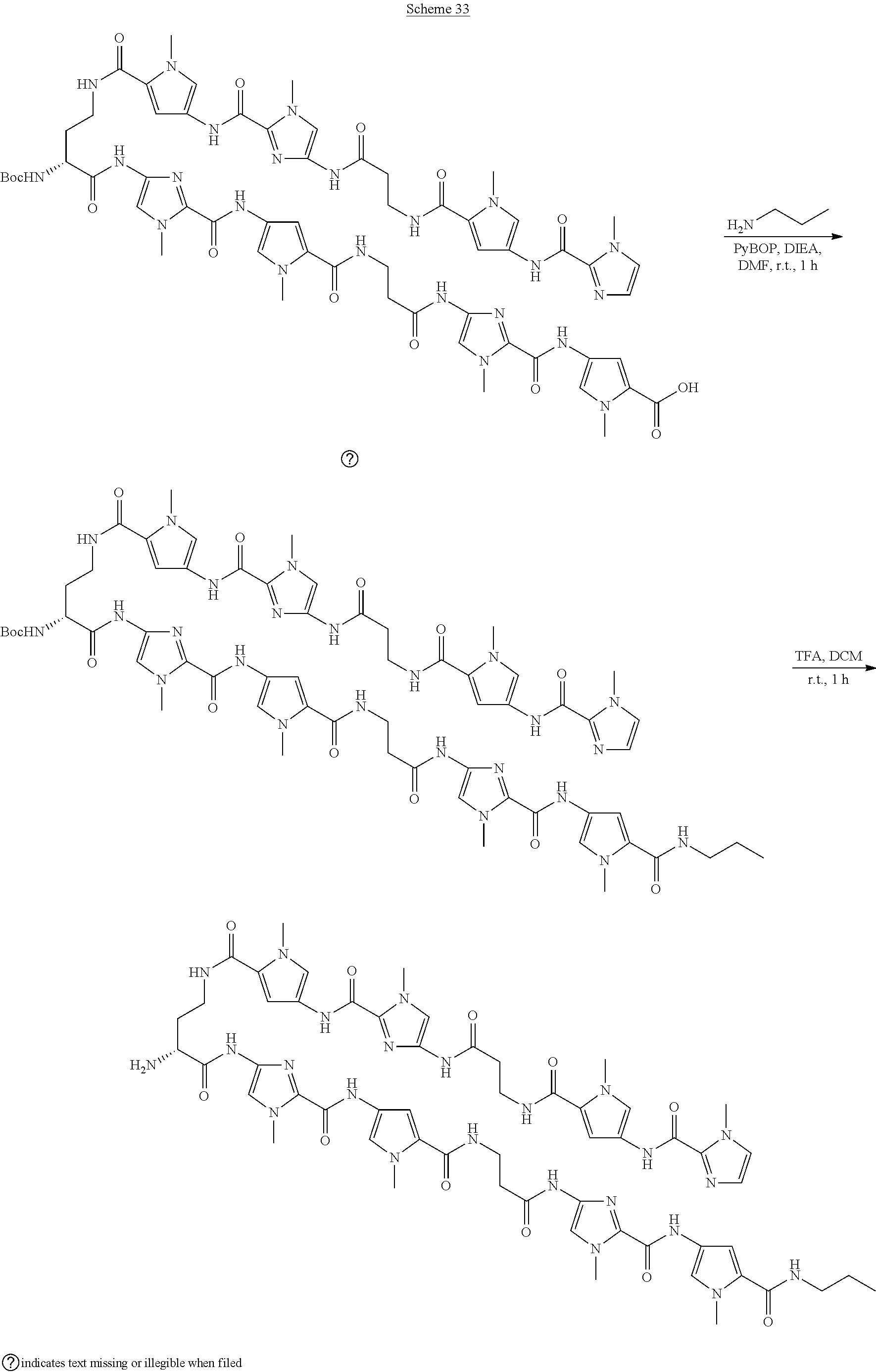

### Step 1: Synthesis of tert-butyl N—[(R)-1-[(1-methyl-2-{[1-methyl-5-({2-[(1-methyl-2-{[1-methyl-5-(propylcarbamoyl)pyrrol-3-yl]carbamoyl}imidazol-4-yl) carbamoyl]ethyl}carbamoyl)pyrrol-3-yl]carbamoyl}imidazol-4-yl)carbamoyl]-3-({1-methyl-4-[1-methyl-4-(3-{[[1-methyl-4-(1-methylimidazole-2-amido)pyrrol-2-yl]formamido}propanamido)imidazole-2-amido]pyrrol-2-yl}formamido)propyl]carbamate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate, 50.00 mg of 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl] formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylic acid was used, 50.00 mg of tert-butyl N-[(1R)-1-[(1-methyl-2-{[1-methyl-5-({2-[(1-methyl-2-{[1-methyl-5-(propylcarbamoyl)pyrrol-3-yl]carbamoyl}imidazol-4-yl)carbamoyl]ethyl}carbamoyl)pyrrol-3-yl]carbamoyl}imidazol-4-yl)carbamoyl]-3-({1-methyl-4-[1-methyl-4-(3-{[1-methyl-4-(1-methylimidazole-2-amido)pyrrol-2-yl]formamido}propanamido)imidazole-2-amido] pyrrol-2-yl}formamido)propyl]carbamate was obtained as yellow oil (96.99% yield). LC/MS: mass calcd. For C₆₂H₇₈N₂₄O₁₃: 1366.62, found: 1368.30[M+H]⁺.

### Step 2: Synthesis of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methyl-N-[1-methyl-5-({2-[(1-methyl-2-{[1-methyl-5-(propylcarbamoyl)pyrrol-3-yl]carbamoyl}imidazol-4-yl)carbamoyl]ethyl}carbamoyl)pyrrol-3-yl]imidazole-2-carboxamide

The procedure was the same as 3-amino-N-propylpropanamide. 50.00 mg of tert-butyl N-[(1R)-1-[(1-methyl-2-{[1-methyl-5-({2-[(1-methyl-2-{[1-methyl-5-(propylcarbamoyl)pyrrol-3-yl]carbamoyl}imidazol-4-yl) carbamoyl]ethyl}carbamoyl)pyrrol-3-yl]carbamoyl}imidazol-4-yl)carbamoyl]-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propyl]carbamate was used, 50.00 mg of 4-[(2R)-2-amino-4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]-1-methyl-N-[1-methyl-5-({2-[(1-methyl-2-{[1-methyl-5-(propylcarbamoyl)pyrrol-3-yl]carbamoyl}imidazol-4-yl)carbamoyl]ethyl} carbamoyl)pyrrol-3-yl]imidazole-2-carboxamide was obtained as yellow oil. LC/MS: mass calcd. For C₅₇H₇₀N₂₄O₁₁: 1266.57, found: 1267.95[M+H]⁺.

### Example 34. Synthesis of 3-[(1-methyl-4-{1-methyl-4-[3-({1-methyl-4-[4-({1-methyl-4-[1-methyl-4-(1-methyl-4-{3-[(1-methylimidazol-2-yl)formamido]propanamido}pyrrole-2-amido)imidazole-2-amido]pyrrol-2-yl}formamido)butanamido]imidazol-2-yl]formamido)propanamido]pyrrole-2-amido}imidazol-2-yl)formamido]propanoic acid (Compound 147)

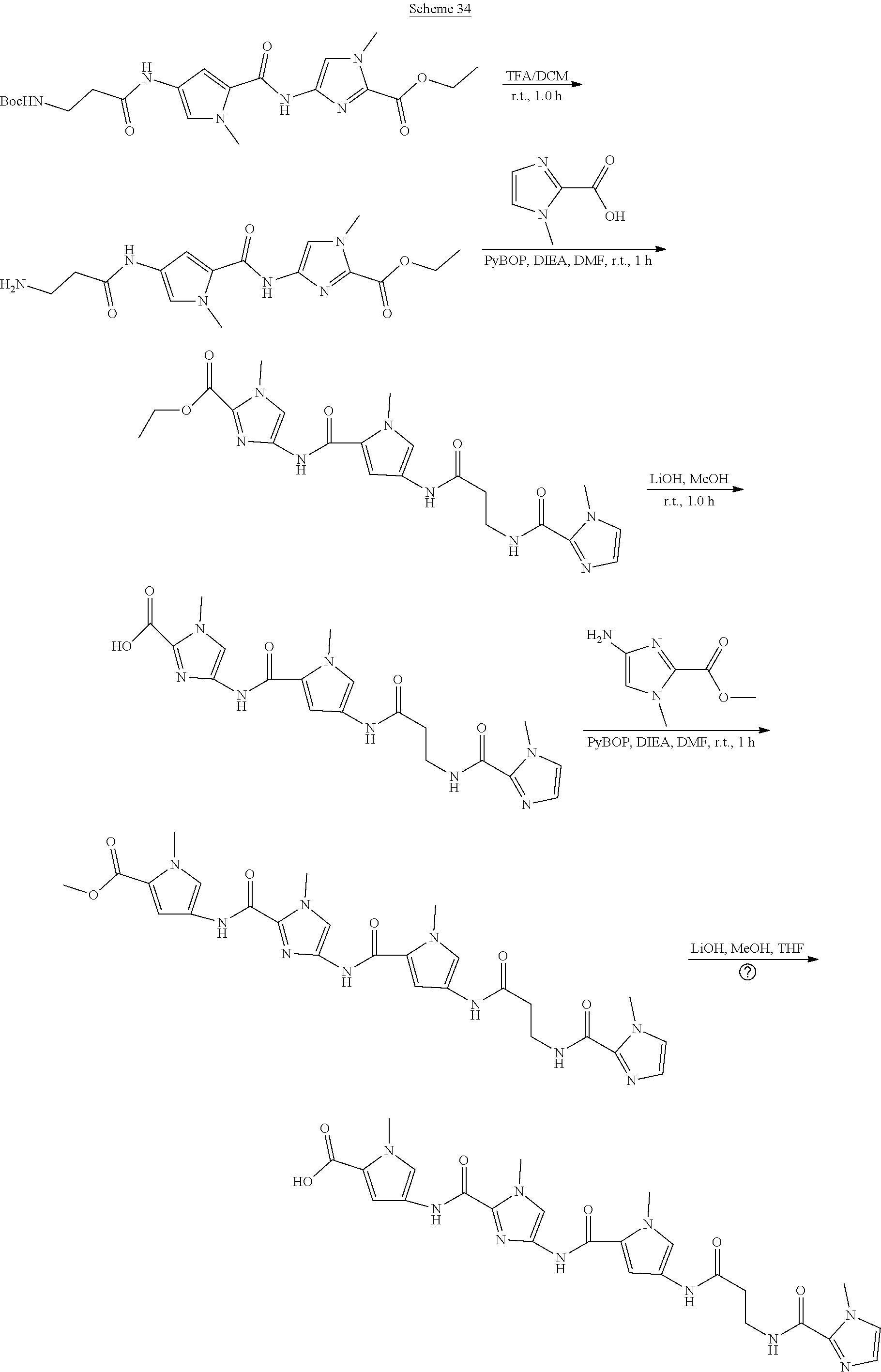

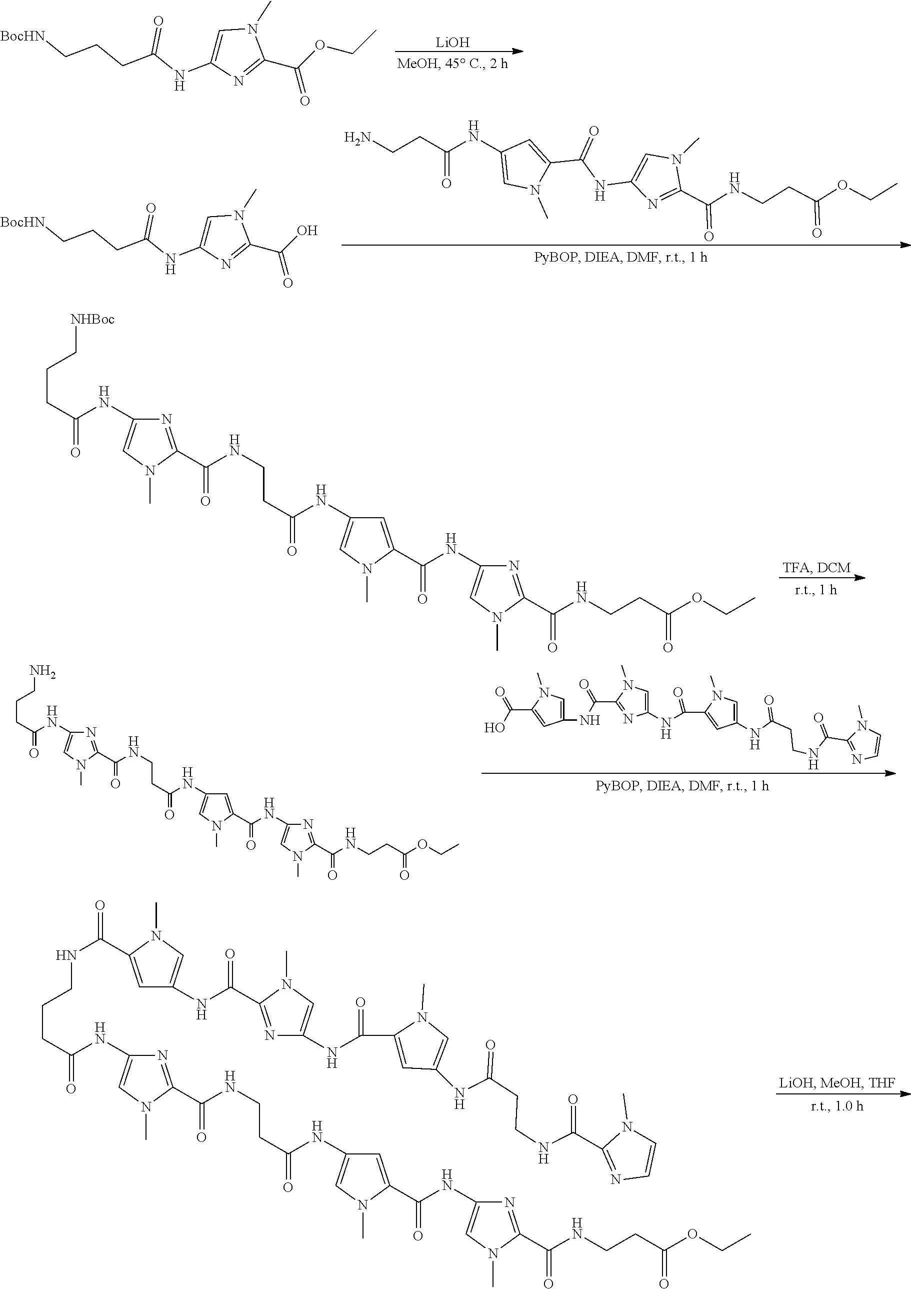

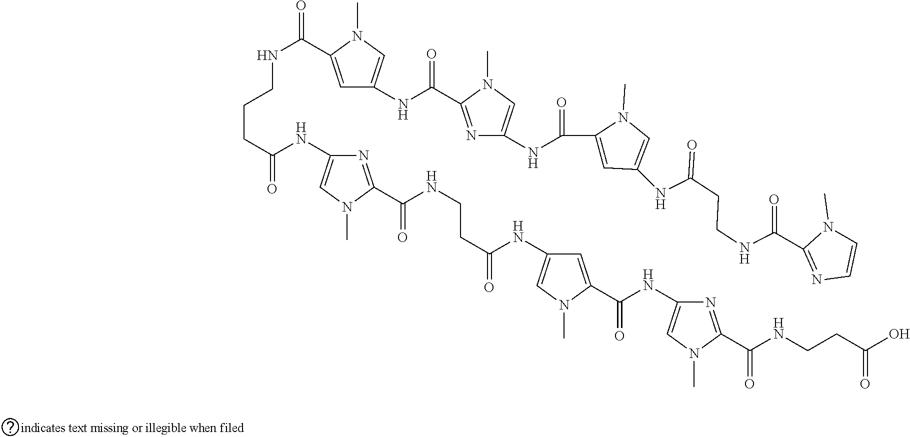 [SMILES: Cn1cc(NC(=O)c2nc(NC(=O)c3cc(NC(=O)CCNC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1]

### Step 1: Synthesis of ethyl 4-[4-(3-aminopropanamido)-1-methylpyrrole-2-amido]-1-methylimidazole-2-carboxylate

The procedure was the same as 3-amino-N-propylpropanamide. 1.20 g of ethyl 4-(4-{3-[(tert-butoxycarbonyl) amino] propanamido}-1-methylpyrrole-2-amido)-1-methylimidazole-2-carboxylate was used, 1.20 g crude of ethyl 4-[4-(3-aminopropanamido)-1-methylpyrrole-2-amido]-1-methylimidazole-2-carboxylate was obtained as yellow oil. LC/MS: mass calcd. For C₁₆H₂₂N₆O₄:362.17, found: 363.20 [M+H]⁺.

### Step 2: Synthesis of ethyl 4-[(2R)-2-[(tert-butoxycarbonyl) amino]-4-({1-methyl-4-[1-methyl-4-(3-{[1-methyl-4-(1-methylimidazole-2-amido) pyrrol-2-yl]formamido}propanamido) imidazole-2-amido]pyrrol-2-yl}formamido) butanamido]-1-methylimidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate. 938.00 mg of ethyl 4-[4-(3-aminopropanamido)-1-methylpyrrole-2- amido]-1-methylimidazole-2-carboxylate was used, 1.30 g of ethyl 4-[(2R)-2-[(tert-butoxycarbonyl) amino]-4-({1-methyl-4-[1-methyl-4-(3-{[1-methyl-4-(1-methylimidazole-2-amido) pyrrol-2-yl]formamido}propanamido) imidazole-2-amido]pyrrol- 2-yl}formamido) butanamido]-1-methylimidazole-2-carboxylate was obtained as yellow solid (72.72% yield of two steps). LC/MS: mass calcd. For C₂₁H₂₆N₈O₅: 470.20, found: 471.40[M+H]⁺.

### Step 3: Synthesis of 1-methyl-4-(1-methyl-4-{3-[(1-methylimidazol-2-yl) formamido]propanamido}pyrrole-2-amido)imidazole-2-carboxylic acid

The procedure was the same as 4-[3-[(tert-butoxycarbonyl)amino]propanamido]-1-methylimidazole-2-carboxylic acid, but the reaction temperature was r.t., the reaction time was 1.0 h. 1.10 g of ethyl 1-methyl-4-(1-methyl-4-{3-[(1-methylimidazol-2-yl)formamido]propanamido}pyrrole-2-amido)imidazole-2-carboxylate was used, 800.00 mg of 1-methyl-4-(1-methyl-4-{3-[(1-methylimidazol-2-yl)formamido]propanamido}pyrrole-2-amido)imidazole-2-carboxylic acid was obtained as yellow solid (73.47% yield). LC/MS: mass calcd. For C₁₉H₂₂N₈O₅: 442.17, found: 443.25 [M+H]⁺.

### Step 4: Synthesis of methyl 1-methyl-4-[1-methyl-4-(1-methyl-4-{3-[(1-methylimidazol-2-yl)formamido]propanamido}pyrrole-2-amido)imidazole-2-amido]pyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 635.00 mg of 1-methyl-4-(1-methyl-4-{3-[(1-methylimidazol-2-yl)formamido]propanamido}pyrrole-2-amido)imidazole-2-carboxylic acid was used, 770.00 mg of methyl 1-methyl-4-[1-methyl-4-(1-methyl-4-{3-[(1-methylimidazol-2-yl)formamido]propanamido}pyrrole-2-amido)imidazole-2-amido]pyrrole-2-carboxylate was obtained as yellow solid (92.72% yield). LC/MS: mass calcd. For C₂₆H₃₀N₁₀O₆: 578.23, found: 579.56[M+H]⁺.

### Step 5: Synthesis of 1-methyl-4-[1-methyl-4-(1-methyl-4-{3-[(1-methylimidazol-2-yl)formamido]propanamido}pyrrole-2-amido)imidazole-2-amido]pyrrole-2-carboxylic acid

The procedure was the same as 4-[3-[(tert-butoxycarbonyl)amino]propanamido]-1-methylimidazole-2-carboxylic acid, but the reaction solvent was THF/MeOH=1/2, the reaction temperature was r.t., the reaction time was 5.0 h. 750.00 mg of methyl 1-methyl-4-[1-methyl-4-(1-methyl-4-{3-[(1-methylimidazol-2-yl)formamido]propanamido}pyrrole-2-amido)imidazole-2-amido] pyrrole-2-carboxylate was used, 540.00 mg of 1-methyl-4-[1-methyl-4-(1-methyl-4-{3-[(1-methylimidazol-2-yl)formamido]propanamido}pyrrole-2-amido)imidazole-2-amido]pyrrole-2-carboxylic acid was obtained as white solid (73.79% yield). LC/MS: mass calcd. For C₂₅H₂₈N₁₀O₆: 564.22, found: 565.25[M+H]⁺.

### Step 6: Synthesis of 4-{4-[(tert-butoxycarbonyl)amino]butanamido}-1-methylimidazole-2-carboxylic acid

The procedure was the same as 4-[3-[(tert-butoxycarbonyl)amino] propanamido]-1-methylimidazole-2-carboxylic acid. 6.30 g of ethyl 4-{4-[(tert-butoxycarbonyl)amino]butanamido}-1-methylimidazole-2-carboxylate was used, 5.60 g of 4-{4-[(tert-butoxycarbonyl)amino]butanamido}-1-methylimidazole-2-carboxylic acid was obtained as white solid (96.53% yield). LC/MS: mass calcd. For C₁₄H₂₂N₄O₅: 326.15, found: 327.10[M+H]⁺.

### Step 7: Synthesis of ethyl 3-{[4-(4-{3-[(4-{4-[(tert-butoxycarbonyl)amino] butanamido}-1-methylimidazol-2-yl)formamido]propanamido}-1-methylpyrrole-2-amido)-1-methylimidazol-2-yl]formamido}propanoate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 813.00 mg of ethyl 3-({4-[4-(3-aminopropanamido)-1-methylpyrrole-2-amido]-1-methylimidazol-2-yl}formamido)propanoate was used, 800.00 mg of ethyl 3-{[4-(4-{3-[(4-{4-[(tert-butoxycarbonyl)amino]butanamido}-1-methylimidazol-2-yl)formamido]propanamido}-1-methylpyrrole-2-amido)-1-methylimidazol-2-yl]formamido}propanoate was obtained as yellow solid (57.50% yield). LC/MS: mass calcd. For C₃₃H₄₇N₁₁O₉: 741.36, found: 742.35[M+H]⁺.

### Step 8: Synthesis of ethyl 3-({4-[4-(3-{[4-(4-aminobutanamido)-1-methylimidazol-2-yl]formamido}propanamido)-1-methylpyrrole-2-amido]-1-methylimidazol-2-yl}formamido)propanoate

The procedure was the same as 3-amino-N-propylpropanamide. 650.00 mg of ethyl 3-{[4-(4-{3-[(4-{4-[(tert-butoxycarbonyl)amino] butanamido}-1-methylimidazol-2-yl)formamido]propanamido}-1-methylpyrrole-2-amido)-1-methylimidazol-2-yl] formamido}propanoate was used, 650.00 mg crude of ethyl 3-({4-[4-(3-{[4-(4-aminobutanamido)-1-methylimidazol-2-yl]formamido} propanamido)-1-methylpyrrole-2-amido]-1-methylimidazol-2-yl}formamido)propanoate was obtained as yellow oil. LC/MS: mass calcd. For C₂₈H₃₉N₁₁O₇: 641.30, found: 642.50[M+H]⁺.

### Step 9: Synthesis of ethyl 3-[(1-methyl-4-{1-methyl-4-[3-({1-methyl-4-[4-({1-methyl-4-[1-methyl-4-(1-methyl-4-{3-[(1-methylimidazol-2-yl)formamido] propanamido}pyrrole-2-amido)imidazole-2-amido]pyrrol-2-yl}formamido)butanamido]imidazol-2-yl}formamido)propanamido]pyrrole-2-amido}imidazol-2-yl) formamido]propanoate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 502.00 mg of 1-methyl-4-[1-methyl-4-(1-methyl-4-{3-[(1-methylimidazol-2-yl)formamido]propanamido}pyrrole-2-amido)imidazole-2-amido]pyrrole-2-carboxylic acid was used, 1.00 g of ethyl 3-[(1-methyl-4-{1-methyl-4-[3-({1-methyl-4-[4-({1-methyl-4-[1-methyl-4-(1-methyl-4-{3-[(1-methylimidazol-2-yl)formamido]propanamido}pyrrole-2-amido)imidazole-2-amido]pyrrol-2-yl} formamido)butanamido]imidazol-2-yl}formamido)propanamido]pyrrole-2-amido}imidazol-2-yl)formamido]propanoate was obtained as yellow solid (80.53% yield). LC/MS: mass calcd. For C₅₃H₆₅N₂₁O₁₂: 1187.51, found: 1188.45[M+H]⁺.

### Step 10: Synthesis of 3-[(1-methyl-4-{1-methyl-4-[3-({1-methyl-4-[4-({1-methyl-4-[1-methyl-4-(1-methyl-4-{3-[(1-methylimidazol-2-yl)formamido]propanamido}pyrrole-2-amido)imidazole-2-amido]pyrrol-2-yl}formamido)butanamido]imidazol-2-yl}formamido)propanamido]pyrrole-2-amido}imidazol-2-yl)formamido] propanoic acid

The procedure was the same as 4-[3-[(tert-butoxycarbonyl)amino] propanamido]-1-methylimidazole-2-carboxylic acid, but the reaction solvent was THF/MeOH=1/2, the reaction temperature was r.t., the reaction time was 1.0 h. 1.00 g of ethyl 3-[(1-methyl-4-{1-methyl-4-[3-({1-methyl-4-[4-({1-methyl-4-[1-methyl-4-(1-methyl-4-{3-[(1-methylimidazol-2-yl)formamido] propanamido}pyrrole-2-amido)imidazole-2-amido]pyrrol-2-yl}formamido)butanamido]imidazol-2-yl}formamido)propanamido]pyrrole-2-amido}imidazol-2-yl) formamido]propanoate was used, 900.00 mg of ethyl 3-[(1-methyl-4-{1-methyl-4-[3-({1-methyl-4-[4-({1-methyl-4-[1-methyl-4-(1-methyl-4-{3-[(1-methylimidazol-2-yl)formamido]propanamido}pyrrole-2-amido)imidazole-2-amido]pyrrol-2-yl}formamido)butanamido]imidazol-2-yl}formamido)propanamido]pyrrole-2-amido}imidazol-2-yl)formamido]propanoate was obtained as yellow solid (82.96% yield). LC/MS: mass calcd. For C₅₁H₆₁N₂₁O₁₂: 1159.48, found: 581.00 [½M+H]⁺.

### Example 35. Synthesis of (S)-4-(4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxamido)-1-methyl-1H-pyrrole-2-carboxylic acid (Compound 66)

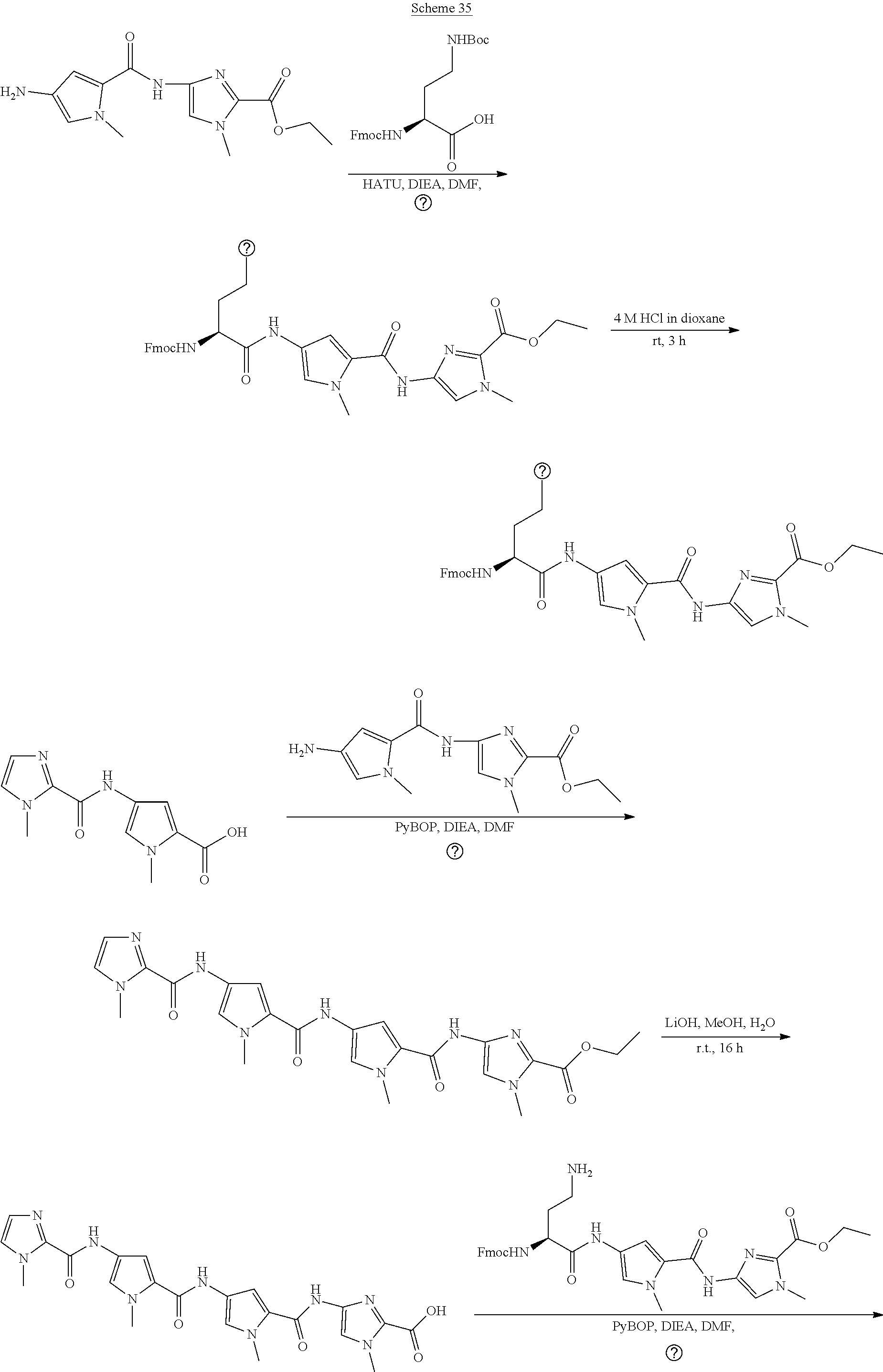

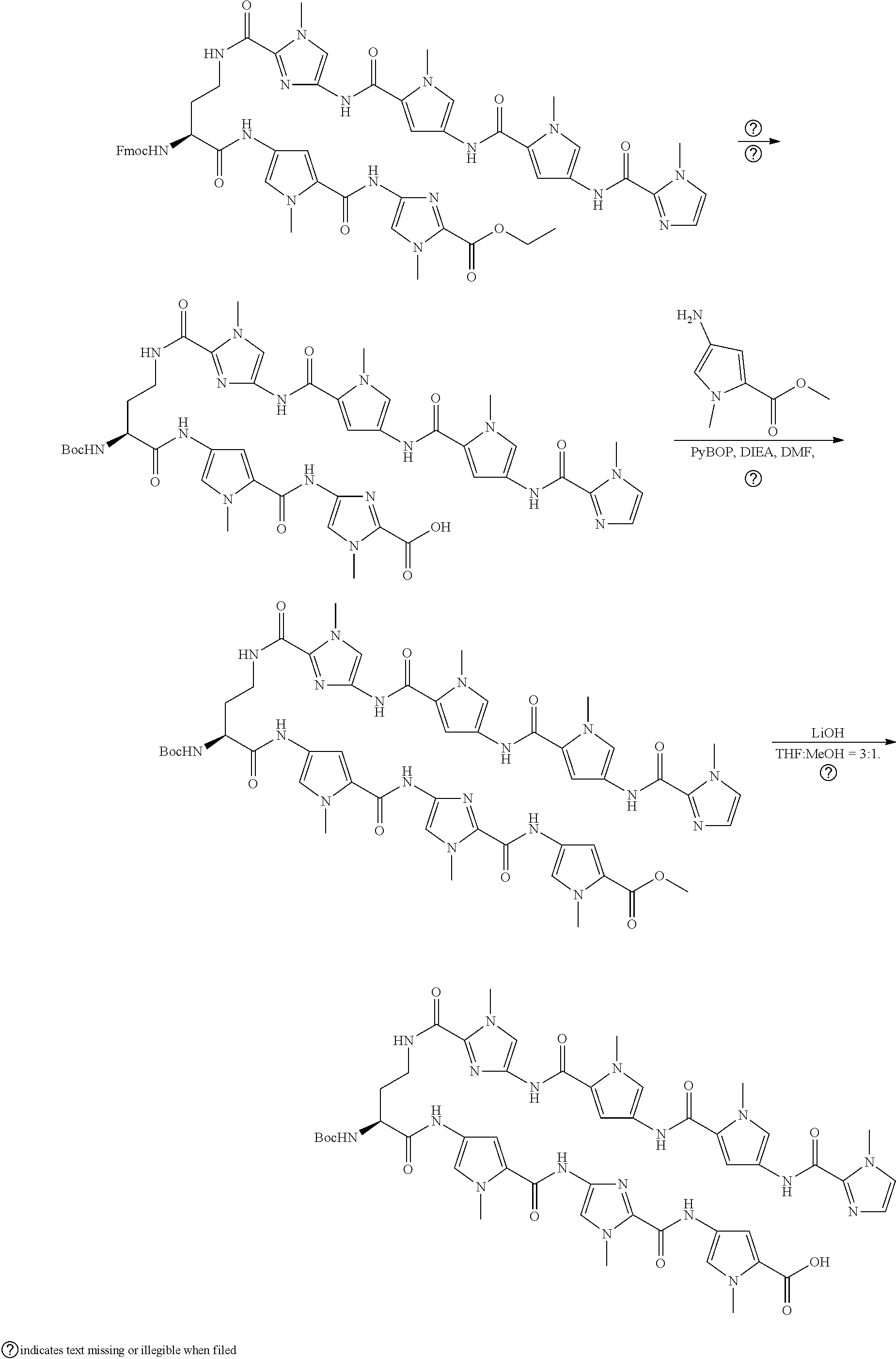

### Step 1: Synthesis of ethyl (S)-4-(4-(2-((((9H-fluoren-9-yl)methoxy)carbonyl) amino)-4-((tert-butoxycarbonyl)amino)butanamido)-1-methyl-1H-pyrrole-2-carboxamido)-1-methyl-1H-imidazole-2-carboxylate

The procedure was the same as ethyl 4-[3-[(tert-butoxycarbonyl)amino]propanamido]-1-methylimidazole-2-carboxylate. 0.91 g of ethyl 4-(4-amino-1-methyl-1H-pyrrole-2-carboxamido)-1-methyl-1H-imidazole-2-carboxylate was used, 1.10 g of ethyl (S)-4-(4-(2-((((9H-fluoren-9-yl)methoxy) carbonyl)amino)-4-((tert-butoxycarbonyl)amino)butanamido)-1-methyl-1H-pyrrole-2-carboxamido)-1-methyl-1H-imidazole-2-carboxylate was obtained as a yellow solid (59.03% yield). LC/MS: mass calcd. For C₃₇H₄₃N₇O₅: 713.32, found: 714.35 [M+H]⁺.

### Step 2: Synthesis of ethyl (S)-4-(4-(2-((((9H-fluoren-9-yl)methoxy)carbonyl) amino)-4-aminobutanamido)-1-methyl-1H-pyrrole-2-carboxamido)-1-methyl-1H-imidazole-2-carboxylate

The procedure was the same as methyl 4-[4-(3-aminopropanamido)-1-methylimidazole-2-amido]-1-methylpyrrole-2-carboxylate hydrochloride, but the reaction time was 3.0 h. 1.15 g of ethyl (S)-4-(4-(2-((((9H-fluoren-9-yl)methoxy)carbonyl)amino)-4-((tert-butoxycarbonyl)amino)butanamido)-1-methyl-1H-pyrrole-2-carboxamido)-1-methyl-1H-imidazole-2-carboxylatee was used, 980.00 mg of ethyl (S)-4-(4-(2-((((9H-fluoren-9-yl)methoxy) carbonyl)amino)-4-aminobutanamido)-1-methyl-1H-pyrrole-2-carboxamido)-1-methyl-1H-imidazole-2-carboxylate was obtained as off-white solid (99.12% yield). LC/MS: mass calcd. For C₃₂H₃₅N₇O₆: 613.26, found: 614.25 [M+H]⁺.

### Step 3: Synthesis of ethyl 1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxylate

The procedure was the same as ethyl 3-{[4-(4-{3-[(tert-butoxycarbonyl)amino] propanamido}-1-methylpyrrole-2-amido)-1-methylimidazol-2-yl]formamido}propanoate, but the reaction time was 6.0 h. 0.50 g of 1-methyl-4-(1-methyl-1H-imidazole-2-carboxamido)-1H-pyrrole-2-carboxylic acid was used, 600.00 mg of ethyl 1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxylate was obtained as a yellow solid(57.12% yield). LC/MS: mass calcd. For C₂₄H₂₇N₉O₅: 521.21, found: 522.25 [M+H]⁺.

### Step 4: Synthesis of 1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxylic acid

The procedure was the same as 4-[3-[(tert-butoxycarbonyl)amino]propanamido]-1-methylimidazole-2-carboxylic acid, but the reaction temperature was room temperature, the reaction time was 16.0 h. 580.00 mg of ethyl 1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxylate was used, 440.00 mg of 1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxylic acid was obtained as a yellow solid (80.17% yield). LC/MS: mass calcd. For C₂₂H₂₃N₉O₅: 493.18, found: 494.25 [M+H]⁺.

### Step 5: Synthesis of ethyl (S)-4-(4-(2-((((9H-fluoren-9-yl)methoxy)carbonyl) amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxylate

The procedure was the same as ethyl 4-(4-{3-[(tert-butoxycarbonyl)amino] propanamido}-1-methylpyrrole-2-amido)-1-methylimidazole-2-carboxylate, but the reaction time was 16.0 h. 430.00 mg of 1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxylic acid was used. 750.00 mg of ethyl (S)-4-(4-(2-((((9H-fluoren-9-yl)methoxy)carbonyl) amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxylate was obtained as a yellow solid (79.03% yield). LC/MS: mass calcd. For C₅₄H₅₆N₁₆O₁₀: 1088.44, found: 1089.50 [M+H]⁺.

### Step 6: Synthesis of (S)-4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxylic acid

To a stirred solution of ethyl (S)-4-(4-(2-((((9H-fluoren-9-yl)methoxy)carbonyl) amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxylate (660.00 mg, 0.61 mmol, 1.00 equiv) in THF (6.00 mL) and MeOH (2.00 mL) were added 0.5 M LiOH (6.06 mL, 3.03 mmol, 5.00 equiv) in portions at room temperature. The resulting mixture was stirred for 48 h at room temperature. To the above mixture was added Boc₂O (396.76 mg, 1.82 mmol, 3.00 equiv) and DMAP (111.05 mg, 0.91 mmol, 1.50 equiv) at room temperature. The resulting mixture was stirred for additional 16 h at room temperature. The resulting mixture was concentrated under vacuum to remove solvent. The resulting mixture was filtered, the filter cake was washed with Et₂O (3×6 mL) to afford (S)-4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxylic acid (400.00 mg, 70.30%) as a yellow solid. LC/MS: mass calcd. For C₄₂H₅₀N₁₆O₁₀: 938.39, found: 939.55 [M+H]⁺.

### Step 7: Synthesis of methyl (S)-4-(4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxamido)-1-methyl-1H-pyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate, but the reaction time was 16.0 h. 350.00 mg of (S)-4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxylic acid was used, 250.00 mg of methyl (S)-4-(4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxamido)-1-methyl-1H-pyrrole-2-carboxylate was obtained as a yellow solid (62.38% yield). LC/MS: mass calcd. For C₄₉H₅₈N₁₈O₁₁: 1074.45, found: 1075.90 [M+H]⁺.

### Step 8: Synthesis of afford (S)-4-(4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxamido)-1-methyl-1H-pyrrole-2-carboxylic acid

To a stirred solution of methyl 4methyl (S)-4-(4-(4-(2-((tert-butoxycarbonyl) amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxamido)-1-methyl-1H-pyrrole-2-carboxylate (60.00 mg, 0.06 mmol, 1.00 equiv) in MeOH (0.60 mL) and THF (1.80 mL) was added 0.5 M LiOH (0.56 mL, 0.28 mmol, 5.00 equiv) at room temperature. The resulting mixture was stirred for 72 h at room temperature. The resulting mixture was concentrated under reduced pressure. The crude product (30.00 mg) was purified by Prep-HPLC with the following conditions (Column: XSelect CSH Prep C18 OBD Column, 19*250 mm, 5 m; Mobile Phase A: Water (0.05% TFA), Mobile Phase B: ACN; Flow rate: 25 mL/min; Gradient: 25% B to 35% B in 10 min, 35% B; Wave Length: 254 nm; RT1 (min): 9.6; Number Of Runs: 4). 3.1 mg of desired product was obtained as a white solid. (5.03% yield). LC/MS: mass calcd. For C₄₈H₅₆N₁₈O₁₁: 1060.44, found: 1061.51 [M+H]⁺.

### Example 36. Synthesis of 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid (Compound 49)

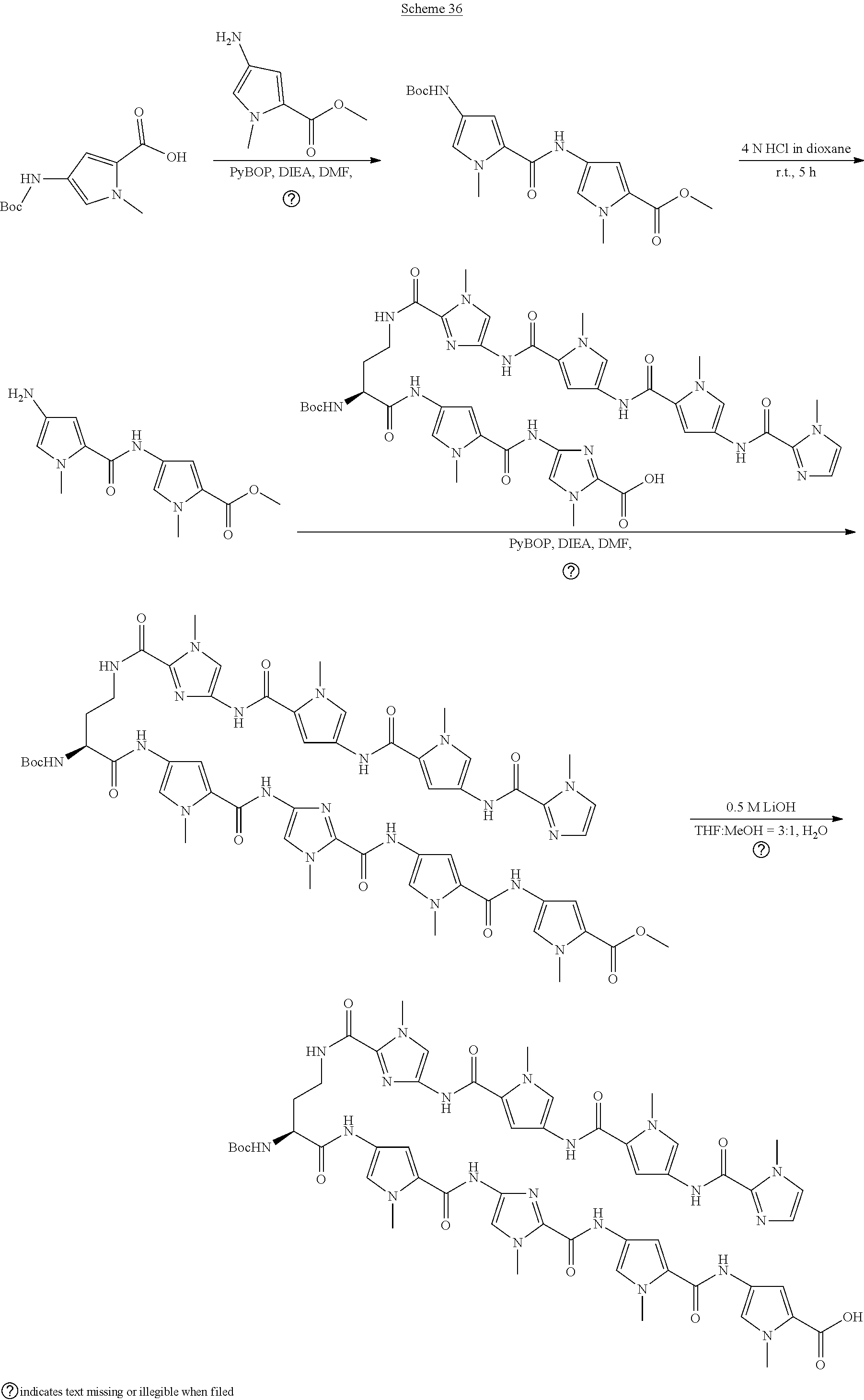

### Step 1: Synthesis of methyl 4-(4-((tert-butoxycarbonyl)amino)-1-methyl-1H-pyrrole-2-carboxamido)-1-methyl-1H-pyrrole-2-carboxylate

The procedure was the same as ethyl 3-{[4-(4-{3-[(tert-butoxycarbonyl)amino] propanamido}-1-methylpyrrole-2-amido)-1-methylimidazol-2-yl]formamido}propanoate, but the reaction temperature was 50° C., the reaction time was 16.0 h. 1.00 g of 4-[(tert-butoxycarbonyl)amino]-1-methylpyrrole-2-carboxylic acid was used. 1.10 g of methyl 4-{4-[(tert-butoxycarbonyl)amino]-1-methylpyrrole-2-amido}-1-methylpyrrole-2-carboxylate was obtained as a yellow solid (70.21% yield). LC/MS: mass calcd. For C₁₈H₂₄N₄O₅: 376.17, found: 377.10 [M+H]⁺.

### Step 2: Synthesis of methyl 4-(4-amino-1-methylpyrrole-2-amido)-1-methylpyrrole-2-carboxylate

To a stirred mixture of methyl 4-{4-[(tert-butoxycarbonyl)amino]-1-methylpyrrole-2-amido}-1-methylpyrrole-2-carboxylate (1.27 g, 3.37 mmol, 1.00 eq) in DCM (5.00 mL) was added HCl (gas) in 1,4-dioxane (4M, 25.00 mL) in portions at room temperature. The resulting mixture was stirred for 5.0 h at room temperature. The precipitated solids were collected by filtration and washed with diethyl ether (3×10 mL). 900.00 mg (96.54% yield) desired product was obtained as a yellow solid. LC/MS: mass calcd. For C₁₃H₁₆N₄O₃: 276.12, found: 277.00 [M+H]⁺.

### Step 3: Synthesis of methyl 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate. 600.00 mg of 4-{4-[(2S)-2-[(tert-butoxycarbonyl)amino]-4-[(1-methyl-4-{1-methyl-4-[1-methyl-4-(1-methylimidazole-2-amido)pyrrole-2-amido] pyrrole-2-amido}imidazol-2-yl)formamido]butanamido]-1-methylpyrrole-2-amido}-1-methylimidazole-2-carboxylic acid was used, 400.00 mg of desired product was obtained as a yellow solid (52.28% yield). LC/MS: mass calcd. For C₅₅H₆₄N₂₀O₁₂: 1196.50, found: 1197.80 [M+H]⁺.

### Step 4: Synthesis of 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid

To a stirred solution of methyl (S)-4-(4-(4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxamido)-1-methyl-1H-pyrrole-2-carboxamido)-1-methyl-1H-pyrrole-2-carboxylate (430.00 mg, 0.36 mmol, 1.00 equiv) in THF (30.00 mL) and MeOH (10.00 mL) were added 0.5 M LiOH (3.60 mL, 5.00 equiv) at room temperature under air atmosphere. The resulting mixture was stirred for 48.0 h at room temperature. The resulting mixture was concentrated under reduced pressure. The residue was purified by reverse flash chromatography with the following conditions: column, silica gel; mobile phase, MeCN in water (0.5% TFA), 10% to 50% gradient in 10 min; detector, UV 254 nm. 150.00 mg of desired product was obtained as a yellow solid (35.30% yield). LC/MS: mass calcd. For C₅₄H₆₂N₂₀O₁₂: 1182.49, found: 1183.90 [M+H]⁺.

### Example 37. Synthesis of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid (Compound 51)

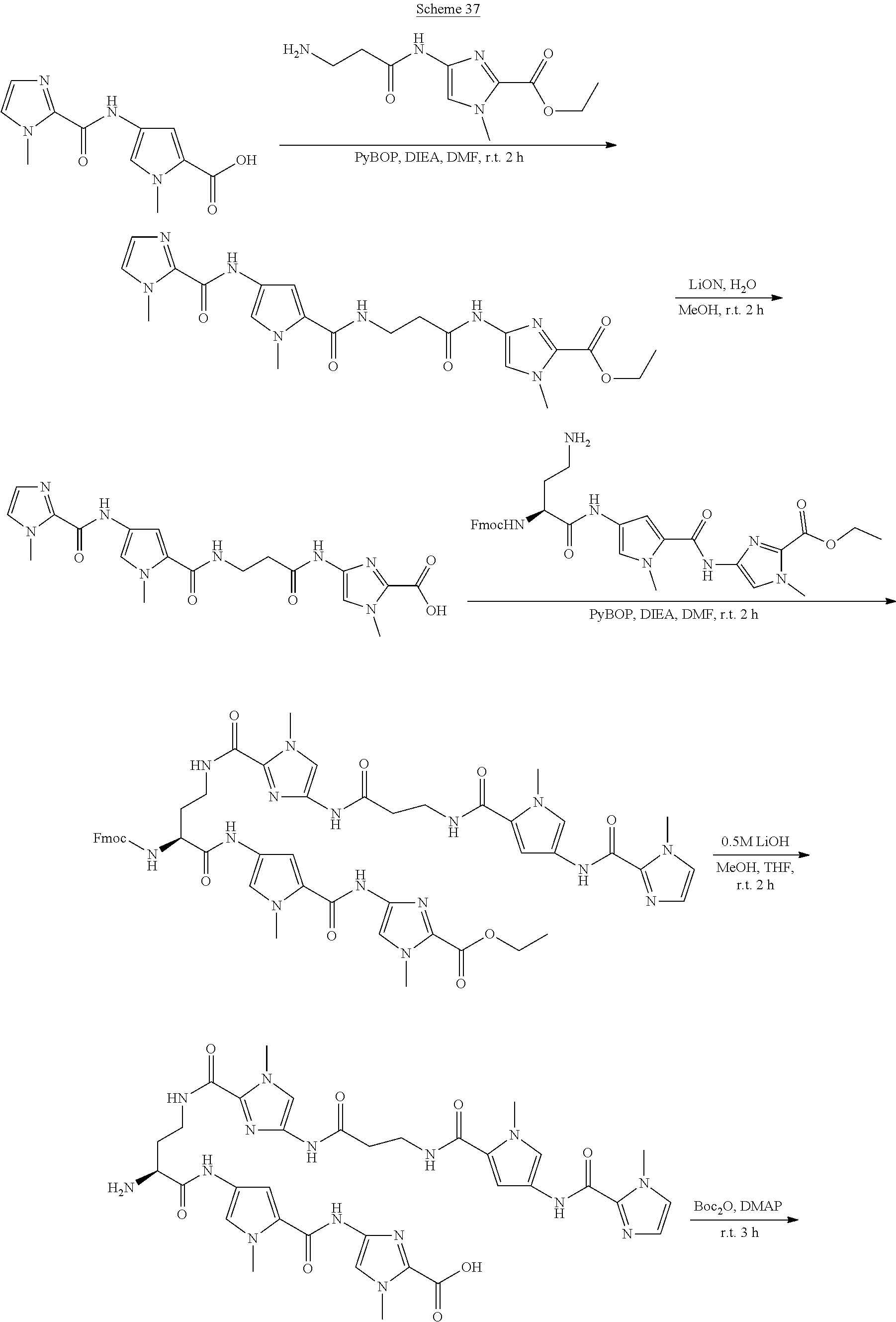

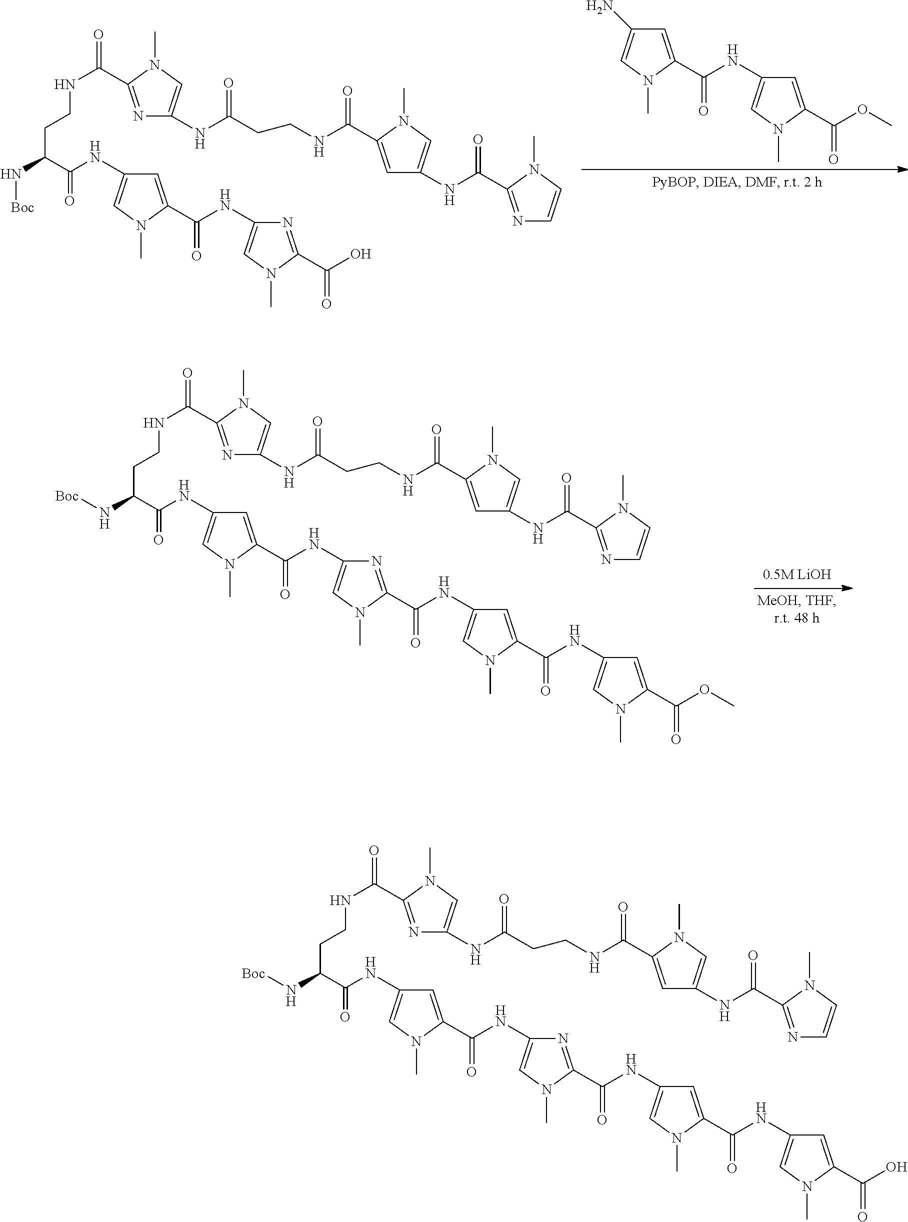 [SMILES: Cn1cc(NC(=O)c2nccn2C)cc1C(=O)NCCC(=O)Nc1cn(C)c(C(=O)NCC[C@H](NC(=O)OC(C)(C)C)C(=O)Nc2cc(C(=O)Nc3cn(C)c(C(=O)O)n3)n(C)c2)n1 ; COC(=O)c1cc(NC(=O)c2cc(N)cn2C)cn1C ; COC(=O)c1cc(NC(=O)c2cc(NC(=O)c3nc(NC(=O)c4cc(NC(=O)[C@H](CCNC(=O)c5nc(NC(=O)CCNC(=O)c6cc(NC(=O)c7nccn7C)cn6C)cn5C)NC(=O)OC(C)(C)C)cn4C)cn3C)cn2C)cn1C ; Cn1cc(NC(=O)c2cc(NC(=O)c3nc(NC(=O)c4cc(NC(=O)[C@H](CCNC(=O)c5nc(NC(=O)CCNC(=O)c6cc(NC(=O)c7nccn7C)cn6C)cn5C)NC(=O)OC(C)(C)C)cn4C)cn3C)cn2C)cc1C(=O)O]

### Step 1: Synthesis of ethyl 1-methyl-4-(3-{[1-methyl-4-(1-methylimidazole-2-amido)pyrrol-2-yl]formamido}propanamido) imidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate, but the reaction time was 2.0 h. 1.50 g of ethyl 4-(3-aminopropanamido)-1-methylimidazole-2-carboxylate was used, 2.00 g of ethyl 1-methyl-4-(3-{[1-methyl-4-(1-methylimidazole-2-amido)pyrrol-2-yl] formamido} propanamido) imidazole-2-carboxylate was obtained as an off-white solid (68.09% yield). LC/MS: mass calcd. For C₂₁H₂₆N₈O₅:470.20, found: 471.40 [M+H]⁺.

### Step 2: Synthesis of 1-methyl-4-(3-{[1-methyl-4-(1-methylimidazole-2-amido) pyrrol-2-yl]formamido}propanamido)imidazole-2-carboxylic acid

The procedure was the same as 4-[3-[(tert-butoxycarbonyl)amino]propanamido]-1-methylimidazole-2-carboxylic acid, but the reaction temperature was r.t., the reaction time was 2.0 h. 2.00 g of ethyl 1-methyl-4-(3-{[1-methyl-4-(1-methylimidazole-2-amido)pyrrol-2-yl]formamido}propanamido)imidazole-2-carboxylate was used, 1.80 g of 1-methyl-4-(3-{[1-methyl-4-(1-methylimidazole-2-amido)pyrrol-2-yl]formamido}propanamido)imidazole-2-carboxylic acid was obtained as an off-white solid (95.71% yield). LC/MS: mass calcd. For C₁₉H₂₂N₈O₅: 442.17, found: 443.10 [M+H]⁺.

### Step 3: Synthesis of ethyl 4-{4-[(2S)-2-{[(9H-fluoren-9-ylmethoxy)carbonyl] amino}-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate, but the reaction time was 2.0 h. 1.60 g of ethyl 4-{4-[(2S)-4-amino-2-{[(9H-fluoren-9-ylmethoxy)carbonyl]amino}butanamido]-1-methylpyrrole-2-amido}-1-methylimidazole-2-carboxylate was used, 1.90 g of ethyl 4-{4-[(2S)-2-{[(9H-fluoren-9-ylmethoxy)carbonyl]amino}-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate was obtained as a light yellow solid (70.20% yield). LC/MS: mass calcd. For C₅H₅₅N₁₅O₁₀: 1037.43, found: 1038.45 [M+H]⁺.

### Step 4: Synthesis of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid

A mixture of ethyl 4-{4-[(2S)-2-{[(9H-fluoren-9-ylmethoxy)carbonyl]amino}-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylate (1.90 g, 1.83 mmol, 1.00 equiv) and LiOH (0.22 g, 9.15 mmol, 5.00 equiv) in MeOH (5.00 mL), THF (15.00 mL) and H₂O (18.30 mL) was stirred for 2.0 h at room temperature. The resulting mixture was used in the next step directly without further purification. LC/MS: mass calcd. For C₃₄H₄₁N₁₅O₅: 787.33, found: 788.40 [M+H]⁺.

### Step 5: Synthesis of 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid

The mixture of 4-{4-[(2S)-2-amino-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.40 g, 1.78 mmol, 1.00 equiv) in MeOH/THF/H₂O (5.00 mL/15.00 mL/18.30 mL) was added di-tert-butyl dicarbonate (0.78 g, 3.55 mmol, 2.00 equiv) and DMAP (0.02 g, 0.18 mmol, 0.10 equiv). The reaction was stirred at room temperature for 3.0 h. The mixture was added with H₂O (30 mL). The mixture was filtered through a celite pad, and the solid was washed with ethyl acetate (3×30 mL) to afford 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid (1.20 g, 76.05%) as a yellow solid. LC/MS: mass calcd. For C₃₉H₄₉N₁₅O₁₀: 887.38, found: 888.45 [M+H]⁺.

### Step 6: Synthesis of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate, but the reaction time was 2 h. 1.20 g of 4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-carboxylic acid was used, 1.10 g of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate was obtained as a yellow solid (71.01% yield). LC/MS: mass calcd. For C₅₂H₆₃N₁₉O₁₂: 1145.49, found: 1146.50 [M+H]⁺.

### Step 7: Synthesis of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid

The procedure was the same as 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid. 1.00 g of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate was used, 400.00 mg of 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid was obtained as a white solid (39.16% yield). LC/MS: mass calcd. For C₅₁H₆₁N₁₉O₁₂: 1131.47, found: 1132.65 [M+H]⁺.

### Example 38. Synthesis of 1-methyl-4-(1-methyl-4-{1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-amido}pyrrole-2-amido)pyrrole-2-carboxylic acid (Compound 148)

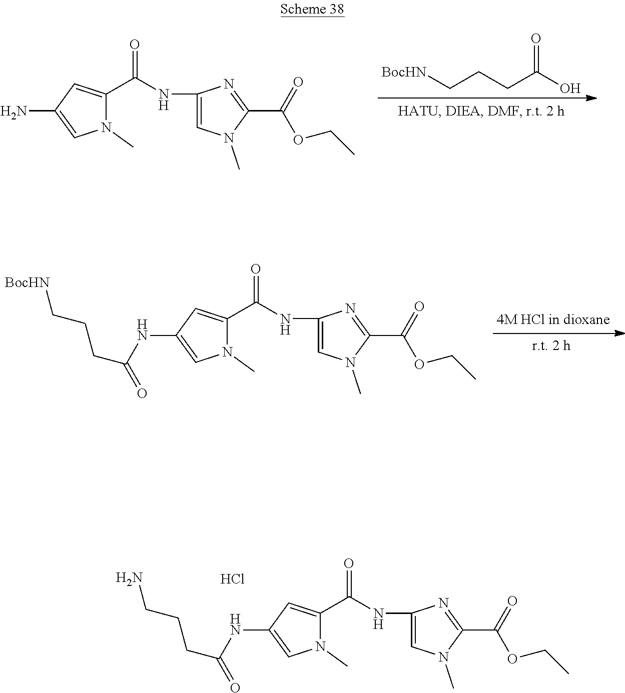 [SMILES: CCOC(=O)c1nc(NC(=O)c2cc(N)cn2C)cn1C ; CC(C)(C)OC(=O)NCCCC(=O)O ; CCOC(=O)c1nc(NC(=O)c2cc(NC(=O)CCCNC(=O)OC(C)(C)C)cn2C)cn1C ; CCOC(=O)c1nc(NC(=O)c2cc(NC(=O)CCCN)cn2C)cn1C ; Cl]

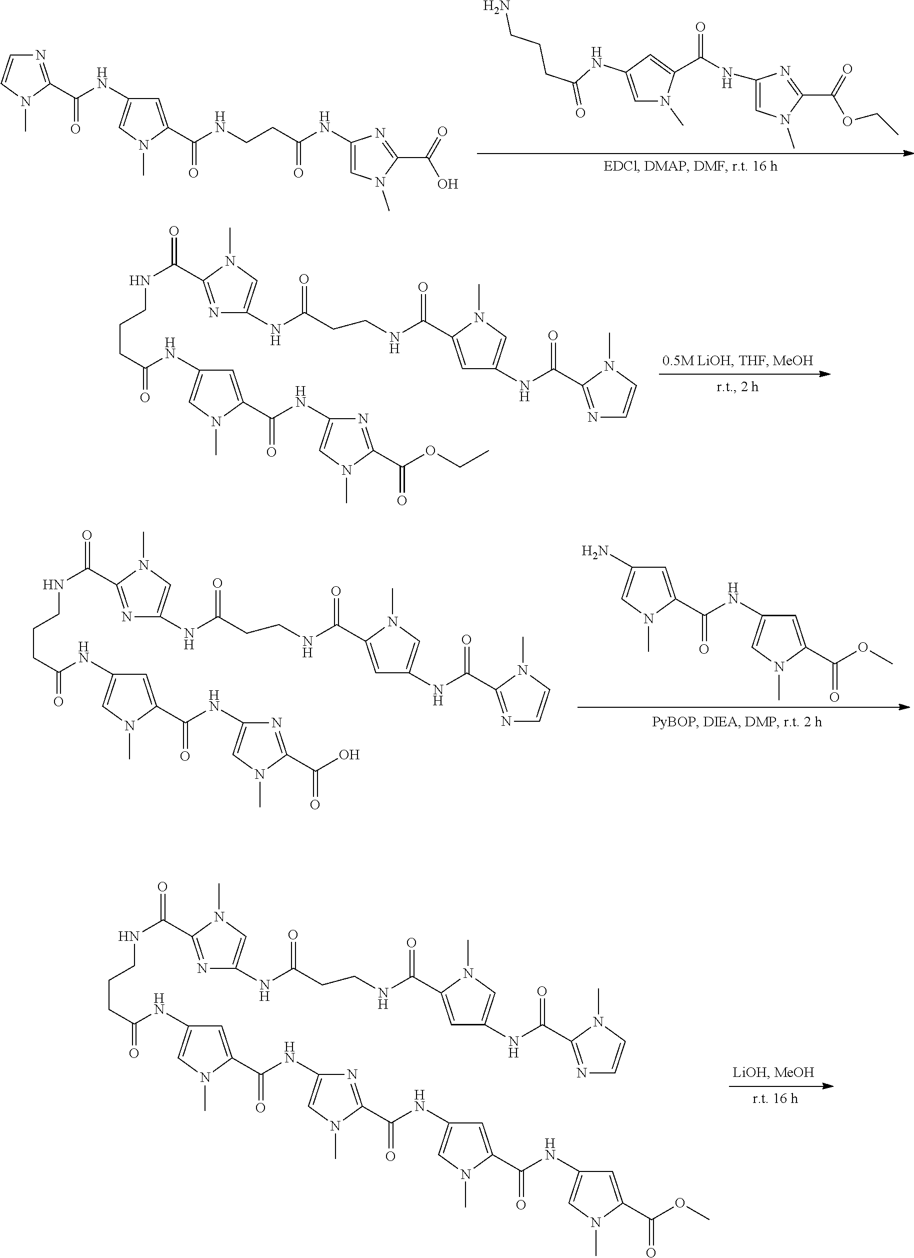 [SMILES: Cn1cc(NC(=O)c2nccn2C)cc1C(=O)NCCC(=O)Nc1cn(C)c(C(=O)O)n1 ; CCOC(=O)c1nc(NC(=O)c2cc(NC(=O)CCCN)cn2C)cn1C ; CCOC(=O)c1nc(NC(=O)c2cc(NC(=O)CCCNC(=O)c3nc(NC(=O)CCNC(=O)c4cc(NC(=O)c5nccn5C)cn4C)cn3C)cn2C)cn1C ; Cn1cc(NC(=O)c2nccn2C)cc1C(=O)NCCC(=O)Nc1cn(C)c(C(=O)NCCCC(=O)Nc2cc(C(=O)Nc3cn(C)c(C(=O)O)n3)n(C)c2)n1 ; COC(=O)c1cc(NC(=O)c2cc(N)cn2C)cn1C ; COC(=O)c1cc(NC(=O)c2cc(NC(=O)c3nc(NC(=O)c4cc(NC(=O)CCCNC(=O)c5nc(NC(=O)CCNC(=O)c6cc(NC(=O)c7nccn7C)cn6C)cn5C)cn4C)cn3C)cn2C)cn1C]

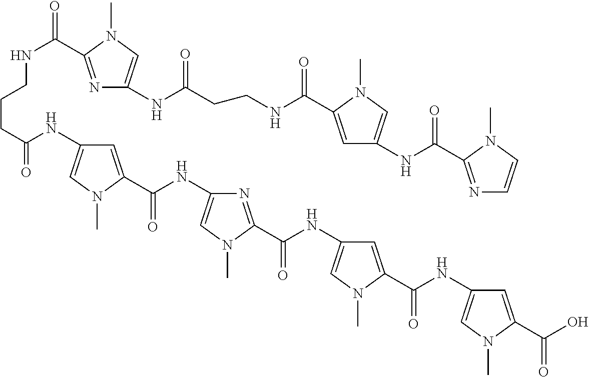 [SMILES: Cn1cc(NC(=O)c2cc(NC(=O)c3nc(NC(=O)c4cc(NC(=O)CCCNC(=O)c5nc(NC(=O)CCNC(=O)c6cc(NC(=O)c7nccn7C)cn6C)cn5C)cn4C)cn3C)cn2C)cc1C(=O)O]

### Step 1: Synthesis of ethyl 4-(4-{4-[(tert-butoxycarbonyl)amino]butanamido}-1-methylpyrrole-2-amido)-1-methylimidazole-2-carboxylate

The procedure was the same as methyl 3-[(4-[3-[(tert-butoxycarbonyl)amino] propanamido]-1-methylimidazol-2-yl)formamido]propanoate, but the reaction time was 2.0 h. 1.00 g of ethyl 4-(4-amino-1-methylpyrrole-2-amido)-1-methylimidazole-2-carboxylate was used, 800.00 mg of ethyl 4-(4-{14-[tert-butoxycarbonyl)amino]butanamido}-1-methylpyrrole-2-amido)-1-methylimidazole-2-carboxylate was obtained as a Brown yellow solid (48.901% yield). LC/MS: mass calcd. For C₂₂H₃₂N₆H₆: 476.24, found: 477.15 [M+H]⁺.

### Step 2: Synthesis of ethyl 4-[4-(4-aminobutanamido)-1-methylpyrrole-2-amido]-1-methylimidazole-2-carboxylate

The procedure was the same as methyl 4-(4-amino-1-methylpyrrole-2-amido)-1-methylpyrrole-2-carboxylate, but the reaction time was 2.0 h. 750.00 mg of ethyl 4-(4-{14-[(tert-butoxycarbonyl)amino]butanamido}-1-methylpyrrole-2-amido)-1-methylimidazole-2-carboxylate was used, 700.00 mg of ethyl 4-[4-(4-aminobutanamido)-1-methylpyrrole-2-amido]-1-methylimidazole-2-carboxylate was obtained as a Brown yellow solid (118.16% yield). LC/MS: mass calcd. For C₁₇H₂₄N₆O₄: 376.19, found: 377.20 [M+H]⁺.

### Step 3: Synthesis of ethyl 1-methyl-4-[1-methyl-4-(4-{[1-methyl-4-(3-[[1-methyl-4-(1-methylimidazole-2-amido)pyrrol-2-yl]formamido]propanamido)imidazol-2-yl]formamido}butanamido)pyrrole-2-amido]imidazole-2-carboxylate

To a mixture of 1-methyl-4-(3-{[1-methyl-4-(1-methylimidazole-2-amido) pyrrol-2-yl]formamido}propanamido)imidazole-2-carboxylic acid (700.00 mg, 1.58 mmol, 1.00 equiv) and ethyl 4-[4-(4-aminobutanamido)-1-methylpyrrole-2-amido]-1-methylimidazole-2-carboxylate (655.10 mg, 1.74 mmol, 1.10 equiv) in DMF (5.00 mL, 64.61 mmol, 40.84 equiv) were added EDCI (1213.21 mg, 6.33 mmol, 4.00 equiv) and DMAP (1546.30 mg, 12.66 mmol, 8.00 equiv). The reaction was stirred at room temperature for 16 h. The reaction mixture was precipitated by the addition of H₂O (20 mL). And the mixture was filtered through a celite pad. The filter cake was purified by reverse flash chromatography with the following conditions: column, C18 column; mobile phase, MeCN in water (0.05% TFA), 10% to 50% gradient in 20 min; detector, UV 254 nm. The fractions were combined and concentrated to afford ethyl 1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-carboxylate (230.00 mg, 18.15%) as a yellow solid. LC/MS: mass calcd. For C₃₆H₄₄N₁₄O₅: 800.35, found: 801.40 [M+H]⁺.

### Step 4: Synthesis of 1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-carboxylic acid

The procedure was the same as 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid, but the reaction time was 2.0 h. 230.00 mg of ethyl 1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-carboxylate was used, 90.00 mg of 1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-carboxylic acid was obtained as a light yellow solid (40.55% yield). LC/MS: mass calcd. For C₃₄H₄₀N₁₄O₅: 772.32, found: 773.35 [M+H]⁺.

### Step 5: Synthesis of methyl 1-methyl-4-(1-methyl-4-{1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-amido}pyrrole-2-amido)pyrrole-2-carboxylate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate, but the reaction time was 2.0 h. 80.00 mg of 1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-carboxylic acid was used, 100.00 mg of methyl 1-methyl-4-(1-methyl-4-{1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-amido}pyrrole-2-amido)pyrrole-2-carboxylate was obtained as a light yellow solid (93.69% yield). LC/MS: mass calcd. For C₄₇H₅₄N₁₈O₁₀: 1030.43, found: 1031.50 [M+H]⁺.

### Step 6: Synthesis of 1-methyl-4-(1-methyl-4-{1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-amido}pyrrole-2-amido)pyrrole-2-carboxylic acid

The procedure was the same as 4-[3-[(tert-butoxycarbonyl)amino]propanamido]-1-methylimidazole-2-carboxylic acid, but the reaction temperature was r.t., the reaction time was 16.0 h. 100.00 mg of methyl 1-methyl-4-(1-methyl-4-{1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-amido}pyrrole-2-amido)pyrrole-2-carboxylate was used, 60.00 mg of 1-methyl-4-(1-methyl-4-{1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-amido}pyrrole-2-amido)pyrrole-2-carboxylic acid was obtained as a light yellow solid (60.83% yield). LC/MS: mass calcd. For C₄₆H₅₂N₁₈O₁₀: 1016.41, found: 1017.40 [M+H]⁺.

### Example 39. Synthesis of 4-[4-(4-{4-[(2S)-2-acetamido-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid (Compound 149)

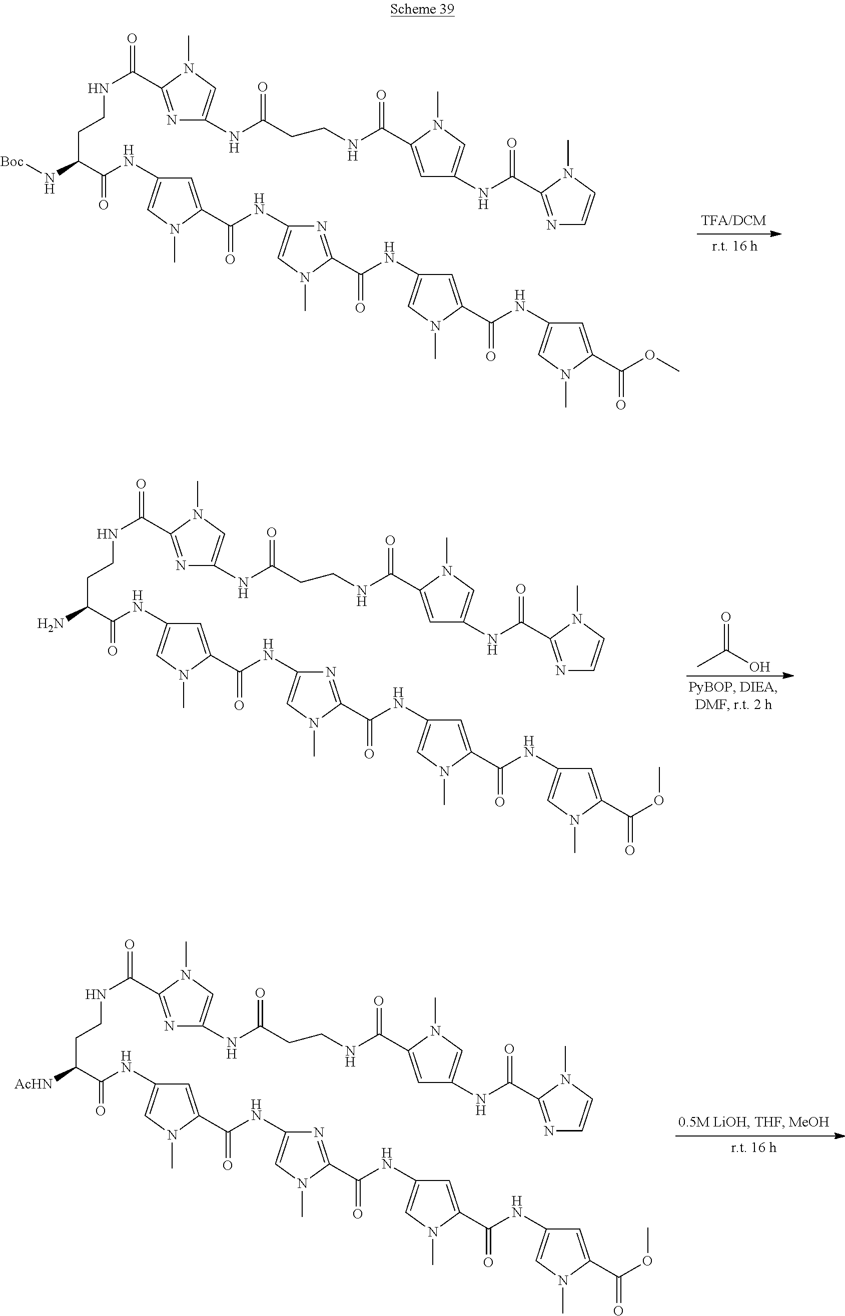

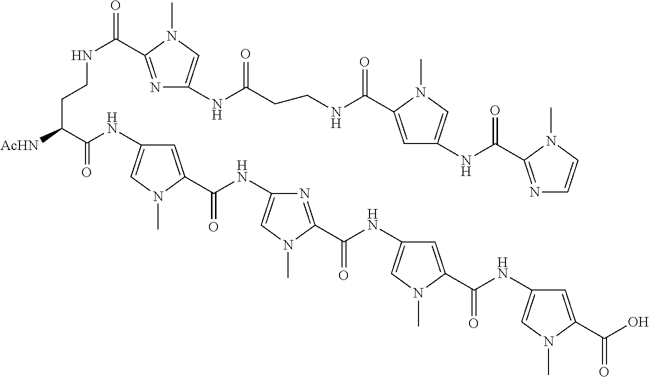 [SMILES: CC(=O)N[C@@H](CCNC(=O)c1nc(NC(=O)CCNC(=O)c2cc(NC(=O)c3nccn3C)cn2C)cn1C)C(=O)Nc1cc(C(=O)Nc2cn(C)c(C(=O)Nc3cc(C(=O)Nc4cc(C(=O)O)n(C)c4)n(C)c3)n2)n(C)c1]

### Step 1: Synthesis of methyl 4-[4-(4-[4-[(2S)-2-amino-4-[[1-methyl-4-(3-[[1methyl-4-(1-methylimidazole-2-amido)pyrrol-2-yl]formamido}propanamido)imidazol-2-yl]formamido]butanamido]-1-methylpyrrole-2-amido}-1-methylimidazole-2-amido)-1-methylpyrrole-2-amido]-1-methylpyrrole-2-carboxylate

A solution of methyl 4-[4-(4-{4-[(2S)-2-[(tert-butoxycarbonyl)amino]-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate (80.00 mg, 0.07 mmol, 1.00 equiv) in CH₂Cl₂ (1.60 mL) was added TFA (0.20 mL). The reaction was stirred for 16 h at room temperature. The mixture was concentrated under reduced pressure. The residue was purified by reverse flash chromatography with the following conditions: column, C18 column; mobile phase, MeCN in water (0.05% TFA), 10% to 60% gradient in 25 min; detector, UV 254 nm to afford methyl 4-[4-(4-{4-[(2S)-2-amino-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate (60.00 mg, 82.18%) as a yellow solid. LC/MS: mass calcd. For C₄₇H₅₅N₁₉O₁₀: 1045.44, found: 1046.90 [M+H]⁺.

### Step 2: Synthesis of methyl 4-[4-(4-{4-[(2S)-2-acetamido-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate, but the reaction time was 2.0 h. 60.00 mg of methyl 4-[4-(4-{4-[(2S)-2-amino-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate was used, 50.00 mg of methyl 4-[4-(4-{4-[(2S)—2-acetamido-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylate was obtained as a yellow solid (80.11% yield). LC/MS: mass calcd. For C₄₉H₅₇N₁₉O₁₁: 1087.45, found: 1088.55 [M+H]⁺.

### Step 3: Synthesis of 4-[4-(4-{4-[(2S)-2-acetamido-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid

The procedure was the same as 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid. 50.00 mg of methyl 4-[4-(4-{4-[(2S)-2-acetamido-4-{[1-methyl-4-(3-{[1-methyl-4-(1-methylimidazole-2-amido)pyrrol-2-yl]formamido} propanamido)imidazol-2-yl]formamido}butanamido]-1-methylpyrrole-2-amido}-1-methylimidazole-2-amido)-1-methylpyrrole-2-amido]-1-methylpyrrole-2-carboxylate was used, 35.00 mg of 4-[4-(4-{4-[(2S)-2-acetamido-4-{[1-methyl-4-(3-{[1-methyl-4-(1-methylimidazole-2-amido)pyrrol-2-yl]formamido}propanamido)imidazol-2-yl]formamido}butanamido]-1-methylpyrrole-2-amido}-1-methylimidazole-2-amido)-1-methylpyrrole-2-amido]-1-methylpyrrole-2-carboxylic acid was obtained as a light yellow solid (70.91% yield). LC/MS: mass calcd. For C₄₈H₅₅N₁₉0u: 1073.43, found: 1074.60 [M+H]⁺.

### Example 40. Synthesis of (R)-4-(4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxamido)-1-methyl-1H-pyrrole-2-carboxylic acid (Compound 61)

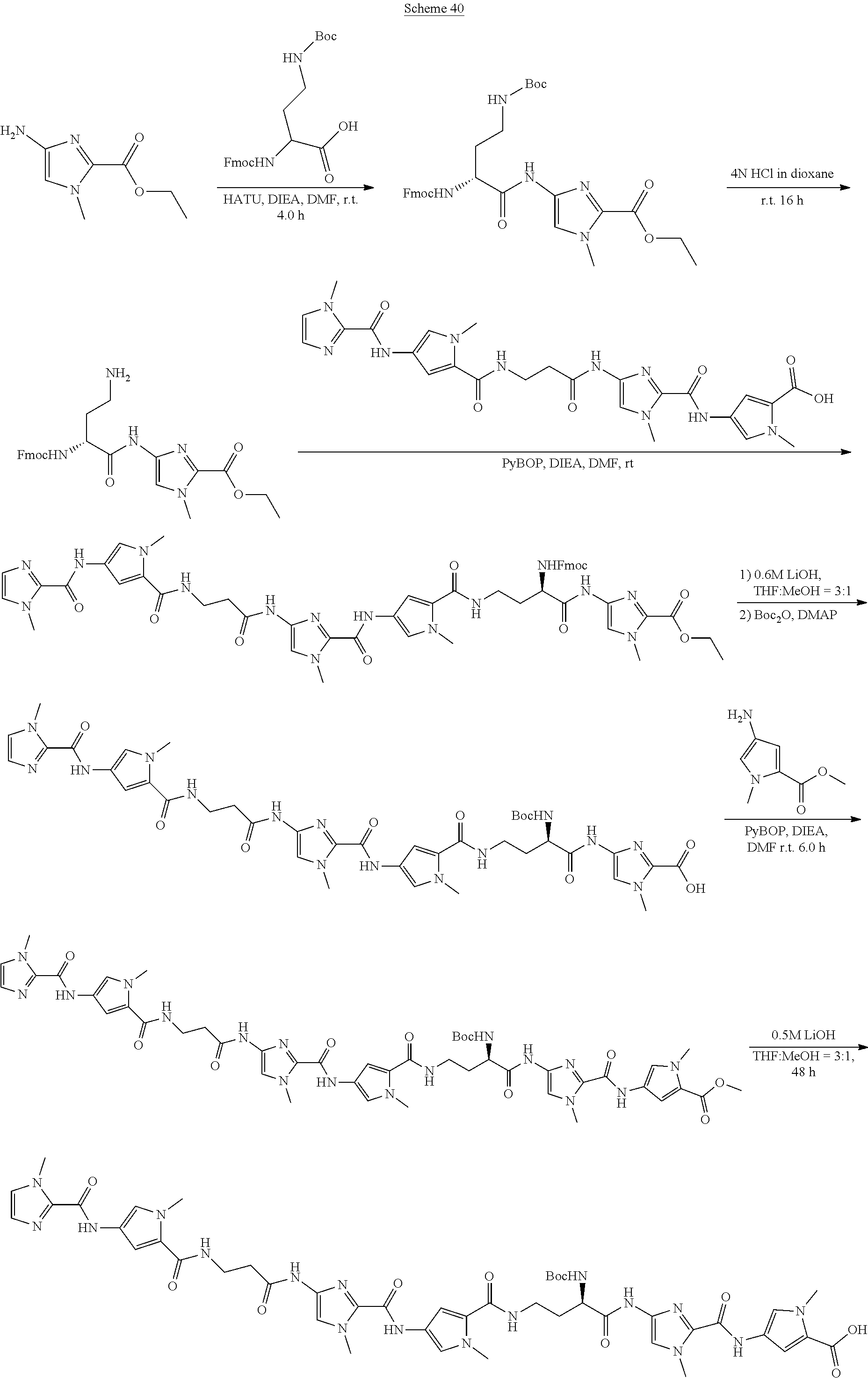

### Step 1: Synthesis of ethyl (R)-4-(2-((((9H-fluoren-9-yl)methoxy)carbonyl)amino)-4-((tert-butoxycarbonyl)amino)butanamido)-1-methyl-1H-imidazole-2-carboxylate

The procedure was the same as methyl 3-[(4-[3-[(tert-butoxycarbonyl)amino] propanamido]-1-methylimidazol-2-yl)formamido]propanoate, but the reaction time was 4.0 h. 500.00 mg of ethyl 4-amino-1-methyl-1H-imidazole-2-carboxylate was used, 750.00 mg of ethyl (R)-4-(2-((((9H-fluoren-9-yl) methoxy)carbonyl)amino)-4-((tert-butoxycarbonyl)amino)butanamido)-1-methyl-1H-imidazole-2-carboxylate was obtained as yellow oil (42.89% yield). LC/MS: mass calcd. For C₃₁H₃₇N₅O₇: 591.27, found: 592.20 [M+H]⁺.

### Step 2: Synthesis of ethyl (R)-4-(2-((((9H-fluoren-9-yl)methoxy)carbonyl) amino)-4-aminobutanamido)-1-methyl-1H-imidazole-2-carboxylate

The procedure was the same as methyl 4-[4-(3-aminopropanamido)-1-methylimidazole-2-amido]-1-methylpyrrole-2-carboxylate hydrochloride, but the reaction time was 16.0 h. 664.00 mg of ethyl (R)-4-(2-((((9H-fluoren-9-yl)methoxy)carbonyl)amino)-4-((tert-butoxycarbonyl)amino)butanamido)-1-methyl-1H-imidazole-2-carboxylate was used, 542.00 mg of ethyl (R)-4-(2-((((9H-fluoren-9-yl)methoxy)carbonyl)amino)-4-aminobutanamido)-1-methyl-1H-imidazole-2-carboxylate was obtained as yellow oil (98.25% yield). LC/MS: mass calcd. For C₂₆H₂₉N₅O₅: 491.22, found: 492.20[M+H]⁺.

### Step 3: Synthesis of ethyl (R)-4-(2-((((9H-fluoren-9-yl)methoxy)carbonyl) amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate, but the reaction time was 6.0 h, 640.00 mg of ethyl (R)-4-(2-((((9H-fluoren-9-yl)methoxy)carbonyl)amino)-4-aminobutanamido)-1-methyl-1H-imidazole-2-carboxylate was used, 1.10 g of ethyl (R)-4-(2-((((9H-fluoren-9-yl)methoxy)carbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxylate was obtained as a yellow solid (97.59% yield). LC/MS: mass calcd. For C₅₁H₅₅N₁₅O₁₀: 1037.43, found: 1038.50 [M+H]⁺.

### Step 4: Synthesis of (R)-4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxylic acid

The procedure was the same as (S)-4-(4-(2-((tert-butoxycarbonyl)amino)-4-(1-methyl-4-(1-methyl-4-(1-methyl-4-(1-methyl-1H-imidazole-2-carboxamido)-1H-pyrrole-2-carboxamido)-1H-pyrrole-2-carboxamido)-1H-imidazole-2-carboxamido)butanamido)-1-methyl-1H-pyrrole-2-carboxamido)-1-methyl-1H-imidazole-2-carboxylic acid. 1.12 g of ethyl 4-[(2R)-2-{[(9H-fluoren-9-ylmethoxy) carbonyl]amino}-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylate was used, 780.00 mg of (R)-4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxylic acid was obtained as yellow oil (81.79% yield). LC/MS: mass calcd. For C₃₉H₄₉N₁₅O₁₀: 887.38, found: 888.40 [M+H]⁺.

### Step 5: Synthesis of methyl (R)-4-(4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxamido)-1-methyl-1H-pyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (PH-DTP-PA04-OH-12), but the reaction time was 6.0 h. 530.00 mg of (R)-4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxylic acid was used, 350.00 mg of methyl (R)-4-(4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxamido)-1-methyl-1H-pyrrole-2-carboxylate was obtained as a brown yellow solid (57.26% yield). LC/MS: mass calcd. For C₄₆H₅₇N₁₇O₁₁: 1023.44, found: 1024.60 [M+H]⁺.

### Step 6: Synthesis of (R)-4-(4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxamido)-1-methyl-1H-pyrrole-2-carboxylic acid

The procedure was the same as 4-[4-(4-{4-[(2S)-2-[(tert-butoxycarbonyl)amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid. 350.00 mg of methyl (R)-4-(4-(2-((tert-butoxycarbonyl)amino)-4-(1-methyl-4-(1-methyl-4-(3-(1-methyl-4-(1-methyl-1H-imidazole-2-carboxamido)-1H-pyrrole-2-carboxamido)propanamido)-1H-imidazole-2-carboxamido)-1H-pyrrole-2-carboxamido)butanamido)-1-methyl-1H-imidazole-2-carboxamido)-1-methyl-1H-pyrrole-2-carboxylate was used, 240.00 mg of desired product was obtained as a brown solid (69.52% yield). LC/MS: mass calcd. For C₄₅H₅₅N₁₇O₁₁: 1009.43, found: 1010.45[M+H]⁺.

### Example 41. Synthesis of 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylic acid(Compound 152)

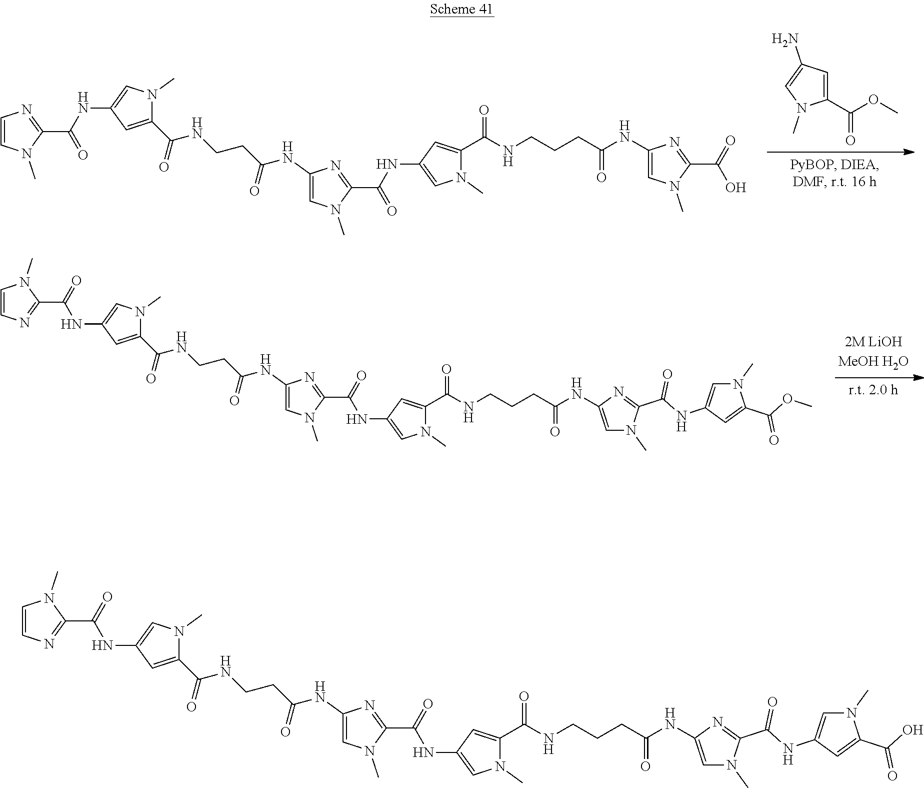 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)O)n1 ; COC(=O)c1cc(N)cn1C ; COC(=O)c1cc(NC(=O)c2nc(NC(=O)CCCNC(=O)c3cc(NC(=O)c4nc(NC(=O)CCNC(=O)c5cc(NC(=O)c6nccn6C)cn5C)cn4C)cn3C)cn2C)cn1C ; Cn1cc(NC(=O)c2nc(NC(=O)CCCNC(=O)c3cc(NC(=O)c4nc(NC(=O)CCNC(=O)c5cc(NC(=O)c6nccn6C)cn5C)cn4C)cn3C)cn2C)cc1C(=O)O]

### Step 1: Synthesis of methyl 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate, but the reaction time was 3.0 h. 600.00 mg of 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid was used. 740.00 mg of methyl 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylate was obtained as a white solid (44.04% yield). LC/MS: mass calcd. For C₄₁H₄₈N₁₆O₉: 908.94, found: 909.45[M+H]⁺.

### Step 2: Synthesis of 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylic acid

The procedure was the same as 4-[3-[(tert-butoxycarbonyl)amino]propanamido]-1-methylimidazole-2-carboxylic acid. 740.00 mg of methyl 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylate was used. 800.00 mg of 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylic acid was obtained as a yellow solid (93.33% yield). LC/MS: mass calcd. For C₄₀H₄₆N₁₁O₉: 894.36, found: 895.35[M+H]⁺.

### Example 42. Synthesis of 4-{4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrole-2-carboxylic acid (Compound 153)

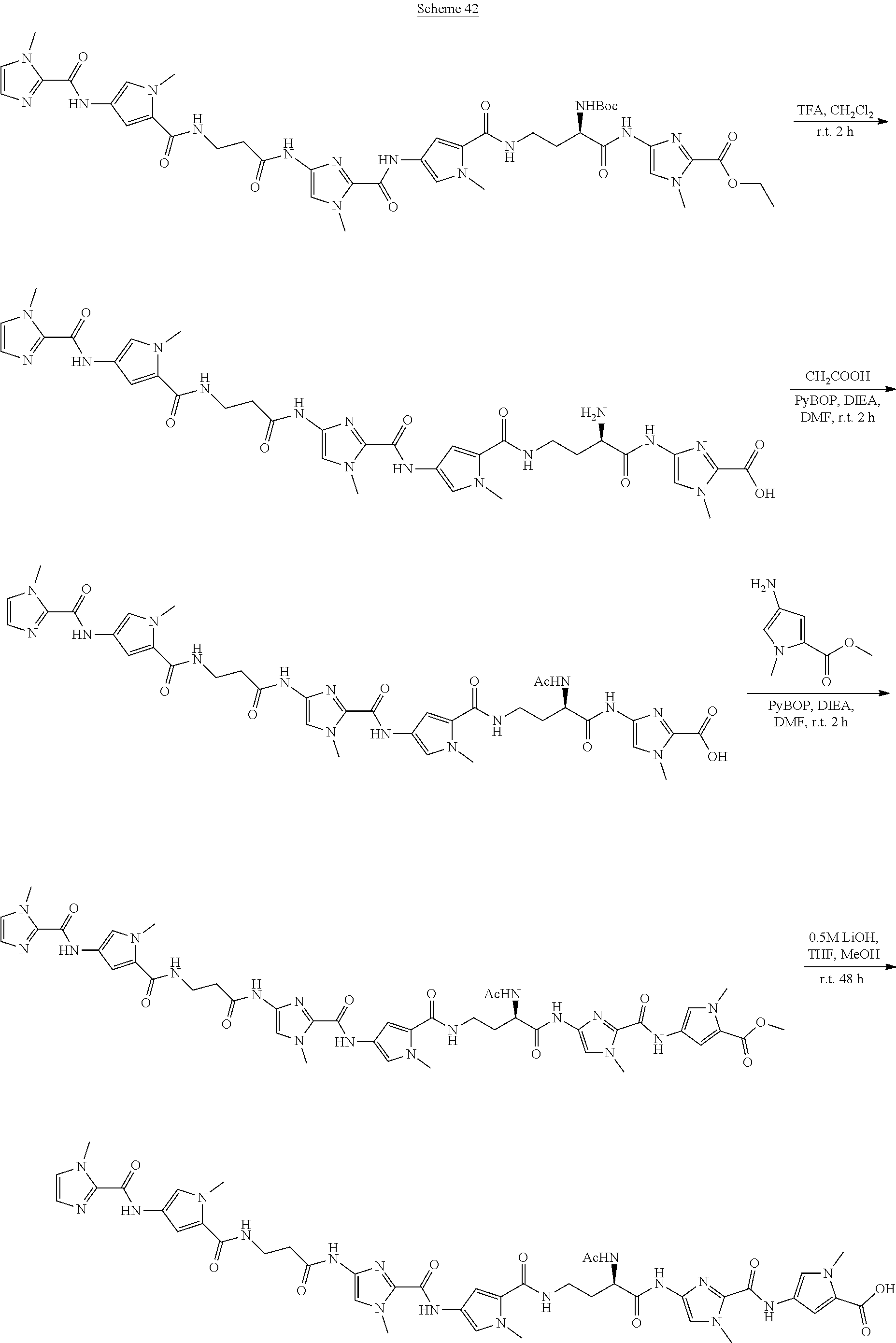

### Step 1: Synthesis of 4-[(2R)-2-amino-4-({1-methyl-4-[1-methyl-4-(3-{{1-methyl-4-(1-methylimidazole-2-amido) pyrrol-2-yl]formamido}propanamido) imidazole-2-amido]pyrrol-2-yl}formamido}butanamido]-1-methylimidazole-2-carboxylic acid

The procedure was the same as methyl 4-[4-(4-{4-[(2S)-2-amino-4-{[1-methyl-4-(3-{[1-methyl-4-(1-methylimidazole-2-amido)pyrrol-2-yl]formamido}propanamido)imidazol-2-yl}formamido}butanamido]-1-methylpyrrole-2-amido}-1-methylimidazole-2-amido)-1-methylpyrrole-2-amido]-1-methylpyrrole-2-carboxylate, but the reaction time was 2.0 h. 100.00 mg of 4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid was used, 90.00 mg crude of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid was obtained as a light yellow solid. LC/MS: mass calcd. For C₃₄H₄₁N₁₅O₈: 787.33, found: 788.30 [M+H]⁺.

### Step 2: Synthesis of 4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate, but the reaction time was 2.0 h. 90.00 mg of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid was used, 55.00 mg of 4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid was obtained as a light yellow solid (58.02% yield). LC/MS: mass calcd. For C₃₆H₄₃N₁₅O₉: 829.34, found: 830.45 [M+H]⁺.

### Step 3: Synthesis of methyl 4-{4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrole-2-carboxylate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate, but the reaction time was 2.0 h. 55.00 mg of 4-[(2R)-2-amino-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-carboxylic acid was used, 50.00 mg of methyl 4-{4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrole-2-carboxylate was obtained as a light yellow solid (78.10% yield). LC/MS: mass calcd. For C₄₃H₅₁N₁₇O₁₀: 965.40, found: 966.45 [M+H]⁺.

### Step 4: Synthesis of 4-{4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrole-2-carboxylic acid

The procedure was the same as 4-[4-(4-{4-[(2S)-2-[(tert-butoxycarbonyl) amino]-4-[(1-methyl-4-{1-methyl-4-[1-methyl-4-(1-methylimidazole-2-amido)pyrrole-2-amido]pyrrole-2-amido}imidazol-2-yl)formamido]butanamido]-1-methylpyrrole-2-amido}-1-methylimidazole-2-amido)-1-methylpyrrole-2-amido]-1-methylpyrrole-2-carboxylic acid. 50.00 mg of methyl 4-{4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrole-2-carboxylate was used, 32.00 mg of 4-{4-[(2R)-2-acetamido-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrole-2-carboxylic acid was obtained as a light yellow solid (64.94% yield). LC/MS: mass calcd. For C₄₂H₄₉N₁₇O₁₀: 951.38, found: 952.50 [M+H]⁺.

### Example 43. Synthesis of 1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-carboxylic acid (Compound 154)

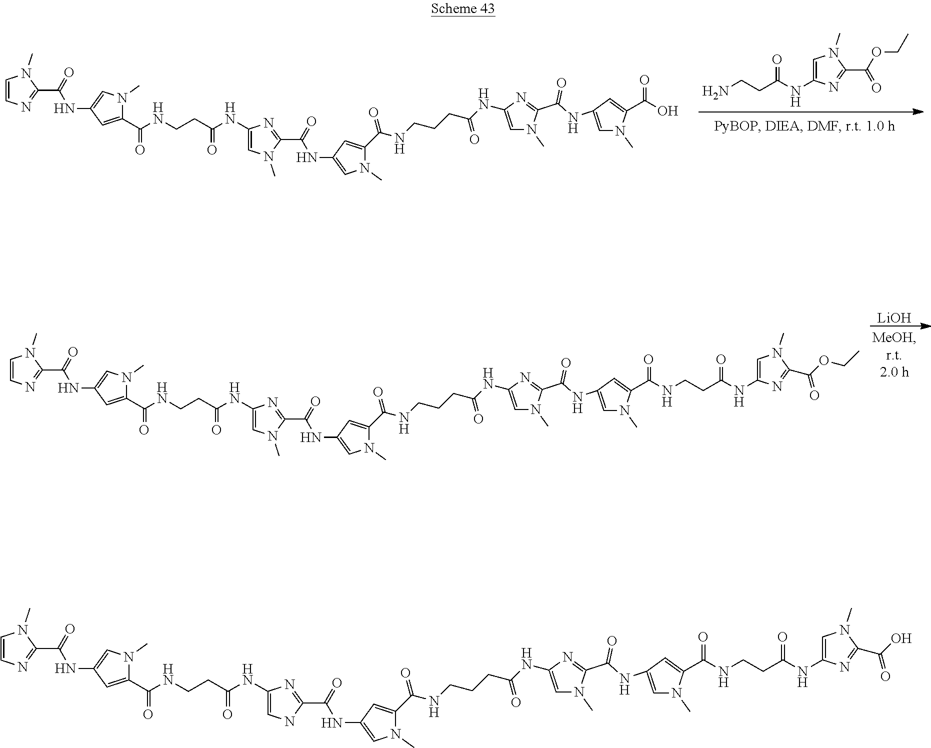 [SMILES: Cn1cc(NC(=O)c2nc(NC(=O)CCCNC(=O)c3cc(NC(=O)c4nc(NC(=O)CCNC(=O)c5cc(NC(=O)c6nccn6C)cn5C)cn4C)cn3C)cn2C)cc1C(=O)O ; CCOC(=O)c1nc(NC(=O)CCN)cn1C ; "LiOH" ; CCOC(=O)c1nc(NC(=O)CCNC(=O)c2cc(NC(=O)c3nc(NC(=O)CCCNC(=O)c4cc(NC(=O)c5nc(NC(=O)CCNC(=O)c6cc(NC(=O)c7nccn7C)cn6C)cn5C)cn4C)cn3C)cn2C)cn1C ; Cn1cc(NC(=O)c2nc(NC(=O)CCNC(=O)c3cc(NC(=O)c4nccn4C)cn3C)c[nH]2)cc1C(=O)NCCCC(=O)Nc1cn(C)c(C(=O)Nc2cc(C(=O)NCCC(=O)Nc3cn(C)c(C(=O)O)n3)n(C)c2)n1]

### Step 1: Synthesis of ethyl 1-methyl-4-{3-[(1-methyl-4-{1-methyl-4-[4-([1-methyl-4-{1-methyl-4-(3-[[1-methyl-4-(1-methylimidazole-2-amido)pyrrol-2-yl]formamido]propanamido)imidazole-2-amido]pyrrol-2-yl}formamido)butanamido]imidazole-2-amido}pyrrol-2-yl)formamido]propanamido}imidazole-2-carboxylate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate. 300.00 mg of 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylic acid was used, 170.00 mg of ethyl 1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-carboxylate was obtained as a brown yellow solid (76.55% yield). LC/MS: mass calcd. For C₅₀H₆₀N₂₀O₁₁: 1117.16, found: 1117.50 [M+H]⁺.

### Step 2: Synthesis of 1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-carboxylic acid

The procedure was the same as 4-[3-[(tert-butoxycarbonyl)amino]propanamido]-1-methylimidazole-2-carboxylic acid, but the reaction time was 4.0 h. 160.00 mg of ethyl 1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-carboxylate was used, 51.00 mg of 1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-carboxylic acid was obtained as a yellow solid. (32.70% yield). LC/MS: mass calcd. For C₄₈H₅₆N₂₀O₁₁: 1088.44, found: 1089.45 [M+H]⁺.

### Example 44. Synthesis of 1-methyl-4-[1-methyl-4-(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-amido)pyrrole-2-amido]imidazole-2-carboxylic acid (Compound 90)

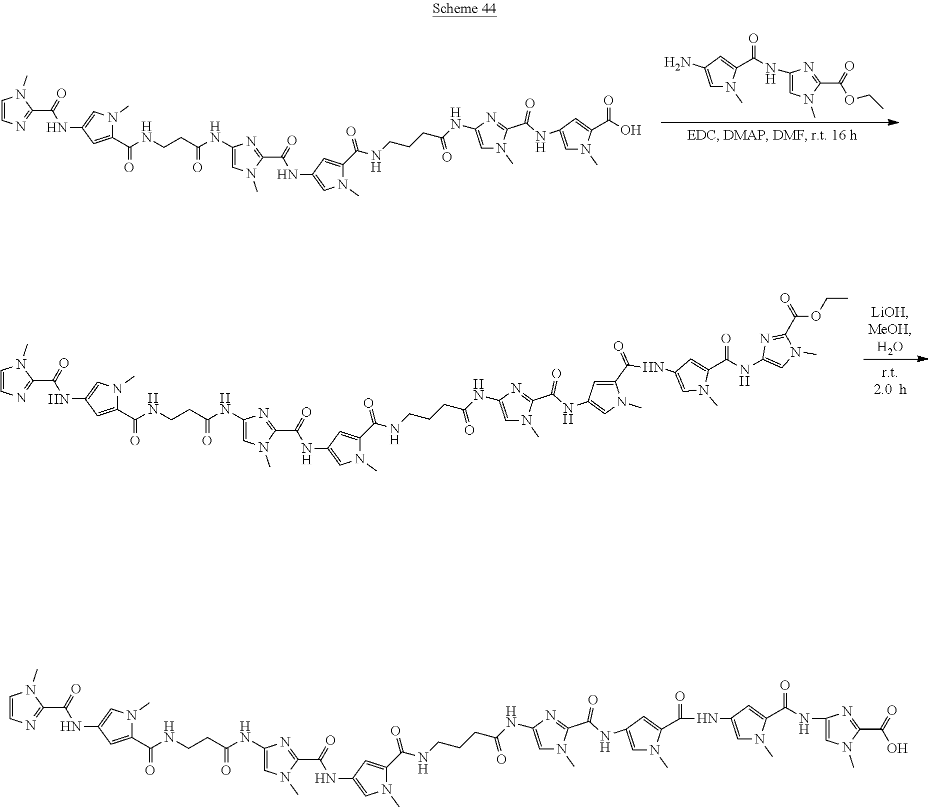 [SMILES: Cn1cc(NC(=O)c2nc(NC(=O)CCCNC(=O)c3cc(NC(=O)c4nc(NC(=O)CCNC(=O)c5cc(NC(=O)c6nccn6C)cn5C)cn4C)cn3C)cn2C)cc1C(=O)O ; CCOC(=O)c1nc(NC(=O)c2cc(N)cn2C)cn1C ; CCOC(=O)c1nc(NC(=O)c2cc(NC(=O)c3cc(NC(=O)c4nc(NC(=O)CCCNC(=O)c5cc(NC(=O)c6nc(NC(=O)CCNC(=O)c7cc(NC(=O)c8nccn8C)cn7C)cn6C)cn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)Nc2cc(C(=O)Nc3cc(C(=O)Nc4cn(C)c(C(=O)O)n4)n(C)c3)n(C)c2)n1]

### Step 1: Synthesis of ethyl 1-methyl-4-[1-methyl-4-(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-amido)pyrrole-2-amido]imidazole-2-carboxylate

The procedure was the same as ethyl 1-methyl-4-[1-methyl-4-(4-{[1-methyl-4-(3-{[1-methyl-4-(1-methylimidazole-2-amido)pyrrol-2-yl]formamido}propanamido)imidazol-2-yl]formamido}butanamido)pyrrole-2-amido]imidazole-2-carboxylate. 300.00 mg of 1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-carboxylic acid was used, 180.00 mg of ethyl 1-methyl-4-[1-methyl-4-(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-amido)pyrrole-2-amido]imidazole-2-carboxylate was obtained as a brown solid (45.96% yield). LC/MS: mass calcd. For C₅₃H₆₁N₂₁O₁: 1168.21, found: 1143.40 [M+H]⁺.

### Step 2: Synthesis of 1-methyl-4-[1-methyl-4-(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-amido)pyrrole-2-amido]imidazole-2-carboxylic acid

The procedure was the same as 3-[[1-methyl-4-(1-methylimidazole-2-amido) imidazol-2-yl]formamido]propanoic acid. 140.00 mg of ethyl 1-methyl-4-[1-methyl-4-(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-amido)pyrrole-2-amido]imidazole-2-carboxylate was used, 50.00 mg of 1-methyl-4-[1-methyl-4-(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrole-2-amido)pyrrole-2-amido]imidazole-2-carboxylic acid was obtained as a brown solid. (36.59% yield). LC/MS: mass calcd. For C₅₁H₅₇N₂₁O₁₁: 1139.45, found: 1140.55 [M+H]⁺.

### Example 45. Synthesis of bis(1-methyl-4-[3-({1-methyl-4-[1-methyl-4-(4-{[1-methyl-4-(1-methyl-4-{1-methyl-4-[1-methyl-4-(1-methylimidazole-2-amido]pyrrole-2-amido]pyrrole-2-amido}imidazole-2-amido)pyrrol-2-yl]formamido}butanamido)imidazole-2-amido]pyrrol-2-yl}formamido)propanamido]imidazole-2-carboxylic acid (Compound 91)

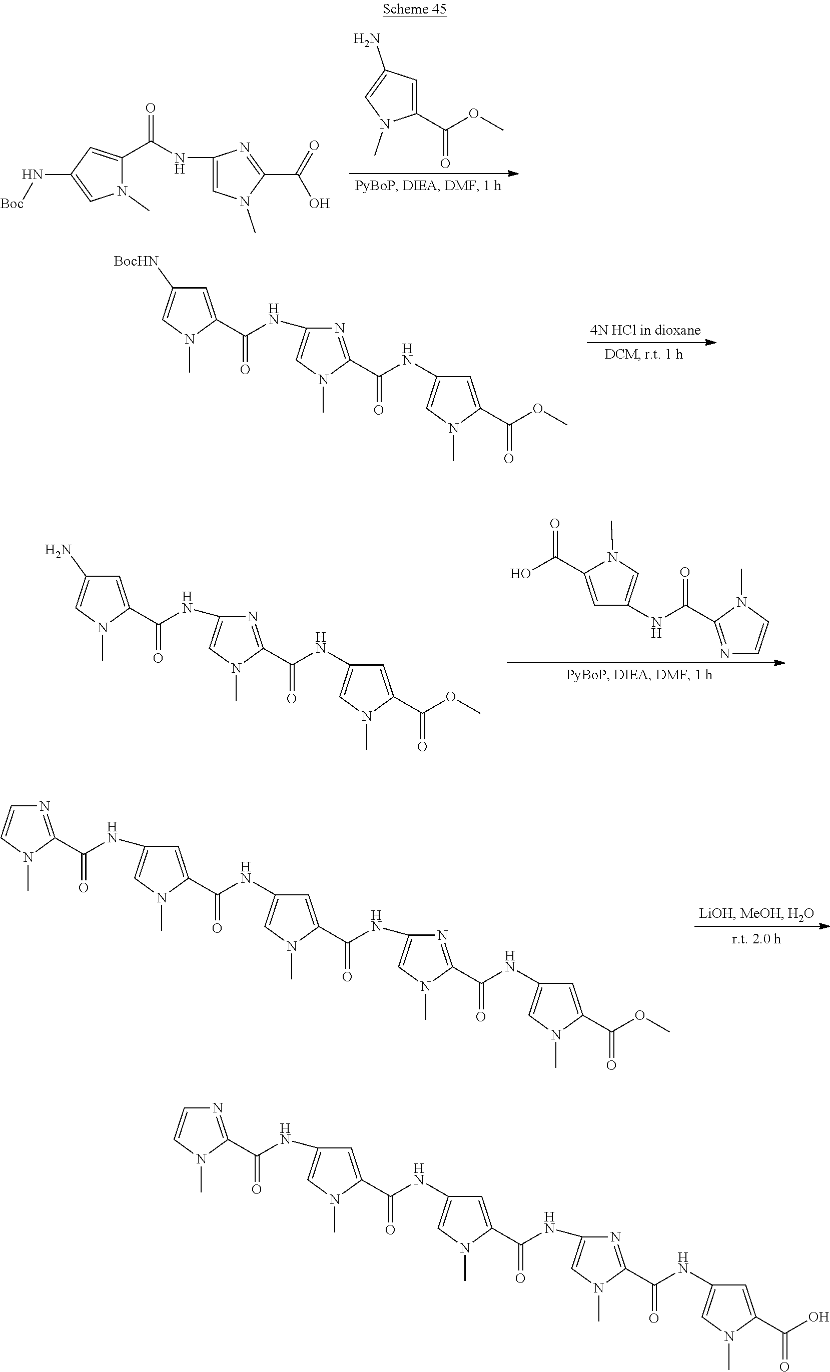

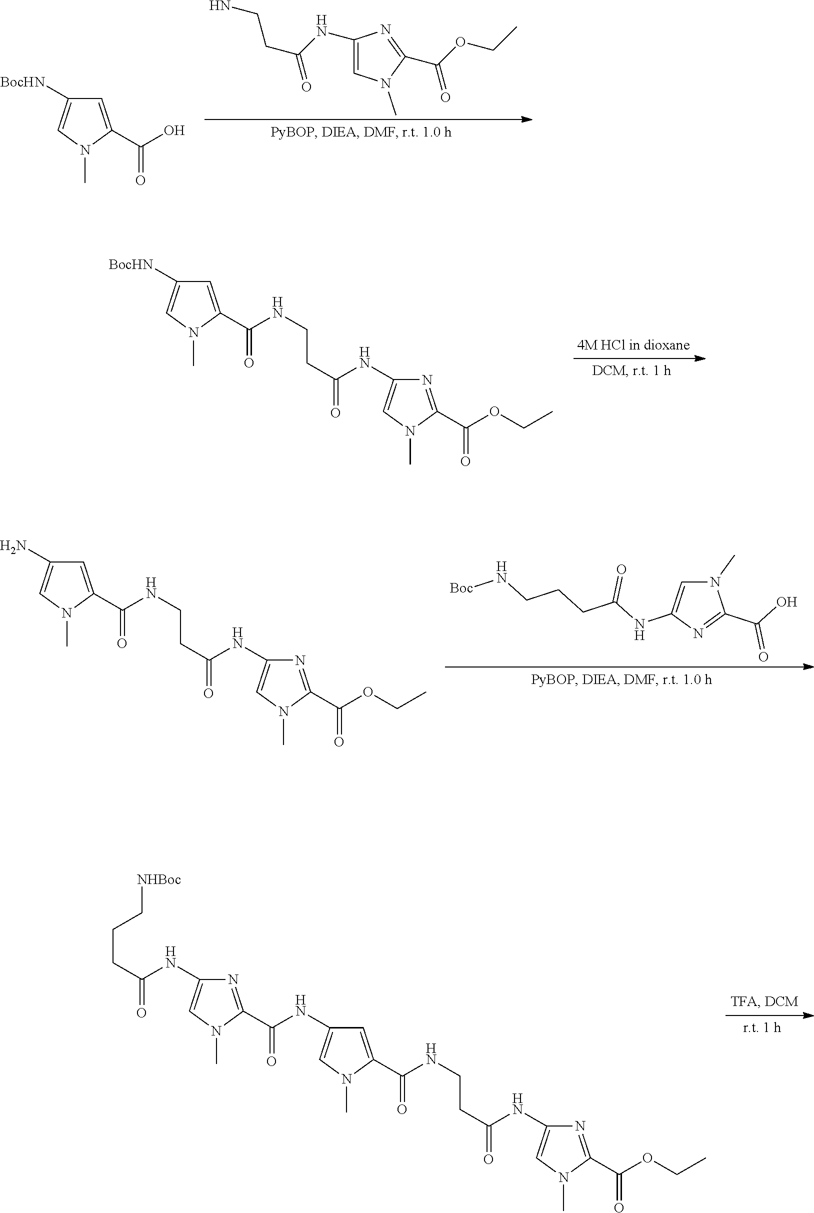 [SMILES: Cn1cc(NC(=O)OC(C)(C)C)cc1C(=O)O ; CCOC(=O)c1nc(NC(=O)CCN)cn1C ; CCOC(=O)c1nc(NC(=O)CCNC(=O)c2cc(NC(=O)OC(C)(C)C)cn2C)cn1C ; CCOC(=O)c1nc(NC(=O)CCNC(=O)c2cc(N)cn2C)cn1C ; Cn1cc(NC(=O)CCCNC(=O)OC(C)(C)C)nc1C(=O)O ; CCOC(=O)c1nc(NC(=O)CCNC(=O)c2cc(NC(=O)c3nc(NC(=O)CCCNC(=O)OC(C)(C)C)cn3C)cn2C)cn1C]

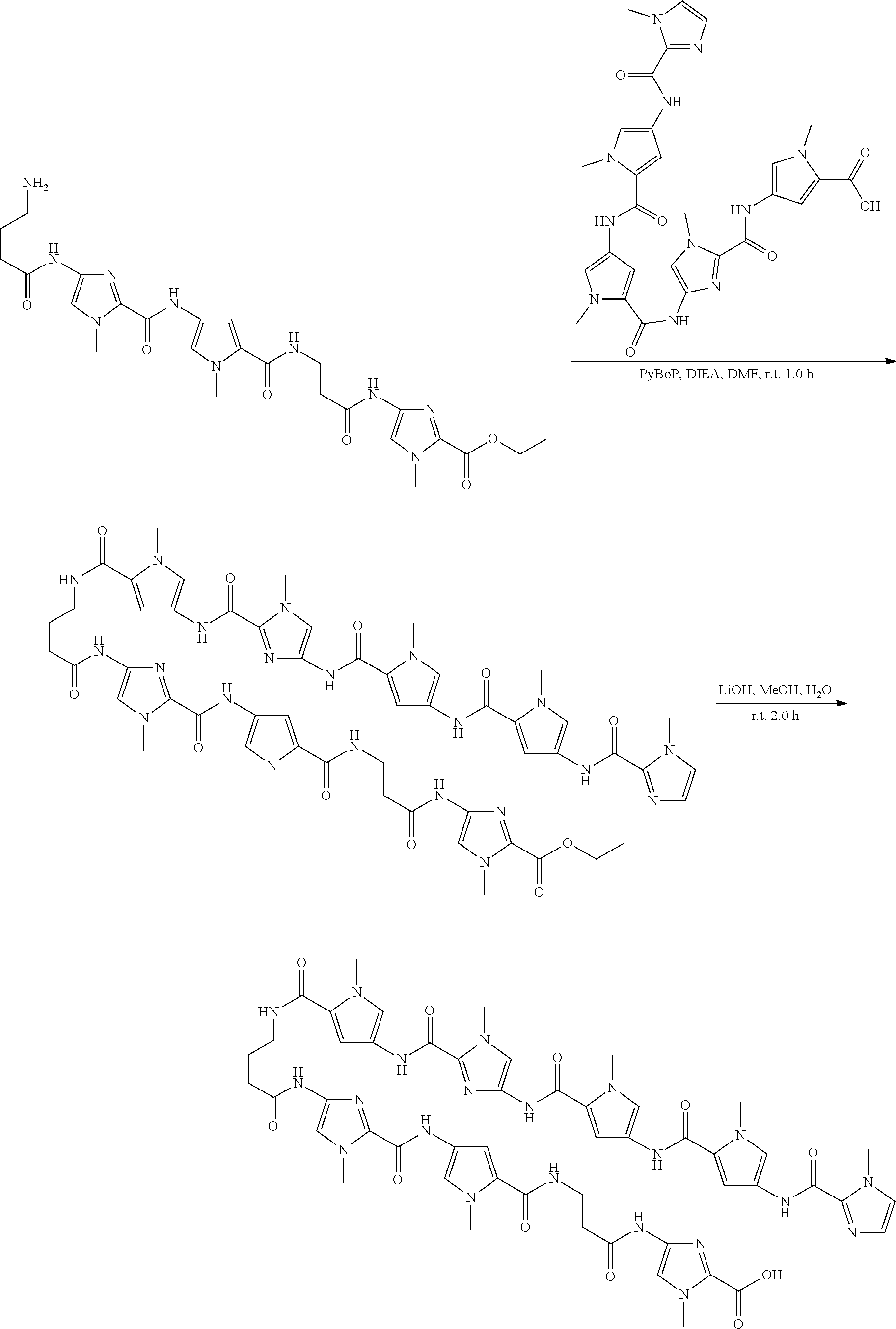 [SMILES: CCOC(=O)c1nc(NC(=O)CCNC(=O)c2cc(NC(=O)c3nc(NC(=O)CCCN)cn3C)cn2C)cn1C ; Cn1cc(NC(=O)c2nc(NC(=O)c3cc(NC(=O)c4cc(NC(=O)c5nccn5C)cn4C)cn3C)cn2C)cc1C(=O)O ; CCOC(=O)c1nc(NC(=O)CCNC(=O)c2cc(NC(=O)c3nc(NC(=O)CCCNC(=O)c4cc(NC(=O)c5nc(NC(=O)c6cc(NC(=O)c7cc(NC(=O)c8nccn8C)cn7C)cn6C)cn5C)cn4C)cn3C)cn2C)cn1C ; Cn1cc(NC(=O)c2nc(NC(=O)c3cc(NC(=O)c4cc(NC(=O)c5nccn5C)cn4C)cn3C)cn2C)cc1C(=O)NCCCC(=O)Nc1cn(C)c(C(=O)Nc2cc(C(=O)NCCC(=O)Nc3cn(C)c(C(=O)O)n3)n(C)c2)n1]

### Step 1: Synthesis of methyl 4-(4-{4-[(tert-butoxycarbonyl)amino]-1-methylpyrrole-2-amido}-1-methylimidazole-2-amido)-1-methylpyrrole-2-carboxylate

The procedure was the same as methyl 4-(4-{13-[(4-{14-[(2R)-2-[(tert-butoxycarbonyl)amino]-4-({1-methyl-4-[1-methyl-4-(3-1 {[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate. 1.17 g of methyl 4-(4-{14-[(tert-butoxycarbonyl)amino]-1-methylpyrrole-2-amido}-1-methylimidazole-2-amido)-1-methylpyrrole-2-carboxylate was obtained as a white solid (85.27% yield). LC/MS: mass calcd. For C₂₃H₂₉N₇O₆: 499.52, found: 500.25 [M+H]⁺.

### Step 2: Synthesis of methyl 4-[4-(4-amino-1-methylpyrrole-2-amido)-1-methylimidazole-2-amido]-1-methylpyrrole-2-carboxylate

The procedure was the same as methyl 4-[4-(3-aminopropanamido)-1-methylimidazole-2-amido]-1-methylpyrrole-2-carboxylate hydrochloride. 1.10 g of methyl 4-(4-{14-[(tert-butoxycarbonyl)amino]-1-methylpyrrole-2-amido}-1-methylimidazole-2-amido)-1-methylpyrrole-2-carboxylate was used. 0.80 g of methyl 4-[4-(4-amino-1-methylpyrrole-2-amido)-1-methylimidazole-2-amido]-1-methylpyrrole-2-carboxylate was obtained as a white solid (64.98% yield). LC/MS: mass calcd. For C₁₈H₂₁N₇O₄: 399.41, found: 400.15 [M+H]⁺.

### Step 3: Synthesis of methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 769.73 mg of methyl 4-[4-(4-amino-1-methylpyrrole-2-amido)-1-methylimidazole-2-amido]-1-methylpyrrole-2-carboxylate was used, 0.80 g of methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate was obtained as a yellow solid (45.43% yield). LC/MS: mass calcd. For C₂₉H₃₁N₁₁O₆: 629.64, found: 630.20[M+H]⁺.

### Step 4: Synthesis of 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylic acid

The procedure was the same as 4-[3-[(tert-butoxycarbonyl)amino] propanamido]-1-methylimidazole-2-carboxylic acid, but the reaction time was 2.0 h. 0.80 g of methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate was used. 200.00 mg of 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylic acid was obtained as a white solid (24.30% yield). LC/MS: mass calcd. For C₂₁H₃₀N₆O₆: 615.61, found: 616.20[M+H]⁺.

### Step 5: Synthesis of ethyl 4-[3-({4-[(tert-butoxycarbonyl)amino]-1-methylpyrrol-2-yl}formamido)propanamido]-1-methylimidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate. 1.50 g of 4-[(tert-butoxycarbonyl)amino]-1-methylpyrrole-2-carboxylic acid was used. 2.00 g of ethyl 4-[3-({4-[(tert-butoxycarbonyl)amino]-1-methylpyrrol-2-yl}formamido)propanamido]-1-methylimidazole-2-carboxylate was obtained as a white solid (79.14% yield). LC/MS: mass calcd. For C₂₁H₃₀N₆O₆: 462.51, found: 463.20[M+H]⁺.

### Step 6: Synthesis of ethyl 4-[3-({4-[(1,2,3-benzotriazol-1-yloxy)amino]-1-methylpyrrol-2-yl}formamido)propanamido]-1-methylimidazole-2-carboxylate

The procedure was the same as methyl 4-[4-(3-aminopropanamido)-1-methylimidazole-2-amido]-1-methylpyrrole-2-carboxylate hydrochloride. 1.20 g of ethyl 4-[3-({4-[(tert-butoxycarbonyl)amino]-1-methylpyrrol-2-yl}formamido)propanamido]-1-methylimidazole-2-carboxylate was used. 0.95 g of ethyl 4-{3-[(4-amino-1-methylpyrrol-2-yl)formamido]propanamido}-1-methylimidazole-2-carboxylate was obtained as a yellow oil (95.97% yield). LC/MS: mass calcd. For C₆H₂₂N₆O₄: 362.17, found: 363.10[M+H]⁺.

### Step 7: Synthesis of ethyl 4-(3-{[4-(4-{4-[(tert-butoxycarbonyl)amino] butanamido}-1-methylimidazole-2-amido)-1-methylpyrrol-2-yl]formamido}propanamido)-1-methylimidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate. 250.00 mg of ethyl 4-{3-[(4-amino-1-methylpyrrol-2-yl) formamido]propanamido}-1-methylimidazole-2-carboxylate was used. 250.00 mg of ethyl 4-(3-{[4-(4-{4-[(tert-butoxycarbonyl)amino]butanamido}-1-methylimidazole-2-amido)-1-methylpyrrol-2-yl]formamido}propanamido)-1-methylimidazole-2-carboxylate was obtained as a yellow solid (46.10% yield). LC/MS: mass calcd. For C₃₀H₄₂N₁₀O₅: 670.32, found: 671.35[M+H]⁺.

### Step 8: Synthesis of ethyl 4-[3-({4-[4-(4-aminobutanamido)-1-methylimidazole-2-amido]-1-methylpyrrol-2-yl}formamido)propanamido]-1-methylimidazole-2-carboxylate

The procedure was the same as 3-amino-N-propylpropanamide. 230 mg of ethyl 4-(3-{[4-(4-{4-[(tert-butoxycarbonyl)amino] butanamido}-1-methylimidazole-2-amido)-1-methylpyrrol-2-yl]formamido}propanamido)-1-methylimidazole-2-carboxylate was used, 200.00 mg of ethyl 4-[3-({4-[4-(4-aminobutanamido)-1-methylimidazole-2-amido]-1-methylpyrrol-2-yl} formamido)propanamido]-1-methylimidazole-2-carboxylate was obtained as a white solid. (96.85% yield). LC/MS: mass calcd. For C₂₅H₃₄N₁₀O₆: 570.27, found: 571.30[M+H]⁺.

### Step 9: Synthesis of ethyl 1-methyl-4-[3-({1-methyl-4-[1-methyl-4-(4-{[1-methyl-4-(1-methyl-4-{1-methyl-4-[1-methyl-4-(1-methylimidazole-2-amido)pyrrole-2-amido]pyrrole-2-amido}imidazole-2-amido)pyrrol-2-yl]formamido}butanamido)imidazole-2-amido]pyrrol-2-yl}formamido)propanamido]imidazole-2-carboxylate

The procedure was the same as ethyl 1-methyl-4-[4-({1-methyl-4-[1-methyl-4-(3-{[1-methyl-4-(1-methylimidazole-2-amido)pyrrol-2-yl]formamido}propanamido) imidazole-2-amido]pyrrol-2-yl}formamido)butanamido]imidazole-2-carboxylate. 350.37 mg of ethyl 4-[3-({4-[4-(4-aminobutanamido)-1-methylimidazole-2-amido]-1-methylpyrrol-2-yl}formamido)propanamido]-1-methylimidazole-2-carboxylate was used, 200.00 mg of ethyl 1-methyl-4-[3-({1-methyl-4-[1-methyl-4-(4-{[1-methyl-4-(1-methyl-4-{1-methyl-4-[1-methyl-4-(1-methylimidazole-2-amido)pyrrole-2-amido]pyrrole-2-amido}imidazole-2-amido)pyrrol-2-yl]formamido}butanamido)imidazole-2-amido]pyrrol-2-yl}formamido)propanamido]imidazole-2-carboxylate was obtained as a yellow solid. (54.58% yield). LC/MS: mass calcd. For C₅₃H₆₁N₂₁O₁₁: 1167.49, found: 1168.60[M+H]⁺.

### Step 10: Synthesis of bis(1-methyl-4-[3-({1-methyl-4-[1-methyl-4-(4-{[1-methyl-4-(1-methyl-4-{1-methyl-4-[1-methyl-4-(1-methylimidazole-2-amido)pyrrole-2-amido]pyrrole-2-amido}imidazole-2-amido)pyrrol-2-yl]formamido}butanamido)imidazole-2-amido]pyrrol-2-yl}formamido)propanamido]imidazole-2-carboxylic acid)

To a stirred mixture of bis(ethyl 1-methyl-4-[3-({1-methyl-4-[1-methyl-4-(4-{[1-methyl-4-(1-methyl-4-{1-methyl-4-[1-methyl-4-(1-methylimidazole-2-amido)pyrrole-2-amido]pyrrole-2-amido}imidazole-2-amido)pyrrol-2-yl]formamido}butanamido)imidazole-2-amido]pyrrol-2-yl}formamido)propanamido]imidazole-2-carboxylate) (190.00 mg, 0.08 mmol, 1.00 equiv) and MeOH (10.00 mL) was added LiOH·H₂O (2.00 mL) in portions at room temperature for 2.0 h. The crude product was purified by Prep-HPLC with the following conditions (Column: XBridge Prep Phenyl OBD Column, 19*250 mm, 5 m; Mobile Phase A: Water (10.00 mmol/L NH₄HCO₃), Mobile Phase B: ACN; Flow rate: 25 mL/min; Gradient: 20% B to 32% B in 10 min, 32% B; Wave Length: 254 nm; RT1(min): 8.2; Number Of Runs: 5). 150.00 mg desired product was obtained as a white solid (80.89% yield). LC/MS: mass calcd. For C₅₃H₆₁N₂₁O₁₁: 1139.45, found: 1140.55[M+H]⁺.

### Example 46. Synthesis of 3-{[1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl]formamido)butanamido]imidazole-2-amido}pyrrol-2-yl]formamido]propanamido}imidazole-2-amido)pyrrol-2-yl]formamido}propanoic acid (Compound 118)

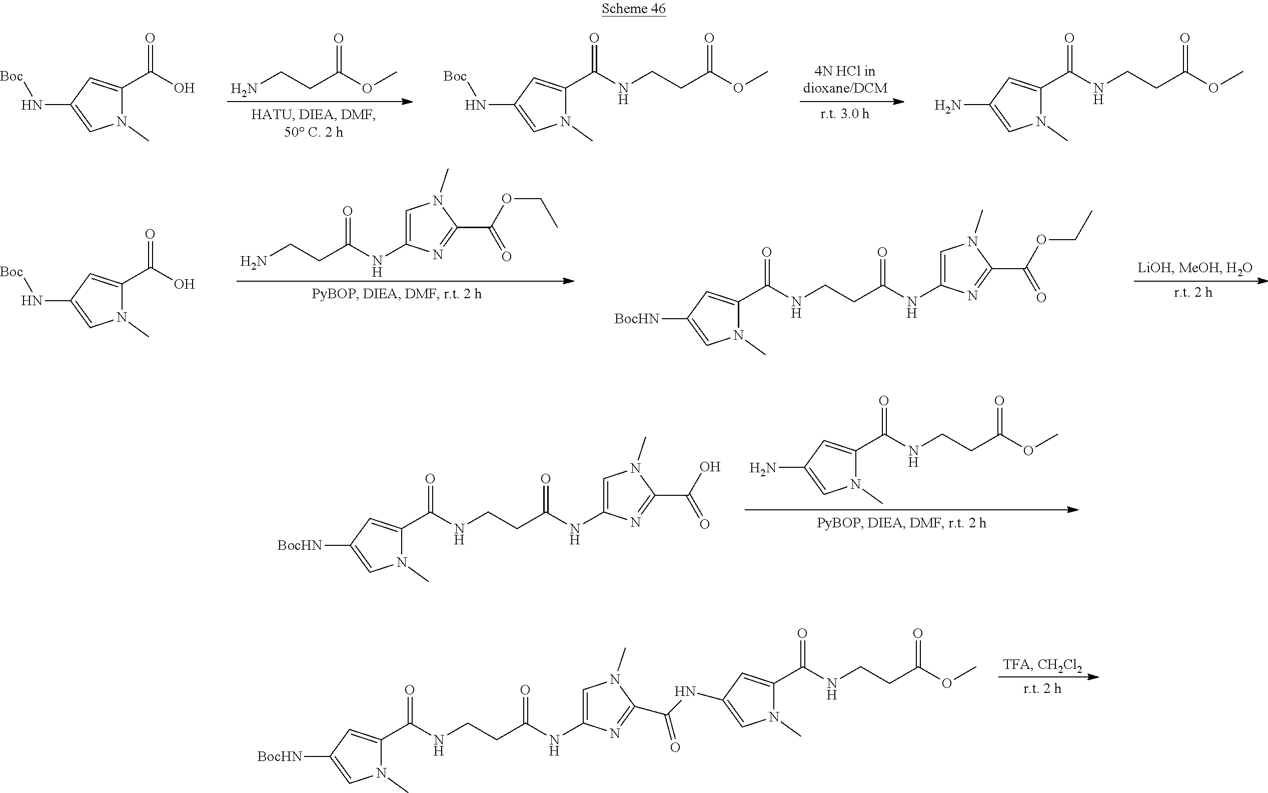

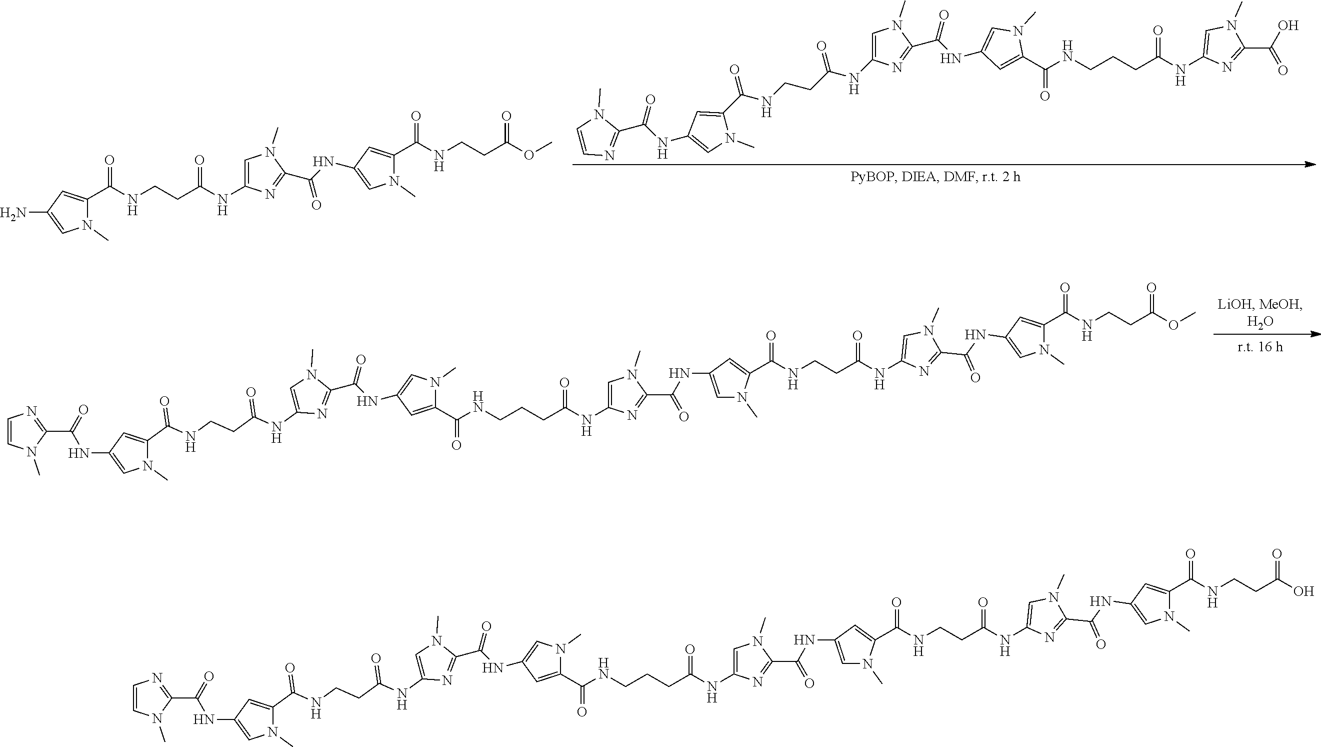

### Step 1: Synthesis of methyl 3-({4-[(tert-butoxycarbonyl)amino]-1-methylpyrrol-2-yl}formamido)propanoate

The procedure was the same as ethyl 3-{[4-(4-{13-[(tert-butoxycarbonyl) amino]propanamido}-1-methylpyrrole-2-amido)-1-methylimidazol-2-yl]formamido}propanoate, but the reaction temperature was 50° C., the reaction time was 2.0 h. 3.00 g of 4-[(tert-butoxycarbonyl)amino]-1-methylpyrrole-2-carboxylic acid was used, 3.40 g of methyl 3-({4-[(tert-butoxycarbonyl)amino]-1-methylpyrrol-2-yl}formamido)propanoate was obtained as a white solid (83.69% yield). LC/MS: mass calcd. For C₁₅H₂₃N₃O₅: 325.16, found: 326.20 [M+H]⁺.

### Step 2: Synthesis of methyl 3-(4-amino-1-methyl-1H-pyrrole-2-carboxamido) propanoate

The procedure was the same as 3-amino-N-propylpropanamide, but the reaction time was 3.0 h. 2.40 g of methyl 3-({4-[(tert-butoxycarbonyl)amino]-1-methylpyrrol-2-yl}formamido)propanoate was used, 1.60 g of methyl 3-(4-amino-1-methyl-1H-pyrrole-2-carboxamido)propanoate was obtained as yellow oil (96.30% yield). LC/MS: mass calcd. For C₁₀H₁₅N₃O₃: 225.11, found: 226.15 [M+H]⁺.

### Step 3: Synthesis of ethyl 4-[3-({4-[(tert-butoxycarbonyl)amino]-1-methylpyrrol-2-yl}formamido)propanamido]-1-methylimidazole-2-carboxylate

The procedure was the same as ethyl 4-(4-{3-[(tert-butoxycarbonyl)amino] propanamido}-1-methylpyrrole-2-amido)-1-methylimidazole-2-carboxylate, but the reaction time was 2.0 h. 2.40 g of 4-[(tert-butoxycarbonyl)amino]-1-methylpyrrole-2-carboxylic acid was obtained, 2.80 g of ethyl 4-[3-({4-[(tert-butoxycarbonyl)amino]-1-methylpyrrol-2-yl}formamido) propanamido]-1-methylimidazole-2-carboxylate was obtained as yellow oil (72.73% yield). LC/MS: mass calcd. For C₂₁H₃₀N₆O₆:

### Step 4: Synthesis of 4-[3-({4-[(tert-butoxycarbonyl)amino]-1-methylpyrrol-2-yl}formamido)propanamido]-1-methylimidazole-2-carboxylic acid

The procedure was the same as 4-[3-[(tert-butoxycarbonyl)amino]propanamido]-1-methylimidazole-2-carboxylic acid, but the reaction temperature was r.t. 2.80 g of ethyl 4-[3-({4-[(tert-butoxycarbonyl)amino]-1-methylpyrrol-2-yl}formamido)propanamido]-1-methylimidazole-2-carboxylate was used, 1.90 g of 4-[3-({4-[(tert-butoxycarbonyl)amino]-1-methylpyrrol-2-yl} formamido)propanamido]-1-methylimidazole-2-carboxylic acid was obtained as a yellow solid (72.24% yield). LC/MS: mass calcd. For C₁₉H₂₆N₆O₆: 434.19, found: 435.20 [M+H]⁺.

### Step 5: Synthesis of methyl 3-[(4-{4-[3-({4-[(tert-butoxycarbonyl)amino]-1-methylpyrrol-2-yl}formamido)propanamido]-1-methylimidazole-2-amido}-1-methylpyrrol-2-yl)formamido]propanoate

The procedure was the same as ethyl 4-(4-{3-[(tert-butoxycarbonyl)amino] propanamido}-1-methylpyrrole-2-amido)-1-methylimidazole-2-carboxylate, but the reaction time was 2.0 h. 1.00 g of 4-[3-({4-[(tert-butoxycarbonyl)amino]-1-methylpyrrol-2-yl}formamido)propanamido]-1-methylimidazole-2-carboxylic acid was used, 920.00 mg of methyl 3-[(4-{4-[3-({4-[(tert-butoxycarbonyl)amino]-1-methylpyrrol-2-yl}formamido)propanamido]-1-methylimidazole-2-amido}-1-methylpyrrol-2-yl)formamido]propanoate was obtained as a yellow solid (62.29% yield). LC/MS: mass calcd. For C₂₉H₃₉N₉O₈: 641.29, found: 642.30 [M+H]⁺.

### Step 6: Synthesis of methyl 3-{[4-(4-{3-[(4-amino-1-methylpyrrol-2-yl)formamido]propanamido}-1-methylimidazole-2-amido)-1-methylpyrrol-2-yl]formamido}propanoate

A mixture of methyl 3-[(4-{4-[3-({4-[(tert-butoxycarbonyl)amino]-1-methylpyrrol-2-yl}formamido)propanamido]-1-methylimidazole-2-amido}-1-methylpyrrol-2-yl)formamido]propanoate (850.00 mg, 1.33 mmol, 1.00 equiv) in CH₂Cl₂ (10.00 mL) was added TFA (2.00 mL). The reaction was stirred for 2.0 h at room temperature. The reaction mixture was concentrated under reduced pressure. The residue was purified by silica gel column chromatography, eluted with CH₂C₁₂/MeOH (10:1) to afford methyl 3-{[4-(4-{3-[(4-amino-1-methylpyrrol-2-yl)formamido] propanamido}-1-methylimidazole-2-amido)-1-methylpyrrol-2-yl]formamido}propanoate (520.00 mg, 72.49%) as a yellow solid. LC/MS: mass calcd. For C₂₄H₃₁N₉O₆: 541.24, found: 542.20 [M+H].

### Step 7: Synthesis of methyl 3-{[1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrol-2-yl]formamido}propanoate

The procedure was the same as methyl 4-(4-{3-[(4-{4-[(2R)-2-[(tert-butoxycarbonyl)amino]-4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]-1-methylimidazole-2-amido}-1-methylpyrrol-2-yl)formamido]propanamido}-1-methylimidazole-2-amido)-1-methylpyrrole-2-carboxylate, but the reaction time was 2.0 h. 300.00 mg of methyl 3-{[4-(4-{3-[(4-amino-1-methylpyrrol-2-yl)formamido]propanamido}-1-methylimidazole-2-amido)-1-methylpyrrol-2-yl]formamido}propanoate was used, 320.00 mg of methyl 3-{[1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrol-2-yl]formamido}propanoate was obtained as a yellow solid (44.56% yield). LC/MS: mass calcd. For C₅₈H₆₉N₂₃O₁₃: 1295.54, found: 1296.80 [M+H]⁺.

### Step 8: Synthesis of 3-{[1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrol-2-yl]formamido}propanoic acid

The procedure was the same as 4-[3-[(tert-butoxycarbonyl)amino]propanamido]-1-methylimidazole-2-carboxylic acid, but the reaction temperature was r.t., the reaction time was 16.0 h. 320.00 mg of methyl 3-{[1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrol-2-yl]formamido}propanoate was used, 170.00 mg of 3-{[1-methyl-4-(1-methyl-4-{3-[(1-methyl-4-{1-methyl-4-[4-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-amido}pyrrol-2-yl)formamido]propanamido}imidazole-2-amido)pyrrol-2-yl]formamido}propanoic acid was obtained as a yellow solid (53.71% yield). LC/MS: mass calcd. For C₅₇H₆₇N₂₃O₁₃: 1281.53, found: 1282.55[M+H]⁺.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A process for synthesizing a polyamide of Formula (I), or a pharmaceutically acceptable salt thereof, comprising reacting intermediate 24 with intermediate 34 to obtain a polyamide of Formula (I), the process comprising the steps: 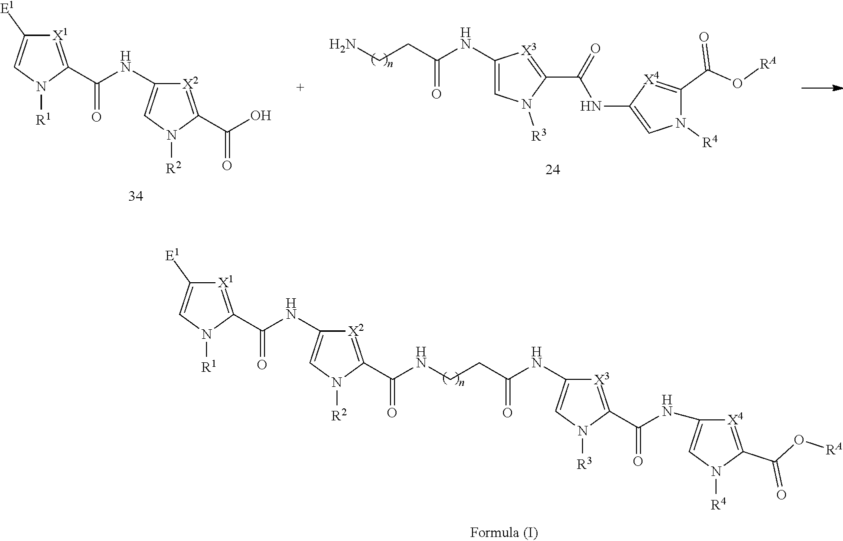
wherein,
n is 1, 2, or 3;
X¹, X², X³, and X⁴ are each independently CH or N;
E¹ is L-R^{B}; wherein
L is bond, —O—, —NH—, —C(O)—, —NHC(O)—, or —NHC(O)NH—;
R^{B} is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, or one or more AA; wherein each AA is independently a naturally occurring amino acid;
R^{A} is H or C₁-C₆ alkyl; and
R¹, R², R³, and R⁴ are each independently H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₁-C₂₀ hydroxyalkyl, optionally substituted C₁-C₂₀ haloalkyl, optionally substituted C₃-C₈ cycloalkyl, or optionally substituted 3 to 8-membered heterocycloalkyl.

2. The process of claim 1, wherein synthesizing intermediate 26 by the following process comprising the steps: 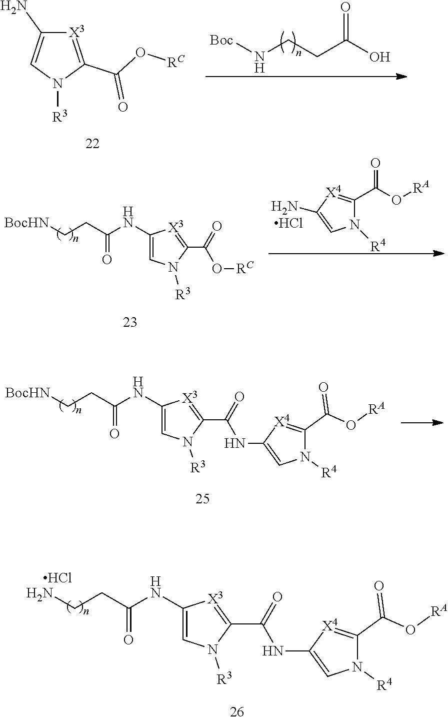
wherein, R^{C} is H or C₁-C₆ alkyl.

3. The process of claim 1, wherein synthesizing intermediate 34 by the following steps:

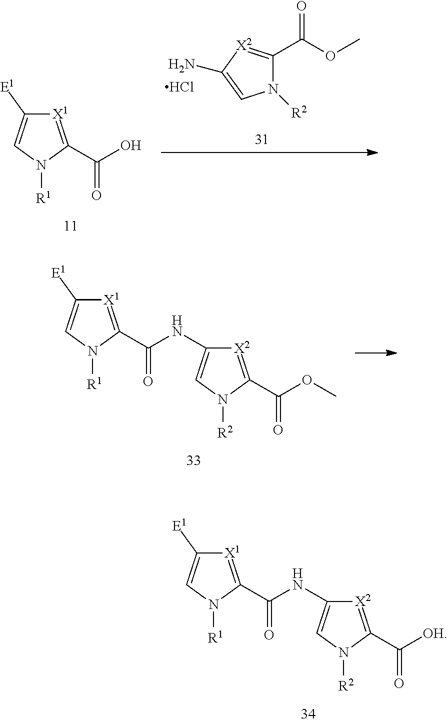

4. The process of any one of claims 1-3, optionally further comprising the following process to synthesize a polyamide of Formula (II), or a pharmaceutically acceptable salt thereof, the process comprising the steps: 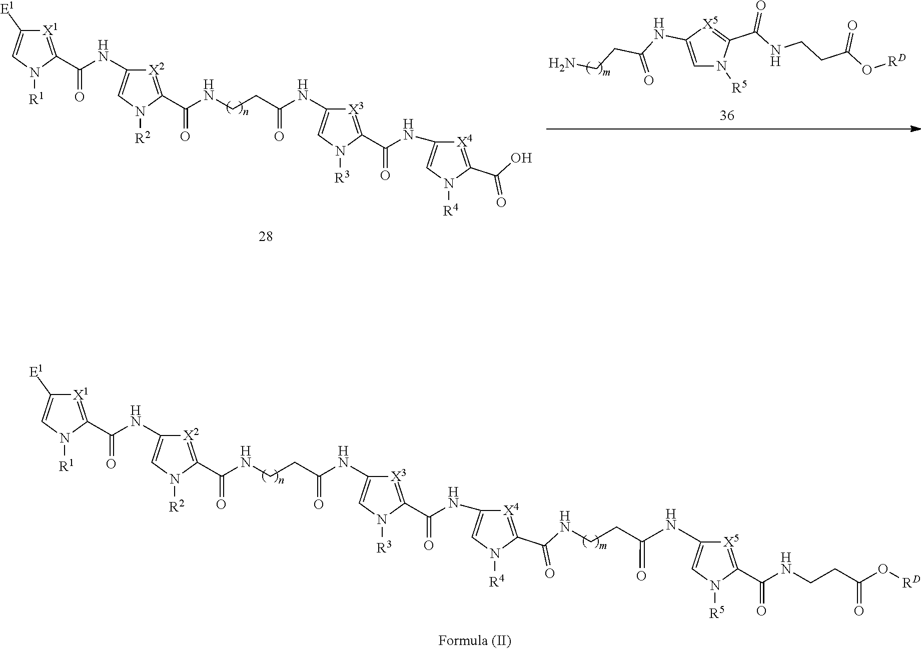
wherein,
X⁵ is CH or N;
R⁵ is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₁-C₂₀ hydroxyalkyl, optionally substituted C₁-C₂₀ haloalkyl, optionally substituted C₃-C₈ cycloalkyl, or optionally substituted 3 to 8-membered heterocycloalkyl;
R^{D} is H or C₁-C₆ alkyl; and
m is 1, 2, or 3.

5. The process of any one of claims 1-4, wherein:
X¹ and X³ are each N; and
X² and X⁴ are each CH.

6. The process of any one of claims 1-4, wherein X¹, X², X³ and X⁴ are each N.

7. The process of any one of claims 1-4, wherein X¹, X², X³ and X⁴ are each CH.

8. The process of any one of claims 1-7, wherein X⁵ is N.

9. The process of any one of claims 1-7, wherein X⁵ is CH.

10. The process of any one of claims 1-9, wherein R¹, R², R³, R⁴, and R⁵ are each independently H or an optionally substituted C₁-C₂₀ alkyl.

11. The process of claim 10, wherein R¹, R², R³, R⁴, and R⁵ are each independently methyl, ethyl, iso-propyl, iso-butyl, or tert-butyl.

12. The process of claim 11, wherein R¹, R², R³, R⁴, and R⁵ are each methyl.

13. The process of any one of claims 1-12, wherein n is 1 and m is 1.

14. The process of any one of claims 1-13, wherein L is bond and R^{B} is H.

15. The process of any one of claims 1-13, wherein L is —NHC(O)— and R^{B} is an optionally substituted C₁-C₂₀ alkyl or optionally substituted C₁-C₂₀ heteroalkyl.

16. The process of claim 15, wherein the heteroalkyl is a polyethylene glycol (PEG).

17. The process of any one of claims 1-16, wherein R^{A} is C₁-C₆ alkyl.

18. The process of claim 17, wherein R^{A} is methyl or ethyl.

19. The process of any one of claims 1-16, wherein R^{A} is H.

20. The process of any one of claims 4-19, wherein R^{D} is C₁-C₆ alkyl.

21. The process of claim 20, wherein R^{D} is methyl or ethyl.

22. The process of any one of claims 1-21, wherein the process comprises a solution phase synthesis.

23. A process for synthesizing a polyamide of Formula (II), or a pharmaceutically acceptable salt thereof, comprising reacting intermediate 28 with intermediate 36 to obtain a polyamide of Formula (II), the process comprising the steps: 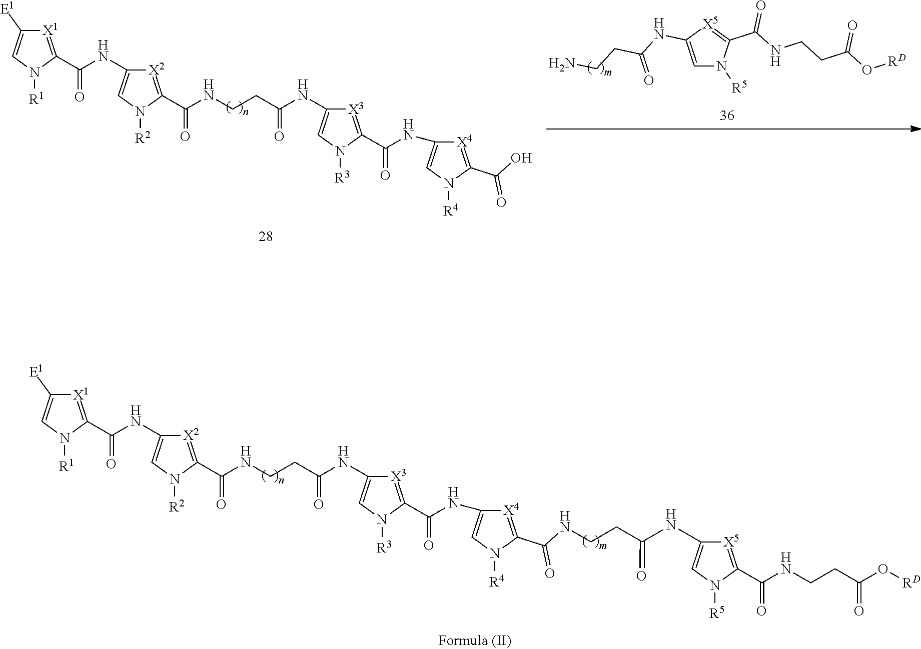
wherein,
n is 1, 2, or 3;
m is 1, 2, or 3;
X¹, X², X³, X⁴, and X⁵ are each independently CH or N;
E¹ is L-R^{B}, wherein
L is bond, —O—, —NH—, —C(O)—, —NHC (O)—, or —NHC (O) NH—;
R^{B} is H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, or one or more AA; wherein each AA is independently a naturally occurring amino acid;
R^{D} is H or C₁-C₆ alkyl; and
R¹, R², R³, R⁴, and R⁵ are each independently H or an optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₁-C₂₀ hydroxyalkyl, optionally substituted C₁-C₂₀ haloalkyl, optionally substituted C₃-C₈ cycloalkyl, or optionally substituted 3 to 8-membered heterocycloalkyl.

24. The process of claim 23, wherein:
X¹ and X³ are each N;
X² and X⁴ are each CH; and
X⁵ is N or CH.

25. The process of claim 23 or 24, wherein R¹, R², R³, R⁴, and R⁵ are independently H or an optionally substituted C₁-C₂₀ alkyl.

26. The process of claim 25, wherein R¹, R², R³, R⁴, and R⁵ are each independently methyl, ethyl, iso-propyl, iso-butyl, or tert-butyl.

27. The process of claim 26, wherein R¹, R², R³, R⁴, and R⁵ are each methyl.

28. The process of any one of claims 23-27, wherein L is bond and R^{B} is H.

29. The process of any one of claims 23-27, wherein L is —NHC(O)— and R^{B} is an optionally substituted C₁-C₂₀ alkyl or optionally substituted C₁-C₂₀ heteroalkyl.

30. The process of any one of claims 23-29, wherein m is 1 and n is 1.

31. The process of any one of claims 23-30, wherein R^{D} is C₁-C₆ alkyl.

32. The process of claim 31, wherein R^{D} is methyl or ethyl.

33. The process of any one of claims 4-32, wherein the compound of Formula (II), or a pharmaceutically acceptable salt thereof is selected from:

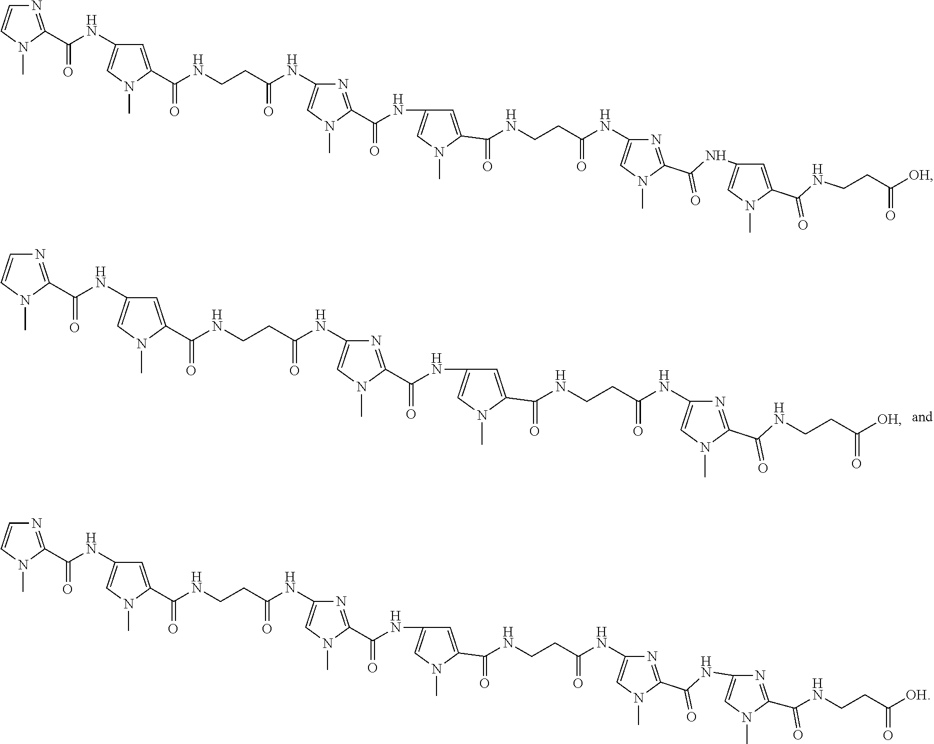 [SMILES: Cn1cc(NC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)cc1C(=O)NCCC(=O)O ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1 ; Cn1cc(NC(=O)c2nccn2C)cc1C(=O)NCCC(=O)Nc1cn(C)c(C(=O)Nc2cc(C(=O)NCCC(=O)Nc3cn(C)c(C(=O)Nc4cn(C)c(C(=O)NCCC(=O)O)n4)n3)n(C)c2)n1]

34. A polyamide, or a pharmaceutically acceptable salt thereof, selected from:

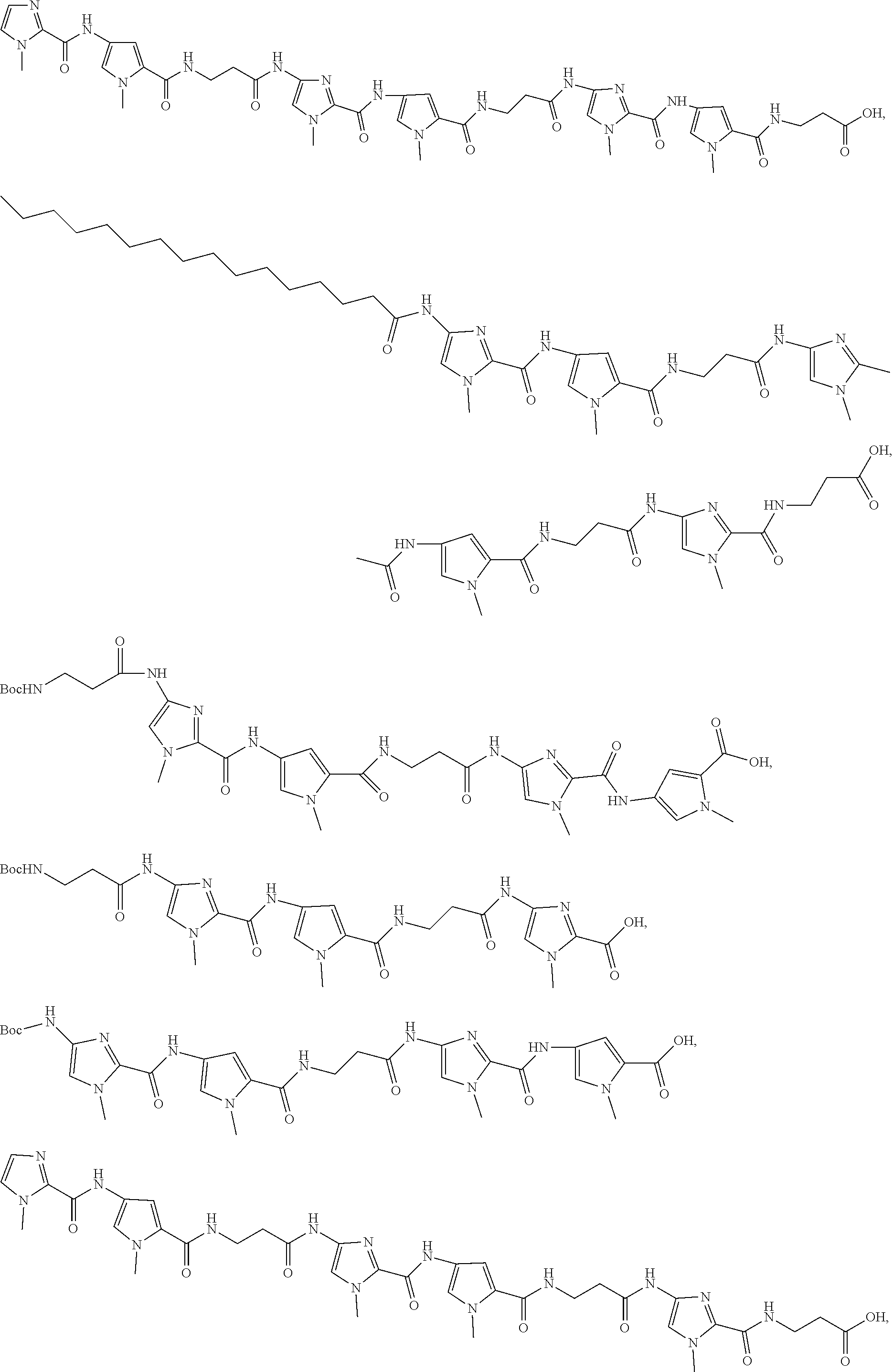

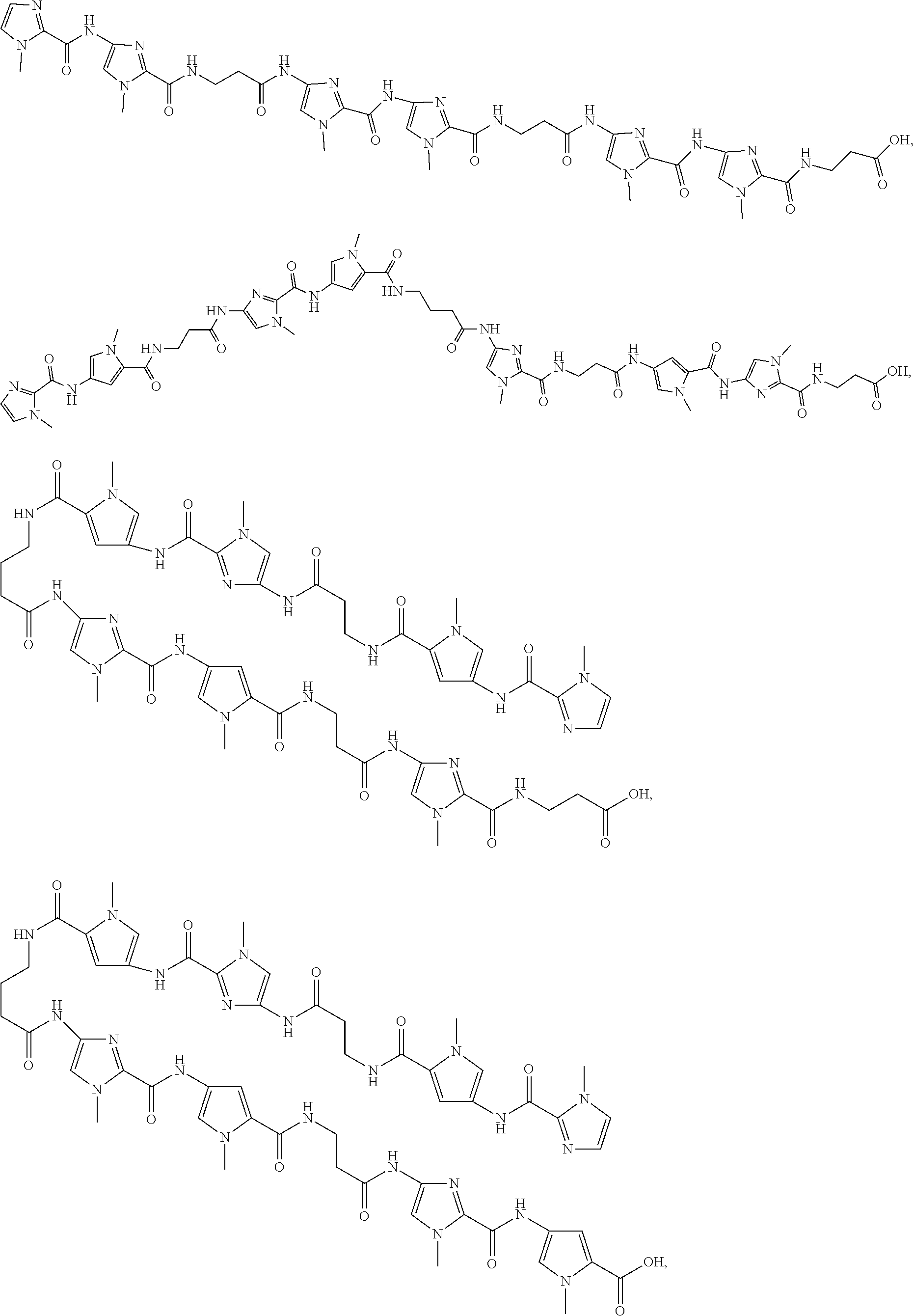

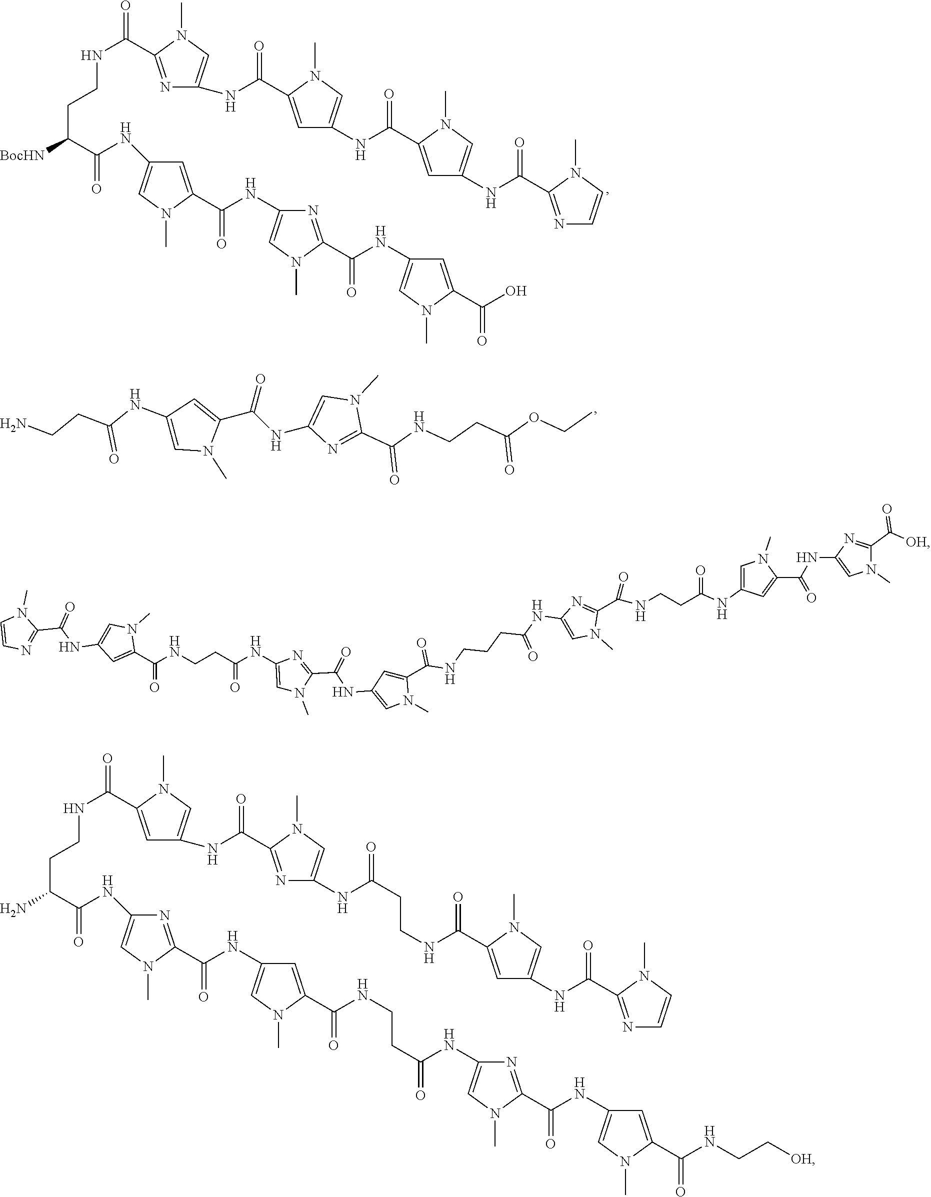

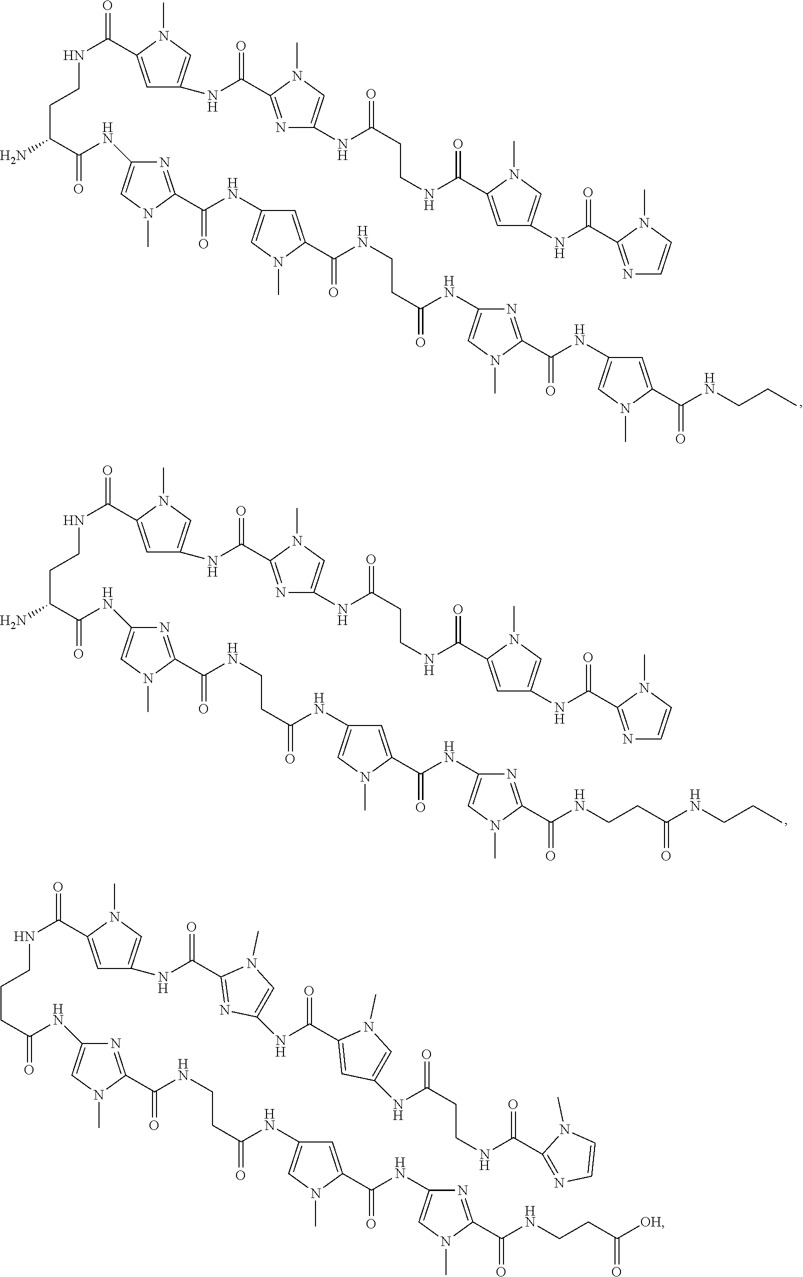 [SMILES: CCCNC(=O)c1cc(NC(=O)c2nc(NC(=O)CCNC(=O)c3cc(NC(=O)c4nc(NC(=O)[C@H](N)CCNC(=O)c5cc(NC(=O)c6nc(NC(=O)CCNC(=O)c7cc(NC(=O)c8nccn8C)cn7C)cn6C)cn5C)cn4C)cn3C)cn2C)cn1C ; CCCNC(=O)CCNC(=O)c1nc(NC(=O)c2cc(NC(=O)CCNC(=O)c3nc(NC(=O)[C@H](N)CCNC(=O)c4cc(NC(=O)c5nc(NC(=O)CCNC(=O)c6cc(NC(=O)c7nccn7C)cn6C)cn5C)cn4C)cn3C)cn2C)cn1C ; Cn1cc(NC(=O)c2nc(NC(=O)c3cc(NC(=O)CCNC(=O)c4nccn4C)cn3C)cn2C)cc1C(=O)NCCCC(=O)Nc1cn(C)c(C(=O)NCCC(=O)Nc2cc(C(=O)Nc3cn(C)c(C(=O)NCCC(=O)O)n3)n(C)c2)n1]

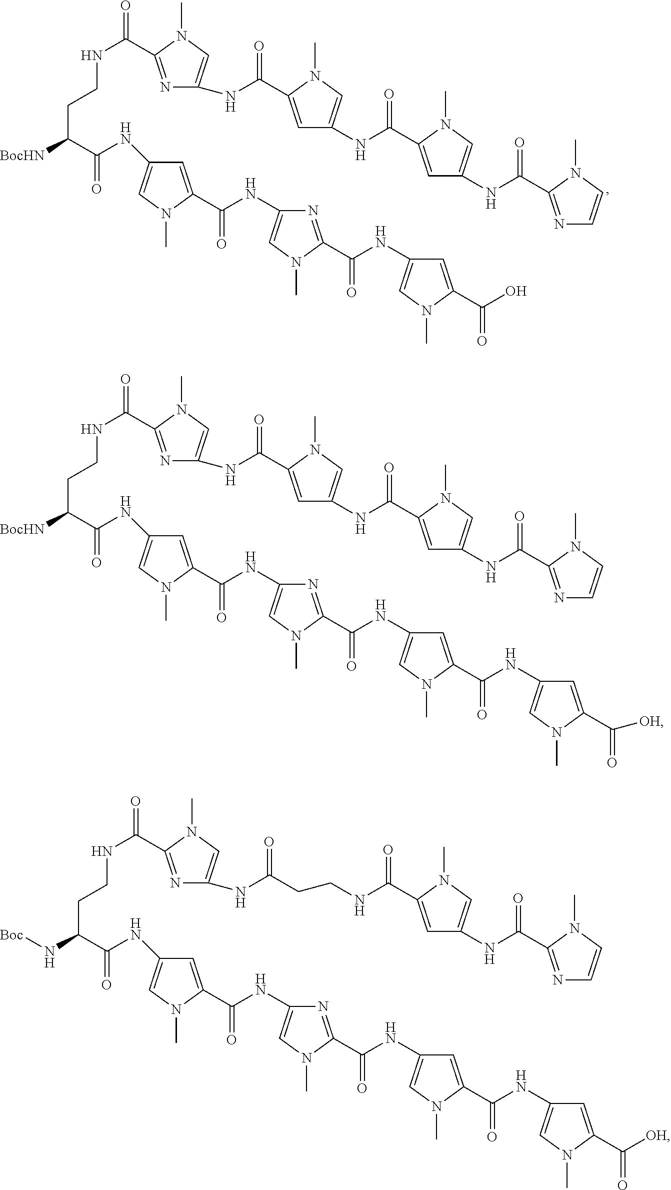 [SMILES: Cn1cc(NC(=O)c2nc(NC(=O)c3cc(NC(=O)[C@H](CCNC(=O)c4nc(NC(=O)c5cc(NC(=O)c6cc(NC(=O)c7nccn7C)cn6C)cn5C)cn4C)NC(=O)OC(C)(C)C)cn3C)cn2C)cc1C(=O)O ; Cn1cc(NC(=O)c2cc(NC(=O)c3nc(NC(=O)c4cc(NC(=O)[C@H](CCNC(=O)c5nc(NC(=O)c6cc(NC(=O)c7cc(NC(=O)c8nccn8C)cn7C)cn6C)cn5C)NC(=O)OC(C)(C)C)cn4C)cn3C)cn2C)cc1C(=O)O ; Cn1cc(NC(=O)c2cc(NC(=O)c3nc(NC(=O)c4cc(NC(=O)[C@H](CCNC(=O)c5nc(NC(=O)CCNC(=O)c6cc(NC(=O)c7nccn7C)cn6C)cn5C)NC(=O)OC(C)(C)C)cn4C)cn3C)cn2C)cc1C(=O)O]

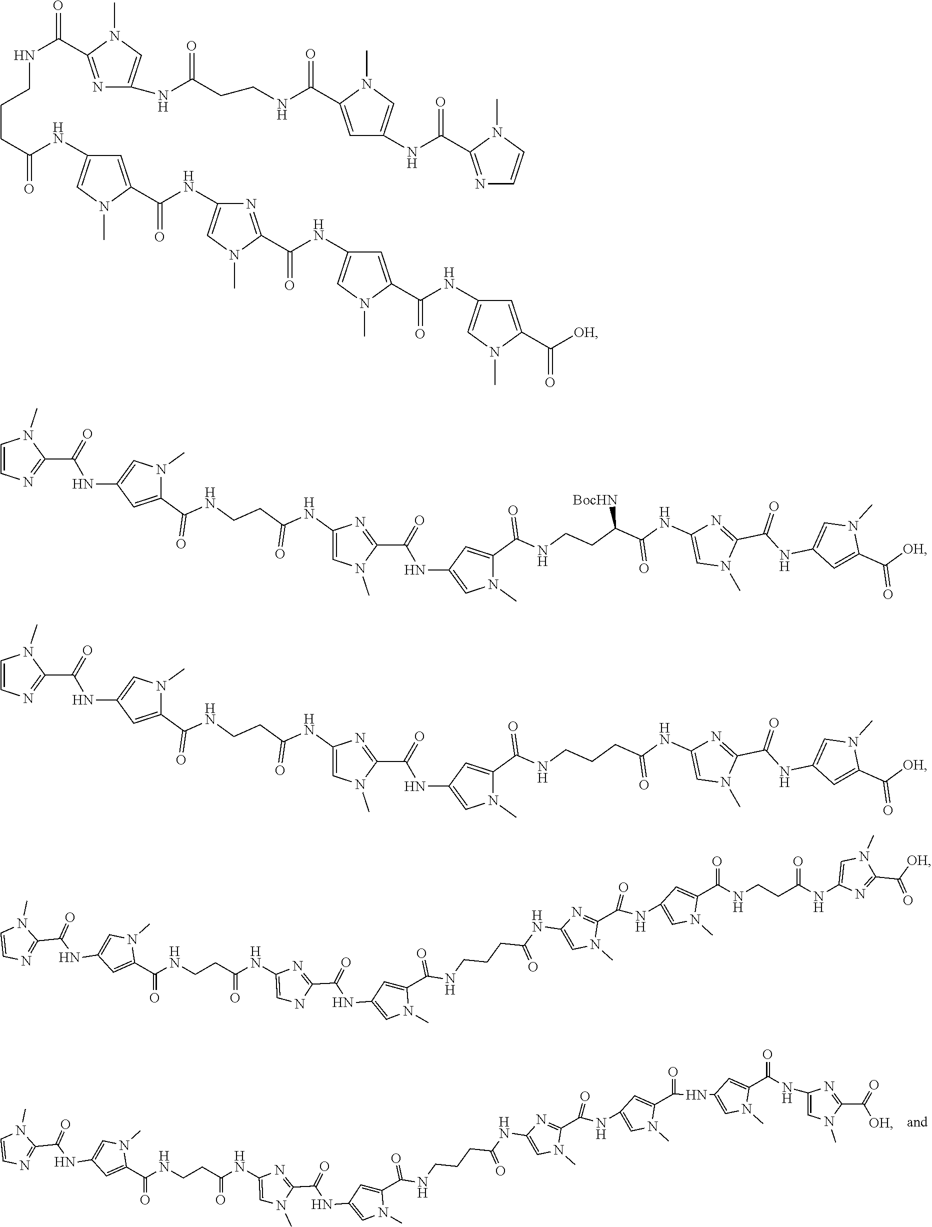

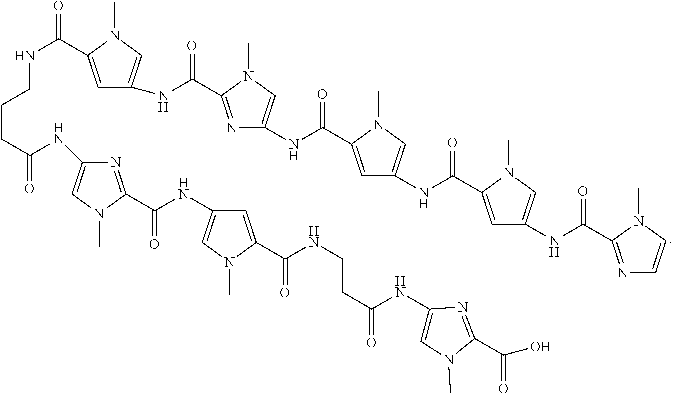 [SMILES: Cn1cc(NC(=O)c2nc(NC(=O)c3cc(NC(=O)c4cc(NC(=O)c5nccn5C)cn4C)cn3C)cn2C)cc1C(=O)NCCCC(=O)Nc1cn(C)c(C(=O)Nc2cc(C(=O)NCCC(=O)Nc3cn(C)c(C(=O)O)n3)n(C)c2)n1]

35. The polyamide of claim 34, wherein the polyamide or a pharmaceutically acceptable salt thereof is:

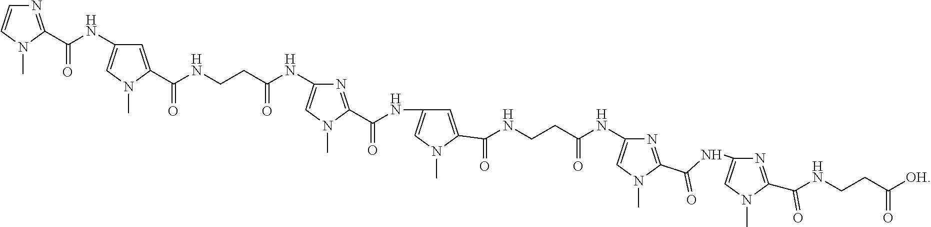 [SMILES: Cn1cc(NC(=O)c2nccn2C)cc1C(=O)NCCC(=O)Nc1cn(C)c(C(=O)Nc2cc(C(=O)NCCC(=O)Nc3cn(C)c(C(=O)Nc4cn(C)c(C(=O)NCCC(=O)O)n4)n3)n(C)c2)n1]

36. The polyamide of claim 34, wherein the polyamide or a pharmaceutically acceptable salt thereof is:

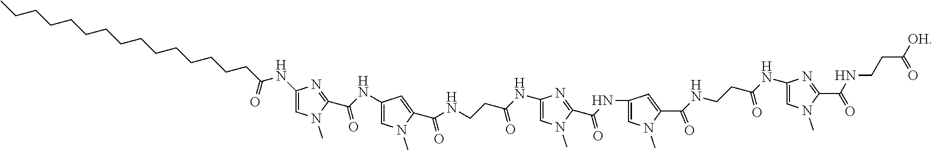 [SMILES: CCCCCCCCCCCCCCCC(=O)Nc1cn(C)c(C(=O)Nc2cc(C(=O)NCCC(=O)Nc3cn(C)c(C(=O)Nc4cc(C(=O)NCCC(=O)Nc5cn(C)c(C(=O)NCCC(=O)O)n5)n(C)c4)n3)n(C)c2)n1]

37. The polyamide of claim 34, wherein the polyamide or a pharmaceutically acceptable salt thereof is:

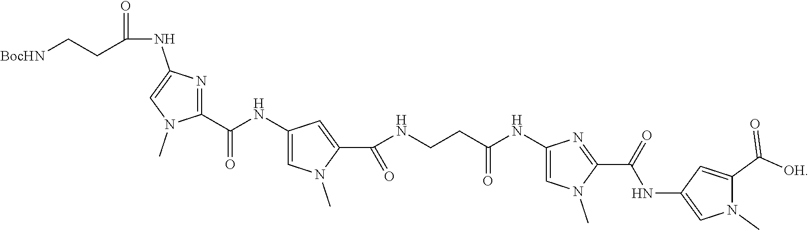 [SMILES: Cn1cc(NC(=O)c2nc(NC(=O)CCNC(=O)c3cc(NC(=O)c4nc(NC(=O)CCNC(=O)OC(C)(C)C)cn4C)cn3C)cn2C)cc1C(=O)O]

38. The polyamide of claim 34, wherein the polyamide or a pharmaceutically acceptable salt thereof is:

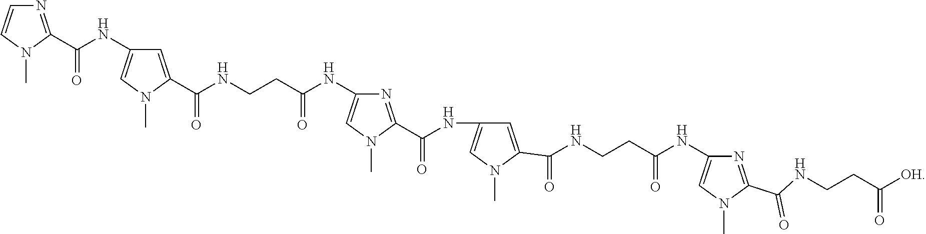 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1]

39. The polyamide of claim 34, wherein the polyamide or a pharmaceutically acceptable salt thereof is:

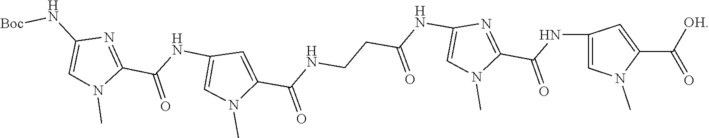 [SMILES: Cn1cc(NC(=O)c2nc(NC(=O)CCNC(=O)c3cc(NC(=O)c4nc(NC(=O)OC(C)(C)C)cn4C)cn3C)cn2C)cc1C(=O)O]

40. The polyamide of claim 34, wherein the polyamide or a pharmaceutically acceptable salt thereof is:

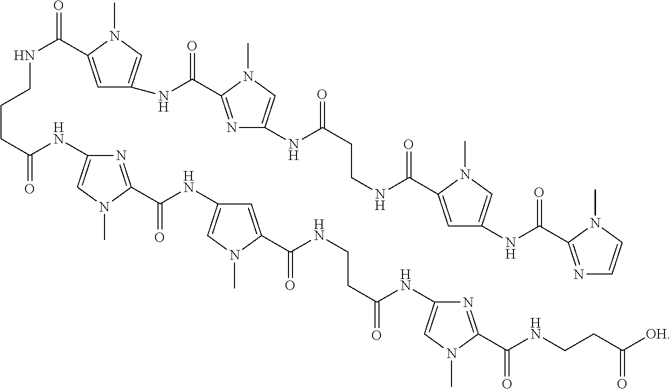 [SMILES: Cn1cc(NC(=O)c2nc(NC(=O)CCNC(=O)c3cc(NC(=O)c4nccn4C)cn3C)cn2C)cc1C(=O)NCCCC(=O)Nc1cn(C)c(C(=O)Nc2cc(C(=O)NCCC(=O)Nc3cn(C)c(C(=O)NCCC(=O)O)n3)n(C)c2)n1]

41. The polyamide of claim 34, wherein the polyamide or a pharmaceutically acceptable salt thereof is: 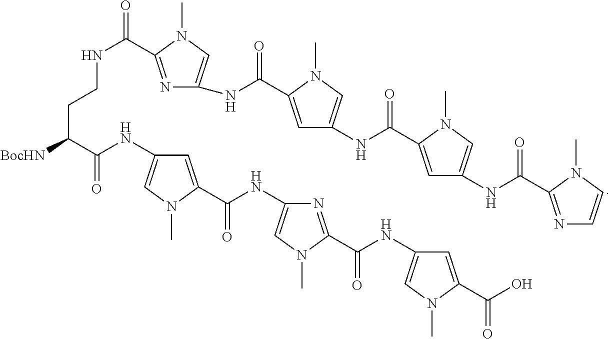 [SMILES: Cn1cc(NC(=O)c2nc(NC(=O)c3cc(NC(=O)[C@H](CCNC(=O)c4nc(NC(=O)c5cc(NC(=O)c6cc(NC(=O)c7nccn7C)cn6C)cn5C)cn4C)NC(=O)OC(C)(C)C)cn3C)cn2C)cc1C(=O)O]
